(19) United States
(12) Patent Application Publication
(10) Pub. No.: US 2025/0325534 A1
(43) Pub. Date: Oct. 23, 2025

(54) MICROBIOCIDAL TETRAHYDROISOQUINOLINE DERIVATIVES

(71) Applicant: SYNGENTA CROP PROTECTION AG, Basel (CH)

(72) Inventors: Christopher Charles SCARBOROUGH, Stein (CH); Andrew EDMUNDS, Stein (CH); Aurelien BIGOT, Stein (CH); Clemens LAMBERTH, Stein (CH); Daniel STIERLI, Stein (CH); Myriem EL QACEMI, Stein (CH); Atul MAHAJAN, Corlim Ilhas (IN); Nicola COMPAGNONE, Stein (CH); Alexandre Franco Jean Camille LUMBROSO, Stein (CH); Simon WILLIAMS, Stein (CH); Pierre Joseph Marcel JUNG, Stein (CH); Peter FINKBEINER, Stein (CH); Martin POULIOT, Stein (CH)

(73) Assignee: SYNGENTA CROP PROTECTION AG, Basel (CH)

(21) Appl. No.: 18/566,394

(22) PCT Filed: May 24, 2022
(86) PCT No.: PCT/EP2022/064094

§ 371 Date: Dec. 1, 2023

(30) Foreign Application Priority Data
Jun. 1, 2021 (IN) 202111024417
Oct. 13, 2021 (EP) 21202529.0

(51) Int. Cl. A61K 31/4725; A01P 3/00; C07D 401/14; C07D 413/14; C07D 417/14

(52) U.S. Cl. CPC A61K 31/4725; A01P 3/00; C07D 401/14; C07D 413/14; C07D 417/14

(57) ABSTRACT

A compound of formula (I) wherein the substituents are as defined in claim 1, and the agrochemically acceptable salts, stereoisomers, enantiomers, tautomers and N-oxides of those compounds, which can be used as fungicides.

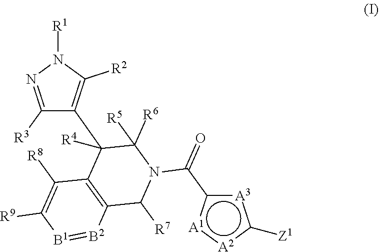

The present invention relates to microbiocidal tetrahydroisoquinoline derivatives, e.g. as active ingredients, which have microbiocidal activity, in particular fungicidal activity. The invention also relates to preparation of these tetrahydroisoquinoline derivatives, to intermediates useful in the preparation of these tetrahydroisoquinoline derivatives, to the preparation of these intermediates, to agrochemical compositions which comprise at least one of the dihydroisoquinoline derivatives, to preparation of these compositions and to the use of the tetrahydroisoquinoline derivatives or compositions in agriculture or horticulture for controlling or preventing infestation of plants, harvested food crops, seeds or non-living materials by phytopathogenic microorganisms, in particular fungi. According to a first aspect of the present invention, there is provided a use of a compound of formula (I) as a fungicide:

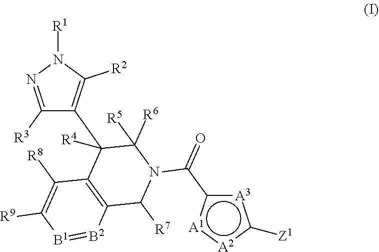

wherein
R¹ is selected from the group consisting of hydrogen, C₁-C₄ alkyl, C₂-C₄ alkenyl, C₂-C₄ alkynyl and C₃-C₆ cycloalkyl;
R² is selected from the group consisting of hydrogen, halogen, C₁-C₄ alkyl, C₂-C₄ alkenyl, C₂-C₄ alkynyl, C₁-C₄ haloalkyl, C₃-C₆ cycloalkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl and C₁-C₄ alkoxycarbonyl;
R³ and R⁴ are independently selected from the group consisting of hydrogen, halogen and C₁-C₄ alkyl;
R⁵ and R⁶ are independently selected from the group consisting of hydrogen and C₁-C₄ alkyl;
R⁷ is selected from the group consisting of hydrogen, C₁-C₄ alkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, C₁-C₄ alkoxycarbonyl, N-methoxy-N-methyl-carbonyl, C₁-C₄ alkylaminocarbonyl, di(C₁-C₄ alkylamino)carbonyl, phenyl, 5- or 6-membered heteroaryl and C₃-C₆ cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl or C₁-C₄ alkoxy;
B¹ is CR¹⁰ or N;
B² is CR¹¹ or N;
R⁸, R⁹, R¹⁰ and R¹¹ are independently selected from the group consisting of hydrogen, halogen, C₁-C₄ alkyl, C₁-C₄ haloalkyl, C₁-C₄ alkoxy, C₁-C₄ haloalkoxy, C₂-C₄ alkenyloxy, C₂-C₄ alkynyloxy, C₁-C₄ alkylsulfanyl, C₁-C₄ alkylsulfinyl, C₁-C₄ alkylsulfonyl, C₁-C₄ alkoxy-C₁-C₄ alkyl, C₁-C₄ alkoxycarbonyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, hydroxy, trifluoromethylsulfonyloxy, cyano, carboxy, phenyl, 5- or 6-membered heteroaryl and C₃-C₆ cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl or C₁-C₄ alkoxy;
A¹, A² and A³ are independently selected from the group consisting of CR¹², N, NR¹³, O and S, with the proviso that at least one of A¹, A² and A³ is selected from N, O and S, and that no more than one of A¹, A² and A³ is O or S;
R¹² is selected from the group consisting of hydrogen, C₁-C₄ alkyl, C₂-C₄ alkenyl and C₂-C₄ alkynyl;
R¹³ is selected from the group consisting of hydrogen, C₁-C₄ alkyl, C₂-C₄ alkenyl and C₂-C₄ alkynyl; and
Z¹ is selected from the group consisting of C₁-C₄ alkyl, phenyl, 5- or 6-membered heteroaryl and C₃-C₆-cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl, C₁-C₄ alkoxy, C₁-C₄ haloalkoxy, C₁-C₄ alkylsulfanyl, C₁-C₄ alkylsulfinyl, C₁-C₄ alkylsulfonyl or C₂-C₄ alkynyl;
or an agrochemically acceptable salt, stereoisomer, enantiomer, tautomer or N-oxide thereof.

According to this particular aspect of the invention, the use may exclude methods for the treatment of the human or animal body by surgery or therapy.

Surprisingly, it has been found that the compounds of formula (I) have, for practical purposes, a very advantageous level of biological activity for protecting plants against diseases that are caused by fungi.

According to a second aspect of the invention, there is provided a method of controlling or preventing infestation of useful plants by phytopathogenic microorganisms, wherein a fungicidally effective amount of a compound of formula (I) according to the invention, or a composition comprising the compound of formula (I), is applied to the plants, to parts thereof or the locus thereof.

As used herein, the term “halogen” or “halo” refers to fluorine (fluoro), chlorine (chloro), bromine (bromo) or iodine (iodo), preferably fluorine, chlorine or bromine.

As used herein, cyano means a —CN group.

As used herein, the term “hydroxyl” or “hydroxy” means an —OH group.

As used herein, oxo means an ═O group, e.g., sulfinyl (—S(O)—) or sulfonyl (—S(O)₂—) oxygen.

As used herein, the term “C₁-C₄ alkyl” refers to a straight or branched hydrocarbon chain radical consisting solely of carbon and hydrogen atoms, containing no unsaturation, having from one to four carbon atoms, and which is attached to the rest of the molecule by a single bond. The terms “C₁-C₃ alkyl”, “C₃-C₄ alkyl” and “C₁-C₂ alkyl” are to be construed accordingly. Examples of C₁-C₄ alkyl include, but are not limited to, methyl, ethyl, n-propyl, 1-methylethyl (isopropyl), n-butyl, and 1,1-dimethylethyl (t-butyl). A “C₁-C₄ alkylene” group refers to the corresponding definition of C₁-C₄ alkyl, except that such radical is attached to the rest of the molecule by two single bonds. Examples of C₁-C₄ alkylene, are —CH₂— and —CH₂CH₂—.

As used herein, the term “C₂-C₄ alkenyl” refers to a straight or branched hydrocarbon chain radical group consisting solely of carbon and hydrogen atoms, containing at least one double bond that can be of either the (E) or (Z) configuration, having from two to four carbon atoms, which is attached to the rest of the molecule by a single bond. The term “C₃-C₄ alkenyl” is to be construed accordingly. Examples of C₂-C₄ alkenyl include, but are not limited to, ethenyl and prop-1-enyl.

As used herein, the term “C₂-C₄ alkynyl” refers to a straight or branched hydrocarbon chain radical group consisting solely of carbon and hydrogen atoms, containing at least one triple bond, having from two to four carbon atoms, and which is attached to the rest of the molecule by a single bond. The term “C₃-C₄ alkynyl” is to be construed accordingly. Examples of C₃-C₄ alkynyl include, but are not limited to, ethynyl, prop-1-ynyl, propargyl (prop-2-ynyl), but-1-ynyl and 3-methyl-but-1-ynyl.

As used herein, the term “C₁-C₄ haloalkyl” refers to a C₁-C₄ alkyl radical as generally defined above substituted by one or more of the same or different halogen atoms. Examples of C₁-C₄ haloalkyl include, but are not limited to fluoromethyl, fluoroethyl, chloroethyl, difluoromethyl, dichloroethyl, trifluoromethyl, fluoropropyl, chloropropyl, difluoropropyl, dichloropropyl, trifluoropropyl, trichloropropyl, 2,2-difluoroethyl, 2,2-dichloroethyl, 2,2,2-trifluoroethyl, and 3,3,3-trifluoropropyl.

As used herein, the term “C₁-C₄ alkoxy” refers to a radical of the formula RₐO— where Rₐ is a C₁-C₄ alkyl radical as generally defined above. The terms “C₁-C₃ alkoxy” and “C₁-C₂ alkoxy” are to be construed accordingly. Examples of C₁-C₄ alkoxy include, but are not limited to, methoxy, ethoxy, propoxy, iso-propoxy, and t-butoxy.

As used herein, the term “C₁-C₄ alkoxy-C₁-C₄ alkyl” refers to radical of the formula R_{b}—O—Rₐ— where R_{b} is a C₁-C₄ alkyl radical as generally defined above, and Rₐ is a C₁-C₄ alkylene radical as generally defined above.

As used herein, the term “C₁-C₄ alkylcarbonyl” refers to a radical of the formula —C(O)Rₐ where Rₐ is a C₁-C₄ alkyl radical as generally defined above.

As used herein, the term “C₁-C₄ alkoxycarbonyl” refers to a radical of the formula —C(O)ORₐ where Rₐ is a C₁-C₄ alkyl radical as generally defined above.

As used herein, the term “C₁-C₄ alkylaminocarbonyl” refers to a radical of the formula —C(O)NHRₐ where Rₐ is a C₁-C₄ alkyl radical as generally defined above.

As used herein, the term “di(C₁-C₄ alkylamino)carbonyl” refers to a radical of the formula —C(O)NRₐ(Rₐ) where each Rₐ is a C₁-C₄ alkyl radical, which may be the same or different, as generally defined above.

As used herein, the term “C₂-C₄ alkenyloxy” refers to a radical of the formula —ORₐ where Rₐ is a C₂-C₄ alkenyl radical as generally defined above.

As used herein, the term “C₂-C₄ alkynyloxy” refers to a radical of the formula —ORₐ where Rₐ is a C₂-C₄ alkynyl radical as generally defined above.

As used herein, the term “C₃-C₆ cycloalkyl” refers to a stable, monocyclic ring radical which is saturated or partially unsaturated and contains 3 to 6 carbon atoms. The terms “C₃-C₄ cycloalkyl” and “C₃-C₅cycloalkyl” are to be construed accordingly. Examples of C₃-C₆ cycloalkyl include, but are not limited to, cyclopropyl, cyclobutyl, cyclopentyl, cyclohexyl, cyclopenten-1-yl, cyclopenten-3-yl, and cyclohexen-3-yl.

As used herein, the term “C₃-C₆ cycloalkylC₁-C₄ alkyl” refers to a C₃-C₆ cycloalkyl ring as defined above attached to the rest of the molecule by a C₁-C₄ alkylene radical as defined above. Examples of C₃-C₆cycloalkylC₁-C₄ alkyl include, but are not limited to, cyclopropyl-methyl, cyclobutyl-ethyl, and cyclopentyl-methyl.

As used herein, the term “N—C₁-C₄ alkoxy-C—C₁-C₄ alkyl-carbonimidoyl” refers to a radical of the formula —C(Rₐ)═NO(R_{b}) where Rₐ is a C₁-C₄ alkyl radical as generally defined above, and R_{b} is a C₁-C₄ alkyl radical as generally defined above.

As used herein the term “N-hydroxy-C—C₁-C₄ alkyl-carbonimidoyl” refers to a radical of the formula —C(Rₐ)═NOH where Rₐ is a C₁-C₄ alkyl radical as generally defined above.

Examples of a 5- or 6-membered heteroaryl ring, which comprise 1 to 4 heteroatoms independently selected from nitrogen, oxygen and sulfur, include, but are not limited to, pyridyl, pyrimidyl, pyrrolyl, pyrazolyl, furyl, thienyl, imidazolyl, isoxazolyl, oxazolyl, thiazolyl, isothiazolyl, triazolyl, oxadiazolyl, thiadiazolyl, tetrazolyl, pyrazinyl, pyridazinyl and triazinyl.

The compounds of formula (I) or the intermediate compounds of formula (III) or (IV) according to the invention, which have at least one basic centre, can form, for example, acid addition salts, for example with strong inorganic acids such as mineral acids, for example perchloric acid, sulfuric acid, nitric acid, a phosphorus acid or a hydrohalic acid, with strong organic carboxylic acids, such as C₁-C₄ alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄-alkane- or arylsulfonic acids which are unsubstituted or substituted, for example by halogen, for example methane- or p-toluenesulfonic acid.

The compounds of formula (I) or the intermediate compounds of formula (III) or (IV) according to the invention, which have at least one acidic group, can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

The presence of one or more possible asymmetric carbon atoms in a compound of formula (I) according to the invention means that the compounds may occur in chiral isomeric forms, i.e., enantiomeric or diastereomeric forms. Also, atropisomers may occur as a result of restricted rotation about a single bond. Formula (I) is intended to include all those possible isomeric forms and mixtures thereof. The present invention includes all those possible isomeric forms and mixtures thereof for a compound of formula (I) according to the invention. Likewise, a compound of formula (I) is intended to include all possible tautomers (including lactam-lactim tautomerism and keto-enol tautomerism) where present. The present invention includes all possible tautomeric forms for a compound of formula (I) according to the invention.

In each case, the compounds of formula (I) according to the invention are in free form, in oxidized form as an N-oxide, in covalently hydrated form, or in salt form, e.g., an agronomically usable or agrochemically acceptable salt form. N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991. The compounds of formula (I) according to the invention also include hydrates, which may be formed during salt formation.

The following list provides definitions, including preferred definitions, for substituents R¹, R², R³, R⁴, R⁵, R⁶, R⁷, R⁸, R⁹, R¹⁰, R¹¹, R¹², R¹³, A¹, A², A³, B¹, B² and Z¹ with reference to the compounds of formula (I) of the present invention. For any one of these substituents, any of the definitions given below may be combined with any definition of any other substituent given below or elsewhere in this document.

In an embodiment of the invention, R¹ is selected from the group consisting of hydrogen, C₁-C₄ alkyl and C₂-C₄ alkynyl. Preferably, R¹ is selected from the group consisting of hydrogen, methyl, ethyl and isopropyl. More preferably R¹ is selected from the group consisting of hydrogen, methyl and ethyl.

Even more preferably, R¹ is methyl.

In an embodiment of the invention, R² is selected from the group consisting of hydrogen, halogen, C₁-C₄ alkyl, C₃-C₆cycloalkyl, C₁-C₄ alkylcarbonyl, N—C₁-C₄ alkoxy-C—C₁-C₄ alkyl-carbonimidoyl and N-hydroxy-C—C₁-C₄ alkyl-carbonimidoyl. Preferably, R² is selected from the group consisting of hydrogen, halogen, methyl, ethyl, cyclopropyl, C₁-C₂ alkylcarbonyl, N—C₁-C₂ alkoxy-C—C₁-C₂ alkyl-carbonimidoyl and N-hydroxy-C—C₁-C₂ alkyl-carbonimidoyl. More preferably, R² is selected from the group consisting of hydrogen, bromine, fluorine, chlorine, methyl, ethyl, cyclopropyl, acetyl, —C(CH₃)═NOCH₃, —C(CH₃)═NOCH₂CH₃ and —C(CH₃)═NOH. Even more preferably, R² is selected from the group consisting of hydrogen, bromine, fluorine, chlorine, methyl, acetyl and —C(CH₃)═NOCH₃, Still even more preferably, R² is selected from the group consisting of hydrogen, fluorine, chlorine and methyl.

In an embodiment of the invention, R³ is selected from the group consisting of hydrogen, halogen and C₁-C₄ alkyl. Preferably, R³ is selected from the group consisting of hydrogen, fluorine, chlorine, methyl and ethyl. More preferably, R³ is selected from the group consisting of hydrogen and methyl.

In an embodiment of the invention, R⁴ is selected from the group consisting of hydrogen, halogen and C₁-C₄ alkyl. Preferably, R⁴ is selected from the group consisting of hydrogen, fluorine, chlorine, methyl, ethyl and isopropyl. More preferably R⁴ is selected from the group consisting of hydrogen, fluorine, chlorine, methyl and ethyl. Even more preferably, R⁴ is selected from the group consisting of hydrogen and methyl.

In an embodiment of the invention, R⁵ and R⁶ are independently selected from the group consisting of hydrogen, methyl and ethyl. Preferably R⁵ and R⁶ are independently selected from the group consisting of hydrogen and methyl.

In an embodiment of the invention, R⁷ is selected from the group consisting of hydrogen, methyl, ethyl, n-propyl, isopropyl, acetyl, —C(CH₃)═NOCH₃, —C(CH₃)═NOCH₂CH₃, —C(CH₃)═NOH, methylaminocarbonyl, dimethylaminocarbonyl, methoxycarbonyl, ethoxycarbonyl, N-methoxy-N-methyl-carbonyl, methylaminocarbonyl, dimethylaminocarbonyl, phenyl, 2-cyanophenyl, 3-cyanophenyl, 4-cyanophenyl, [4-(trifluoromethyl)pyrazol-1-yl], [3-(trifluoromethyl)pyrazol-1-yl], (3-cyanopyrazol-1-yl), (4-cyanopyrazol-1-yl), (5-chloropyrazol-1-yl), (5-fluoropyrazol-1-yl), (3,5-dimethylpyrazol-1-yl), (5-methylpyrazol-1-yl), pyrazol-1-yl, cyclopropyl and 1-cyanocyclopropyl. Preferably, R⁷ is selected from the group consisting of hydrogen, methyl, acetyl, —C(CH₃)═NOCH₃, —C(CH₃)═NOCH₂CH₃, —C(CH₃)═NOH, methoxycarbonyl, ethoxycarbonyl, N-methoxy-N-methyl-carbonyl, methylaminocarbonyl, dimethylaminocarbonyl, phenyl, 2-cyanophenyl, 3-cyanophenyl, 4-cyanophenyl, [4-(trifluoromethyl)pyrazol-1-yl], [3-(trifluoromethyl)pyrazol-1-yl], (3-cyanopyrazol-1-yl), (4-cyanopyrazol-1-yl), (5-chloropyrazol-1-yl), (5-fluoropyrazol-1-yl), (3,5-dimethylpyrazol-1-yl), (5-methylpyrazol-1-yl), pyrazol-1-yl, cyclopropyl and 1-cyanocyclopropyl. More preferably, R⁷ is selected from the group consisting of hydrogen, methyl, acetyl, —C(CH₃)═NOCH₃, —C(CH₃)═NOCH₂CH₃, —C(CH₃)═NOH, phenyl, 4-cyanophenyl, pyrazol-1-yl, cyclopropyl and 1-cyanocyclopropyl. Even more preferably, R⁷ is selected from the group consisting of hydrogen, methyl, cyclopropyl and 1-cyanocyclopropyl. Still more preferably, R⁷ is selected from the group consisting of hydrogen, methyl and cyclopropyl.

In another embodiment of the invention, R⁷ is selected from the group consisting of hydrogen, C₁-C₄ alkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, C₁-C₄ alkoxycarbonyl, N-methoxy-N-methyl-carbonyl, phenyl, 4-cyanophenyl, cyclopropyl and 1-cyanocyclopropyl. Preferably, R⁷ is selected from the group consisting of hydrogen, methyl, acetyl, —C(CH₃)═NOCH₃, —C(CH₃)═NOCH₂CH₃, —C(CH₃)═NOH, methoxycarbonyl, ethoxycarbonyl, N-methoxy-N-methyl-carbonyl, phenyl and cyclopropyl. More preferably, R⁷ is selected from the group consisting of hydrogen and methyl.

In another embodiment of the invention, R⁷ is selected from the group consisting of hydrogen, C₁-C₄ alkyl, C₁-C₄ alkylcarbonyl, C₁-C₄ alkoxycarbonyl, N—C₁-C₄ alkoxy-C—C₁-C₄ alkyl-carbonimidoyl and phenyl, Preferably R⁷ is selected from the group consisting of hydrogen, methyl, ethyl, propyl, isopropyl, acetyl, methoxycarbonyl, —C(CH₃)═NOCH₃ and phenyl. More preferably R⁷ is selected from the group consisting of hydrogen, methyl and acetyl.

In an embodiment of the invention, B¹ is CR¹⁰ and B² is CR¹¹ or B¹ is N and B² is CR¹¹ or B¹ is CR¹⁰ and B² is N. Preferably, B¹ is CR¹⁰ and B² is CR¹¹.

In an embodiment of the invention, R⁸ and R¹¹ are independently selected from the group consisting of hydrogen, halogen and C₁-C₄ alkyl. Preferably, R⁸ and R¹¹ are independently selected from the group consisting of hydrogen, chlorine, fluorine and methyl. More preferably, R⁸ and R¹¹ are hydrogen.

In another embodiment of the invention, R⁸ and R¹¹ are independently selected from the group consisting of hydrogen, halogen and C₁-C₄ alkyl. Preferably, R⁸ and R¹¹ are independently selected from the group consisting of hydrogen and halogen. More preferably, R⁸ and R¹¹ are independently selected from the group consisting of hydrogen, chlorine, bromine and fluorine.

In an embodiment of the invention, R⁹ and R¹⁰ are independently selected from the group consisting of hydrogen, halogen, C₁-C₃ alkyl, C₁-C₂ haloalkyl, C₁-C₃ haloalkoxy, C₁-C₄ alkoxy, C₂-C₃ alkenyloxy, C₂-C₃ alkynyloxy, C₁-C₂ alkylsulfanyl, C₁-C₂ alkylsulfinyl, C₁-C₂ alkylsulfonyl, C₁-C₂ alkoxy-C₁-C₂ alkyl, C₁-C₃ alkoxycarbonyl, C₁-C₂ alkylcarbonyl, N—C₁-C₂ alkoxy-C—C₁-C₂ alkyl-carbonimidoyl, N-hydroxy-C—C₁-C₂ alkyl-carbonimidoyl hydroxy, C₁-C₂ alkylaminocarbonyl, di(C₁-C₂ alkylamino)carbonyl, trifluoromethylsulfonyloxy, cyano, carboxy, phenyl, 2-cyanophenyl, 3-cyanophenyl, 4-cyanophenyl, 2-methylphenyl, 3-methylphenyl, 4-methylphenyl, [4-(trifluoromethyl)pyrazol-1-yl], [3-(trifluoromethyl)pyrazol-1-yl], (3-cyanopyrazol-1-yl), (4-cyanopyrazol-1-yl), (5-chloropyrazol-1-yl), (4-chloropyrazol-1-yl), (3-chloropyrazol-1-yl), (5-fluoropyrazol-1-yl), (4-fluoropyrazol-1-yl), (3-fluoropyrazol-1-yl), (3,5-dimethylpyrazol-1-yl), (5-methylpyrazol-1-yl), (4-methylpyrazol-1-yl), (3-methylpyrazol-1-yl), pyrazol-1-yl, cyclopropyl and 1-cyanocyclopropyl. Preferably, R⁹ and R¹⁰ are independently selected from the group consisting of hydrogen, chloro, fluoro, bromo, methyl, ethyl, difluoromethyl, trifluoromethyl, difluoromethoxy, trifluoromethoxy, 2,2-difluoroethoxy, 2,2,2-trifluoroethoxy, methoxy, ethoxy, propoxy, allyloxy, prop-2-ynoxy, methylsulfanyl, methylsulfinyl, methylsulfonyl, methoxymethyl, ethoxymethyl, 2-methoxyethoxymethyl, methoxycarbonyl, ethoxycarbonyl, tert-butoxycarbonyl, acetyl, propanoyl, —C(CH₃)═NOCH₃, —C(CH₃)═NOCH₂CH₃, —C(CH₃)═NOH, methylaminocarbonyl, di(methylamino)carbonyl, trifluoromethylsulfonyloxy, cyano, carboxy, phenyl, 2-cyanophenyl, 3-cyanophenyl, 4-cyanophenyl, 2-methylphenyl, 3-methylphenyl, 4-methylphenyl, [4-(trifluoromethyl)pyrazol-1-yl], [3-(trifluoromethyl)pyrazol-1-yl], (3-cyanopyrazol-1-yl), (4-cyanopyrazol-1-yl), (5-chloropyrazol-1-yl), (4-chloropyrazol-1-yl), (3-chloropyrazol-1-yl), (5-fluoropyrazol-1-yl), (4-fluoropyrazol-1-yl), (3-fluoropyrazol-1-yl), (3,5-dimethylpyrazol-1-yl), (5-methylpyrazol-1-yl), (4-methylpyrazol-1-yl), (3-methylpyrazol-1-yl), pyrazol-1-yl, cyclopropyl and 1-cyanocyclopropyl. More preferably, R⁹ and R¹⁰ are independently selected from the group consisting of hydrogen, chloro, fluoro, methyl, trifluoromethyl, difluoromethoxy, trifluoromethoxy, 2,2-difluoroethoxy, 2,2,2-trifluoroethoxy, methoxy, propoxy, allyloxy, prop-2-ynoxy, methylsulfanyl, methylsulfinyl, methylsulfonyl, methoxymethyl, 2-methoxyethoxymethyl, methoxycarbonyl, acetyl, propanoyl, —C(CH₃)═NOCH₃, —C(CH₃)═NOCH₂CH₃, —C(CH₃)═NOH, methylaminocarbonyl, di(methylamino)carbonyl, trifluoromethylsulfonyloxy, cyano, carboxy, phenyl, 2-cyanophenyl, 3-cyanophenyl, 4-cyanophenyl, [4-(trifluoromethyl)pyrazol-1-yl], [3-(trifluoromethyl)pyrazol-1-yl], (3-cyanopyrazol-1-yl), (4-cyanopyrazol-1-yl), (5-chloropyrazol-1-yl), (4-chloropyrazol-1-yl), (3-chloropyrazol-1-yl), (5-fluoropyrazol-1-yl), (4-fluoropyrazol-1-yl), (3-fluoropyrazol-1-yl), (3,5-dimethylpyrazol-1-yl), (5-methylpyrazol-1-yl), (4-methylpyrazol-1-yl), (3-methylpyrazol-1-yl), pyrazol-1-yl, cyclopropyl and 1-cyanocyclopropyl.

In another embodiment of the invention, R9 and R¹⁰ are independently selected from the group consisting of hydrogen, halogen, hydroxy, C₁-C₃ alkyl, C₁-C₂ haloalkyl, C₁-C₃ haloalkoxy, C₁-C₄ alkoxy, C₁-C₃ alkoxycarbonyl, cyano and phenyl. Preferably R⁹ and R¹⁰ are independently selected from the group consisting of hydrogen, bromine, chlorine, fluorine, hydroxy, methyl, trifluoromethyl, difluoromethoxy, trifluoromethoxy, 2,2-difluoroethoxy, 2,2,2-trifluoroethoxy, methoxy, cyano and phenyl. More preferably R⁹ and R¹⁰ are independently selected from the group consisting of hydrogen, bromine, chlorine, fluorine, hydroxy, methyl, trifluoromethyl, difluoromethoxy, 2,2-difluoroethoxy, 2,2,2-trifluoroethoxy, methoxy, cyano and phenyl. In an embodiment of the invention, A¹ and A² are independently selected from the group consisting of CR¹², N and O and A³ is CR¹², N, O or S, preferably A¹ and A² are independently selected from the group consisting of N and O and A³ is CR¹², O or S, with the proviso that at least one of A¹, A² and A³ is N or O and that no more than one of A¹, A² and A³ is O.

In an embodiment of the invention, R¹² is hydrogen or C₁-C₄ alkyl, preferably hydrogen or methyl. In an embodiment of the invention, R¹³ is hydrogen or C₁-C₄ alkyl, preferably hydrogen or methyl. In an embodiment of the invention, Z¹ is selected from the group consisting of 1-methylpyrazol-4-yl, 2,3,4-trifluorophenyl, 2,3-difluorophenyl, 3,4-difluorophenyl, 2,4,6-trifluorophenyl, 2,4-difluorophenyl, 2,5-difluorophenyl, 2-fluoro-4-methoxy-phenyl, 2-fluoro-4-methylsulfonyl-phenyl, 2-fluorophenyl, 3-fluorophenyl, 4-fluorophenyl, 2-furyl, 2-thienyl, 3-thienyl, 2-methylphenyl, 3-methylphenyl, 4-methylphenyl, 2-chlorophenyl, 3-chlorophenyl, 4-chlorophenyl, 3-methoxyphenyl, 4-ethynyl-2-fluoro-phenyl, 4-fluoro-2-methoxy-phenyl, cyclopropyl, 1-methylcyclopropyl, cyclobutyl, cyclohexyl, cyclopentyl, methyl, n-propyl, and phenyl.

In another embodiment of the invention, Z¹ is selected from the group consisting of C₃-C₄ alkyl, phenyl, 5- or 6-membered heteroaryl and C₃-C₆-cycloalkyl, wherein the 5- or 6-membered heteroaryl comprises at least one heteroatom selected from N, O and S, and wherein any of said phenyl, 5- or 6-membered heteroaryl and C₃-C₆-cycloalkyl are optionally substituted by 1, 2 or 3 substituents independently selected from halogen, C₁-C₄ alkyl, C₁-C₄ haloalkyl, C₁-C₄ alkoxy, C₁-C₄ haloalkoxy, C₁-C₄ alkylsulfonyl and C₂-C₄ alkynyl. Preferably Z¹ is selected from the group consisting of n-propyl, isopropyl, n-butyl, sec-butyl, tert-butyl, phenyl, 2-chlorophenyl, 3-chlorophenyl, 4-chlorphenyl, 2-fluorophenyl, 3-fluorophenyl, 4-fluorophenyl, 2,3-difluorophenyl, 2,4-difluorophenyl, 3,4-difluorophenyl, 2-fluoro-4-chloro-phenyl, 2,3,4-trifluorophenyl, 2,4,6-trifluorophenyl, 2-fluoro-4-methoxy-phenyl, 2-fluoro-4-methylsulfonyl-phenyl, 4-fluoro-2-methoxy-phenyl, 4-ethynyl-2-fluoro-phenyl, 4-trifluoromethyl-phenyl, 2-furyl, 2-thienyl, 3-thienyl, m-tolyl, o-tolyl, p-tolyl, 4-ethylphenyl, 3-methoxyphenyl, 4-pyridyl, cyclobutyl, cyclohexyl, cyclopentyl, cyclopropyl, 1-methylcyclopropyl, 1,5-dimethylpyrazol-4-yl and 1-methylpyrrol-2-yl.

In one embodiment, in a compound of formula (I) according to the present invention, R¹ is hydrogen, C₁-C₄ alkyl, C₂-C₄ alkenyl, C₂-C₄ alkynyl or C₃-C₆ cycloalkyl; R² is hydrogen, halogen, C₁-C₄ alkyl, C₂-C₄ alkenyl, C₂-C₄ alkynyl, C₁-C₄ haloalkyl, C₃-C₆ cycloalkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl or C₁-C₄ alkoxycarbonyl;

R³ and R⁴ are independently from each other hydrogen, halogen or C₁-C₄ alkyl;

R⁵ and R⁶ are independently from each other hydrogen or C₁-C₄ alkyl;

R⁷ is hydrogen, C₁-C₄ alkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, C₁-C₄ alkoxycarbonyl, N-methoxy-N-methyl-carbonyl, C₁-C₄ alkylaminocarbonyl, di(C₁-C₄ alkylamino)carbonyl, phenyl, 5- or 6-membered heteroaryl or C₃-C₆ cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl and C₁-C₄ alkoxy;

B¹ is CR¹⁰ or N;

B² is CR¹¹ or N;

R⁸, R⁹, R¹⁰ and R¹¹ are independently from each other hydrogen, halogen, C₁-C₄ alkyl, C₁-C₄ haloalkyl, C₁-C₄ alkoxy, C₁-C₄ haloalkoxy, C₂-C₄ alkenyloxy, C₂-C₄ alkynyloxy, C₁-C₄ alkylsulfanyl, C₁-C₄ alkylsulfinyl, C₁-C₄ alkylsulfonyl, C₁-C₄ alkoxy-C₁-C₄ alkyl, C₁-C₄ alkoxycarbonyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, hydroxy, trifluoromethylsulfonyloxy, cyano, carboxy, phenyl, 5- or 6-membered heteroaryl or C₃-C₆ cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl and C₁-C₄ alkoxy;

A¹, A² and A³ are independently from each other CR¹², N, NR¹³, O and S, with the proviso that at least one of A¹, A² and A³ is selected from N, O and S, and that no more than one of A¹, A² and A³ is O or S.

R¹² is hydrogen, C₁-C₄ alkyl, C₂-C₄ alkenyl or C₂-C₄ alkynyl;

R¹³ is of hydrogen, C₁-C₄ alkyl, C₂-C₄ alkenyl or C₂-C₄ alkynyl; and

Z¹ is C₃-C₄ alkyl, phenyl, 5- or 6-membered heteroaryl or C₃-C₆-cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl, C₁-C₄ alkoxy, C₁-C₄ haloalkoxy, C₁-C₄ alkylsulfanyl, C₁-C₄ alkylsulfinyl, C₁-C₄ alkylsulfonyl and C₂-C₄ alkynyl.

In an embodiment of the invention, the compound of formula (I) may be a compound of formula (I-A):

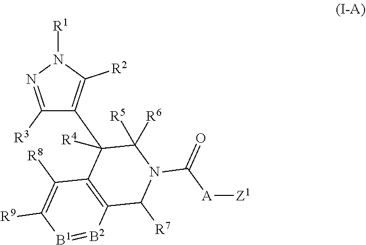

wherein
A is selected from the group consisting of:

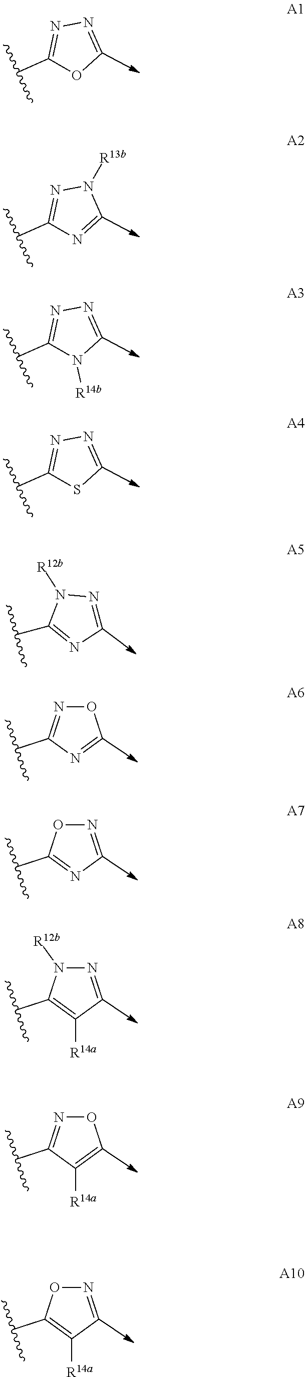

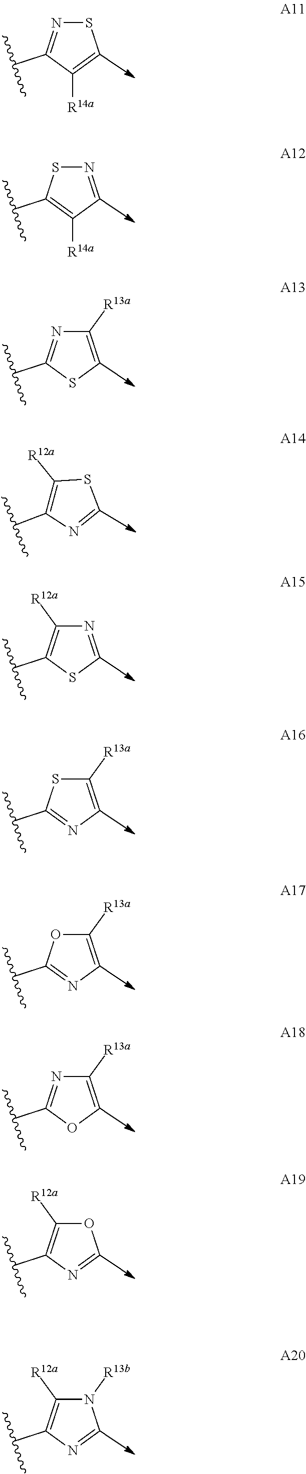

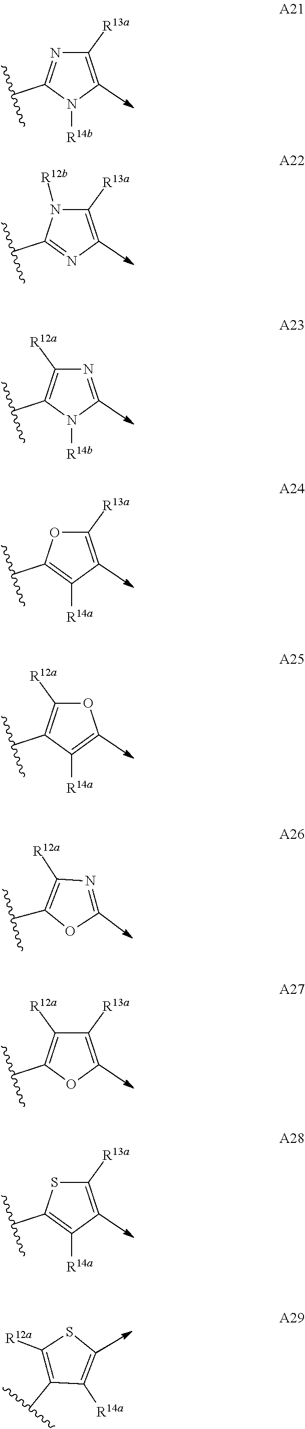

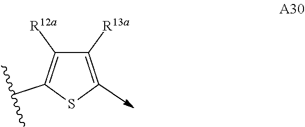

wherein

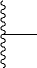

indicates the bond to the C(═O) group and the arrow the bond to the Z¹ group,
R¹, R², R³, R⁴, R⁵, R⁶, R⁷, R⁸, R⁹, R¹⁰, R¹¹, B¹, B² and Z¹ are as defined for the compounds of formula (I) according to the present invention, and
R^{12a}, R^{13a}, R^{14a}, R^{12b}, R^{13b} and R^{14b} are independently selected from the group consisting of hydrogen, C₁-C₄ alkyl, C₂-C₄ alkenyl and C₂-C₄ alkynyl.

In an embodiment of the invention, in the compound of formula (I-A) A is selected from the group consisting of:

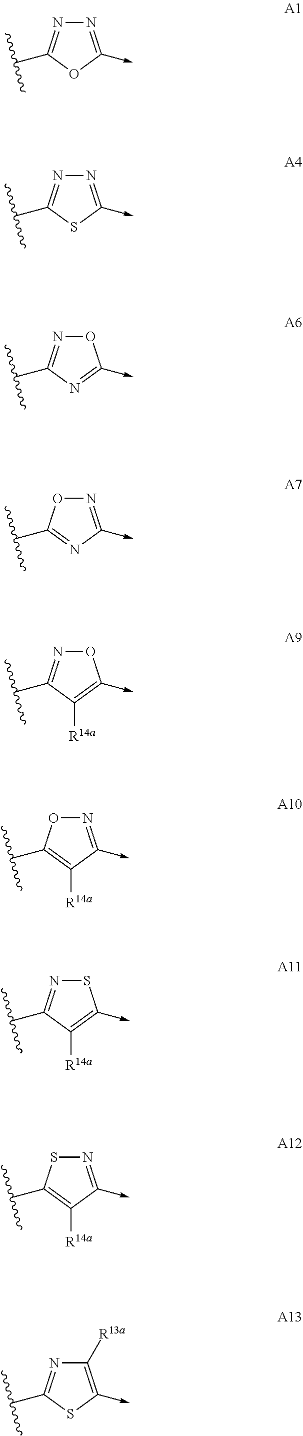

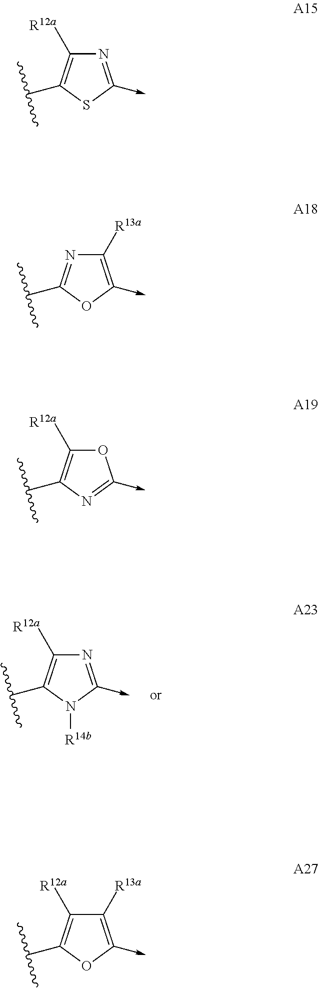

wherein

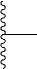

indicates the bond to the C(═O) group and the arrow the bond to the Z¹ group, and R^{12a}, R^{13a} and R^{14a} are independently from each other hydrogen or C₁-C₄ alkyl.

In another embodiment of the invention, in the compound of formula (I-A) A is selected from the group consisting of:

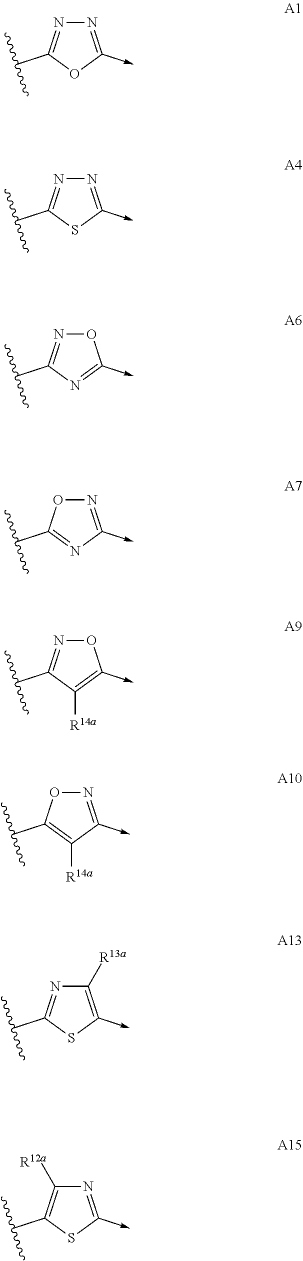

wherein

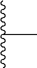

indicates the bond to the C(═O) group and the arrow the bond to the Z¹ group, and
R^{12a}, R^{13a} and R^{14a} are independently selected from the group consisting of hydrogen, C₁-C₄ alkyl, C₂-C₄ alkenyl and C₂-C₄ alkynyl.

In another embodiment of the invention, in the compound of formula (I-A) A is selected from the group consisting of:

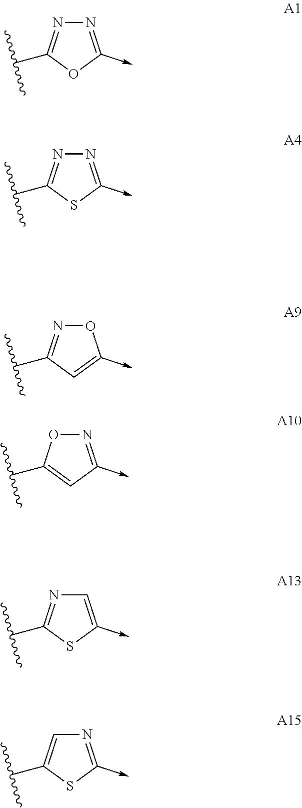

wherein

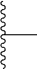

indicates the bond to the C(═O) group and the arrow the bond to the Z¹ group.

In another embodiment of the invention, in the compound of formula (I-A) A is selected from the group consisting of

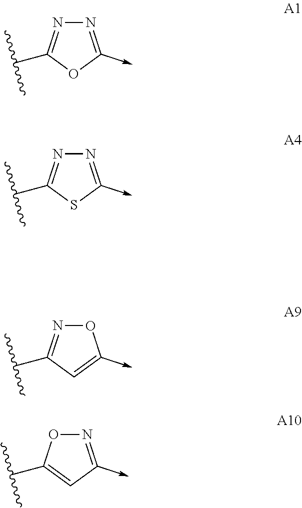

wherein

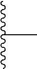

indicates the bond to the C(═O) group and the arrow the bond to the Z¹ group.

In another embodiment of the invention, in the compound of formula (I-A) A is selected from the group consisting of

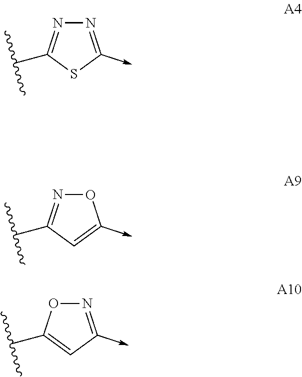

wherein

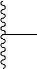

indicates the bond to the C(═O) group and the arrow the bond to the Z¹ group.

In an embodiment of the invention, R^{12a}, R^{13a}, R^{14a}, R^{12b}, R^{13b} and R^{14b} are independently selected from the group consisting of hydrogen and methyl.

In another embodiment of the invention, R^{12a}, R^{13a}, R^{14a}, R^{12b}, R^{13b} and R^{14b} are hydrogen.

In another embodiment of the invention, R^{12a}, R^{13a}, R^{14a}, R^{12b}, R^{13b} and R^{14b} are methyl.

In an embodiment of the invention, the compound of formula (I-A) may be a compound of formula (I-A1) wherein B¹ and B² are CH; and A is defined as for compound (I-A):

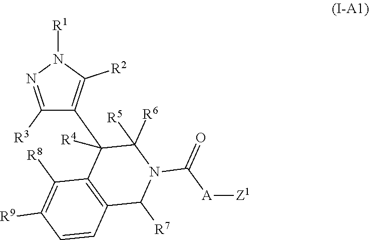

wherein R¹, R², R³, R⁴, R⁵, R⁶, R⁷, R⁸, R⁹ and Z¹ are as defined for the compounds of formula (I) according to the present invention.

In a variant of this embodiment of the invention, the compound of formula (I-A) may be a compound of formula (I-A2) wherein B¹ and B² are CH, R⁴, R⁵, R⁶ and R⁷ are hydrogen and A is defined as for compound (I-A):

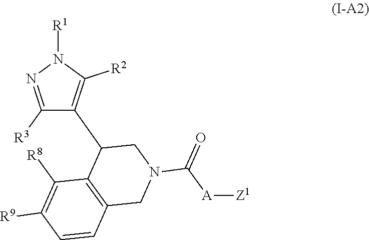

wherein R¹, R², R³, R⁸, R⁹ and Z¹ are as defined for the compounds of formula (I) according to the present invention.

Preferably, in the compound of formula (I-A2) of the invention,
R¹ is hydrogen, C₁-C₄ alkyl or C₂-C₄ alkynyl, preferably hydrogen, methyl or ethyl,
R² and R³ are independently from each other hydrogen, halogen such as bromine or chlorine, methyl, —C(O)OCH₂CH₃, N-methoxy-C-methyl-carbonimidoyl or —COCH₃,
R⁸ and R⁹ are independently from each other hydrogen, halogen such as fluorine or chlorine, cyano, methyl, trifluoromethyl, methoxy, —C(O)OCH₃ or —SO₂CH₃,
A as defined for the compounds of formula (I-A) and
Z¹ are as defined for the compounds of formula (I) according to the present invention.

In another variant of this embodiment of the invention, the compound of formula (I-A) may be a compound of formula (I-A3) wherein B¹ and B² are CH, R⁴, R⁵ and R⁶ are hydrogen and A is defined as for compound (I-A):

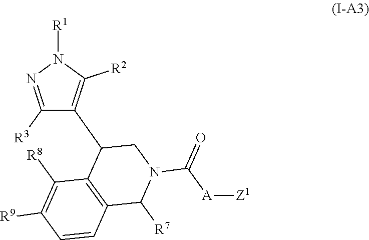

wherein
R¹, R², R³, R⁸, R⁹ and Z¹ are as defined for the compounds of formula (I) according to the present invention, and
R⁷ is C₁-C₄ alkyl.

Preferably, in the compound of formula (I-A3) of the invention,
R¹ is methyl,
R² and R³ are hydrogen or methyl,
R⁷ is methyl, ethyl or n-propyl or iso-propyl or phenyl or —C(O)OCH₃,
R⁸ and R⁹ are hydrogen or methoxy,
A is defined as for compound (i-A), and
Z¹ are as defined for the compounds of formula (I) according to the present invention.

In another variant of this embodiment of the invention, the compound of formula (I-A) may be a compound of formula (I-A4) wherein B¹ and B² are CH, R⁴, R⁵ and R⁷ are hydrogen, R⁶ is methyl and A is defined as for compound (I-A):

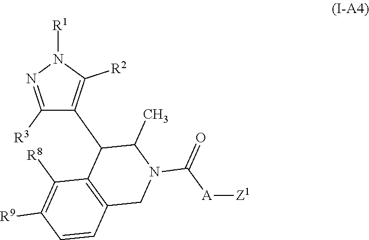

wherein R¹, R², R³, R⁸, R⁹ and Z¹ are as defined for the compounds of formula (I) according to the present invention.

Preferably, in the compound of formula (I-A4) of the invention,
R¹ is methyl,
R² and R³ are hydrogen or methyl,
R⁸ and R⁹ are hydrogen,
A is defined as for compound (I-A), and
Z¹ are as defined for the compounds of formula (I) according to the present invention.

In another variant of this embodiment of the invention, the compound of formula (I-A) may be a compound of formula (I-A5) wherein B¹ and B² are CH, R⁴ and R⁷ are hydrogen and R⁵ and R⁶ are methyl:

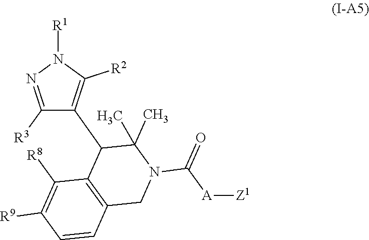

wherein R¹, R², R³, R⁸, R⁹ and Z¹ are as defined for the compounds of formula (I) according to the present invention, and
wherein A is defined as for compound (I-A) according to the present invention.

Preferably, in the compound of formula (I-A5) of the invention,
R¹ is methyl,
R² and R³ are hydrogen or methyl,
R⁸ and R⁹ are hydrogen,
A is defined as for compound (I-A), and
Z¹ are as defined for the compounds of formula (I) according to the present invention.

In another variant of this embodiment of the invention, the compound of formula (I-A) may be a compound of formula (I-A6) wherein B¹ and B² are CH and R⁵, R⁶ and R⁷ are hydrogen:

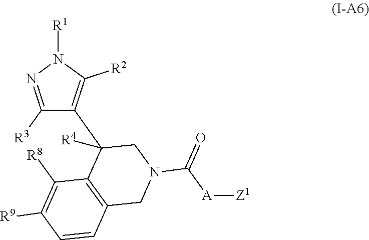

wherein
R¹, R², R³, R⁸, R⁹ and Z¹ are as defined for the compounds of formula (I) according to the present invention,
A is defined as for compound (I-A), and
R⁴ is C₁-C₄ alkyl.

Preferably, in the compound of formula (I-A6) of the invention,
R¹ is methyl,
R² and R³ are hydrogen or methyl,
R⁴ is methyl,
R⁸ and R⁹ are hydrogen,
A is defined as for compound (I-A), and
Z¹ are as defined for the compounds of formula (I) according to the present invention.

In another variant of this embodiment of the invention, the compound of formula (I-A) may be a compound of formula (I-A7) wherein B¹ and B² are CH and R⁵ and R⁶ are hydrogen:

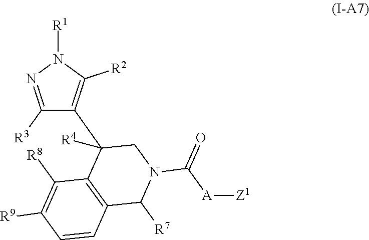

wherein
R¹, R², R³, R⁸, R⁹ and Z¹ are as defined for the compounds of formula (I) according to the present invention,
A is defined as for compound (I-A), and
R⁴ and R⁷ are C₁-C₄ alkyl.

Preferably, in the compound of formula (I-A7) of the invention,
R¹ is methyl,
R² and R³ are hydrogen, halogen such as fluorine, or methyl,
R⁴ and R⁷ are methyl,
R⁸ and R⁹ are hydrogen, hydroxy, methoxy, difluoromethoxy, 2,2-difluoroethoxy or 2,2,2-trifluoroethoxy,
A is defined as for compound (I-A), and
Z¹ are as defined for the compounds of formula (I) according to the present invention.

In another variant of this embodiment of the invention, the compound of formula (I-A) may be a compound of formula (I-A8) wherein B² is CH and R⁴, R⁵, R⁶, R⁷, R⁸ and R⁹ are hydrogen:

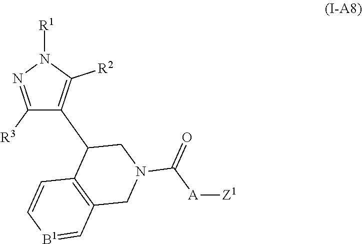

wherein
B¹ is CR¹⁰, and
R¹, R², R³, R¹⁰ and Z¹ are as defined for the compounds of formula (I) according to the present invention, and
A is defined as for compound (I-A).

Preferably, in the compound of formula (I-A8) of the invention,
R¹ is methyl,
R² and R³ are independently from each other hydrogen, halogen such as fluorine, or methyl,
R¹⁰ is halogen such as fluorine, methyl, trifluoromethyl, methoxy or phenyl,
A is defined as for compound (I-A), and
Z¹ are as defined for the compounds of formula (I) according to the present invention.

In another variant of this embodiment of the invention, the compound of formula (I-A) may be a compound of formula (I-A9) wherein B¹ is CH and R⁴, R⁵, R⁶, R⁷, R³ and R⁹ are hydrogen:

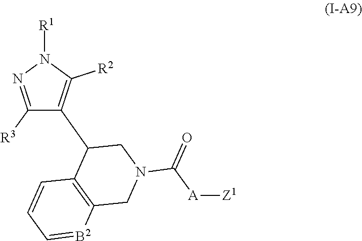

wherein
B² is CR¹¹, and
R¹, R², R³, R¹¹ and Z¹ are as defined for the compounds of formula (I) according to the present invention, and
A is defined as for compound (I-A).

Preferably, in the compound of formula (I-A9) of the invention,
R¹ is methyl,
R² and R³ are independently from each other hydrogen, halogen such as fluorine, or methyl,
R¹¹ is halogen such as fluorine, methyl, trifluoromethyl, methoxy or phenyl,
A is defined as for compound (I-A), and
Z¹ are as defined for the compounds of formula (I) according to the present invention.

In another variant of this embodiment of the invention, the compound of formula (I-A) may be a compound of formula (I-A10) wherein B³¹ is CH, B² is N and R⁴, R⁵, R⁶, R⁷, R³ and R⁹ are hydrogen:

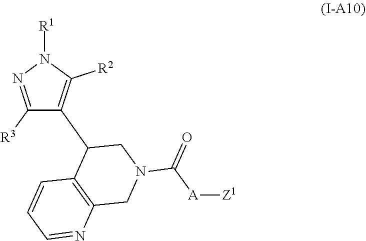

wherein R¹, R², R³ and Z¹ are as defined for the compounds of formula (I) according to the present invention, and
A is defined as for compound (I-A).

Preferably, in the compound of formula (I-A10) of the invention,
R¹ is methyl,
R² and R³ are independently from each other hydrogen, halogen such as fluorine, or methyl,
A is defined as for compound (I-A), and
Z¹ are as defined for the compounds of formula (I) according to the present invention.

The presence of one or more possible asymmetric carbon atoms in any of the compounds of formula (I), (I-A) and (I-A1) to (I-A10) according to the invention means that the compounds may occur in chiral isomeric forms, i.e., enantiomeric or diastereomeric forms.

More preferably, the compound of formula (I) according to the invention is selected from compounds listed in any one of Tables A-1 to A-32 or in Table T1.

According to a third aspect of the invention, there is provided a compound of formula (I) as defined in any one of the embodiments of the invention, with the proviso that the compound of formula (I) is not

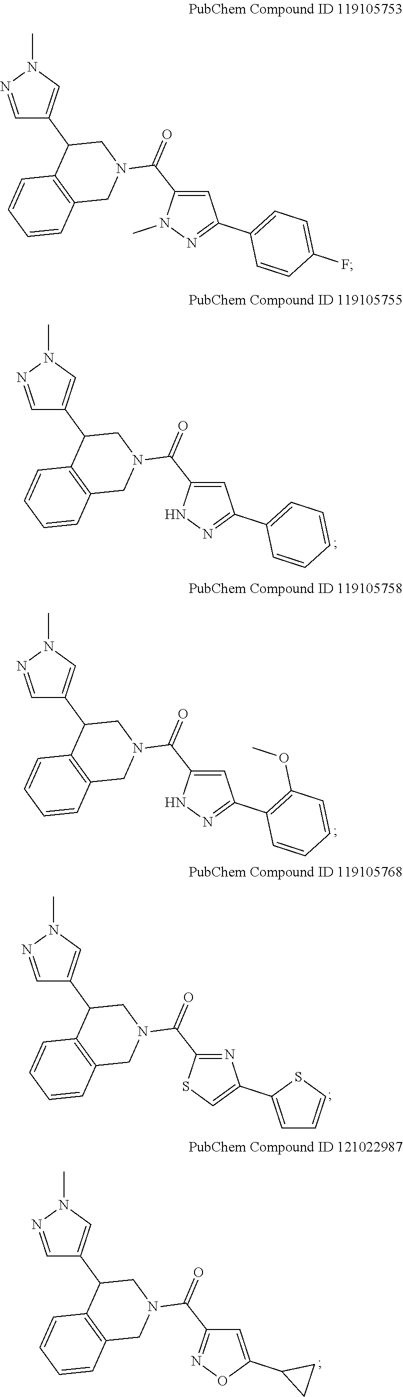 [SMILES: Cn1cc(C2CN(C(=O)c3cc(-c4ccc(F)cc4)nn3C)Cc3ccccc32)cn1 ; Cn1cc(C2CN(C(=O)c3cc(-c4ccccc4)n[nH]3)Cc3ccccc32)cn1 ; COc1ccccc1-c1cc(C(=O)N2Cc3ccccc3C(c3cnn(C)c3)C2)[nH]n1 ; Cn1cc(C2CN(C(=O)c3nc(-c4cccs4)cs3)Cc3ccccc32)cn1 ; Cn1cc(C2CN(C(=O)c3cc(C4CC4)on3)Cc3ccccc32)cn1]

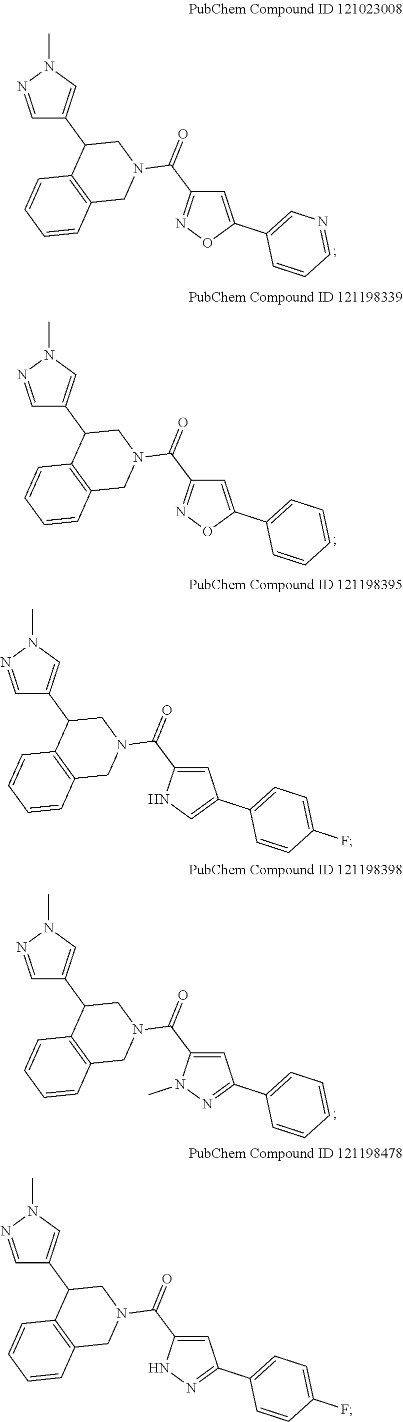 [SMILES: Cn1cc(C2CN(C(=O)c3cc(-c4cccnc4)on3)Cc3ccccc32)cn1 ; Cn1cc(C2CN(C(=O)c3cc(-c4ccccc4)on3)Cc3ccccc32)cn1 ; Cn1cc(C2CN(C(=O)c3cc(-c4ccc(F)cc4)c[nH]3)Cc3ccccc32)cn1 ; Cn1cc(C2CN(C(=O)c3cc(-c4ccccc4)nn3C)Cc3ccccc32)cn1 ; Cn1cc(C2CN(C(=O)c3cc(-c4ccc(F)cc4)n[nH]3)Cc3ccccc32)cn1]

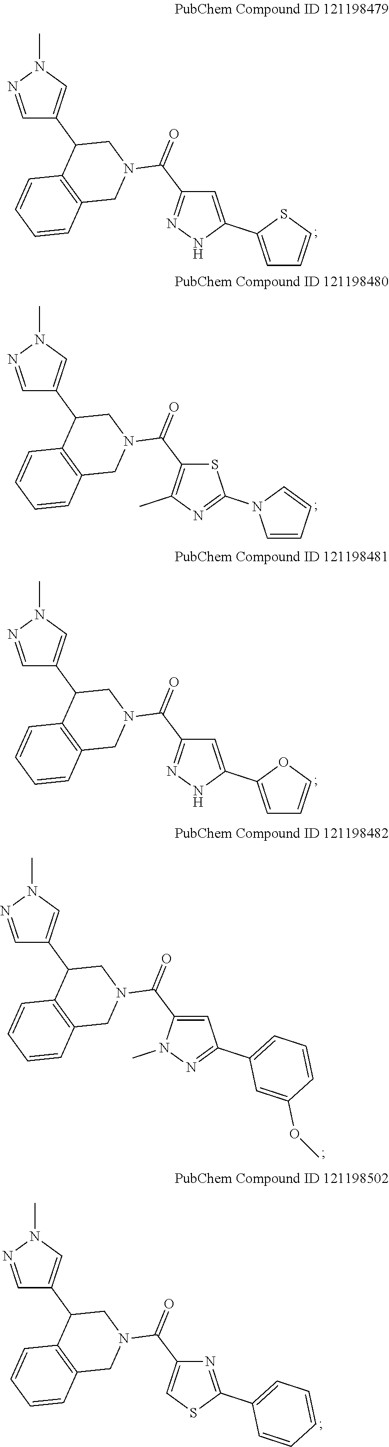 [SMILES: Cn1cc(C2CN(C(=O)c3cc(-c4cccs4)[nH]n3)Cc3ccccc32)cn1 ; Cc1nc(-n2cccc2)sc1C(=O)N1Cc2ccccc2C(c2cnn(C)c2)C1 ; Cn1cc(C2CN(C(=O)c3cc(-c4ccco4)[nH]n3)Cc3ccccc32)cn1 ; COc1cccc(-c2cc(C(=O)N3Cc4ccccc4C(c4cnn(C)c4)C3)n(C)n2)c1 ; Cn1cc(C2CN(C(=O)c3csc(-c4ccccc4)n3)Cc3ccccc32)cn1]

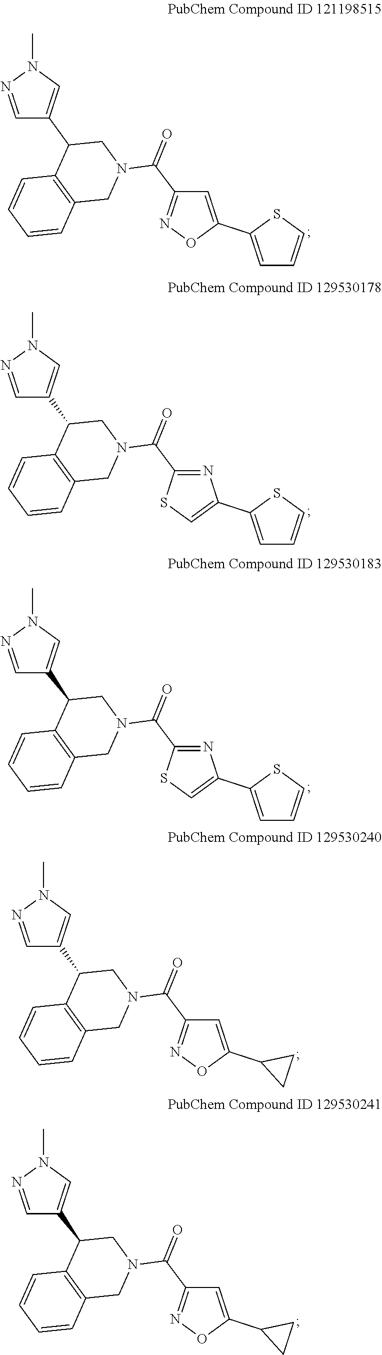 [SMILES: Cn1cc(C2CN(C(=O)c3cc(-c4cccs4)on3)Cc3ccccc32)cn1 ; Cn1cc([C@@H]2CN(C(=O)c3nc(-c4cccs4)cs3)Cc3ccccc32)cn1 ; Cn1cc([C@H]2CN(C(=O)c3nc(-c4cccs4)cs3)Cc3ccccc32)cn1 ; Cn1cc([C@@H]2CN(C(=O)c3cc(C4CC4)on3)Cc3ccccc32)cn1 ; Cn1cc([C@H]2CN(C(=O)c3cc(C4CC4)on3)Cc3ccccc32)cn1]

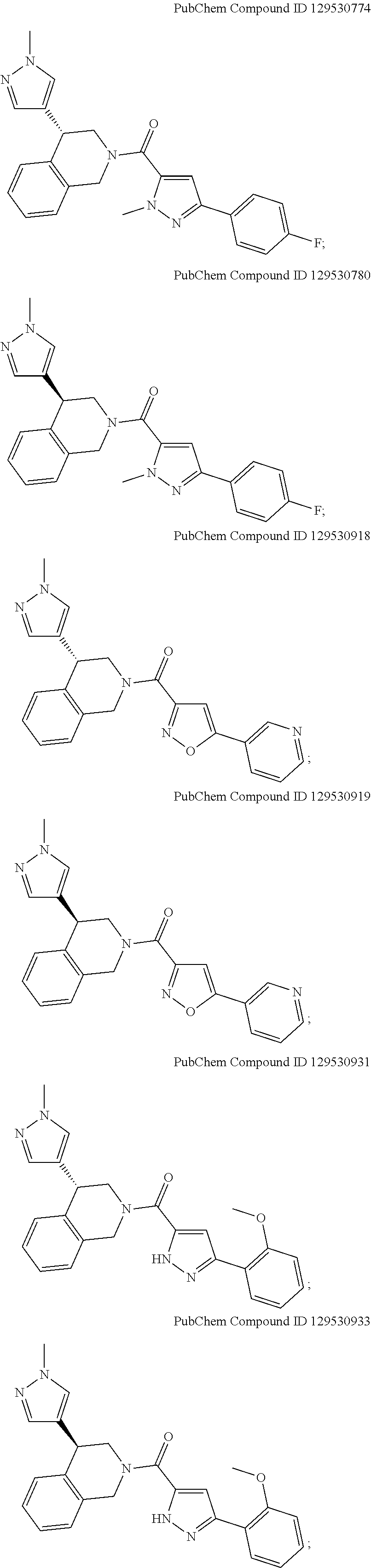 [SMILES: Cn1cc([C@@H]2CN(C(=O)c3cc(-c4ccc(F)cc4)nn3C)Cc3ccccc32)cn1 ; Cn1cc([C@H]2CN(C(=O)c3cc(-c4ccc(F)cc4)nn3C)Cc3ccccc32)cn1 ; Cn1cc([C@@H]2CN(C(=O)c3cc(-c4cccnc4)on3)Cc3ccccc32)cn1 ; Cn1cc([C@H]2CN(C(=O)c3cc(-c4cccnc4)on3)Cc3ccccc32)cn1 ; COc1ccccc1-c1cc(C(=O)N2Cc3ccccc3[C@H](c3cnn(C)c3)C2)[nH]n1 ; COc1ccccc1-c1cc(C(=O)N2Cc3ccccc3[C@@H](c3cnn(C)c3)C2)[nH]n1]

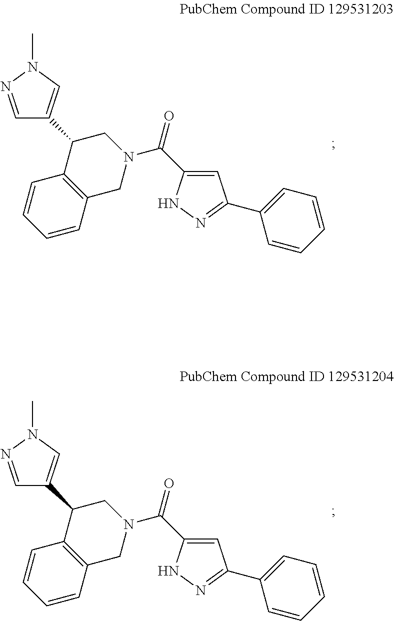 [SMILES: Cn1cc([C@@H]2CN(C(=O)c3cc(-c4ccccc4)n[nH]3)Cc3ccccc32)cn1 ; Cn1cc([C@H]2CN(C(=O)c3cc(-c4ccccc4)n[nH]3)Cc3ccccc32)cn1]

or an agrochemically acceptable salt, stereoisomer, enantiomer, tautomer or N-oxide thereof.

The PubChem Compound ID given for the above disclaimed compounds refers to the identification number of each compound in the PubChem website https://pubchem.ncbi.nlm.nih.gov/.

In one embodiment, in a compound of formula (I) according to the present invention,
R¹ is hydrogen, C₁-C₄ alkyl, C₂-C₄ alkenyl, C₂-C₄ alkynyl or C₃-C₆ cycloalkyl;
R² is hydrogen, halogen, C₁-C₄ alkyl, C₂-C₄ alkenyl, C₂-C₄ alkynyl, C₁-C₄ haloalkyl, C₃-C₆ cycloalkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl or C₁-C₄ alkoxycarbonyl;
R³ and R⁴ are independently from each other hydrogen, halogen or C₁-C₄ alkyl;
R⁵ and R⁶ are independently from each other hydrogen or C₁-C₄ alkyl;
R⁷ is hydrogen, C₁-C₄ alkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, C₁-C₄ alkoxycarbonyl, N-methoxy-N-methyl-carbonyl, C₁-C₄ alkylaminocarbonyl, di(C₁-C₄ alkylamino)carbonyl, phenyl, 5- or 6-membered heteroaryl or C₃-C₆ cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl and C₁-C₄ alkoxy;
B¹ is CR¹⁰ or N;
B² is CR¹¹ or N;
R⁸, R⁹, R¹⁰ and R¹¹ are independently from each other hydrogen, halogen, C₁-C₄ alkyl, C₁-C₄ haloalkyl, C₁-C₄ alkoxy, C₁-C₄ haloalkoxy, C₂-C₄ alkenyloxy, C₂-C₄ alkynyloxy, C₁-C₄ alkylsulfanyl, C₁-C₄ alkylsulfinyl, C₁-C₄ alkylsulfonyl, C₁-C₄ alkoxy-C₁-C₄ alkyl, C₁-C₄ alkoxycarbonyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, hydroxy, trifluoromethylsulfonyloxy, cyano, carboxy, phenyl, 5- or 6-membered heteroaryl or C₃-C₆ cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl and C₁-C₄ alkoxy;
A¹, A² and A³ are independently from each other CR¹², N, NR¹³, O and S, with the proviso that at least one of A¹, A² and A³ is selected from N, O and S, and that no more than one of A¹, A² and A³ is O or S;
R¹² is hydrogen, C₁-C₄ alkyl, C₂-C₄ alkenyl or C₂-C₄ alkynyl;
R¹³ is of hydrogen, C₁-C₄ alkyl, C₂-C₄ alkenyl or C₂-C₄ alkynyl; and
Z¹ is C₃-C₄ alkyl, phenyl, 5- or 6-membered heteroaryl or C₃-C₆-cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl, C₁-C₄ alkoxy, C₁-C₄ haloalkoxy, C₁-C₄ alkylsulfanyl, C₁-C₄ alkylsulfinyl, C₁-C₄ alkylsulfonyl and C₂-C₄ alkynyl,
with the proviso that said compound of formula (I) is not any of the above disclaimed compounds with the following identification numbers PubChem Compound ID 119105753, PubChem Compound ID 119105755, PubChem Compound ID 119105758, PubChem Compound ID 119105768, PubChem Compound ID 121022987, PubChem Compound ID 121023008, PubChem Compound ID 121198339, PubChem Compound ID 121198395, PubChem Compound ID 121198398, PubChem Compound ID 121198478, PubChem Compound ID 121198479, PubChem Compound ID 121198480, PubChem Compound ID 121198481, PubChem Compound ID 121198482, PubChem Compound ID 121198502, PubChem Compound ID 121198515, PubChem Compound ID 129530178, PubChem Compound ID 129530183, PubChem Compound ID 129530240, PubChem Compound ID 129530241, PubChem Compound ID 129530774, PubChem Compound ID 129530780, PubChem Compound ID 129530918, PubChem Compound ID 129530919, PubChem Compound ID 129530931, PubChem Compound ID 129530933, PubChem Compound ID 129531203, PubChem Compound ID 129531204.

In one embodiment, in a compound of formula (I) according to the present invention,
R¹ is hydrogen, C₁-C₄ alkyl, C₂-C₄ alkenyl, C₂-C₄ alkynyl or C₃-C₆ cycloalkyl;
R² is hydrogen, halogen, C₁-C₄ alkyl, C₂-C₄ alkenyl, C₂-C₄ alkynyl, C₁-C₄ haloalkyl, C₃-C₆ cycloalkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl or C₁-C₄ alkoxycarbonyl;
R³ and R⁴ are independently from each other hydrogen, halogen or C₁-C₄ alkyl;
R⁵ and R⁶ are independently from each other hydrogen or C₁-C₄ alkyl;
R⁷ is hydrogen, C₁-C₄ alkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, C₁-C₄ alkoxycarbonyl, N-methoxy-N-methyl-carbonyl, C₁-C₄ alkylaminocarbonyl, di(C₁-C₄ alkylamino)carbonyl, phenyl, 5- or 6-membered heteroaryl or C₃-C₆ cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl and C₁-C₄ alkoxy;
B¹ is CR¹⁰ or N;
B² is CR¹¹ or N;
R⁸, R⁹, R¹⁰ and R¹¹ are independently from each other hydrogen, halogen, C₁-C₄ alkyl, C₁-C₄ haloalkyl, C₁-C₄ alkoxy, C₁-C₄ haloalkoxy, C₂-C₄ alkenyloxy, C₂-C₄ alkynyloxy, C₁-C₄ alkylsulfanyl, C₁-C₄ alkylsulfinyl, C₁-C₄ alkylsulfonyl, C₁-C₄ alkoxy-C₁-C₄ alkyl, C₁-C₄ alkoxycarbonyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, hydroxy, trifluoromethylsulfonyloxy, cyano, carboxy, phenyl, 5- or 6-membered heteroaryl or C₃-C₆ cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl and C₁-C₄ alkoxy;
A¹, A² and A³ are independently from each other CR¹², N, NR¹³, O and S, with the proviso that at least one of A¹, A² and A³ is selected from N, O and S, and that no more than one of A¹, A² and A³ is O or S; R¹² is hydrogen, C₁-C₄ alkyl, C₂-C₄ alkenyl or C₂-C₄ alkynyl;
R¹³ is of hydrogen, C₁-C₄ alkyl, C₂-C₄ alkenyl or C₂-C₄ alkynyl; and
Z¹ is n-propyl, phenyl, 5- or 6-membered heteroaryl or C₃-C₆-cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl, C₁-C₄ alkoxy, C₁-C₄ haloalkoxy, C₁-C₄ alkylsulfanyl, C₁-C₄ alkylsulfinyl, C₁-C₄ alkylsulfonyl and C₂-C₄ alkynyl,
with the proviso that said compound of formula (I) is not any of the above disclaimed compounds with the following identification numbers PubChem Compound ID 119105753, PubChem Compound ID 119105755, PubChem Compound ID 119105758, PubChem Compound ID 119105768, PubChem Compound ID 121022987, PubChem Compound ID 121023008, PubChem Compound ID 121198339, PubChem Compound ID 121198395, PubChem Compound ID 121198398, PubChem Compound ID 121198478, PubChem Compound ID 121198479, PubChem Compound ID 121198480, PubChem Compound ID 121198481, PubChem Compound ID 121198482, PubChem Compound ID 121198502, PubChem Compound ID 121198515, PubChem Compound ID 129530178, PubChem Compound ID 129530183, PubChem Compound ID 129530240, PubChem Compound ID 129530241, PubChem Compound ID 129530774, PubChem Compound ID 129530780, PubChem Compound ID 129530918, PubChem Compound ID 129530919, PubChem Compound ID 129530931, PubChem Compound ID 129530933, PubChem Compound ID 129531203, PubChem Compound ID 129531204.

In another embodiment, in a compound of formula (I) according to the present invention,
R¹ is C₁-C₄ alkyl,
R² is hydrogen, halogen, C₁-C₄ alkyl, C₁-C₄ alkylcarbonyl or N—C₁-C₄ alkoxy-C—C₁-C₄ alkyl-carbonimidoyl,
R³ and R⁴ are independently from each other hydrogen or C₁-C₄ alkyl,
R⁵ and R⁶ are hydrogen or C₁-C₄ alkyl,
R⁷ is hydrogen, C₁-C₄ alkyl, C₁-C₄ alkylcarbonyl, C₁-C₄ alkoxycarbonyl, N—C₁-C₄ alkoxy-C—C₁-C₄ alkyl-carbonimidoyl or phenyl,
B¹ is CR¹⁰ or N;
B² is CR¹¹ or N;
R³ and R¹¹ are independently from each other hydrogen, halogen or C₁-C₄ alkyl,
R⁹ and R¹⁰ are independently from each other hydrogen, halogen, hydroxy, C₁-C₃ alkyl, C₁-C₂ haloalkyl, C₁-C₃ haloalkoxy, C₁-C₄ alkoxy, C₁-C₃ alkoxycarbonyl, cyano or phenyl,
A¹ and A² are independently from each other CR¹², N and O and A³ is CR¹², N, O or S, with the proviso that at least one of A¹, A² and A³ is N or O and that no more than one of A¹, A² and A³ is O;
R¹² is hydrogen or C₁-C₄ alkyl, and
Z¹ is C₃-C₄ alkyl, phenyl, 5- or 6-membered heteroaryl or C₃-C₆-cycloalkyl, wherein the 5- or 6-membered heteroaryl comprises at least one heteroatom selected from N, O and S, or wherein any of said phenyl, 5- or 6-membered heteroaryl or C₃-C₆-cycloalkyl are optionally substituted by 1, 2 or 3 substituents independently selected from halogen, C₁-C₄ alkyl, C₁-C₄ haloalkyl, C₁-C₄ alkoxy, C₁-C₄ haloalkoxy, C₁-C₄ alkylsulfonyl or C₂-C₄ alkynyl.

In one embodiment, in a compound of formula (I) according to the present invention
R¹ is hydrogen or C₁-C₄ alkyl,
R² is hydrogen, halogen, C₁-C₄ alkyl, C₃-C₆cycloalkyl, C₁-C₄ alkylcarbonyl, N—C₁-C₄ alkoxy-C₁-C₄ alkyl-carbonimidoyl or N-hydroxy-C₁-C₄ alkyl-carbonimidoyl,
R³ and R⁴ are independently from each other hydrogen, halogen or C₁-C₄ alkyl,
R⁵ and R⁶ are independently from each other hydrogen, methyl or ethyl,
R⁷ is hydrogen, C₁-C₄ alkyl, C₁-C₄ alkylcarbonyl, N—C₁-C₄ alkoxy-C₁-C₄ alkyl-carbonimidoyl, N-hydroxy-C₁-C₄ alkyl-carbonimidoyl, C₁-C₄ alkoxycarbonyl, N-methoxy-N-methyl-carbonyl, phenyl, 4-cyanophenyl, cyclopropyl and 1-cyanocyclopropyl,
B¹ is CR¹⁰;
B² is CR¹¹,
R⁸ and R¹¹ are independently from each other hydrogen, halogen or C₁-C₄ alkyl, R⁹ and R¹⁰ are independently from each other hydrogen, chloro, fluoro, methyl, trifluoromethyl, difluoromethoxy, trifluoromethoxy, 2,2-difluoroethoxy, 2,2,2-trifluoroethoxy, methoxy, propoxy, allyloxy, prop-2-ynoxy, methylsulfanyl, methylsulfinyl, methylsulfonyl, methoxymethyl, 2-methoxyethoxymethyl, methoxycarbonyl, acetyl, propanoyl, —C(CH₃)═NOCH₃, —C(CH₃)═NOCH₂CH₃, —C(CH₃)═NOH, methylaminocarbonyl, di(methylamino)carbonyl, trifluoromethylsulfonyloxy, cyano, carboxy, phenyl, 2-cyanophenyl, 3-cyanophenyl, 4-cyanophenyl, [4-(trifluoromethyl)pyrazol-1-yl], [3-(trifluoromethyl)pyrazol-1-yl], (3-cyanopyrazol-1-yl), (4-cyanopyrazol-1-yl), (5-chloropyrazol-1-yl), (4-chloropyrazol-1-yl), (3-chloropyrazol-1-yl), (5-fluoropyrazol-1-yl), (4-fluoropyrazol-1-yl), (3-fluoropyrazol-1-yl), (3,5-dimethylpyrazol-1-yl), (5-methylpyrazol-1-yl), (4-methylpyrazol-1-yl), (3-methylpyrazol-1-yl), pyrazol-1-yl, cyclopropyl or 1-cyanocyclopropyl, A¹ and A² are independently from each other CR¹², N or O and A³ is CR¹², N, O or S, with the proviso that at least one of A¹, A² and A³ is N or O and that no more than one of A¹, A² and A³ is O,
R¹² is hydrogen or methyl,
Z¹ is 1-methylpyrazol-4-yl, 2,3,4-trifluorophenyl, 2,3-difluorophenyl, 3,4-difluorophenyl, 2,4,6-trifluorophenyl, 2,4-difluorophenyl, 2,5-difluorophenyl, 2-fluoro-4-methoxy-phenyl, 2-fluoro-4-methylsulfonyl-phenyl, 2-fluorophenyl, 3-fluorophenyl, 4-fluorophenyl, 2-furyl, 2-thienyl, 3-thienyl, 2-methylphenyl, 3-methylphenyl, 4-methylphenyl, 2-chlorophenyl, 3-chlorophenyl, 4-chlorophenyl, 3-methoxyphenyl, 4-ethynyl-2-fluoro-phenyl, 4-fluoro-2-methoxy-phenyl, cyclopropyl, 1-methylcyclopropyl, cyclobutyl, cyclohexyl, cyclopentyl, methyl, n-propyl, or phenyl.

In another embodiment, in a compound of formula (I) according to the present invention
R¹ is C₁-C₄ alkyl,
R² is hydrogen, halogen, C₁-C₄ alkyl, C₁-C₄ alkylcarbonyl or N—C₁-C₄ alkoxy-C—C₁-C₄ alkyl-carbonimidoyl,
R³ and R⁴ are independently from each other hydrogen or C₁-C₄ alkyl,
R⁵ and R⁶ are hydrogen or C₁-C₄ alkyl,
R⁷ is hydrogen, C₁-C₄ alkyl, C₁-C₄ alkylcarbonyl, C₁-C₄ alkoxycarbonyl, N—C₁-C₄ alkoxy-C—C₁-C₄ alkyl-carbonimidoyl or phenyl,
B¹ is CR¹⁰ or N;
B² is CR¹¹ or N;
R⁸ and R¹¹ are independently from each other hydrogen, halogen or C₁-C₄ alkyl,
R⁹ and R¹⁰ are independently from each other hydrogen, halogen, hydroxy, C₁-C₃ alkyl, C₁-C₂ haloalkyl, C₁-C₃ haloalkoxy, C₁-C₄ alkoxy, C₁-C₃ alkoxycarbonyl, cyano or phenyl,
A¹ and A² are independently from each other CR¹², N and O and A³ is CR¹², N, O or S, with the proviso that at least one of A¹, A² and A³ is N or O and that no more than one of A¹, A² and A³ is O;
R¹² is hydrogen or C₁-C₄ alkyl, and
Z¹ is C₃-C₄ alkyl, phenyl, 5- or 6-membered heteroaryl or C₃-C₆-cycloalkyl, wherein the 5- or 6-membered heteroaryl comprises at least one heteroatom selected from N, O and S, or wherein any of said phenyl, 5- or 6-membered heteroaryl or C₃-C₆-cycloalkyl are optionally substituted by 1, 2 or 3 substituents independently selected from halogen, C₁-C₄ alkyl, C₁-C₄ haloalkyl, C₁-C₄ alkoxy, C₁-C₄ haloalkoxy, C₁-C₄ alkylsulfonyl or C₂-C₄ alkynyl.

In one embodiment compound of formula (I) may be a compound (I-B)
wherein B¹ is CR¹⁰ and B² is CR¹¹; and
wherein R¹, R², R³, R⁴, R⁵, R⁶, R⁷, R⁸, R⁹, R¹⁰, and R¹¹ correspond to the same definitions as for the compounds of formula (I) according to the present invention.

Preferably in compound (I-B)
R¹ is C₁-C₄ alkyl,
R² is hydrogen, halogen, C₁-C₄ alkyl, C₁-C₄ alkylcarbonyl or N—C₁-C₄ alkoxy-C—C₁-C₄ alkyl-carbonimidoyl,
R³ and R⁴ are independently from each other hydrogen or C₁-C₄ alkyl,
R⁵ and R⁶ are hydrogen or C₁-C₄ alkyl,
R⁷ is hydrogen, C₁-C₄ alkyl, C₁-C₄ alkylcarbonyl, C₁-C₄ alkoxycarbonyl, N—C₁-C₄ alkoxy-C—C₁-C₄ alkyl-carbonimidoyl or phenyl,
R⁸ and R¹¹ are independently from each other hydrogen, halogen or C₁-C₄ alkyl,
R⁹ and R¹⁰ are independently from each other hydrogen, halogen, hydroxy, C₁-C₃ alkyl, C₁-C₂ haloalkyl, C₁-C₃ haloalkoxy, C₁-C₄ alkoxy, C₁-C₃ alkoxycarbonyl, cyano or phenyl,
A¹ and A² are independently from each other CR¹², N and O and A³ is CR¹², N, O or S, with the proviso that at least one of A¹, A² and A³ is N or O and that no more than one of A¹, A² and A³ is O;
R¹² is hydrogen or C₁-C₄ alkyl, and
Z¹ is C₃-C₄ alkyl, phenyl, 5- or 6-membered heteroaryl or C₃-C₆-cycloalkyl, wherein the 5- or 6-membered heteroaryl comprises at least one heteroatom selected from N, O and S, or wherein any of said phenyl, 5- or 6-membered heteroaryl or C₃-C₆-cycloalkyl are optionally substituted by 1, 2 or 3 substituents independently selected from halogen, C₁-C₄ alkyl, C₁-C₄ haloalkyl, C₁-C₄ alkoxy, C₁-C₄ haloalkoxy, C₁-C₄ alkylsulfonyl or C₂-C₄ alkynyl.

In one embodiment compound of formula (I) may be a compound (I-B)
wherein B¹ is CR¹⁰ and B² is CR¹¹ and wherein R⁵ and R⁶ are hydrogen; and
wherein R¹, R², R³, R⁴, R⁷, R⁸, R⁹, R¹⁰, and R¹¹ correspond to the same definitions as for the compounds of formula (I) according to the present invention.

Preferably in compound (I-B)
R¹ is C₁-C₄ alkyl,
R² is hydrogen, halogen or C₁-C₄ alkyl,
R³ and R⁴ are independently from each other hydrogen or C₁-C₄ alkyl,
R⁷ is hydrogen or C₁-C₄ alkyl, and
R⁸, R⁹, R¹⁰, R¹¹, A¹, A², A³, R¹², R¹³ and Z¹ are as defined for the compounds of formula (I) according to the present invention.

In another embodiment compound of formula (I) may be a compound (I-B)
wherein B¹ is CR¹⁰ and B² is CR¹¹, and wherein R⁴, R⁵, R⁶, and R⁷ are hydrogen; and
wherein R¹, R², R³, R⁸, R⁹, R¹⁰, and R¹¹ correspond to the same definitions as for the compounds of formula (I) according to the present invention.

Preferably in compound (I-B)
R¹ is C₁-C₄ alkyl,
R² is hydrogen, halogen or C₁-C₄ alkyl,
R³ is hydrogen or C₁-C₄ alkyl, and
R⁸, R⁹, R¹⁰, R¹¹, A¹, A², A³, R¹², R¹³ and Z¹ are as defined for the compounds of formula (I) according to the present invention.

In still another embodiment compound of formula (I) may be a compound (I-B)
wherein B¹ is CR¹⁰ and B² is CR¹¹, and wherein R⁷ is methyl; and
wherein R¹, R², R³, R⁴, R⁵, R⁶, R⁸, R⁹, R¹⁰, and R¹¹ correspond to the same definitions as for the compounds of formula (I) according to the present invention.

Preferably in compound (I-B)
R¹ is C₁-C₄ alkyl,
R² is hydrogen, halogen or C₁-C₄ alkyl,
R³ and R⁴ are independently from each other hydrogen or C₁-C₄ alkyl,
R⁵ and R⁶ are hydrogen, and
R⁸, R⁹, R¹⁰, R¹¹, A¹, A², A³, R¹², R¹³ and Z¹ are as defined for the compounds of formula (I) according to the present invention.

According to a fourth aspect of the invention, there is provided an agrochemical composition comprising a fungicidally effective amount of a compound of formula (I) according to the invention. Such an agricultural composition may further comprise at least one additional active ingredient and/or an agrochemically-acceptable diluent or carrier.

According to a fifth aspect of the invention, there is provided an intermediate compound of formula (III) or a salt thereof:

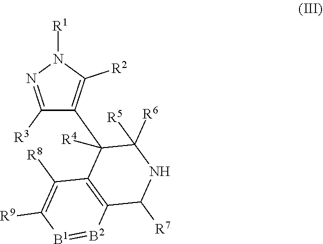

wherein R¹, R², R³, R⁴, R⁵, R⁶, R⁷, R⁸, R⁹, B¹ and B² correspond to the same definitions as for the compounds of formula (I) according to the present invention.

The intermediate compounds of formula (III) possess the same definitions for R¹, R², R³, R⁴, R⁵, R⁶, R⁷, R⁸, R⁹, B¹ and B² as for the compounds of formula (I) according to the invention and their corresponding preferences.

In one embodiment intermediate compound of formula (III) may be a compound (III-c)
wherein B¹ is CR¹⁰ and B² is CR¹¹; and
wherein R¹, R², R³, R⁴, R⁵, R⁶, R⁷, R⁸, R⁹, R¹⁰, and R¹¹ correspond to the same definitions as for the compounds of formula (I) according to the present invention.

The presence of one or more possible asymmetric carbon atoms in a compound of formula (III) according to the invention means that the compounds may occur in chiral isomeric forms, i.e., enantiomeric or diastereomeric forms.

According to a sixth aspect of the invention, there is provided an intermediate compound of formula

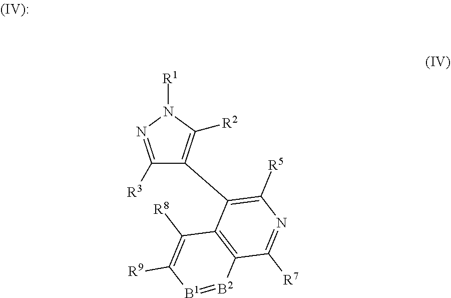

wherein R¹, R², R³, R⁵, R⁷, R⁸, R⁹, B¹ and B² correspond to the same definitions as for the compounds of formula (I) according to the present invention.

The intermediate compounds of formula (IV) possess the same definitions for R¹, R², R³, R⁵, R⁷, R⁸, R⁹, B¹ and B² as for the compounds of formula (I) according to the invention and their corresponding preferences.

In one embodiment intermediate compound of formula (IV) may be a compound (IV-a)
wherein B¹ is CR¹⁰ and B² is CR¹¹; and
wherein R¹, R², R³, R⁵, R⁶, R⁷, R⁸, R⁹, R¹⁰, and R¹¹ correspond to the same definitions as for the compounds of formula (I) according to the present invention.

The presence of one or more possible asymmetric carbon atoms in a compound of formula (IV) according to the invention means that the compounds may occur in chiral isomeric forms, i.e., enantiomeric or diastereomeric forms.

According to a seventh aspect of the invention, there is provided an intermediate compound of formula (XVIII):

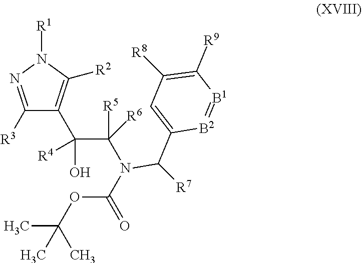

wherein R¹, R², R³, R⁴, R⁵, R⁶, R⁷, R⁸, R⁹, B¹ and B² correspond to the same definitions as for the compounds of formula (I) according to the present invention.

The intermediate compounds of formula (XVIII) possess the same definitions for R¹, R², R³, R⁴, R⁵, R⁶, R⁷, R⁸, R⁹, B¹ and B² as for the compounds of formula (I) according to the invention and their corresponding preferences.

In one embodiment intermediate compound of formula (XVIII) may be a compound (XVIII-a) wherein B¹ is CR¹⁰ and B² is CR¹¹; and wherein R¹, R², R³, R⁴, R⁵, R⁶, R⁷, R⁸, R⁹, R¹⁰, and R¹¹ correspond to the same definitions as for the compounds of formula (I) according to the present invention.

The presence of one or more possible asymmetric carbon atoms in a compound of formula (XVIII) according to the invention means that the compounds may occur in chiral isomeric forms, i.e., enantiomeric or diastereomeric forms.

According to a eighth aspect of the invention, there is provided an intermediate compound of formula (XIX):

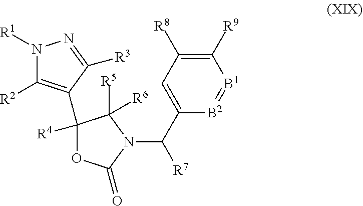

wherein R¹, R², R³, R⁴, R⁵, R⁶, R⁷, R⁸, R⁹, B¹ and B² correspond to the same definitions as for the compounds of formula (I) according to the present invention.

The intermediate compounds of formula (XIX) possess the same definitions for R¹, R², R³, R⁴, R⁵, R⁶, R⁷, R⁸, R⁹, B¹ and B² as for the compounds of formula (I) according to the invention and their corresponding preferences.

In one embodiment intermediate compound of formula (XIX) may be a compound (XIX-a)
wherein B¹ is CR¹⁰ and B² is CR¹¹; and
wherein R¹, R², R³, R⁴, R⁵, R⁶, R⁷, R⁸, R⁹, R¹⁰, and R¹¹ correspond to the same definitions as for the compounds of formula (I) according to the present invention.

In one embodiment intermediate compound of formula (XIX) may be a compound (XIX-b)
wherein R⁴ is hydrogen, B¹ is CR¹⁰ and B² is CR¹¹; and
wherein R¹, R², R³, R⁵, R⁶, R⁷, R⁸, R⁹, R¹⁰, and R¹¹ correspond to the same definitions as for the compounds of formula (I) according to the present invention.

The presence of one or more possible asymmetric carbon atoms in a compound of formula (XIX) according to the invention means that the compounds may occur in chiral isomeric forms, i.e., enantiomeric or diastereomeric forms.

According to a ninth aspect of the invention, there is provided an intermediate compound of formula (XX):

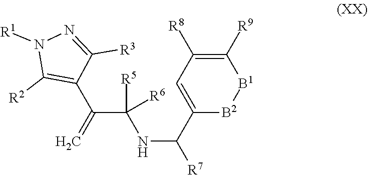

wherein R¹, R², R³, R⁵, R⁶, R⁷, R⁸, R⁹, B¹ and B² correspond to the same definitions as for the compounds of formula (I) according to the present invention.

The intermediate compounds of formula (XX) possess the same definitions for R¹, R², R³, R⁵, R⁶, R⁷, R⁸, R⁹, B¹ and B² as for the compounds of formula (I) according to the invention and their corresponding preferences.

In one embodiment intermediate compound of formula (XX) may be a compound (XX-a) wherein B¹ is CR¹⁰ and B² is CR¹¹; and wherein R¹, R², R³, R⁵, R⁶, R⁷, R⁸, R⁹, R¹⁰, and R¹¹ correspond to the same definitions as for the compounds of formula (I) according to the present invention.

The presence of one or more possible asymmetric carbon atoms in a compound of formula (XX) according to the invention means that the compounds may occur in chiral isomeric forms, i.e., enantiomeric or diastereomeric forms.

Certain intermediates described in the above schemes are novel and as such form a further aspect of the invention.

The compounds of formula (I) according to the present invention can be made as shown in the following Schemes 1 to 12, in which, unless otherwise stated, the definition of each variable is as defined above for a compound of formula (I).

In particular, compounds of formula (I), wherein R⁴ and R⁶ are hydrogen and R⁵ is hydrogen or methyl, can be made as shown in the following Schemes 1 to 7, in which, unless otherwise stated, the definition of each variable is as defined above for a compound of formula (I).

In any of the Schemes 1 to 12 below, the presence of one or more possible asymmetric carbon atoms in a compound of formula (I) according to the invention means that the compounds may occur in chiral isomeric forms, i.e., enantiomeric or diastereomeric forms.

Compounds of formula (I) may be prepared by a person skilled in the art following known methods. More specifically, compounds of formula (I) may be prepared from compounds of formula (III) or a salt thereof, wherein R¹, R², R³, R⁴, R⁵, R⁶, R⁷, R⁸, R⁹, B¹ and B² are as defined above for the compound of formula (I) by reaction with a compound of formula (II), wherein A¹, A², A³ and Z¹ are as defined above for the compound of formula (I). This reaction is shown in Scheme 1.

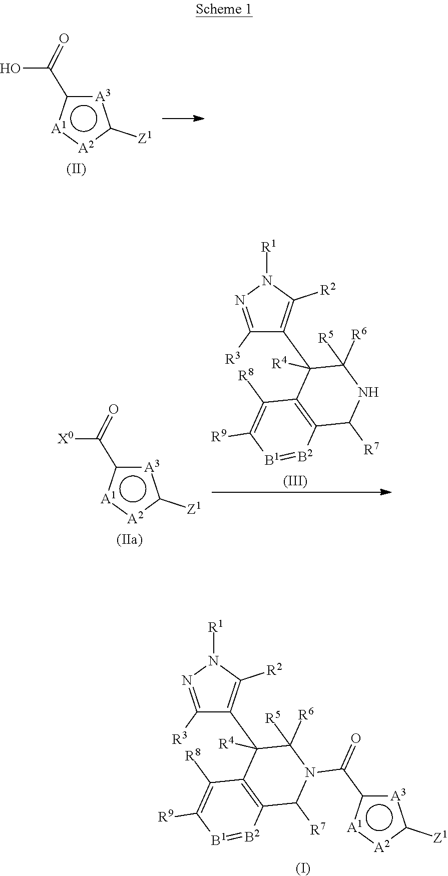

In Scheme 1, compounds of formula (II), wherein A¹, A², A³ and Z¹ are as defined above for the compound of formula (I), are activated to compounds of formula (IIa) by methods known to a person skilled in the art and described, for example, in Tetrahedron, 61 (46), 10827-10852, 2005. For example, compounds of formula (IIa), where X⁰ is halogen, are formed by treatment of compounds of formula (II) with, for example, oxalyl chloride or thionyl chloride in the presence of catalytic quantities of N,N-dimethylformamide (DMF) in inert solvents such as methylene dichloride or tetrahydrofuran (THF) at temperatures between 20° C. to 100° C., preferably 25° C. Treatment of compounds of formula (IIa) with compounds of formula (III), wherein R¹, R², R³, R⁴, R⁵, R⁶, R⁷, R⁸, R⁹, B¹ and B² are as defined above for the compound of formula (I), optionally in the presence of a base, e.g. triethylamine or pyridine, leads to compounds of formula (I). Alternatively, compounds of formula (I) may be prepared by treatment of compounds of formula (II) with dicyclohexyl carbodiimide (DCC), 1-ethyl-3-(3-dimethylaminopropyl)carbodiimide (EDC) or 1-[bis(dimethylamino)methylene]-1H-1,2,3-triazolo[4,5-b]pyridinium 3-oxide hexafluorophosphate (HATU) to give the activated compound of formula (IIa), wherein X⁰ is X⁰¹, X⁰² or X⁰³ as set forth below, in an inert solvent, e.g. pyridine, DMF, acetonitrile, CH₂Cl₂ or THF, optionally in the presence of a base, e.g. triethylamine, at temperatures between 30° C. and 180° C. Finally, a compound of formula (II) can also be activated by reaction with a coupling reagent such as propanephosphonic acid anhydride (T3P) to provide compounds of formula (IIa), wherein X⁰ is X⁰⁴ as set forth below, as described for example in Synthesis 2013, 45, 1569. Further reaction with an amine (or a salt thereof) of the compound of formula (III) leads to compounds of formula (I).

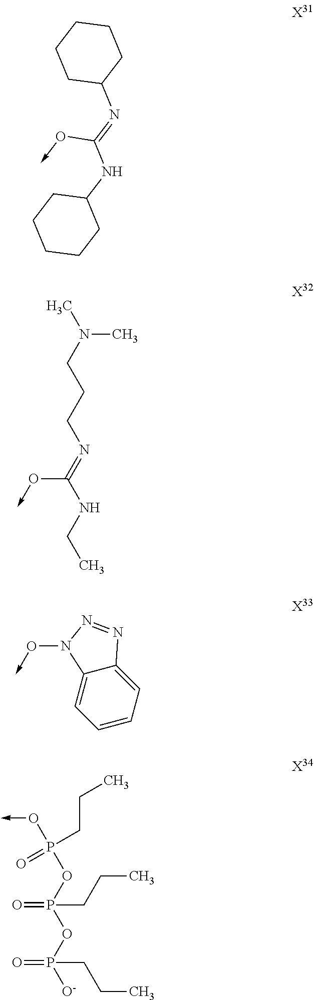

Compounds of formula (IIIa), wherein R⁴ and R⁶ are hydrogen, R⁵ is hydrogen or methyl and R¹, R², R³, R⁷, R⁸, R⁹, B¹ and B² are as defined above for the compound of formula (I), may be prepared by a person skilled in the art following known methods.

For example, compounds of formula (IIIa), wherein R⁴ and R⁶ are hydrogen, R⁵ is hydrogen or methyl and R¹, R², R³, R⁷, R⁸, R⁹, B¹ and B² are as defined above for the compound of formula (I), may be prepared from compounds of formula (IVa), wherein R⁴ and R⁶ are hydrogen, R⁵ is hydrogen or methyl and R¹, R², R³, R⁷, R⁸, R⁹, B¹ and B² are as defined above for the compound of formula (I), by treatment with a reducing agent such as NaBH₃CN and an acid, for example hydrochloric acid, or acetic acid in a protic solvent such as methanol or ethanol and the like. Such reactions are well known in the literature and analogous reactions have been described for example in Deng, Zeping et al, CN103772278, and Synthesis (1979), 4, 281-3 Alternatively, compounds of formula (IIIa) may be prepared from compounds of formula (IV by reduction with hydrogen in the presence of a suitable metal catalyst, such as Pd, Ir, Rh with a suitable ligand, e.g. diphosphine[1,2-bis(diphenylphosphino)ethane (dppe), 1,3-bis(diphenylphosphino)propane (dppp) or 1,4-bis(diphenylphosphino)butane (dppb)]. Similar reactions have been reported for example in Reaction Kinetics and Catalysis Letters (2007), 92, 99-104. This reaction is shown in Scheme 2.

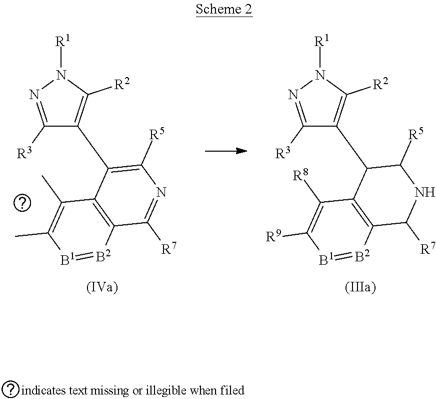

Alternatively, compounds of formula (IIIa) may be prepared as shown in Scheme 4. As shown in Scheme 3, compounds of formula (IIIb), wherein R⁴, R⁶ and R⁷ are hydrogen, R⁵ is hydrogen or methyl and R¹, R², R³, R⁸, R⁹, B¹ and B² are as defined above for the compound of formula (I), can be converted to compounds of formula (V), wherein R⁴, R⁶ and R⁷ are hydrogen, R⁵ is hydrogen or methyl and R¹, R², R³, R⁸, R⁹, B¹ and B² are as defined above for the compound of formula (I), by treatment of compounds of formula (IIIb) with a compound of formula (VI), wherein X⁰ is a leaving group, such as halogen, and R⁰ is C₁-C₄ alkyl, by methods known to a person skilled in the art and by those described in Scheme 1. Alternatively, compounds of formula (V) may be prepared by treatment with an anhydride of formula (R⁰CO)₂O, wherein R⁰ is C₁-C₄ alkyl, in an inert solvent such as methylene chloride, THF or 2-methyl-THF, optionally in the presence of a base, such as triethylamine or dimethylaminopyridine at temperatures between 0° C. and 60° C. Compounds of formula (V) are then metalated with a base, for example an alkyl metal base, such as tert-butyl lithium, and an additive such as N,N,N′,N′-tetramethylethylenediamine (TMEDA) at low temperature, for example −78° C. to room temperature, in an inert polar solvent such as THF or 2-methyl-THF. Subsequent treatment of the anion of formula (V) formed under such conditions with an electrophile of formula R^{X}—X⁰, wherein X⁰ is as previously defined and R^{X} is C₁-C₄ alkyl, C₁-C₄ alkylcarbonyl, C₁-C₄ alkoxycarbonyl, N-methoxy-N-methyl-carbonyl, C₁-C₄ alkylaminocarbonyl, di(C₁-C₄ alkylamino)carbonyl or C₃-C₆ cycloalkyl, wherein the C₃-C₆-cycloalkyl is optionally substituted by 1, 2 or 3 substituents independently selected from halogen, cyano, C₁-C₄ alkyl, C₁-C₄ haloalkyl and C₁-C₄ alkoxy, yields compounds of formula (Va), wherein R⁴ and R⁶ are hydrogen, R⁵ is hydrogen or methyl, R⁰ is C₁-C₄ alkyl and R¹, R², R³, R⁷, R⁸, R⁹, B¹ and B² are as defined above for the compound of formula (I). This reaction is shown in Scheme 3.

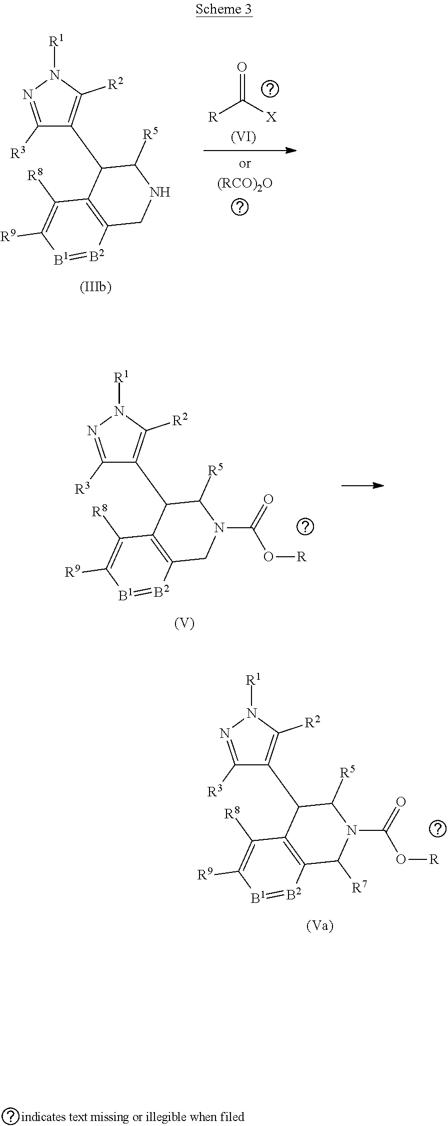

Compounds of formula (Va) may be converted to compounds of formula (IIIa), wherein R⁴ and R⁶ are hydrogen, R⁵ is hydrogen or methyl and R¹, R², R³, R⁷, R⁸, R⁹, B¹ and B² are as defined above for the compound of formula (I), by methods known to a person skilled in the art. For example, compounds of formula (Va), wherein R⁰ is tert-butyl, may be treated with an organic or inorganic acid such as trifluoroacetic acid or HCl to give compounds of formula (IIIa). This reaction is shown in Scheme 4.

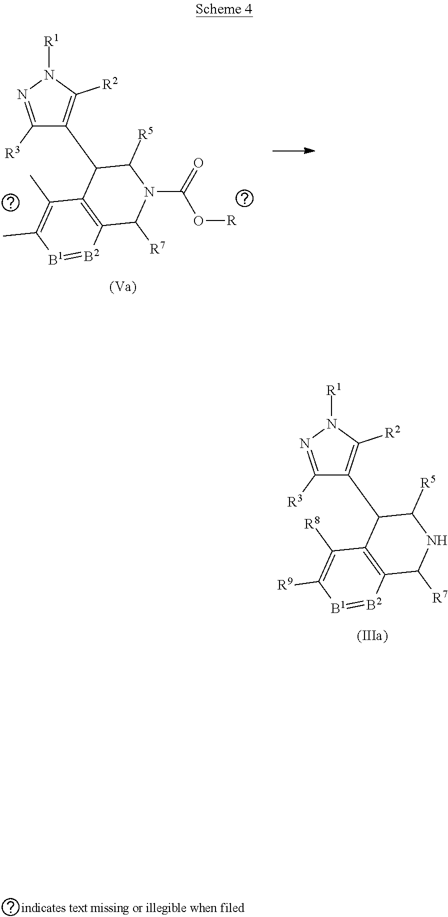

Compounds of formula (IVa), wherein R⁴ and R⁶ are hydrogen, R⁵ is hydrogen or methyl and R¹, R², R³, R⁷, R⁸, R⁹, B¹ and B² are as defined above for the compound of formula (I), may be prepared by reacting compounds of formula (VIII), wherein R¹, R² and R³ are as defined above for the compound of formula (I) and X⁰ is halogen, preferably chlorine, bromine or iodine, with compounds of formula (VII), wherein R⁵ is hydrogen or methyl and R⁷, R⁸, R⁹, B¹ and B² are as defined above for the compound of formula (I), by means of a C—C bond formation reaction typically under palladium-catalyzed (alternatively nickel-catalyzed) cross-coupling conditions. This reaction is shown in Scheme 5.

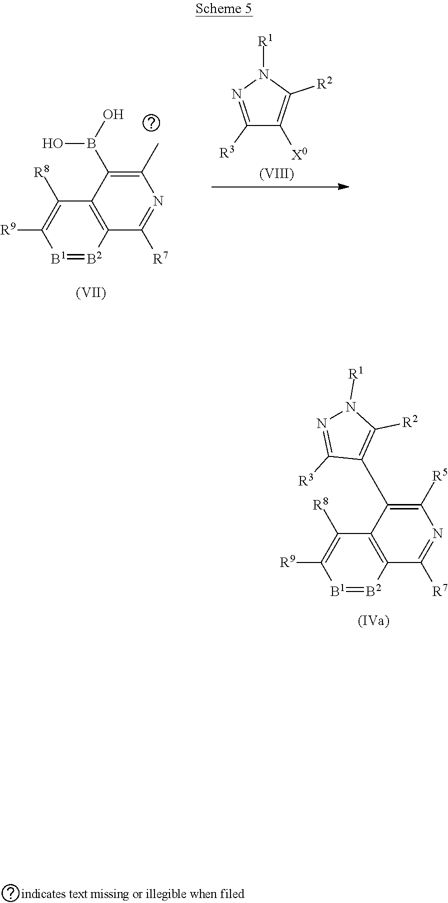

Suzuki-Miyaura cross-coupling reactions between compounds of formula (VIII) and compound of formula (VII) are well known to a person skilled in the art and are usually carried out in the presence of a palladium catalyst, such as tetrakis(triphenylphosphine)-palladium(0) or [1,1′-bis(diphenylphosphino)ferrocene]palladium(II) dichloride dichloromethane complex, and a base, such as sodium or potassium carbonate, in a solvent, such as N,N-dimethylformamide, dioxane or dioxane-water mixtures, at temperatures between room temperature and 160° C., optionally under microwave heating conditions, and preferably under inert atmosphere. Such reactions have been reviewed for example in J. Organomet. Chem. 576, 1999, 147-168. A person skilled in the art will also recognize that the reaction can be reversed, i.e. by reacting a compound of formula (X), wherein R¹, R² and R³ are as defined above for the compound of formula (I), with a compound of formula (IX), wherein R⁵ is hydrogen or methyl, R⁷, R⁸, R⁹, B¹ and B² are as defined above for the compound of formula (I) and X⁰ is halogen, preferably chlorine, bromine or iodine, to provide a compound of formula (IVa), wherein R⁴ and R⁶ are hydrogen, R⁵ is hydrogen or methyl and R¹, R², R³, R⁷, R⁸, R⁹, B¹ and B² are as defined above for the compound of formula (I). This reaction is shown in Scheme 6.

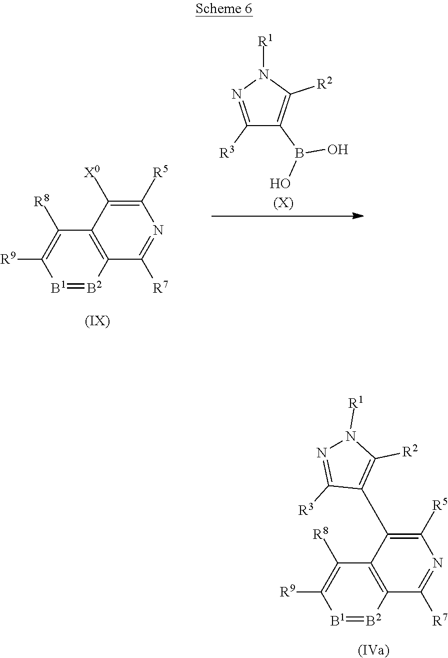

A further cross-coupling chemistry, namely C—H activation, can also be used to prepare compounds of formula (IVa), wherein R⁴ and R⁶ are hydrogen, R⁵ is hydrogen or methyl and R¹, R², R³, R⁷, R⁸, R⁹, B¹ and B² are as defined above for the compound of formula (I). This reaction is shown in Scheme 7.

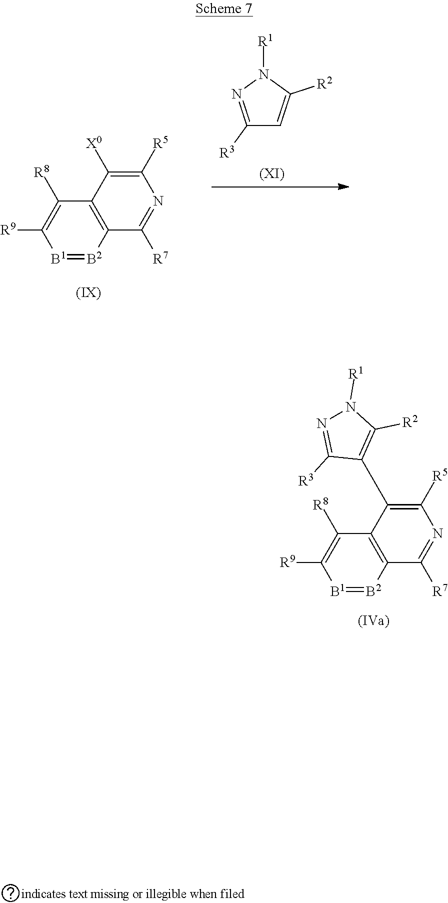

As shown in Scheme 7, compounds of formula (IX), wherein R⁵ is hydrogen or methyl, R⁷, R⁸, R⁹, B¹ and B² are as defined above for the compound of formula (I) and X⁰ is halogen, preferably chlorine, bromine or iodine, are reacted with compounds of formula (XI), wherein R¹, R² and R³ are as defined above for the compound of formula (I), in the presence of a palladium catalyst, typically palladium acetate Pd(OAc)₂, a suitable ligand, for example 1,10-phenanthroline, in the presence of a base such as cesium carbonate or potassium carbonate, in inert solvents such as chlorobenzene, toluene or xylene at temperatures between room temperature and 180° C., optionally under microwave heating conditions, preferably under inert atmosphere. Similar reactions have been reported in the literature for example in Chemical Science (2013), 4, 2374-2379.

Also, compounds of formula (III) may be prepared from compounds of formula (XVI). This reaction is shown in Scheme 8.

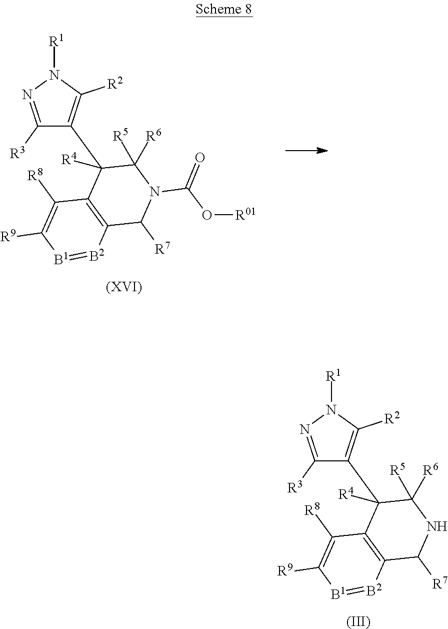

As shown in Scheme 8, compounds of formula (III) may be prepared by a person skilled in the art by a carbamate deprotection reaction of compounds of formula (XVI), wherein R¹, R², R³, R⁴, R⁵, R⁶, R⁷, R⁸, R⁹, B¹ and B² are as defined above for the compound of formula (I) and R⁰¹ may be a member of a common carbamate protecting group substituent, for example methyl, tert-butyl, allyl, 2,2,2-trichloroethyl or benzyl. For example, when R⁰¹ is methyl, a suitable solvent such as dichloromethane and a suitable reagent such as iodotrimethylsilane may be employed to afford the product upon heating at temperatures between room temperature and 200° C., preferably between 20° C. and the boiling point of the reaction mixture as described, for example, in Journal of the American Chemical Society 1992, 114, 5959. The compounds of formula (III) thus obtained are converted to compounds of formula (I) as shown in Scheme 1.

Compounds of formula (XVI), wherein R¹, R², R³, R⁴, R⁵, R⁶, R⁷, R⁸, R⁹, B¹ and B² are as defined above for the compound of formula (I) and wherein R⁰¹ is as described above, may be formed by a Pictet-Spengler reaction between an aldehyde (including formaldehyde in its various forms) of formula (XV), wherein R⁷ is as defined above for the compound of formula (I), and a compound of formula (XIV), wherein R¹, R², R³, R⁴, R⁵, R⁶, R⁸, R⁹, B¹ and B² are as defined above for the compound of formula (I) and wherein R⁰¹ is as described above, by combination with an acid in a suitable solvent, for example as described in Tetrahedron 1987, 43, 439. This reaction is shown in Scheme 9.

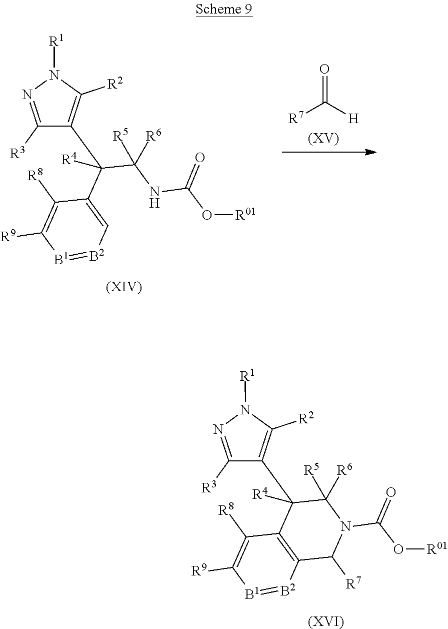

Compounds of formula (XIV), wherein R¹, R², R³, R⁴, R⁵, R⁶, R⁸, R⁹, B¹ and B² are as defined above for the compound of formula (I) and wherein R⁰¹ is as described above, may be prepared by a reaction between amines of formula (XIII), wherein R¹, R², R³, R⁴, R⁵, R⁶, R⁸, R⁹, B¹ and B² are as defined above for the compound of formula (I), and a suitable protecting reagent such as methyl chloroformate, optionally in the presence of a base such as triethylamine or pyridine, in a suitable solvent such as dichloromethane at temperatures between −20° C. and the boiling point of the mixture, as for example described in Organic & Biomolecular Chemistry 2016 14, 6853. This reaction is shown in Scheme 10.

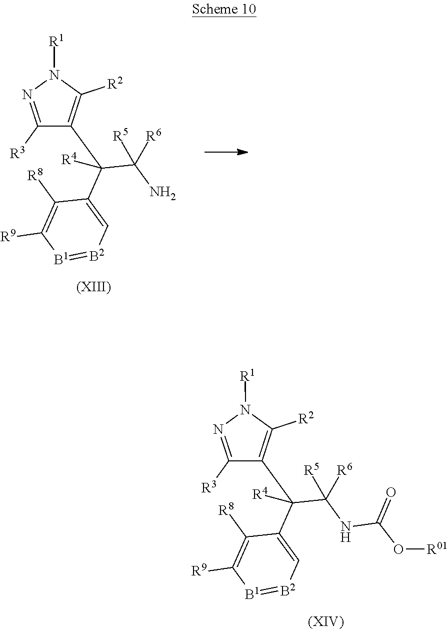

Compounds of formula (XIII), or salts thereof, wherein R¹, R², R³, R⁴, R⁵, R⁶, R⁸, R⁹, B¹ and B² are as defined above for the compound of formula (I), may be prepared by a person skilled in the art by a reaction between nitriles of formula (XII), wherein R¹, R², R³, R⁴, R⁸, R⁹, B¹ and B² are as defined above for the compound of formula (I), and a suitable nucleophile such as (dimethyl sulfide)dihydroboron (BMS) in a suitable aprotic solvent such as tetrahydrofuran, for example as described in The Journal of Organic Chemistry 1981 47, 3153. Alternatively, Grignard reagents R⁵MgBr or R⁶MgBr, wherein R⁵ and R⁶ are as defined above for the compound of formula (I), may be added as nucleophiles to compounds of formula (XII), sequentially or simultaneously, to allow more highly substituted amines of formula (XIII) to be prepared. Such Grignard additions to nitriles are carried out in an inert solvent such as diethyl ether, tert-butylmethyl ether, and cyclopentyl methyl ether in the presence of a Lewis acid such as Ti(O-ⁱPr)₄ (see Synlett (2007) (4), 652-654). This reaction is shown in Scheme 11.

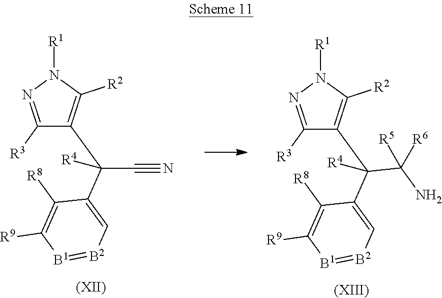

Compounds of formula (XII), wherein R¹, R², R³, R⁴, R⁸, R⁹, B¹ and B² are as defined above for the compound of formula (I), may be prepared by a person skilled in the art following known methods. More specifically, compounds of formula (XII), and intermediates thereof, may be prepared from compounds of formula (XVII) as shown in Scheme 12.

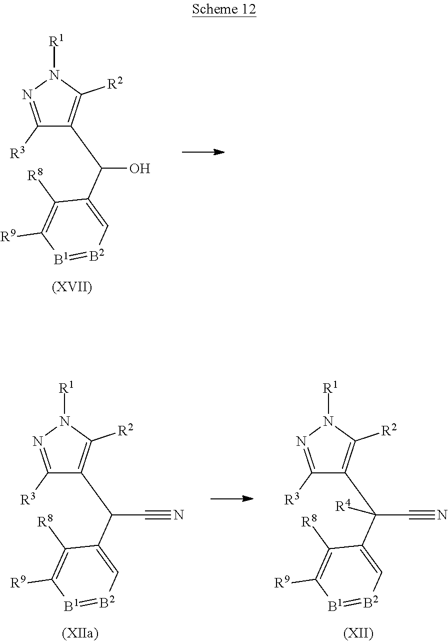

For example, compounds of formula (XII), wherein R¹, R², R³, R⁸, R⁹, B¹ and B² are as defined above for the compound of formula (I) and R⁴ is different from hydrogen, may be prepared by a person skilled in the art by deprotonation of compound of formula (XIIa) wherein R⁴ is hydrogen and R¹, R², R³, R⁸, R⁹, B¹ and B² are as defined above for the compound of formula (I), using a strong base such as n-butyl lithium or sodium hydride at cryogenic temperatures in an inert solvent such as tetrahydrofuran, followed by addition of a suitable alkylating agent R⁴—X, wherein X is halogen, for example iodomethane.

Compounds of formula (XIIa), wherein R⁴ is hydrogen and R¹, R², R³, R⁸, R⁹, B¹ and B² are as defined above for the compound of formula (I), may be prepared from alcohols of formula (XVII) by treatment with cyanotrimethylsilane (TMSCN) in the presence of a base such as lithium carbonate in a nonpolar solvent such as dichloromethane at temperatures between 0° C. and the boiling point of the reaction mixture. Such transformations are well known in the literature under a variety of conditions, for example as described in Organic Letters 2008 10, 4570 and references therein. This reaction is shown in Scheme 12.

Alternatively, compounds of formula (III), wherein R¹, R², R³, R⁴, R⁸, R⁹, B¹ and B² are as defined above for the compound of formula (I), may be prepared from compounds of formula (XVIII), compounds of formula (XIX) or compound of formula (XX) as shown in Scheme 13.

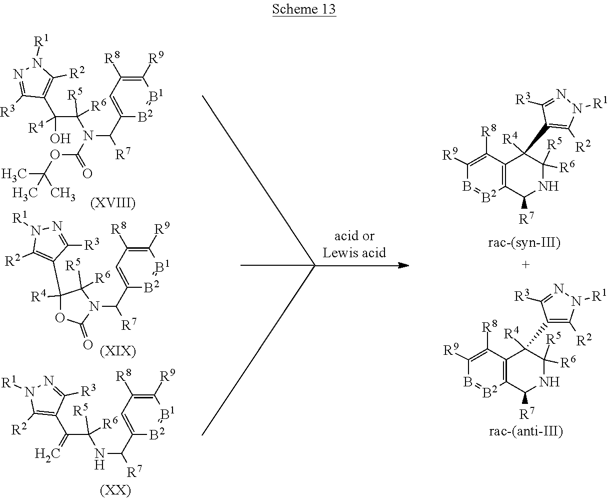

The intermediates compounds (XVIII), (XIX), and (XX) are novel and as such form a further aspect of the invention.

Compounds of formula (III) may be prepared by treating compounds of formula (XVIII), wherein R¹, R², R³, R⁴, R⁵, R⁶, R⁷, R³, R⁹, B¹ and B² correspond to the same definitions as for the compounds of formula (I) according to the present invention with a strong acid. For example compounds of formula (III), wherein B¹ is CR¹⁰, B² is CR¹¹, R¹ is C₁-C₄ alkyl, R² is hydrogen, halogen, or C₁-C₄ alkyl, R³ is hydrogen, R⁴, R⁵, R⁶, R⁷ are hydrogen or C₁-C₄ alkyl, and R⁸, R⁹, R¹⁰, and R¹¹ are as previously defined under formula (I), namely compounds of formula (III-c) may be prepared by treating compounds of formula (XVIII-a)

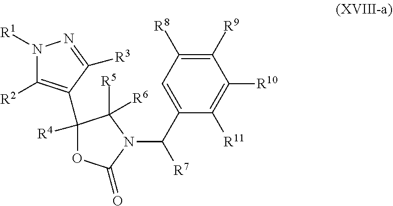

wherein R¹ is C₁-C₄ alkyl, R² is hydrogen, halogen, or C₁-C₄ alkyl, R³ is hydrogen, R⁴, is hydrogen or C₁-C₄ alkyl, R⁵, R⁶, R⁷ are hydrogen or C₁-C₄ alkyl, and R⁸, R⁹, R¹⁰, and R¹¹ are as previously defined under formula (I), with strong acids, for example sulphuric, hydrochloric, hydrobromic, trifluoroacetic, triflic, or methane sulphonic acids and the like, or Lewis acids, such as aluminium trichloride, or bismuth(III)triflate, in an inert solvent such as chlorobenzene, nitrobenzene at temperature between 0° C. to 180° C. to yield compounds of formula (IIIc). These are converted to compounds of formula (I) as previously described vide supra.

Those skilled in the art will realize that such cyclisation's can proceed through intermediates such as compounds of formula (XIX). For example compounds of formula (III-c), wherein B¹ is CR¹⁰, B² is CR¹¹, R¹ is C₁-C₄ alkyl, R² is hydrogen, halogen, or C₁-C₄ alkyl, R³ is hydrogen, R⁴, R⁵, R⁶, R^{7a} are hydrogen or C₁-C₄ alkyl, and R⁸, R⁹, R¹⁰, and R¹¹ are as previously defined under formula (I), may be prepared through cyclisation through intermediates such as compounds of formula (XIX-a) wherein R¹ is C₁-C₄ alkyl, R² is hydrogen, halogen, or C₁-C₄ alkyl, R³ is hydrogen, R⁴, is hydrogen or C₁-C₄ alkyl, R⁵, R⁶, R⁷ are hydrogen or C₁-C₄ alkyl, and R⁸, R⁹, R¹⁰, and R¹¹ are as previously defined under formula (I).

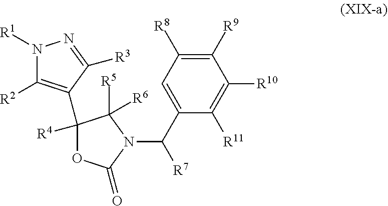

Alternatively compounds of formula (III), wherein R⁴ is methyl and R¹, R², R³, R⁵, R⁶, R⁷, R⁸, R⁹, B¹ and B² correspond to the same definitions as for the compounds of formula (I) may be prepared by treating compounds of formula (XVIII), wherein R⁴ is methyl and R¹, R², R³, R⁵, R⁶, R⁷, R⁸, R⁹, B¹ and B² correspond to the same definitions as for the compounds of formula (I) according to the present invention by treatment with a strong acid and such cyclisation can proceed through intermediates such as compounds of formula (XX), wherein R⁴ is methyl and R¹, R², R³, R⁵, R⁶, R⁷, R⁸, R⁹, B¹ and B² correspond to the same definitions as for the compounds of formula (I) according to the present invention. For example compounds of formula (III-c), wherein B¹ is CR¹⁰, B² is CR¹¹, R¹ is C₁-C₄ alkyl, R² is hydrogen, halogen, or C₁-C₄ alkyl, R³ is hydrogen, R⁴ is methyl, R⁵, R⁶, R^{7a} are hydrogen or C₁-C₄ alkyl, and R⁸, R⁹, R¹⁰, and R¹¹ are as previously defined under formula (I), may be prepared through intermediates such as compounds of formula (XX-a) wherein R¹ is C₁-C₄ alkyl, R² is hydrogen, halogen, or C₁-C₄ alkyl, R³ is hydrogen, R⁴ is C₁-methyl, R⁵, R⁶, R⁷ are hydrogen or C₁-C₄ alkyl, and R⁸, R⁹, R¹⁰, and R¹¹ are as previously defined under formula (I).

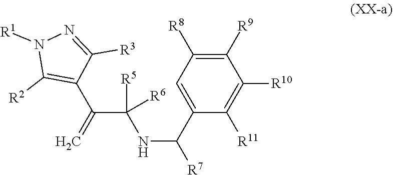

and wherein the substituents R¹, R², R³, R⁵, R⁶, R⁷, R⁸, R⁹, R¹⁰, and R¹¹ in these compounds are as previously described.

Depending on the conditions of the reaction these intermediates can be isolated and/or converted further directly to compounds of formula (III). Those skilled in the art will also realize that when R⁷ is C₁-C₄ alkyl, mixtures of diastereoisomers racemic-(syn-IIIc) and racemic-(anti-IIIc) can be obtained in ratios that can be controlled to direct preferential formation of one isomer over the other (scheme 13). Compounds (XVIII), (XIX), and (XX), wherein R¹, R², R³, R⁴, R⁵, R⁶, R⁷, R⁸, R⁹, B¹ and B² correspond to the same definitions as for the compounds of formula (I) according to the present invention may be prepared from compounds of formula (XXI), which easily prepared by those skilled in the art or can be purchased.

For example compounds of formula (XVIII-a) and (XIX-a), wherein R¹ is C₁-C₄ alkyl, R² is hydrogen, halogen, or C₁-C₄ alkyl, R³ is hydrogen, R⁴, is hydrogen or C₁-C₄ alkyl, R⁵, R⁶, R⁷ are hydrogen or C₁-C₄ alkyl, and R⁸, R⁹, R¹⁰, and R¹¹ are as previously defined under formula (I) can be prepared as shown in the following scheme 14 through Friedel-crafts acylation, followed by Grignard-reaction and cyclisation, and as described in the experimental section. Alternatively compounds of formula (XVIII-b) and (XIX-b), wherein R¹ is C₁-C₄ alkyl, R² is hydrogen, halogen, or C₁-C₄ alkyl, R³ is hydrogen, R⁴ is hydrogen, R⁵, R⁶, R⁷ are hydrogen or C₁-C₄ alkyl, and R⁸, R⁹, R¹⁰, and R¹¹ are as previously defined under formula (I) can be prepared through Friedel-crafts acylation, followed by reduction with a hydride source, for example sodium borohydride, and cyclisation as shown in the following scheme 14, and as described in the experimental section

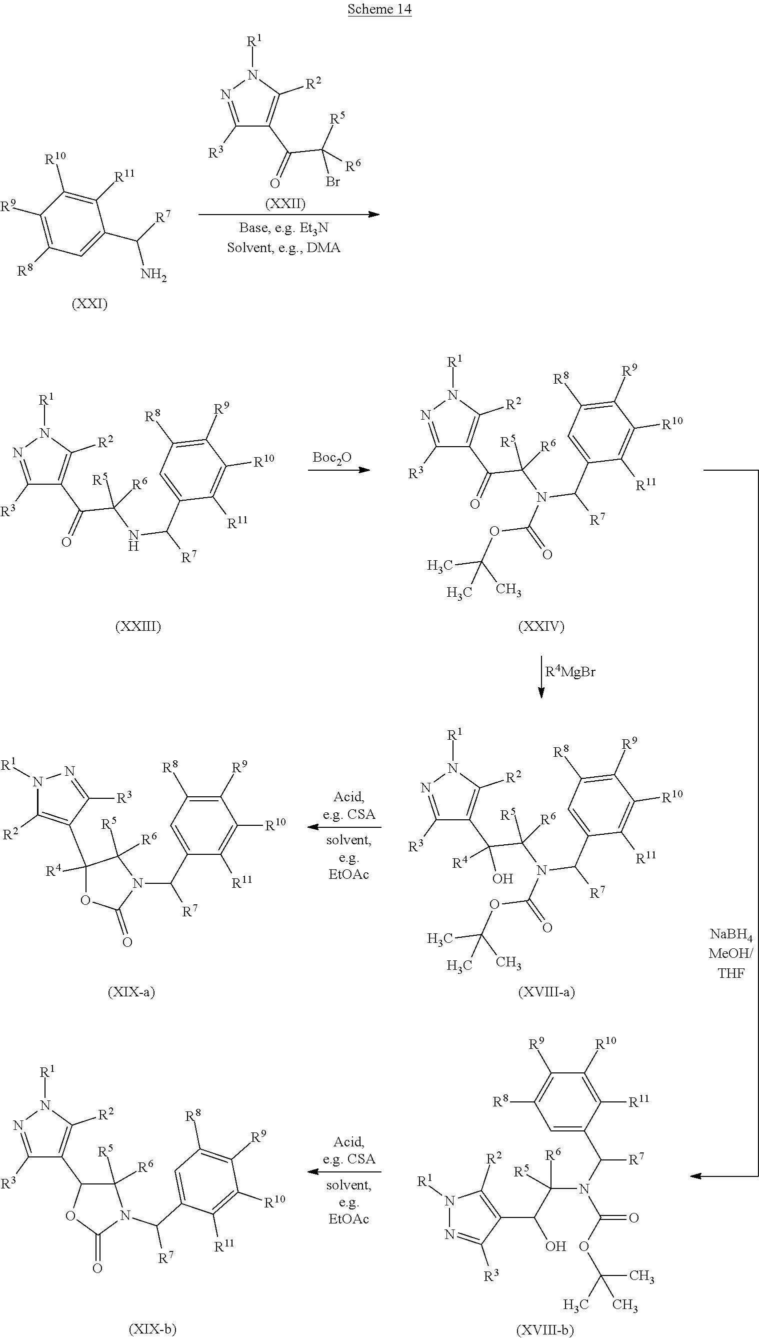

As shown in scheme 14, a benzyl amine of formula (XXI) wherein R⁷, R⁸, R⁹, R¹⁰ and R¹¹ correspond to the same definitions as for the compounds of formula (I) according to the present invention is used to alkylate a compound of formula (XXII), in the presence of a base, such as triethylamine (Et₃N) in an inert solvent, such as DMF or DMA. The compound (XXIII) so obtained, may be isolated, or treated directly with BOC-anhydride (Boc₂-O) in situ, to give a compound of formula (XXIV). Compound of formula XXIV can be reduced with a hydride source such as sodium tetrahydridoborate (NaBH₄) in MeOH/THF, to give the target molecule (XVIII-b), which can then be cyclized with, for example with an acid, e.g. camphor sulphonic acid (CSA) in a solvent such as ethylacetate (EtOAc), to give compounds of formula (XIX-b). Alternatively, compounds of formula (XXIV) can be reacted with a Grignard reagent R⁴MgBr, wherein R⁴ is C₁-C₄ alkyl, in an inert ethereal solvent such as THF to give compounds of formula (XVIII-a), which can the cyclized with an acid, e.g. camphorsulphonic acid in, a solvent such as EtOAc, to give compounds of formula (XIX-a). In compounds (XIX-a) and (XVIII-a) R¹ is C₁-C₄ alkyl, R² is hydrogen, halogen, or C₁-C₄ alkyl, R⁴ is hydrogen or C₁-C₄ alkyl, R⁵, R⁶, and R⁷ are hydrogen or C₁-C₄ alkyl, and R⁸, R⁹, R¹⁰, and R¹¹ are as previously defined under formula (I).

A further aspect of this Friedel-Crafts chemistry should be noted. If the chemistry is carried out starting with a chiral amine (XXI-a), wherein R⁷ is C₁-C₄ alkyl and R⁸, R⁹, R¹⁰ and R¹¹ correspond to the same definitions as for the compounds of formula (I) according to the present invention, the stereochemistry is retained in compound (III-d), wherein R⁷ is C₁-C₄ alkyl and R¹, R², R³, R⁴, R⁵, R⁶, R⁸, R⁹, R¹⁰ and R¹¹ correspond to the same definitions as for the compounds of formula (I) and as well in the final compounds of formula (I). This is illustrated below in scheme 15, for when R⁷ is methyl:

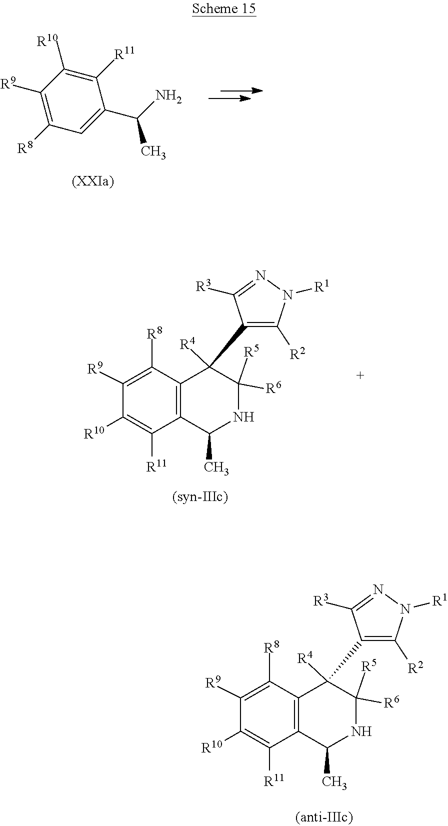

Compounds of formula (II) are commercially available or are readily prepared by compounds known in the state of the art. Compounds of formula (XVII) may be prepared by methods known to a person skilled in the art. Compounds of formula XXI and XXII are easily prepared by those skilled in the art or can be purchased.

Salts of compounds of formula (I) may be prepared in a manner known per se. Thus, for example, acid addition salts of compounds of formula (I) are obtained by treatment with a suitable acid or a suitable ion exchanger reagent and salts with bases are obtained by treatment with a suitable base or with a suitable ion exchanger reagent.

Salts of compounds of formula (I) can be converted in the customary manner into the free compounds (1), acid addition salts, for example, by treatment with a suitable basic compound or with a suitable ion exchanger reagent and salts with bases, for example, by treatment with a suitable acid or with a suitable ion exchanger reagent.

Salts of compounds of formula (I) can be converted in a manner known per se into other salts of compounds of formula (I), acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula (I), which have salt-forming properties, can be obtained in free form or in the form of salts.

The compounds of formula (I) and, where appropriate, the tautomer's thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule, the invention relates to the pure isomers and also to all isomer mixtures which are possible and is to be understood in each case in this sense hereinabove and herein below, even when stereochemical details are not mentioned specifically in each case. Diastereomeric mixtures or racemic mixtures of compounds of formula (I), in free form or in salt form, which can be obtained depending on which starting materials and procedures have been chosen can be separated in a known manner into the pure diastereomers or racemates on the basis of the physicochemical differences of the components, for example by fractional crystallization, distillation and/or chromatography.

Enantiomeric mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents. Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

It is advantageous to isolate or synthesize in each case the biologically more effective isomer, for example enantiomer or diastereomer, or isomer mixture, for example enantiomer mixture or diastereomer mixture, if the individual components have a different biological activity.

As an example, compounds with more than one asymmetric carbon atoms may exist in diastereomeric forms which can be optionally separated using for example supercritical fluid chromatography (SFC) chromatography with chiral columns. Such diastereomers can show a different fungicidal activity profile, but all isomers and diastereomers form part of this invention. The relationship between enantiomers and diastereomers is illustrated in the scheme below (Scheme 16):

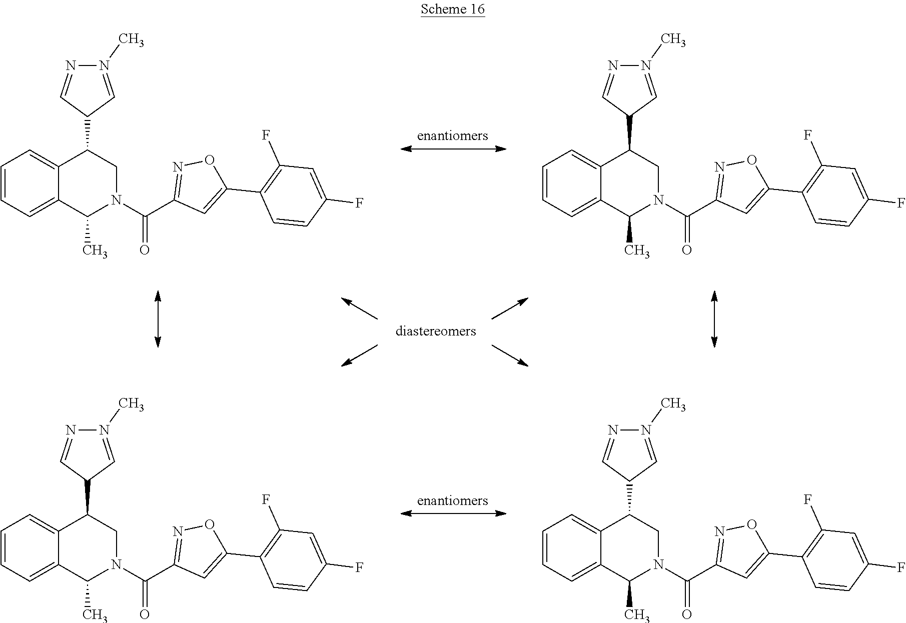

The compounds of formula (I) and, where appropriate, the tautomers thereof, in each case in free form or in salt form, can, if appropriate, also be obtained in the form of hydrates and/or include other solvents, for example those which may have been used for the crystallization of compounds which are present in solid form.

As already indicated, surprisingly, it has now been found that the compounds of formula (I) of the present invention have, for practical purposes, a very advantageous level of biological activity for protecting plants against diseases that are caused by fungi.

The compounds of formula (I) according to the invention can be used in the agricultural sector and related fields of use, e.g., as active ingredients for controlling plant pests or on non-living materials for the control of spoilage microorganisms or organisms potentially harmful to man. The novel compounds are distinguished by excellent activity at low rates of application, by being well tolerated by plants and by being environmentally safe. They have very useful curative, preventive and systemic properties and can be used for protecting numerous cultivated plants. The compounds of formula (I) can be used to inhibit or destroy the pests that occur on plants or parts of plants (fruit, blossoms, leaves, stems, tubers, roots) of different crops of useful plants, while at the same time protecting also those parts of the plants that grow later, e.g., from phytopathogenic microorganisms.

The present invention further relates to a method for controlling or preventing infestation of plants or plant propagation material and/or harvested food crops susceptible to microbial attack by treating plants or plant propagation material and/or harvested food crops wherein an effective amount a compound of formula (I) according to the invention is applied to the plants, to parts thereof or the locus thereof.

It is also possible to use a compound of formula (I) according to the invention as a fungicide. The term “fungicide” as used herein means a compound that controls, modifies, or prevents the growth of fungi. The term “fungicidally effective amount” where used means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

It may also be possible to use compounds of formula (I) according to the invention as dressing agents for the treatment of plant propagation material, e.g., seed, such as fruits, tubers or grains, or plant cuttings, for the protection against fungal infections as well as against phytopathogenic fungi occurring in the soil. The propagation material can be treated with a composition comprising a compound of formula (I) before planting: seed, for example, can be dressed before being sown. The active compounds of formula (I) can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore, the compounds of formula (I) according to the invention can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

The compounds of formula (I) according to the invention are for example, effective against fungi and fungal vectors of disease as well as phytopathogenic bacteria and viruses. These fungi and fungal vectors of disease as well as phytopathogenic bacteria and viruses are for example: Absidia corymbifera, Alternaria spp including Alternaria solani, Aphanomyces spp, Ascochyta spp, Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botryotinia fuckeliana, Botrytis spp. including Botrytis cinerea, Candida spp. including C. albicans, C. glabrata, C. krusei, C. lusitaniae, C. parapsilosis, C. tropicalis, Cephaloascus fragrans, Ceratocystis spp, Cercospora spp. including Cercospora arachidicola, Cercospora kikuchii, Cercospora sojina, Cercosporidium personatum, Cladosporium spp including Cladosporium cucumerinum, Claviceps purpurea, Coccidioides immitis, Cochliobolus spp, Colletotrichum spp. including Colletotrichum musae, Colletotrichum asianum, Corynespora cassiicola, Cryptococcus neoformans, Diaporthe spp, Didymella spp including Didymella bryoniae, Drechslera spp, Elsinoe spp, Epidermophyton spp, Erwinia amylovora, Erysiphe spp. including Erysiphe cichoracearum, Eutypa lata, Fusarium spp. including Fusarium culmorum, Fusarium graminearum, Fusarium langsethiae, Fusarium moniliforme, Fusarium oxysporum, Fusarium proliferatum, Fusarium subglutinans, Fusarium solani, Gaeumannomyces graminis, Gibberella fujikuroi, Gloeodes pomigena, Gloeosporium musarum, Glomerella cingulate, Glomerella lagenarium, Guignardia bidwellii, Gymnosporangium juniperi-virginianae, Helminthosporium spp, Hemileia spp, Histoplasma spp. including H. capsulatum, Laetisaria fuciformis, Leptographium lindbergi, Leveillula taurica, Lophodermium seditiosum, Microdochium nivale, Microsporum spp, Monilinia spp, Mucor spp, Mycosphaerella spp. including Mycosphaerella graminicola, Mycosphaerella pomi, Oncobasidium theobromaeon, Ophiostoma piceae, Paracoccidioides spp, Penicillium spp. including P. digitatum, P. italicum, Petriellidium spp, Peronosclerospora spp. Including P. maydis, P. philippinensis and P. sorghi, Peronospora spp, Phaeosphaeria nodorum, Phakopsora pachyrhizi, Phellinus igniarus, Phialophora spp, Phoma spp, Phomopsis viticola, Phytophthora spp. including P. infestans, Plasmopara spp. including Plasmopara halstedii, Plasmopara viticola, Pleospora spp., Podosphaera spp. including P. leucotricha, Polymyxa graminis, Polymyxa betae, Pseudocercosporella herpotrichoides, Pseudomonas spp, Pseudoperonospora spp. including P. cubensis, P. humuli, Pseudopeziza tracheiphila, Puccinia Spp. including Puccinia hordei, Puccinia recondita, Puccinia striiformis, Puccinia triticina, Pyrenopeziza spp, Pyrenophora spp including Pyrenophora teres, Pyricularia spp. including Pyricularia oryzae, Pythium spp. including P. ultimum, Ramularia spp, Rhizoctonia spp including Rhizoctonia solani, Rhizomucor pusillus, Rhizopus arrhizus, Rhynchosporium spp, Scedosporium spp. including S. apiospermum and S. prolificans, Schizothyrium pomi, Sclerotinia spp including Sclerotinia sclerotiorum, Sclerotium spp, Septoria spp, including Septoria nodorum, Septoria tritici, Sphaerotheca macularis, Sphaerotheca fusca (Sphaerotheca fuliginea), Sporothrix spp, Stagonospora nodorum, Stemphylium spp, Stereum hirsutum, Thanatephorus cucumeris, Thielaviopsis basicola, Tilletia spp, Trichoderma spp. including T. harzianum, T. pseudokoningii, T. viride, Trichophyton spp, Typhula spp, Uncinula necator, Urocystis spp, Ustilago spp, Venturia spp. including Venturia inaequalis, Verticillium spp, and Xanthomonas spp.

The compounds of formula (I) according to the invention may be used for example on turf, ornamentals, such as flowers, shrubs, broad-leaved trees or evergreens, for example conifers, as well as for tree injection, pest management and the like.

Within the scope of present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes. The term “useful plants” is to be understood as also including useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex 1* and LibertyLink®.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard* (maize variety that expresses a CryIA(b) toxin); YieldGard Rootworm* (maize variety that expresses a CryIIIB(b1) toxin); YieldGard Plus* (maize variety that expresses a CryIA(b) and a CryIIIB(b1) toxin); Starlink* (maize variety that expresses a Cry9(c) toxin); Herculex 1* (maize variety that expresses a CryIF(a2) toxin and the enzyme phosphinothricin N-acetyltransferase (PAT) to achieve tolerance to the herbicide glufosinate ammonium); NuCOTN 33B* (cotton variety that expresses a CryIA(c) toxin); Bollgard 1* (cotton variety that expresses a CryIA(c) toxin); Bollgard II® (cotton variety that expresses a CryIA(c) and a CryIIA(b) toxin); VIPCOT* (cotton variety that expresses a VIP toxin); NewLeaf* (potato variety that expresses a CryIIIA toxin); Nature-Gard* Agrisure® GT Advantage (GA21 glyphosate-tolerant trait), Agrisure® CB Advantage (Bt11 corn borer (CB) trait), Agrisure® RW (corn rootworm trait) and Protecta*.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins from Bacillus cereus or Bacillus popilliae; or insecticidal proteins from Bacillus thuringiensis, such as d-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3 hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by δ-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810). Examples of such toxins or transgenic plants capable of synthesising such toxins are disclosed, for example, in EP-A-0 374 753, WO93/07278, WO95/34656, EP-A-0 427 529, EP-A-451 878 and WO 03/052073.

The processes for the preparation of such transgenic plants are generally known to a person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard* (maize variety that expresses a Cry1Ab toxin); YieldGard Rootworm* (maize variety that expresses a Cry3Bb1 toxin); YieldGard Plus* (maize variety that expresses a Cry1Ab and a Cry3Bb1 toxin); Starlink* (maize variety that expresses a Cry9C toxin); Herculex 1* (maize variety that expresses a CryI Fa2 toxin and the enzyme phosphinothricin N-acetyltransferase (PAT) to achieve tolerance to the herbicide glufosinate ammonium); NuCOTN 33B* (cotton variety that expresses a Cry1Ac toxin); Bollgard 1* (cotton variety that expresses a Cry1Ac toxin); Bollgard II® (cotton variety that expresses a Cry1Ac and a Cry2Ab toxin); VipCot* (cotton variety that expresses a Vip3A and a Cry1Ab toxin); NewLeaf* (potato variety that expresses a Cry3A toxin); Nature-Gard*, Agrisure® GT Advantage (GA21 glyphosate-tolerant trait), Agrisure® CB Advantage (Bt11 corn borer (CB) trait) and Protecta*.

Further examples of such transgenic crops are:
1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B 1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

The compounds of formula (I) according to the invention may be used in controlling or preventing phytopathogenic diseases, especially phytopathogenic fungi such as Alternaria solani, Blumeria graminis, Botryotinia fuckeliana, Botrytis cinerea, Cercospora arachidicola, Cercospora kikuchii, Cercospora sojina, Cladosporium cucumerinum, Colletotrichum lagenarium, Corynespora cassiicola, Didymella bryoniae, Fusarium spp, Glomerella lagenarium, Leptosphaeria spp, Leveillula taurica, Microdochium nivale, Plasmopara viticola, Puccinia recondita, Pyrenophora teres, Pyricularia oryzae, Rhizoctonia solani, Sclerotinia sclerotiorum, Septoria nodorum, Septoria tritici, Sphaerotheca fuliginea, Uncinula necator and Venturia inaequalis. In an embodiment of the present invention, the compounds of formula (I) according to the invention may be used in controlling or preventing phytopathogenic diseases, especially phytopathogenic fungi such as Septoria tritici, Pyrenophora teres, Puccinia recondita and Blumeria graminis in cereals; Cercospora arachidicola and Sclerotinia sclerotiorum in field crops; Alternaria solani in fruits and vegetables, e.g. tomatoes and potatoes; Botrytis cinerea in fruits, vegetables and field crops, e.g. strawberries, tomatoes, sunflower, legumes and grapes; Glomerella lagenarium in vegetables, e.g. cucumbers; Uncinula necator in vegetables, e.g. grapes; Venturia inaequalis in fruits, e.g. apples; Rhizoctonia solani in vegetables, e.g. potatoes; Cladosporium cucumerinum, Didymella bryoniae and Sphaerotheca fuliginea in vegetables, e.g. cucumbers; Leveillula taurica in cucumbers and solanaceous vegetables; Fusarium spp in cereals and vegetables; Leptosphaeria spp in cereals.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There can be mentioned for example seeds (in the strict sense), roots, fruits, tubers, bulbs, rhizomes and parts of plants. Germinated plants and young plants which are to be transplanted after germination or after emergence from the soil, may also be mentioned. These young plants can be protected before transplantation by a total or partial treatment by immersion. Preferably “plant propagation material” is understood to denote seeds.

The compounds of formula (I) according to the invention may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from 0.5% to 95% of the concentrate. Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils, and/or stickers such as dextrins, glue or synthetic resins. Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates.

Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and carriers that are useful in formulating the compositions of the invention in the formulation types described above are well known to a person skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc., ethylene glycol, propylene glycol, glycerine and N-methyl-2-pyrrolidinone. Water is generally the carrier of choice for the dilution of concentrates.

Suitable solid carriers include, for example, talc, titanium dioxide, pyrophyllite clay, silica, attapulgite clay, kieselguhr, chalk, diatomaceous earth, lime, calcium carbonate, bentonite clay, fuller's earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2 ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants and sticking agents.

In addition, further, other biocidally active ingredients or compositions may be combined with the compositions of the invention and used in the methods of the invention and applied simultaneously or sequentially with the compositions of the invention. When applied simultaneously, these further active ingredients may be formulated together with the compositions of the invention or mixed in, for example, the spray tank. These further biocidally active ingredients may be fungicides, herbicides, insecticides, bactericides, acaricides, nematicides and/or plant growth regulators.

Pesticidal agents are referred to herein using their common name are known, for example, from “The Pesticide Manual”, 15th Ed., British Crop Protection Council 2009.

In addition, the compositions of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-S-methyl.

The compounds of formula (I) according to the invention are normally used in the form of agrochemical compositions and can be applied to the crop area or plant to be treated, simultaneously or in succession with further compounds. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula (I) according to the invention may be used in the form of (fungicidal) compositions for controlling or protecting against phytopathogenic microorganisms, comprising as active ingredient at least one compound of formula (I) or of at least one preferred individual compound as defined herein, in free form or in agrochemically usable salt form, and at least one of the above-mentioned adjuvants.

The invention therefore provides a composition, preferably a fungicidal composition, comprising at least one compound of formula (I) according to the invention, an agriculturally acceptable carrier and optionally an adjuvant. An agricultural acceptable carrier is for example a carrier that is suitable for agricultural use. Agricultural carriers are well known in the art. Preferably, said composition may comprise at least one or more pesticidally-active compounds, for example an additional fungicidal active ingredient in addition to the compound of formula (I).

The compound of formula (I) according to the invention may be the sole active ingredient of a composition or it may be admixed with one or more additional active ingredients such as a pesticide, fungicide, synergist, herbicide or plant growth regulator where appropriate. An additional active ingredient may, in some cases, result in unexpected synergistic activities.

Examples of suitable additional active ingredients include the following: acylamino acid fungicides, aliphatic nitrogen fungicides, amide fungicides, anilide fungicides, antibiotic fungicides, aromatic fungicides, arsenical fungicides, aryl phenyl ketone fungicides, benzamide fungicides, benzanilide fungicides, benzimidazole fungicides, benzothiazole fungicides, botanical fungicides, bridged diphenyl fungicides, carbamate fungicides, carbanilate fungicides, corazole fungicides, copper fungicides, dicarboximide fungicides, dinitrophenol fungicides, dithiocarbamate fungicides, dithiolane fungicides, furamide fungicides, furanilide fungicides, hydrazide fungicides, imidazole fungicides, mercury fungicides, morpholine fungicides, organophosphorous fungicides, organotin fungicides, oxathiin fungicides, oxazole fungicides, phenylsulfamide fungicides, polysulfide fungicides, pyrazole fungicides, pyridine fungicides, pyrimidine fungicides, pyrrole fungicides, quaternary ammonium fungicides, quinoline fungicides, quinone fungicides, quinoxaline fungicides, strobilurin fungicides, sulfonanilide fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, valinamide fungicides, and zinc fungicides. Examples of suitable additional active ingredients include the following: petroleum oils, 1,1-bis(4-chloro-phenyl)-2-ethoxyethanol, 2,4-dichlorophenyl benzenesulfonate, 2-fluoro-N-methyl-N-1-naphthylacetamide, 4-chlorophenyl phenyl sulfone, acetoprole, aldoxycarb, amidation, amidothioate, amiton, amiton hydrogen oxalate, amitraz, aramite, arsenous oxide, azobenzene, azothoate, benomyl, benoxa-fos, benzyl benzoate, bixafen, brofenvalerate, bromo-cyclen, bromophos, bromopropylate, buprofezin, butocarboxim, butoxycarboxim, butylpyridaben, calcium polysulfide, camphechlor, carbanolate, carbophenothion, cymiazole, chino-methionat, chlorbenside, chlordimeform, chlordimeform hydrochloride, chlorfenethol, chlorfenson, chlorfensulfide, chlorobenzilate, chloromebuform, chloromethiuron, chloropropylate, chlorthiophos, cinerin I, cinerin II, cinerins, closantel, coumaphos, crotamiton, crotoxyphos, cufraneb, cyanthoate, DCPM, DDT, demephion, demephion-O, demephion-S, demeton-methyl, demeton-O, demeton-O-methyl, demeton-S, demeton-S-methyl, demeton-S-methylsulfon, dichlofluanid, dichlorvos, dicliphos, dienochlor, dimefox, dinex, dinex-diclexine, dinocap-4, dinocap-6, dinocton, dino-penton, dinosulfon, dinoterbon, dioxathion, diphenyl sulfone, disulfiram, DNOC, dofenapyn, doramectin, endothion, eprinomectin, ethoate-methyl, etrimfos, fenazaflor, fenbutatin oxide, fenothiocarb, fenpyrad, fen-pyroximate, fenpyrazamine, fenson, fentrifanil, flubenzimine, flucycloxuron, fluenetil, fluorbenside, FMC 1137, formetanate, formetanate hydrochloride, formparanate, gamma-HCH, glyodin, halfenprox, hexadecyl cyclopropanecarboxylate, isocarbophos, jasmolin I, jasmolin II, jodfenphos, lindane, malonoben, mecarbam, mephosfolan, mesulfen, methacrifos, methyl bromide, metolcarb, mexacarbate, milbemycin oxime, mipafox, monocrotophos, morphothion, moxidectin, naled, 4-chloro-2-(2-chloro-2-methyl-propyl)-5-[(6-iodo-3-pyridyl)methoxy]pyridazin-3-one, nifluridide, nikkomycins, nitrilacarb, nitrilacarb 1:1 zinc chloride complex, omethoate, oxydeprofos, oxydisulfoton, pp′-DDT, parathion, permethrin, phenkapton, phosalone, phosfolan, phosphamidon, polychloroterpenes, polynactins, proclonol, promacyl, propoxur, prothidathion, prothoate, pyrethrin I, pyrethrin II, pyrethrins, pyridaphenthion, pyrimitate, quinalphos, quintiofos, R-1492, phosglycin, rotenone, schradan, sebufos, selamectin, sophamide, SSI-121, sulfiram, sulfluramid, sulfotep, sulfur, diflovidazin, tau-fluvalinate, TEPP, terbam, tetradifon, tetrasul, thiafenox, thiocarboxime, thiofanox, thiometon, thioquinox, thuringiensin, triamiphos, triarathene, triazophos, triazuron, trifenofos, trinactin, vamidothion, vaniliprole, bethoxazin, copper dioctanoate, copper sulfate, cybutryne, dichlone, dichlorophen, endothal, fentin, hydrated lime, nabam, quinoclamine, quinonamid, simazine, triphenyltin acetate, triphenyltin hydroxide, crufomate, piperazine, thiophanate, chloralose, fenthion, pyridin-4-amine, strychnine, 1-hydroxy-1H-pyridine-2-thione, 4-(quinoxalin-2-ylamino)benzenesulfonamide, 8-hydroxyquinoline sulfate, bronopol, copper hydroxide, cresol, dipyrithione, dodicin, fenaminosulf, formaldehyde, hydrargaphen, kasugamycin, kasugamycin hydrochloride hydrate, nickel bis(dimethyldithiocarbamate), nitrapyrin, octhilinone, oxolinic acid, oxytetracycline, potassium hydroxyquinoline sulfate, probenazole, streptomycin, streptomycin sesquisulfate, tecloftalam, thiomersal, Adoxophyes orana GV, Agrobacterium radiobacter, Amblyseius spp., Anagrapha falcifera NPV, Anagrus atomus, Aphelinus abdominalis, Aphidius colemani, Aphidoletes aphidimyza, Autographa californica NPV, Bacillus sphaericus Neide, Beauveria brongniartii, Chrysoperla carnea, Cryptolaemus montrouzieri, Cydia pomonella GV, Dacnusa sibirica, Diglyphus isaea, Encarsia formosa, Eretmocerus eremicus, Heterorhabditis bacteriophora and H. megidis, Hippodamia convergens, Leptomastix dactylopii, Macrolophus caliginosus, Mamestra brassicae NPV, Metaphycus helvolus, Metarhizium anisopliae var. acridum, Metarhizium anisopliae var. anisopliae, Neodiprion sertifer NPV and N. lecontei NPV, Orius spp., Paecilomyces fumosoroseus, Phytoseiulus persimilis, Steinernema bibionis, Steinernema carpocapsae, Steinernema feltiae, Steinernema glaseri, Steinernema riobrave, Steinernema riobravis, Steinernema scapterisci, Steinernema spp., Trichogramma spp., Typhlodromus occidentalis, Verticillium lecanii, apholate, bisazir, busulfan, dimatif, hemel, hempa, metepa, methiotepa, methyl apholate, morzid, penfluron, tepa, thiohempa, thiotepa, tretamine, uredepa, (E)-dec-5-en-1-yl acetate with (E)-dec-5-en-1-ol, (E)-tridec-4-en-1-yl acetate, (E)-6-methylhept-2-en-4-ol, (E,Z)-tetradeca-4,10-dien-1-yl acetate, (Z)-dodec-7-en-1-yl acetate, (Z)-hexadec-11-enal, (Z)-hexadec-11-en-1-yl acetate, (Z)-hexadec-13-en-11-yn-1-yl acetate, (Z)-icos-13-en-10-one, (Z)-tetradec-7-en-1-al, (Z)-tetradec-9-en-1-ol, (Z)-tetradec-9-en-1-yl acetate, (7E,9Z)-dodeca-7,9-dien-1-yl acetate, (9Z,11E)-tetradeca-9,11-dien-1-yl acetate, (9Z,12E)-tetradeca-9,12-dien-1-yl acetate, 14-methyloctadec-1-ene, 4-methylnonan-5-ol with 4-methylnonan-5-one, alpha-multistriatin, brevicomin, codlelure, codlemone, cuelure, disparlure, dodec-8-en-1-yl acetate, dodec-9-en-1-yl acetate, dodeca-8,10-dien-1-yl acetate, dominicalure, ethyl 4-methyloctanoate, eugenol, frontalin, grandlure, grandlure I, grandlure II, grandlure III, grandlure IV, hexalure, ipsdienol, ipsenol, japonilure, lineatin, litlure, looplure, medlure, megatomoic acid, methyl eugenol, muscalure, octadeca-2,13-dien-1-yl acetate, octadeca-3,13-dien-1-yl acetate, orfralure, oryctalure, ostramone, siglure, sordidin, sulcatol, tetradec-11-en-1-yl acetate, trimedlure, trimedlure A, trimedlure B1, trimedlure B2, trimedlure C, trunc-call, 2-(octylthio)-ethanol, butopyronoxyl, butoxy(polypropylene glycol), dibutyl adipate, dibutyl phthalate, dibutyl succinate, diethyltoluamide, dimethyl carbate, dimethyl phthalate, ethyl hexanediol, hexaamide, methoquin-butyl, methylneodecanamide, oxamate, picaridin, 1-dichloro-1-nitroethane, 1,1-dichloro-2,2-bis(4-ethylphenyl)-ethane, 1,2-dichloropropane with 1,3-dichloropropene, 1-bromo-2-chloroethane, 2,2,2-trichloro-1-(3,4-dichloro-phenyl)ethyl acetate, 2,2-dichlorovinyl 2-ethylsulfinylethyl methyl phosphate, 2-(1,3-dithiolan-2-yl)phenyl dimethylcarbamate, 2-(2-butoxyethoxy)ethyl thiocyanate, 2-(4,5-dimethyl-1,3-dioxolan-2-yl)phenyl methylcarbamate, 2-(4-chloro-3,5-xylyloxy)ethanol, 2-chlorovinyl diethyl phosphate, 2-imidazolidone, 2-isovalerylindan-1,3-dione, 2-methyl(prop-2-ynyl)aminophenyl methylcarbamate, 2-thiocyanatoethyl laurate, 3-bromo-1-chloroprop-1-ene, 3-methyl-1-phenylpyrazol-5-yl dimethyl-carbamate, 4-methyl(prop-2-ynyl)amino-3,5-xylyl methylcarbamate, 5,5-dimethyl-3-oxocyclohex-1-enyl dimethylcarbamate, acethion, acrylonitrile, aldrin, allosamidin, allyxycarb, alpha-ecdysone, aluminium phosphide, aminocarb, anabasine, athidathion, azamethiphos, Bacillus thuringiensis delta endotoxins, barium hexafluorosilicate, barium polysulfide, barthrin, Bayer 22/190, Bayer 22408, beta-cyfluthrin, beta-cypermethrin, bioethanomethrin, biopermethrin, bis(2-chloroethyl) ether, borax, bromfenvinfos, bromo-DDT, bufencarb, butacarb, butathiofos, butonate, calcium arsenate, calcium cyanide, carbon disulfide, carbon tetrachloride, cartap hydrochloride, cevadine, chlorbicyclen, chlordane, chlordecone, chloroform, chloropicrin, chlorphoxim, chlorprazophos, cis-resmethrin, cismethrin, clocythrin, copper acetoarsenite, copper arsenate, copper oleate, coumithoate, cryolite, CS 708, cyanofenphos, cyanophos, cyclethrin, cythioate, d-tetramethrin, DAEP, dazomet, decarbofuran, diamidafos, dicapthon, dichlofenthion, dicresyl, dicyclanil, dieldrin, diethyl 5-methylpyrazol-3-yl phosphate, dilor, dimefluthrin, dimetan, dimethrin, dimethylvinphos, dimetilan, dinoprop, dinosam, dinoseb, diofenolan, dioxabenzofos, dithicrofos, DSP, ecdysterone, EI 1642, EMPC, EPBP, etaphos, ethiofencarb, ethyl formate, ethylene dibromide, ethylene dichloride, ethylene oxide, EXD, fenchlorphos, fenethacarb, fenitrothion, fenoxacrim, fenpirithrin, fensulfothion, fenthion-ethyl, flucofuron, fosmethilan, fospirate, fosthietan, furathiocarb, furethrin, guazatine, guazatine acetates, sodium tetrathiocarbonate, halfenprox, HCH, HEOD, heptachlor, heterophos, HHDN, hydrogen cyanide, hyquincarb, IPSP, isazofos, isobenzan, isodrin, isofenphos, isolane, isoprothiolane, isoxathion, juvenile hormone I, juvenile hormone II, juvenile hormone III, kelevan, kinoprene, lead arsenate, leptophos, lirimfos, lythidathion, m-cumenyl methylcarbamate, magnesium phosphide, mazidox, mecarphon, menazon, mercurous chloride, mesulfenfos, metam, metam-potassium, metam-sodium, methanesulfonyl fluoride, methocrotophos, methoprene, methothrin, methoxychlor, methyl isothiocyanate, methylchloroform, methylene chloride, metoxadiazone, mirex, naftalofos, naphthalene, NC-170, nicotine, nicotine sulfate, nithiazine, nornicotine, O-5-dichloro-4-iodophenyl O-ethyl ethylphosphonothioate, O,O-diethyl O-4-methyl-2-oxo-2H-chromen-7-yl phosphorothioate, O,O-diethyl O-6-methyl-2-propylpyrimidin-4-yl phosphorothioate, O,O,O′,O′-tetrapropyl dithiopyrophosphate, oleic acid, para-dichlorobenzene, parathion-methyl, pentachlorophenol, pentachlorophenyl laurate, PH 60-38, phenkapton, phosnichlor, phosphine, phoxim-methyl, pirimetaphos, polychlorodicyclopentadiene isomers, potassium arsenite, potassium thiocyanate, precocene I, precocene II, precocene III, primidophos, profluthrin, promecarb, prothiofos, pyrazophos, pyresmethrin, quassia, quinalphos-methyl, quinothion, rafoxanide, resmethrin, rotenone, kadethrin, ryania, ryanodine, sapodilla), schradan, sebufos, SI-0009, thiapronil, sodium arsenite, sodium cyanide, sodium fluoride, sodium hexafluorosilicate, sodium pentachlorophenoxide, sodium selenate, sodium thiocyanate, sulcofuron, sulcofuron-sodium, sulfuryl fluoride, sulprofos, tar oils, tazimcarb, TDE, tebupirimfos, temephos, terallethrin, tetrachloroethane, thicrofos, thiocyclam, thiocyclam hydrogen oxalate, thionazin, thiosultap, thiosultap-sodium, tralomethrin, transpermethrin, triazamate, trichlormetaphos-3, trichloronat, trimethacarb, tolprocarb, triclopyricarb, triprene, veratridine, veratrine, XMC, zetamethrin, zinc phosphide, zolaprofos, and meperfluthrin, tetramethylfluthrin, bis(tributyltin) oxide, bromoacetamide, ferric phosphate, niclosamide-olamine, tributyltin oxide, pyrimorph, trifenmorph, 1,2-dibromo-3-chloropropane, 1,3-dichloropropene, 3,4-dichlorotetrahydrothio-phene 1,1-dioxide, 3-(4-chlorophenyl)-5-methylrhodanine, 5-methyl-6-thioxo-1,3,5-thiadiazinan-3-ylacetic acid, 6-isopentenylaminopurine, 2-fluoro-N-(3-methoxyphenyl)-9H-purin-6-amine, benclothiaz, cytokinins, DCIP, furfural, isamidofos, kinetin, Myrothecium verrucaria composition, tetrachlorothiophene, xylenols, zeatin, potassium ethylxanthate, acibenzolar, acibenzolar-S-methyl, Reynoutria sachalinensis extract, alpha-chlorohydrin, antu, barium carbonate, bisthiosemi, brodifacoum, bromadiolone, bromethalin, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, norbormide, phosacetim, phosphorus, pindone, pyrinuron, scilliroside, sodium fluoro-acetate, thallium sulfate, warfarin, 2-(2-butoxyethoxy)-ethyl piperonylate, 5-(1,3-benzodioxol-5-yl)-3-hexylcyclohex-2-enone, farnesol with nerolidol, verbutin, MGK 264, piperonyl butoxide, piprotal, propyl isomer, S421, sesamex, sesasmolin, sulfoxide, anthraquinone, copper naphthenate, copper oxychloride, dicyclopentadiene, thiram, zinc naphthenate, ziram, imanin, ribavirin, mercuric oxide, thiophanate-methyl, azaconazole, bitertanol, bromuconazole, cyproconazole, difenoconazole, diniconazole, epoxicon-azole, fenbuconazole, fluquinconazole, flusilazole, flutriafol, furametpyr, hexaconazole, imazalil, imiben-con-azole, ipconazole, metconazole, myclobutanil, paclobutrazole, pefurazoate, penconazole, prothioconazole, pyrifenox, prochloraz, propiconazole, pyrisoxazole, simeconazole, tebucon-azole, tetraconazole, triadimefon, triadime-nol, triflumizole, triticonazole, ancymidol, fenarimol, nuarimol, bupirimate, dimethirimol, ethirimol, dodemorph, fenpropidin, fenpropimorph, spiroxamine, tridemorph, cyprodinil, mepanipyrim, pyrimethanil, fenpiclonil, fludioxonil, benalaxyl, furalaxyl, meta-laxyl, R-metalaxyl, ofurace, oxadixyl, carbendazim, debacarb, fuberidazole, thiaben-dazole, chlozolinate, dichlozoline, myclozoline, procymi-done, vinclozoline, boscalid, carboxin, fenfuram, flutolanil, mepronil, oxycarboxin, penthiopyrad, thifluzamide, dodine, iminoctadine, azoxystrobin, dimoxystrobin, enestroburin, fenaminstrobin, flufenoxystrobin, fluoxastrobin, kresoxim-methyl, metomi-nostrobin, trifloxystrobin, orysastrobin, picoxystrobin, pyraclostrobin, pyrametostrobin, pyraoxystrobin, ferbam, mancozeb, maneb, metiram, propineb, zineb, captafol, captan, fluoroimide, folpet, tolylfluanid, bordeaux mixture, copper oxide, mancopper, oxine-copper, nitrothal-isopropyl, edifenphos, iprobenphos, phosdiphen, tolclofos-methyl, anilazine, benthiavalicarb, blasticidin-S, chloroneb, chloro-tha-lo-nil, cyflufenamid, cymoxanil, cyclobutrifluram, diclocymet, diclomezine, dicloran, diethofencarb, dimetho-morph, flumorph, dithianon, ethaboxam, etridiazole, famoxa-done, fenamidone, fenoxanil, ferimzone, fluazinam, fluopicolide, flusulfamide, fluxapyroxad, fenhexamid, fos-etyl-aluminium, hymexazol, iprovalicarb, cyazofamid, methasulfo-carb, metrafenone, pencycuron, phthalide, polyoxins, propamocarb, pyribencarb, proquinazid, pyroquilon, pyriofenone, quinoxyfen, quintozene, tiadinil, triazoxide, tricyclazole, triforine, validamycin, valifenalate, zoxamide, mandipropamid, flubeneteram, isopyrazam, sedaxane, benzovindiflupyr, pydiflumetofen, 3-difluoromethyl-1-methyl-1H-pyrazole-4-carboxylic acid (3′,4′,5′-trifluoro-biphenyl-2-yl)-amide, isoflucypram, isotianil, dipymetitrone, 6-ethyl-5,7-dioxo-pyrrolo[4,5][1,4]dithiino[1,2-c]isothiazole-3-carbonitrile, 2-(difluoromethyl)-N-[3-ethyl-1,1-dimethyl-indan-4-yl]pyridine-3-carboxamide, 4-(2,6-difluorophenyl)-6-methyl-5-phenyl-pyridazine-3-carbonitrile, (R)-3-(difluoromethyl)-1-methyl-N-[1,1,3-trimethylindan-4-yl]pyrazole-4-carboxamide, 4-(2-bromo-4-fluoro-phenyl)-N-(2-chloro-6-fluoro-phenyl)-2,5-dimethyl-pyrazol-3-amine, 4-(2-bromo-4-fluorophenyl)-N—(2-chloro-6-fluorophenyl)-1,3-dimethyl-1H-pyrazol-5-amine, fluindapyr, coumethoxystrobin (jiaxiangjunzhi), Ivbenmixianan, dichlobentiazox, mandestrobin, 3-(4,4-difluoro-3,4-dihydro-3,3-dimethylisoquinolin-1-yl)quinolone, 2-[2-fluoro-6-[(8-fluoro-2-methyl-3-quinolyl)oxy]phenyl]propan-2-ol, oxathiapiprolin, tert-butyl N-[6-[[[(1-methyltetrazol-5-yl)-phenyl-methylene]amino]oxymethyl]-2-pyridyl]carbamate, pyraziflumid, inpyrfluxam, trolprocarb, mefentrifluconazole, ipfentrifluconazole, 2-(difluoromethyl)-N-[(3R)-3-ethyl-1,1-dimethyl-indan-4-yl]pyridine-3-carboxamide, N′-(2,5-dimethyl-4-phenoxy-phenyl)-N-ethyl-N-methyl-formamidine, N′-[4-(4,5-dichlorothiazol-2-yl)oxy-2,5-dimethyl-phenyl]-N-ethyl-N-methyl-formamidine, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate, but-3-ynyl N-[6-[[(Z)-[(1-methyltetrazol-5-yl)-phenyl-methylene]amino]oxymethyl]-2-pyridyl]carbamate, methyl N-[[5-[4-(2,4-dimethylphenyl)triazol-2-yl]-2-methyl-phenyl]methyl]carbamate, 3-chloro-6-methyl-5-phenyl-4-(2,4,6-trifluorophenyl)pyridazine, pyridachlometyl, 3-(difluoromethyl)-1-methyl-N-[1,1,3-trimethylindan-4-yl]pyrazole-4-carboxamide, 1-[2-[[1-(4-chlorophenyl)pyrazol-3-yl]oxymethyl]-3-methyl-phenyl]-4-methyl-tetrazol-5-one, 1-methyl-4-[3-methyl-2-[[2-methyl-4-(3,4,5-trimethylpyrazol-1-yl)phenoxy]methyl]phenyl]tetrazol-5-one, aminopyrifen, ametoctradin, amisulbrom, penflufen, (Z,2E)-5-[1-(4-chlorophenyl)pyrazol-3-yl]oxy-2-methoxyimino-N,3-dimethyl-pent-3-enamide, florylpicoxamid, fenpicoxamid, tebufloquin, ipflufenoquin, quinofumelin, isofetamid, N-[2-[2,4-dichloro-phenoxy]phenyl]-3-(difluoromethyl)-1-methyl-pyrazole-4-carboxamide, N-[2-[2-chloro-4-(trifluoromethyl)phenoxy]phenyl]-3-(difluoromethyl)-1-methyl-pyrazole-4-carboxamide, benzothiostrobin, phenamacril, 5-amino-1,3,4-thiadiazole-2-thiol zinc salt (2:1), fluopyram, flutianil, fluopimomide, pyrapropoyne, picarbutrazox, 2-(difluoromethyl)-N-(3-ethyl-1,1-dimethyl-indan-4-yl)pyridine-3-carboxamide, 2-(difluoromethyl)-N—((3R)-1,1,3-trimethylindan-4-yl)pyridine-3-carboxamide, 4-[[6-[2-(2,4-difluorophenyl)-1,1-difluoro-2-hydroxy-3-(1,2,4-triazol-1-yl)propyl]-3-pyridyl]oxy]benzonitrile, metyltetraprole, 2-(difluoromethyl)-N—((3R)-1,1,3-trimethylindan-4-yl)pyridine-3-carboxamide, α-(1,1-dimethylethyl)-α-[4′-(trifluoromethoxy) [1,1′-biphenyl]-4-yl]-5-pyrimidinemethanol, fluoxapiprolin, enoxastrobin, 4-[[6-[2-(2,4-difluorophenyl)-1,1-difluoro-2-hydroxy-3-(1,2,4-triazol-1-yl)propyl]-3-pyridyl]oxy]benzonitrile, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile, trinexapac, coumoxystrobin, zhongshengmycin, thiodiazole copper, zinc thiazole, amectotractin, iprodione, N-octyl-N′-[2-(octylamino)ethyl]ethane-1,2-diamine, N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-bromo-2-methyl-6-(1-methyl-2-propoxy-ethoxy)-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075); N′-[5-bromo-2-methyl-6-(2-propoxypropoxy)-3-pyridyl]-N-ethyl-N-methyl-formamidine (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896); N-ethyl-N′-[5-methoxy-2-methyl-4-[(2-trifluoromethyl)oxetan-2-yl]phenyl]-N-methyl-formamidine, N-ethyl-N′-[5-methoxy-2-methyl-4-[(2-trifluoromethyl)tetrahydrofuran-2-yl]phenyl]-N-methyl-formamidine (these compounds may be prepared from the methods described in WO2019/110427); N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide (these compounds may be prepared from the methods described in WO2017/153380); 1-(6,7-dimethylpyrazolo[1,5-a]pyridin-3-yl)-4,4,5-trifluoro-3,3-dimethyl-isoquinoline, 1-(6,7-dimethylpyrazolo[1,5-a]pyridin-3-yl)-4,4,6-trifluoro-3,3-dimethyl-isoquinoline, 4,4-difluoro-3,3-dimethyl-1-(6-methylpyrazolo[1,5-a]pyridin-3-yl)isoquinoline, 4,4-difluoro-3,3-dimethyl-1-(7-methylpyrazolo[1,5-a]pyridin-3-yl)isoquinoline, 1-(6-chloro-7-methyl-pyrazolo[1,5-a]pyridin-3-yl)-4,4-difluoro-3,3-dimethyl-isoquinoline (these compounds may be prepared from the methods described in WO2017/025510); 1-(4,5-dimethylbenzimidazol-1-yl)-4,4,5-trifluoro-3,3-dimethyl-isoquinoline, 1-(4,5-dimethylbenzimidazol-1-yl)-4,4-difluoro-3,3-dimethyl-isoquinoline, 6-chloro-4,4-difluoro-3,3-dimethyl-1-(4-methylbenzimidazol-1-yl)isoquinoline, 4,4-difluoro-1-(5-fluoro-4-methyl-benzimidazol-1-yl)-3,3-dimethyl-isoquinoline, 3-(4,4-difluoro-3,3-dimethyl-1-isoquinolyl)-7,8-dihydro-6H-cyclopenta[e]benzimidazole (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 3-ethyl-1-methoxy-1-[[4-[5-(trifluoromethyl)-1,2,4-oxadiazol-3-yl]phenyl]methyl]urea, N-[[4-[5-(trifluoromethyl)-1,2,4-oxadiazol-3-yl]phenyl]methyl]propanamide, 4,4-dimethyl-2-[[4-[5-(trifluoromethyl)-1,2,4-oxadiazol-3-yl]phenyl]methyl]isoxazolidin-3-one, 5,5-dimethyl-2-[[4-[5-(trifluoromethyl)-1,2,4-oxadiazol-3-yl]phenyl]methyl]isoxazolidin-3-one, ethyl 1-[[4-[5-(trifluoromethyl)-1,2,4-oxadiazol-3-yl]phenyl]methyl]pyrazole-4-carboxylate, N,N-dimethyl-1-[[4-[5-(trifluoromethyl)-1,2,4-oxadiazol-3-yl]phenyl]methyl]-1,2,4-triazol-3-amine (these compounds may be prepared from the methods described in WO 2017/055473, WO 2017/055469, WO 2017/093348 and WO 2017/118689); 2-[6-(4-chlorophenoxy)-2-(trifluoromethyl)-3-pyridyl]-1-(1,2,4-triazol-1-yl)propan-2-ol (this compound may be prepared from the methods described in WO 2017/029179); 2-[6-(4-bromophenoxy)-2-(trifluoromethyl)-3-pyridyl]-1-(1,2,4-triazol-1-yl)propan-2-ol (this compound may be prepared from the methods described in WO 2017/029179); 3-[2-(1-chlorocyclopropyl)-3-(2-fluorophenyl)-2-hydroxy-propyl]imidazole-4-carbonitrile (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile (this compound may be prepared from the methods described in WO 2016/156290); (4-phenoxyphenyl)methyl 2-amino-6-methyl-pyridine-3-carboxylate (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone (this compound may be prepared from the methods described in WO 2011/138281) N-methyl-4-[5-(trifluoromethyl)-1,2,4-oxadiazol-3-yl]benzenecarbothioamide; N-methyl-4-[5-(trifluoromethyl)-1,2,4-oxadiazol-3-yl]benzamide; (Z,2E)-5-[1-(2,4-dichlorophenyl)pyrazol-3-yl]oxy-2-methoxyimino-N,3-dimethyl-pent-3-enamide (this compound may be prepared from the methods described in WO 2018/153707); N′-(2-chloro-5-methyl-4-phenoxy-phenyl)-N-ethyl-N-methyl-formamidine; N′-[2-chloro-4-(2-fluorophenoxy)-5-methyl-phenyl]-N-ethyl-N-methyl-formamidine (this compound may be prepared from the methods described in WO 2016/202742); 2-(difluoromethyl)-N-[(3S)-3-ethyl-1,1-dimethyl-indan-4-yl]pyridine-3-carboxamide (this compound may be prepared from the methods described in WO 2014/095675); (5-methyl-2-pyridyl)-[4-[5-(trifluoromethyl)-1,2,4-oxadiazol-3-yl]phenyl]methanone, (3-methylisoxazol-5-yl)-[4-[5-(trifluoromethyl)-1,2,4-oxadiazol-3-yl]phenyl]methanone (these compounds may be prepared from the methods described in WO 2017/220485); 2-oxo-N-propyl-2-[4-[5-(trifluoromethyl)-1,2,4-oxadiazol-3-yl]phenyl]acetamide (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate (this compound may be prepared from the methods described in WO 2018/158365); 2,2-difluoro-N-methyl-2-[4-[5-(trifluoromethyl)-1,2,4-oxadiazol-3-yl]phenyl]acetamide, N—[(E)-methoxyiminomethyl]-4-[5-(trifluoromethyl)-1,2,4-oxadiazol-3-yl]benzamide, N—[(Z)-methoxyiminomethyl]-4-[5-(trifluoromethyl)-1,2,4-oxadiazol-3-yl]benzamide, N—[N-methoxy-C-methyl-carbonimidoyl]-4-[5-(trifluoromethyl)-1,2,4-oxadiazol-3-yl]benzamide (these compounds may be prepared from the methods described in WO 2018/202428).

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds (I) for the preparation of these compositions are also a subject of the invention.

Another aspect of the invention is related to the use of a compound of formula (I) according to the invention or of a preferred individual compound as defined herein, of a composition comprising at least one compound of formula (I) or at least one preferred individual compound as defined herein, or of a fungicidal or insecticidal mixture comprising at least one compound of formula (I) or at least one preferred individual compound as defined herein, in admixture with other fungicides or insecticides as described above, for controlling or preventing infestation of plants, e.g. useful plants such as crop plants, propagation material thereof, e.g. seeds, harvested crops, e.g. harvested food crops, or non-living materials by insects or by phytopathogenic microorganisms, preferably fungal organisms.

A further aspect of invention is related to a method of controlling or preventing an infestation of plants, e.g. useful plants such as crop plants, propagation material thereof, e.g. seeds, harvested crops, e.g. harvested food crops, or of non-living materials by phytopathogenic or spoilage microorganisms or organisms potentially harmful to man, especially fungal organisms, which comprises the application of a compound of formula (I) according to the invention or of a preferred individual compound as defined herein as active ingredient to the plants, to parts of the plants or to the locus thereof, to the propagation material thereof, or to any part of the non-living materials.

Controlling or preventing means reducing infestation by insects or by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, or insects which comprises the application of a compound of formula (I) according to the invention, or an agrochemical composition which contains at least one compound of formula (I), is foliar application. The frequency of application and the rate of application will depend on the risk of infestation by the corresponding pathogen or insect. However, the compounds of formula (I) according to the invention can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula (I) may also be applied to seeds (coating) by impregnating the seeds or tubers either with a liquid formulation of the fungicide or coating them with a solid formulation.

A formulation, e.g. a composition containing the compound of formula (I) according to the invention and, if desired, a solid or liquid adjuvant or monomers for encapsulating the compound of formula (I), may be prepared in a known manner, typically by intimately mixing and/or grinding the compound with extenders, for example solvents, solid carriers and, optionally, surface active compounds (surfactants).

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds. When the combinations of the present invention are used for treating seed, rates of 0.001 to 50 g of a compound of formula (I) per kg of seed, preferably from 0.01 to 10 g per kg of seed are generally sufficient.

Suitably, a composition comprising a compound of formula (I) according to the present invention is applied either preventative, meaning prior to disease development or curative, meaning after disease development.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula (I) according to the invention optionally together with other active agents, particularly microbicides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent.

Whereas commercial products will preferably be formulated as concentrates, the end user will normally employ diluted formulations.

Whereas it is preferred to formulate commercial products as concentrates, the end user will normally use dilute formulations.

The disclosure in the present application makes available each and every combination of embodiments disclosed herein.

The compounds according to the following Tables A-1 to A-32 may be prepared according to the methods described above. The examples which follow are intended to illustrate the invention and show preferred compounds of formula (I). In any of Tables A-1 to A-32 below, the presence of one or more possible asymmetric carbon atoms in a compound of formula (I) according to the invention means that the compounds may occur in chiral isomeric forms, i.e., enantiomeric or diastereomeric forms.

TABLE A
This table discloses 12 compounds of formula (Ia) according to the
invention:
(Ia)

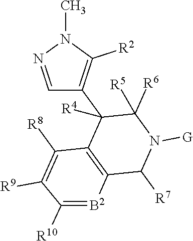

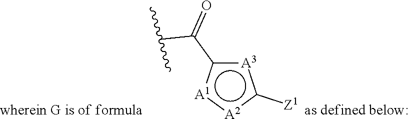

| Index | G |
|---|---|
| G1 | [5-(2,4-difluorophenyl)isoxazole-3- |
| | carbonyl] |
| G2 | [5-(4-fluorophenyl)isoxazole-3- |
| | carbonyl] |
| G3 | [5-(2,4-dichlorophenyl)isoxazole-3- |
| | carbonyl] |
| G4 | [5-(4-chlorophenyl)isoxazole-3- |
| | carbonyl] |
| G5 | [3-(2,4-difluorophenyl)isoxazole-5- |
| | carbonyl] |
| G6 | [3-(4-fluorophenyl)isoxazole-5- |
| | carbonyl] |
| G7 | [3-(2,4-dichlorophenyl)isoxazole-5- |
| | carbonyl] |
| G8 | [3-(4-chlorophenyl)isoxazole-5- |
| | carbonyl] |
| G9 | [5-(2,4-difluorophenyl)-1,3,4- |
| | thiadiazole-2-carbonyl] |
| G10 | [5-(4-fluorophenyl)-1,3,4-thiadiazole- |
| | 2-carbonyl] |
| G11 | [5-(2,4-dichlorophenyl)-1,3,4- |
| | thiadiazole-2-carbonyl] |
| G12 | [5-(4-chlorophenyl)-1,3,4-thiadiazole- |
| | 2-carbonyl] |

The following compounds represent specific compounds or formula (Ia) as described in Tables A-1 to A-32 wherein G is as defined in Table A. For instance, the compound A-1.G1 represents a compound of formula (Ia) wherein R², R⁴, R⁵, R⁶, R⁷, R⁸, R⁹, R¹⁰ and B² are as defined in Table A-1 and G is G1 as defined in Table A.

Table A-1: This table provides 12 compounds A-1.G1 to A-1.G12 of formula (Ia) wherein R², R⁴, R⁵, R⁶, R⁷, R⁸, R⁹, R¹⁰ are H, B² is CH and G is as defined in Table A. For example, compound A-1.G9 has the following structure:

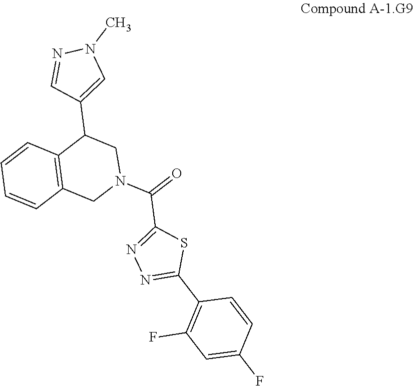 [SMILES: Cn1cc(C2CN(C(=O)c3nnc(-c4ccc(F)cc4F)s3)Cc3ccccc32)cn1]

Table A-2: This table provides 12 compounds A-2.G1 to A-2.G12 of formula (Ia) wherein R² is CH₃, R⁴, R⁵, R⁶, R⁷, R⁸, R⁹, R¹⁰ are H, B² is CH and G is as defined in Table A. For example, compound A-2.G1 has the following structure:

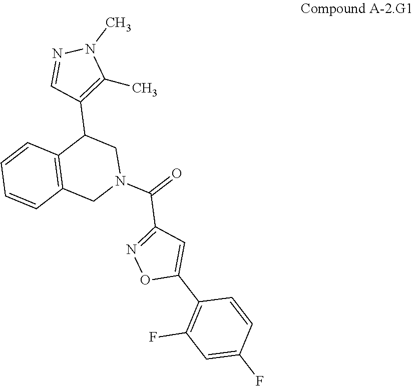 [SMILES: Cc1c(C2CN(C(=O)c3cc(-c4ccc(F)cc4F)on3)Cc3ccccc32)cnn1C]

Table A-3: This table provides 12 compounds A-3.G1 to A-3.G12 of formula (Ia) wherein R² is F, R⁴, R⁵, R⁶, R⁷, R⁸, R⁹, R¹⁰ are H, B² is CH and G is as defined in Table A.

Table A-4: This table provides 12 compounds A-4.G1 to A-4.G12 of formula (Ia) wherein R² is C₁, R⁴, R⁵, R⁶, R⁷, R⁸, R⁹, R¹⁰ are H, B² is CH and G is as defined in Table A.

Table A-5: This table provides 12 compounds A-5.G1 to A-5.G12 of formula (Ia) wherein R² is cyclopropyl, R⁴, R⁵, R⁶, R⁷, R⁸, R⁹, R¹⁰ are H, B² is CH and G is as defined in Table A.

Table A-6: This table provides 12 compounds A-6.G1 to A-6.G12 of formula (Ia) wherein R² is COCH₃, R⁴, R⁵, R⁶, R⁷, R⁸, R⁹, R¹⁰ are H, B² is CH and G is as defined in Table A.

Table A-7: This table provides 12 compounds A-7.G1 to A-7.G12 of formula (Ia) wherein R² is C═N(OCH₃)CH₃, R⁴, R⁵, R⁶, R⁷, R⁸, R⁹, R¹⁰ are H, B² is CH and G is as defined in Table A.

Table A-8: This table provides 12 compounds A-8.G1 to A-8.G12 of formula (Ia) wherein R², R⁵, R⁶, R⁷, R⁸, R⁹, R¹⁰ are H, R⁴ is CH₃, B² is CH and G is as defined in Table A. For example, compound A-8.G1 has the following structure:

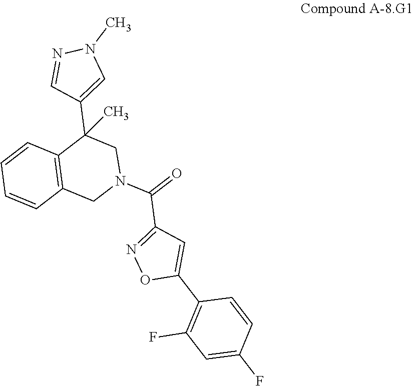 [SMILES: Cn1cc(C2(C)CN(C(=O)c3cc(-c4ccc(F)cc4F)on3)Cc3ccccc32)cn1]

Table A-9: This table provides 12 compounds A-9.G1 to A-9.G12 of formula (Ia) wherein R⁵, R⁶, R⁷, R⁸, R⁹, R¹⁰ are H, R² and R⁴ are CH₃, B² is CH and G is as defined in Table A.

Table A-10: This table provides 12 compounds A-10.G1 to A-10.G12 of formula (Ia) wherein R⁵, R⁶, R⁷, R⁸, R⁹, R¹⁰ are H, R² and R⁴ are CH₃, B² is N and G is as defined in Table A.

Table A-11: This table provides 6 compounds A-11.G1, A-11.G2, A-11.G5, A-11.G6, A-11.G9 and A-11.G10 of formula (Ia) wherein R², R⁴ and R⁷ are CH₃, R⁵, R⁶, R⁸, R⁹, R¹⁰ are H, B² is CH and G is as defined in Table A. For example, compound A-11.G5 has the following structure:

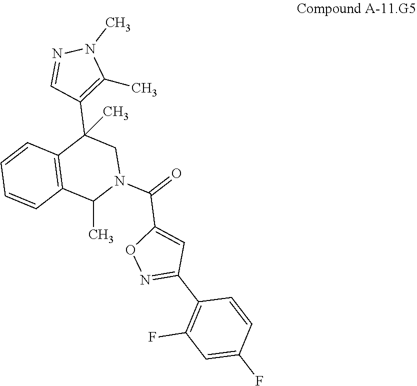 [SMILES: Cc1c(C2(C)CN(C(=O)c3cc(-c4ccc(F)cc4F)no3)C(C)c3ccccc32)cnn1C]

Table A-12: This table provides 6 compounds A-12.G1, A-12.G2, A-12.G5, A-12.G6, A-12.G9 and A-12.G10 of formula (Ia) wherein R² is F, R⁴ and R⁷ are CH₃, R⁵, R⁶, R⁸, R⁹, R¹⁰ are H, B² is CH and G is as defined in Table A.

Table A-13: This table provides 6 compounds A-13.G1, A-13.G2, A-13.G5, A-13.G6, A-13.G9 and A-13.G10 of formula (Ia) wherein R² is C₁, R⁴ and R⁷ are CH₃, R⁵, R⁶, R⁸, R⁹, R¹⁰ are H, B² is CH and G is as defined in Table A.

Table A-14: This table provides 6 compounds A-14.G1, A-14.G2, A-14.G5, A-14.G6, A-14.G9 and A-14.G10 of formula (Ia) wherein R² is cyclopropyl, R⁴ and R⁷ are CH₃, R⁵, R⁶, R⁸, R⁹, R¹⁰ are H, B² is CH and G is as defined in Table A.

Table A-15: This table provides 6 compounds A-15.G1, A-15.G2, A-15.G5, A-15.G6, A-15.G9 and A-15.G10 of formula (Ia) wherein R², R⁴ and R⁷ are CH₃, R⁵, R⁶, R⁸, R⁹, R¹⁰ are H, B² is N and G is as defined in Table A.

Table A-16: This table provides 6 compounds A-16.G1, A-16.G2, A-16.G5, A-16.G6, A-16.G9 and A-16.G10 of formula (Ia) wherein R², R⁴ and R⁷ are CH₃, R⁵, R⁶, R⁹, R¹⁰ are H, R⁸ is OCH₃, B² is CH and G is as defined in Table A.

Table A-17: This table provides 6 compounds A-17.G1, A-17.G2, A-17.G5, A-17.G6, A-17.G9 and A-17.G10 of formula (Ia) wherein R², R⁴ and R⁷ are CH₃, R⁵, R⁶, R⁸, R¹⁰ are H, R⁹ is OCH₃, B² is CH and G is as defined in Table A.

Table A-18: This table provides 6 compounds A-18.G1, A-18.G2, A-18.G5, A-18.G6, A-18.G9 and A-18.G10 of formula (Ia) wherein R², R⁴ and R⁷ are CH₃, R⁵, R⁶, R⁸, R¹⁰ are H, R⁹ is OH, B² is CH and G is as defined in Table A.

Table A-19: This table provides 6 compounds A-19.G1, A-19.G2, A-19.G5, A-19.G6, A-19.G9 and A-19.G10 of formula (Ia) wherein R², R⁴ and R⁷ are CH₃, R⁵, R⁶, R⁸, R¹⁰ are H, R⁹ is CF₃, B² is CH and G is as defined in Table A.

Table A-20: This table provides 6 compounds A-20.G1, A-20.G2, A-20.G5, A-20.G6, A-20.G9 and A-20.G10 of formula (Ia) wherein R², R⁴ and R⁷ are CH₃, R⁵, R⁶, R⁸, R¹⁰ are H, R⁹ is CN, B² is CH and G is as defined in Table A.

Table A-21: This table provides 6 compounds A-21.G1, A-21.G2, A-21.G5, A-21.G6, A-21.G9 and A-21.G10 of formula (Ia) wherein R², R⁴ and R⁷ are CH₃, R⁵, R⁶, R⁸, R⁹ are H, R¹⁰ is OCH₃, B² is CH and G is as defined in Table A.

Table A-22: This table provides 6 compounds A-22.G1, A-22.G2, A-22.G5, A-22.G6, A-22.G9 and A-22.G10 of formula (Ia) wherein R², R⁴ and R⁷ are CH₃, R⁵, R⁶, R⁸, R⁹ are H, R¹⁰ is OH, B² is CH and G is as defined in Table A.

Table A-23: This table provides 6 compounds A-23.G1, A-23.G2, A-23.G5, A-23.G6, A-23.G9 and A-23.G10 of formula (Ia) wherein R², R⁴ and R⁷ are CH₃, R⁵, R⁶, R⁸, R⁹ are H, R¹⁰ is CF₃, B² is CH and G is as defined in Table A.

Table A-24: This table provides 6 compounds A-24.G1, A-24.G2, A-24.G5, A-24.G6, A-24.G9 and A-24.G10 of formula (Ia) wherein R², R⁴ and R⁷ are CH₃, R⁵, R⁶, R⁸, R⁹ are H, R¹⁰ is CN, B² is CH and G is as defined in Table A.

Table A-25: This table provides 6 compounds A-25.G1, A-25.G2, A-25.G5, A-25.G6, A-25.G9 and A-25.G10 of formula (Ia) wherein R², R⁴ and R⁷ are CH₃, R⁵, R⁶, R⁸ are H, R⁹ is OCH₃, R¹⁰ is CF₃, B² is CH and G is as defined in Table A.

Table A-26: This table provides 6 compounds A-26.G1, A-26.G2, A-26.G5, A-26.G6, A-26.G9 and A-26.G10 of formula (Ia) wherein R², R⁴ and R⁷ are CH₃, R⁵, R⁶, R⁸ are H, R⁹ is OCH₃, R¹⁰ is CN, B² is CH and G is as defined in Table A.

Table A-27: This table provides 6 compounds A-27.G1, A-27.G2, A-27.G5, A-27.G6, A-27.G9 and A-27.G10 of formula (Ia) wherein R², R⁴ and R⁷ are CH₃, R⁵, R⁶, R⁸ are H, R⁹ is CF₃, R¹⁰ is OCH₃, B² is CH and G is as defined in Table A.

Table A-28: This table provides 6 compounds A-28.G1, A-28.G2, A-28.G5, A-28.G6, A-28.G9 and A-28.G10 of formula (Ia) wherein R², R⁴ and R⁷ are CH₃, R⁵, R⁶, R⁸ are H, R⁹ is CN, R¹⁰ is OCH₃, B² is CH and G is as defined in Table A.

Table A-29: This table provides 6 compounds A-29.G1, A-29.G2, A-29.G5, A-29.G6, A-29.G9 and A-29.G10 of formula (Ia) wherein R², R⁵, R⁶ and R⁷ are CH₃, R⁴, R⁸, R⁹, R¹⁰ are H, B² is CH and G is as defined in Table A.

Table A-30: This table provides 6 compounds A-30.G1, A-30.G2, A-30.G5, A-30.G6, A-30.G9 and A-30.G10 of formula (Ia) wherein R², R⁴, R⁵, R⁶ and R⁷ are CH₃, R⁸, R⁹, R¹⁰ are H, B² is CH and G is as defined in Table A.

Table A-31: This table provides 6 compounds A-31.G1, A-31.G2, A-31.G5, A-31.G6, A-31.G9 and A-31.G10 of formula (Ia) wherein R², R⁴ and R⁷ are CH₃, R⁵, R⁶, R⁹, R¹⁰ are H, R⁸ is F, B² is CH and G is as defined in Table A.

Table A-32: This table provides 6 compounds A-32.G1, A-32.G2, A-32.G5, A-32.G6, A-32.G9 and A-32.G10 of formula (Ia) wherein R², R⁴ and R⁷ are CH₃, R⁵, R⁶, R⁸, R⁹, R¹⁰ are H, B² is CF and G is as defined in Table A.

### EXAMPLES

The Examples which follow serve to illustrate the invention and are not meant in any way to limit the invention.

The compounds of the invention can be distinguished from known compounds by virtue of greater efficacy at low application rates, which can be verified by a person skilled in the art using the experimental procedures outlined in the Examples, using lower application rates if necessary, for example 60 ppm, 20 ppm or 2 ppm.

Compounds of formula (I) may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile (including improved crop tolerance), improved physico-chemical properties, or increased biodegradability).

Throughout this description, temperatures are given in degrees Celsius and “m.p.” means melting point. LC-MS means Liquid Chromatography Mass Spectroscopy and the description of the apparatus and the methods is as follows.

¹H NMR measurements were recorded on a Brucker 400 MHz spectrometer, chemical shifts are given in ppm relevant to a TMS standard. Spectra measured in deuterated solvents as indicated. Either one of the LC-MS methods below was used to characterize the compounds. The characteristic LC-MS values obtained for each compound were the retention time (“Rt”, recorded in minutes) and the measured molecular ion (M+H)⁺ or (M−H)⁻.

### Method A:

Spectra were recorded on a Mass Spectrometer from Waters Corporation (SQD, SQDII or QDA Single quadrupole mass spectrometer) equipped with an electrospray source (Polarity: positive and negative ions), Capillary: 0.8-3.00 kV, Cone: 5-30 V, Source Temperature: 120-150° C., Desolvation Temperature: 350-600° C., Cone Gas Flow: 50-150 l/h, Desolvation Gas Flow: 650-1000 l/h, Mass range: 100 to 900 Da and an Acquity UPLC from Waters Corporation: Binary pump, heated column compartment, diode-array detector and ELSD. Column: Waters UPLC HSS T3, 1.8 μm, 30×2.1 mm, Temp: 60° C., DAD Wavelength range (nm): 210 to 400, Runtime: 1.5 min; Solvents: A=water+5% MeOH+0.05% HCOOH, B=Acetonitrile+0.05% HCOOH; Flow (ml/min) 0.85, Gradient: 10% B isocratic for 0.2 min, then 10-100% B in 1.0 min, 100% B isocratic for 0.2 min, 100-10% B in 0.05 min, 10% B isocratic for 0.05 min.

Method B: Spectra were recorded on a ACQUITY Mass Spectrometer from Waters (SQD or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30×2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 2.5 min; Flow (ml/min) 0.75.

### Method C:

### Instrumentation:

Mass Spectrometer: Acquity QDA Mass Spectrometer from Waters
HPLC: UPLC ‘H’ class

### Optimized Mass Parameter:

Ionisation method: Electrospray (ESI)
Polarity: Positive and Negative Polarity Switch
Scan Type: Full Scan
Capillary (kV): 0.8
Cone Voltage (V): 25.00
Source Temperature (° C.): 120
Desolvation Gas Flow (L/Hr): 1000
Desolvation Temperature (° C.): 600
Gas Flow @Cone (L/Hr): 50
Mass range: 110 to 850 Da
PDA Wavelength range: 230 to 400 nm

### Optimized Chromatographic Parameter:

### Gradient Conditions:

Solvent A: Water with 0.1% formic acid:Acetonitrile: 95:5 v/v
Solvent B: Acetonitrile with 0.05% formic acid

| Time (minutes) | A (%) | B (%) | Flow rate (ml/min) |
|---|---|---|---|
| 0 | 90 | 10 | 0.6 |
| 0.2 | 90 | 10 | 0.6 |
| 0.3 | 50 | 50 | 0.6 |
| 0.6 | 0 | 100 | 0.6 |
| 1.3 | 0 | 100 | 0.6 |
| 1.4 | 90 | 10 | 0.6 |
| 1.6 | 90 | 10 | 0.6 |

Column: Acquity UPLC HSS T3 C18
Column length: 30 mm
Internal diameter of column: 2.1 mm
Particle Size: 1.8μ
Column oven temperature: 40° C.

### Method D:

### Instrumentation:

Mass Spectrometer: Acquity SQD Mass Spectrometer from Waters
HPLC: UPLC ‘H’ class

### Optimized Mass Parameter:

Ionisation method: Electrospray (ESI)
Polarity: Positive and Negative Polarity Switch
Scan Type: Full Scan
Capillary (kV): 3.00
Cone Voltage (V): 41.00
Source Temperature (° C.): 150
Desolvation Gas Flow (L/Hr): 1000
Desolvation Temperature (° C.): 500
Gas Flow @Cone (L/Hr): 50
Mass range: 110 to 800 Da
PDA Wavelength range: 210 to 400 nm

### Optimized Chromatographic Parameter:

### Gradient Conditions:

Solvent A: Water with 0.1% formic acid:Acetonitrile: 95:5 v/v
Solvent B: Acetonitrile with 0.05% formic acid

| Time (minutes) | A (%) | B (%) | Flow rate (ml/min) |
|---|---|---|---|
| 0.0 | 90 | 10 | 0.6 |
| 0.2 | 90 | 10 | 0.6 |
| 0.3 | 50 | 50 | 0.6 |
| 0.6 | 0 | 100 | 0.6 |
| 1.3 | 0 | 100 | 0.6 |
| 1.4 | 90 | 10 | 0.6 |
| 1.6 | 90 | 10 | 0.6 |

Column: Acquity UPLC HSS T3 C18
Column length: 30 mm
Internal diameter of column: 2.1 mm
Particle Size: 1.8μ
Column oven temperature: 40° C.

### Method E:

### Instrumentation:

Mass Spectrometer: 6410 Triple Quadruple Mass Spectrometer from Agilent Technologies
HPLC: Agilent 1200 Series HPLC

### Optimized Mass Parameter:

Ionisation method: Electrospray (ESI)
Polarity: Positive and Negative Polarity Switch
Scan Type: MS2 Scan
Capillary (kV): 4.00
Fragmentor (V): 100.00
Gas Temperature (° C.): 350
Gas Flow (L/min): 11
Nebulizer Gas (psi): 40
Mass range: 110 to 1000 Da
Detection(VWD): 254 nm

### Optimized Chromatographic Parameter:

### Gradient Conditions:

Solvent A: Water with 0.1% formic acid:Acetonitrile: 95:5 v/v
Solvent B: Acetonitrile with 0.1% formic acid

| Time (minutes) | A (%) | B (%) | Flow rate (ml/min) |
|---|---|---|---|
| 0 | 90 | 10 | 1.8 |
| 0.9 | 0 | 100 | 1.8 |
| 1.8 | 0 | 100 | 1.8 |
| 2.2 | 90 | 10 | 1.8 |
| 2.5 | 90 | 10 | 1.8 |

Column: KINETEX EVO C18
Column length: 50 mm
Internal diameter of column: 4.6 mm
Particle Size: 2.6μ
Column oven temperature: 40° C.

### PREPARATION EXAMPLES

The compounds of formula (I) according to the invention may be prepared using the synthetic techniques described both above and below.

### Example P1: Preparation of [5-(2,4-difluorophenyl)isoxazol-3-yl]-[4-(1-methylpyrazol-4-yl)-3,4-dihydro-1H-isoquinolin-2-yl]methanone (Compound P-180, Table T1)

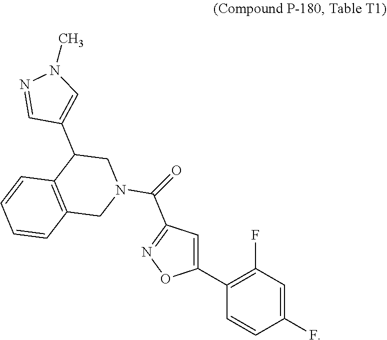 [SMILES: Cn1cc(C2CN(C(=O)c3cc(-c4ccc(F)cc4F)on3)Cc3ccccc32)cn1]

### Step 1: Preparation of 4-(1-methylpyrazol-4-yl)isoquinoline

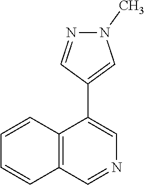 [SMILES: Cn1cc(-c2cncc3ccccc23)cn1]

In a microwave vial, a suspension of 4-bromoisoquinoline (200 mg, 0.942 mmol), 1-methylpyrazole-4-boronic acid hydrochloride (234 mg, 1.41 mmol, 1.50 equiv.) and cesium carbonate (1.23 g, 3.77 mmol, 4.00 equiv.) in 1,4-dioxane (2.8 mL) and water (0.47 mL) was degassed with argon for several minutes, and then 1,1′-bis(diphenylphosphino)ferrocene-palladium(II)dichloride dichloromethane complex (40 mg, 0.047 mmol, 0.050 equiv.) was added. The vial was sealed, and the reaction mixture was heated at 120° C. and stirred for 1 hour under microwave irradiation. After cooling down to room temperature, the reaction mixture was partitioned between an ammonium chloride saturated solution and dichloromethane, the organic layer was separated, and the aqueous layer was extracted twice with dichloromethane. The combined organic layers were dried over sodium sulfate, filtered and concentrated in vacuo. Purification of the crude material by flash chromatography over silica gel (eluting with ethyl acetate in cyclohexane) afforded the title compound as a brown oil (115 mg, 0.550 mmol).

LC-MS (Method A): retention time 0.41 min, m/z 210 [M+H+].

¹H NMR (400 MHz, chloroform-d) δ ppm: 4.06 (s, 3H) 7.62-7.66 (m, 1H) 7.66-7.68 (m, 1H) 7.70-7.76 (m, 1H) 7.78-7.81 (m, 1H) 8.03 (d, J=8.07 Hz, 1H) 8.10-8.18 (m, 1H) 8.51 (s, 1H) 9.20 (s, 1H).

### Step 2: Preparation of 4-(1-methylpyrazol-4-yl)-1,2,3,4 tetrahydroisoquinoline Hydrochloride

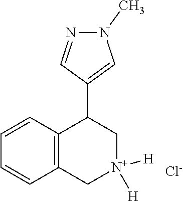 [SMILES: Cn1cc(C2C[NH2+]Cc3ccccc32)cn1 ; [Cl-]]

To a solution of 4-(1-methylpyrazol-4-yl)isoquinoline (prepared as described above in step 1, 115 mg, 0.550 mmol) in methanol (5.5 mL) was added at room temperature sodium cyanoborohydride (218 mg, 3.30 mmol, 6.00 equiv.). The reaction mixture was stirred at room temperature, and then hydrochloric acid (1.25 M in methanol) was added until the pH reached 2-3. After 30 minutes of stirring at room temperature, the reaction mixture was diluted with water and basified with 2N sodium hydroxide. Methanol was evaporated under reduced pressure and the aqueous layer was extracted three times with dichloromethane. The combined organic layers were dried over sodium sulfate, filtered and concentrated in vacuo. The resulting brown oil was treated with 2 M HCl in diethyl ether and concentrated under reduced pressure to afford the title compound (160 mg, 0.642 mmol) which was used in the next step without further purification.

### Step 3: Preparation of [5-(2,4-difluorophenyl)isoxazol-3-yl]-[4-(1-methylpyrazol-4-yl)-3,4-dihydro-1H-isoquinolin-2-yl]methanone (Compound P-180, Table T1)

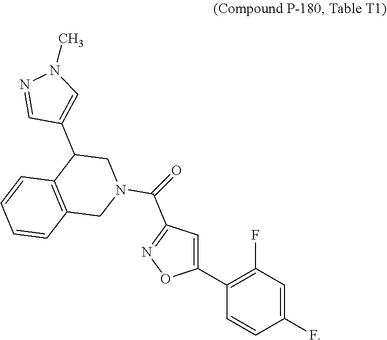 [SMILES: Cn1cc(C2CN(C(=O)c3cc(-c4ccc(F)cc4F)on3)Cc3ccccc32)cn1]

To a solution of 4-(1-methylpyrazol-4-yl)-1,2,3,4 tetrahydroisoquinoline hydrochloride (prepared as described above in step 2, 200 mg, 0.664 mmol) in DMF (6.6 mL) was added at room temperature 1-[bis(dimethylamino)methylene]-1H-1,2,3-triazolo[4,5-b]pyridinium 3-oxide hexafluorophosphate (HATU) (312 mg, 0.797 mmol, 1.20 equiv.), followed by the addition of 5-(2,4-difluorophenyl)isoxazole-3-carboxylic acid (149 mg, 0.664 mmol, 1.00 equiv.) and N,N-diisopropylethylamine (0.464 mL, 2.66 mmol, 4.00 equiv.). The reaction mixture was stirred at room temperature until reaction completion by LC-MS. The reaction mixture was partitioned between an ammonium chloride saturated solution and dichloromethane, the organic layer was separated, and the aqueous layer was extracted twice with dichloromethane. The combined organic layers were dried over sodium sulfate, filtered and concentrated in vacuo. The crude material was dissolved in dimethyl sulfoxide (DMSO) (3 mL) and acidified with few drops of formic acid before purification by reverse-phase chromatography (acetonitrile 30% to 100%), which afforded the desired product as a white solid (222 mg, 0.528 mmol).

LC-MS (Method A): retention time 1.04 min, m/z 421 [M+H+].

### Example P2: Preparation of 5-(2,4-difluorophenyl)isoxazol-3-yl]-[4-methyl-4-(1-methylpyrazol-4-yl)-1,3-dihydroisoquinolin-2-yl]methanone (Compound P-53, Table T1)

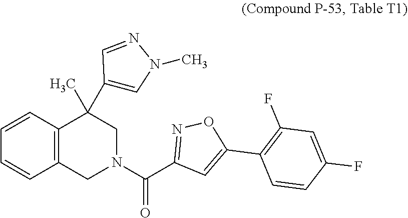 [SMILES: Cn1cc(C2(C)CN(C(=O)c3cc(-c4ccc(F)cc4F)on3)Cc3ccccc32)cn1]

### Step 1: Preparation of (1-methylpyrazol-4-yl)-phenyl-methanol

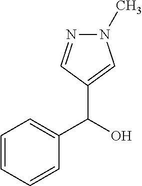

A one necked round bottom flask, equipped with a magnetic stirrer bar, was charged with 1-methyl-1H-pyrazole-4-carbaldehyde (2.20 g, 19.2 mmol) and THF (40 mL). To the colorless solution, 1 M phenylmagnesium bromide in THF (21 mL, 21.1 mmol) was added dropwise at 0-5° C. under argon atmosphere for 15 minutes. After the addition, the ice-bath was removed, and the white suspension was stirred at room temperature for 2.5 hours. The reaction mixture was poured into a NH₄Cl saturated solution (40 mL) and extracted with ethyl acetate (2×40 mL). The combined organic layers were washed with brine, dried over sodium sulfate, and concentrated in vacuo to afford the crude product as a colorless oil. The crude product was purified by combiflash (silica gel, gradient: ethyl acetate in cyclohexane) to afford the desired product (1-methylpyrazol-4-yl)-phenyl-methanol as a colorless oil.

LC-MS (Method A): 189 [M+H]+; retention time: 0.62 min.

¹H NMR (400 MHz, CDCl3) δ ppm 2.89 (br s, 1H) 3.81 (s, 3H) 5.80 (s, 1H) 7.18 (s, 1H) 7.26-7.43 (m, 6H).

### Step 2: Preparation of 2-(1-methylpyrazol-4-yl)-2-phenyl-acetonitrile

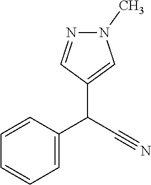

A round-bottom flask, equipped with a magnetic stirrer bar and condenser, was charged with (1-methylpyrazol-4-yl)-phenyl-methanol (prepared as described above in step 1, 3.45 g, 15.6 mmol) and dichloromethane (156 mL). Then, lithium carbonate (0.23 g, 3.1 mmol), trimethylsilyl cyanide (9.0 mL) and iodine (7.23 g, 28.0 mmol) were added successively at room temperature. The mixture was stirred at 35° C. for 1 hour. The reaction mixture was then cooled to room temperature and poured into a sodium thiosulfate saturated solution (250 mL) and extracted with dichloromethane (2×150 mL). The combined organic layers were washed with brine, dried over sodium sulfate, and concentrated in vacuo to afford the crude product which was purified by combiflash (silica gel, gradient: ethyl acetate in cyclohexane) to afford the desired title compound as a yellow oil.

LC-MS (Method A): 198 [M+H]+; retention time: 0.78 min.

¹H NMR (400 MHz, CDCl3) δ ppm 3.87 (s, 3H) 5.09 (s, 1H) 7.32 (s, 1H) 7.33-7.43 (m, 6H).

### Step 3: Preparation of 2-(1-methylpyrazol-4-yl)-2-phenyl-propanenitrile

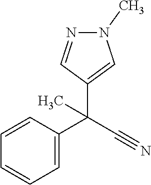 [SMILES: Cn1cc(C(C)(C#N)c2ccccc2)cn1]

A 250 mL-3-necked flask, equipped with a magnetic stirrer bar and condenser, was charged with 2-(1-methylpyrazol-4-yl)-2-phenyl-acetonitrile (prepared as described above in step 2, 3.22 g, 16.3 mmol) and THF (65 mL). A solution of n-butyl lithium in hexane (7.8 mL, 19.6 mmol) was added dropwise at −70° C. under a nitrogen atmosphere. The orange solution was stirred at this temperature for 30 minutes before adding iodomethane (1.54 mL, 24.5 mmol) dropwise at −70° C. The resulting yellow solution was stirred at −78° C. for 5 minutes and then allowed to warm to ambient temperature and stirred for 30 minutes. The reaction mixture was then poured into water (90 mL) and extracted with ethyl acetate. The organic layer was washed with brine, dried over sodium sulfate, and concentrated in vacuo to afford the crude product as an orange oil. This was purified by combiflash (silica gel, gradient: ethyl acetate in cyclohexane) to afford the title compound as a yellow oil.

LC-MS (Method A): 211 [M+H]+; retention time: 0.84 min.

¹H NMR (400 MHz, CDCl3) δ ppm 2.04 (s, 3H) 3.88 (s, 3H) 7.28-7.49 (m, 7H).

### Step 4: Preparation of 2-(1-methylpyrazol-4-yl)-2-phenyl-propan-1-amine

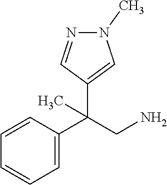 [SMILES: Cn1cc(C(C)(CN)c2ccccc2)cn1]

A 250 mL-3-necked flask, equipped with a magnetic stirrer bar, was charged with 2-(1-methylpyrazol-4-yl)-2-phenyl-propanenitrile (prepared as described above in step 3, 2.82 g, 13.3 mmol) and THF (40 mL). To the yellow solution borane dimethyl sulfide complex (4.0 mL, 40.0 mmol) was added dropwise at room temperature under argon atmosphere and the resulting colorless mixture was stirred at 65° C. for 2 hours. The reaction mixture was cooled to 0° C. before adding hydrochloric acid (8.9 mL, 53.7 mmol) dropwise (strong gas evolution) and the mixture was stirred at 65° C. for 1 hour and allowed to stand overnight at room temperature. The mixture was diluted with water (80 mL), basified with 13 mL 6 M NaOH (pH 12) and then extracted twice with ethyl acetate. The combined organic layers were washed with brine, dried over sodium sulfate, and concentrated in vacuo to give the title compound as a yellow oil which was used without further purification in the next step.

LC-MS (Method A): 216 [M+H]+; retention time: 0.39 min.

### Step 5: Preparation of methyl N-[2-(1-methylpyrazol-4-yl)-2-phenyl-propyl]carbamate

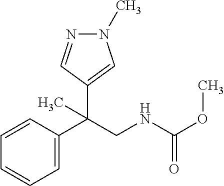 [SMILES: COC(=O)NCC(C)(c1ccccc1)c1cnn(C)c1]

A sealed tube, equipped with a magnetic stirrer bar, was charged with 2-(1-methylpyrazol-4-yl)-2-phenyl-propan-1-amine (prepared as described above in step 4, 3.01 g, 11.2 mmol) and dichloromethane (45 mL). Methyl chloroformate (1.1 mL, 13.4 mmol), followed by triethylamine (4.7 mL, 33.6 mmol) were added dropwise at 0-10° C. under an argon atmosphere. The ice-bath was removed, and the mixture was stirred at room temperature for 1 hour. The reaction mixture was poured into water and the organic phase was separated. The water phase was extracted twice with ethyl acetate. The combined organic layers were dried over sodium sulfate and concentrated in vacuo. The crude material was purified by combiflash (silica gel, gradient: ethyl acetate in cyclohexane) to afford the title compound as a colourless gum.

LC-MS (Method A): 274 [M+H]+; retention time: 0.80 min.

### Step 6: Preparation of methyl 4-methyl-4-(1-methylpyrazol-4-yl)-1,3-dihydroisoquinoline-2-carboxylate

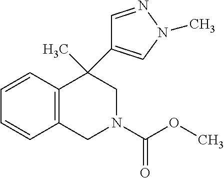 [SMILES: COC(=O)N1Cc2ccccc2C(C)(c2cnn(C)c2)C1]

A one necked round bottom flask, equipped with a magnetic stirrer bar, was charged with methyl N-[2-(1-methylpyrazol-4-yl)-2-phenyl-propyl]carbamate (prepared as described above in step 5, 422 mg, 1.544 mmol), hydrochloric acid (5.00 mL/mmol, 9.26 g, 7.720 mL, 94.0 mmol) and paraformaldehyde (93 mg, 0.978 mmol). The mixture was stirred at room temperature for 40 minutes, where LC-MS analysis showed reaction completion. The reaction mixture was slowly poured into water (30 mL), neutralized with NaHCO₃, and extracted with ethyl acetate (3×20 mL). The combined organic layers were washed with brine, dried over sodium sulfate and concentrated in vacuo to yield the crude title compound as a colorless gum which was used as such without further purification.

LC-MS (Method A): 286 [M+H]+; retention time: 0.87 min.

### Step 7: Preparation of 4-methyl-4-(1-methylpyrazol-4-yl)-2,3-dihydro-1H-isochinoline

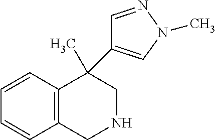 [SMILES: Cn1cc(C2(C)CNCc3ccccc32)cn1]

A one necked round bottom flask, equipped with a magnetic stirrer bar, was charged with methyl 4-methyl-4-(1-methylpyrazol-4-yl)-1,3-dihydroisoquinoline-2-carboxylate (prepared as described above in step 6, 3.86 g, 13.5 mmol), 1,2-dichloroethane (5.00 ml/mmol, 68 ml) and iodotrimethylsilane (8.37 g, 5.69 ml, 40.6 mmol). The mixture was stirred at 60° C. under argon atmosphere for 45 min, where LC-MS analysis showed reaction completion. After cooling to room temperature, the reaction mixture was slowly poured into water (30 mL), neutralized with NaHCO₃, and extracted with ethyl acetate (2×50 mL). The combined organic layers were washed with brine, dried over sodium sulfate and concentrated in vacuo to yield the title compound as a dark orange gum.

LC-MS (Method A): 228 [M+H]+; retention time: 0.61 min.

### Step 8: Preparation of 5-(2,4-difluorophenyl)isoxazol-3-yl]-[4-methyl-4-(1-methylpyrazol-4-yl)-1,3-dihydroisoquinolin-2-yl]methanone (Compound P-53, Table T1)

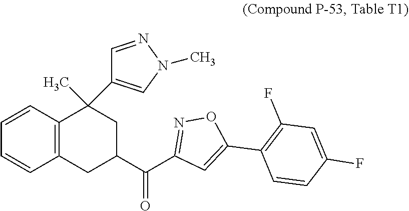 [SMILES: Cn1cc(C2(C)CC(C(=O)c3cc(-c4ccc(F)cc4F)on3)Cc3ccccc32)cn1]

To a solution of 4-methyl-4-(1-methylpyrazol-4-yl)-2,3-dihydro-1H-isoquinoline (prepared as described above in step 7, 1.50 g, 6.60 mmol), in ethyl acetate (27 mL), was added N,N-diisopropylethylamine (2.57 g, 3.40 mL, 19.8 mmol) and 5-(2,4-difluorophenyl)isoxazole-3-carboxylic acid (1.68 g, 7.26 mmol) at room temperature. To this solution was added T3P (7.56 g, 7.07 mL, 11.9 mmol) and the mixture was stirred at room temperature for 60 minutes. After this time, the reaction mixture was diluted with water (20 mL) and then extracted with ethyl acetate (2×20 mL). The combined organic layers were washed with brine, dried over sodium sulfate and concentrated in vacuo. The crude product was purified by combiflash (12 g SiO₂ cartridge, eluting with an EtOAc/cyclohexane gradient) to yield the title compound as a colourless gum.

LC-MS (Method A): 435 [M+H]+; retention time: 1.06 min.

### Examples P3 and P4: Preparation of [5-(2,4-difluorophenyl)isoxazol-3-yl]-[(4S)-4-methyl-4-(1-methylpyrazol-4-yl)-1,3-dihydroisoquinolin-2-yl]methanone (Compound P-48, Table T1) and [5-(2,4-difluorophenyl)isoxazol-3-yl]-[(4R)-4-methyl-4-(1-methylpyrazol-4-yl)-1,3-dihydroisoquinolin-2-yl]methanone (Compound P-49, Table T1)

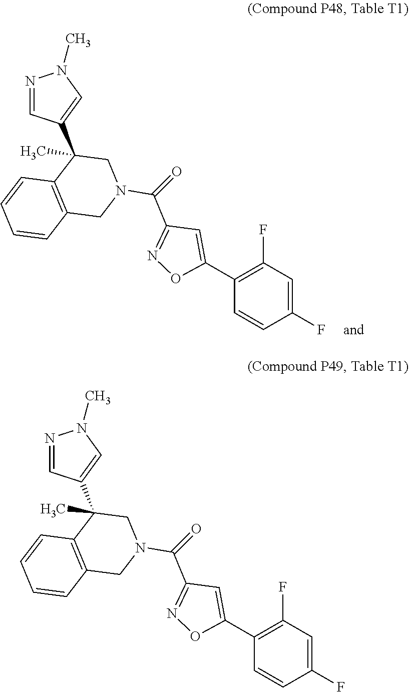 [SMILES: Cn1cc([C@@]2(C)CN(C(=O)c3cc(-c4ccc(F)cc4F)on3)Cc3ccccc32)cn1 ; "and" ; Cn1cc([C@]2(C)CN(C(=O)c3cc(-c4ccc(F)cc4F)on3)Cc3ccccc32)cn1]

A racemic sample of [5-(2,4-difluorophenyl)isoxazol-3-yl]-[4-methyl-4-(1-methylpyrazol-4-yl)-1,3-dihydroisoquinolin-2-yl]methanone (80 mg) was separated into its enantiomers by supercritical fluid chromatography (SFC) chiral HPLC.

### Analytical SFC Method:

SFC:Waters Acquity UPC²/QDa
PDA Detector Waters Acquity UPC²
Column: Daicel SFC CHIRALPAK® IA, 3 μm, 0.3 cm×10 cm, 40° C.
Mobile phase: A: CO2 B: EtOH isocratic: 20% B in 4.8 min
ABPR: 1800 psi
Flow rate: 2.0 ml/min
Detection: 257 nm
Sample concentration: 1 mg/mL in acetonitrile
Injection: 1 μL

### Preparative SFC Method:

Column: Daicel CHIRALPAK® AY, 5 μm, 2.0 cm×25 cm
Mobile phase: A: CO2 B: MeOH isocratic: 30% B
Backpressure: 150 bar
Flow rate: 60 ml/min
GLS pump: −
Detection: UV 257 nm
Sample concentration: 80 mg in 2 ml acetonitrile
Injection: 500 μL
   Two Peaks were Isolated:
Peak 1: 21 mg, white crystals; retention time (min) 3.55; chemical purity (area % at 257 nm)>99%; enantiomeric excess (%)>99%.
Peak 2: 21 mg, white solid; retention time (min) ˜2.72; chemical purity (area % at 245 nm)>99%; enantiomeric excess (%)>99%.
Peak 1 was shown to have the (R) absolute configuration by X-ray analysis, whereas peak 2 was shown to have the (S) absolute configuration by X-ray analysis.
It has been found that the (S) enantiomer (compound P48, Table T1) is more fungicidally active than the (R) isomer (compound P49, Table T1).

### Example P5: Preparation of [5-(2,4-difluorophenyl)isoxazol-3-yl]-[1-methyl-4-(1-methylpyrazol-4-yl)-3,4-dihydro-1H-isoquinolin-2-yl]methanone (Compound P-141, Table T1)

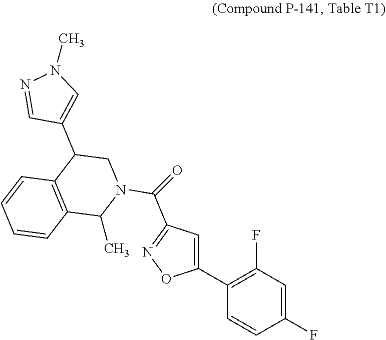 [SMILES: CC1c2ccccc2C(c2cnn(C)c2)CN1C(=O)c1cc(-c2ccc(F)cc2F)on1]

### Step 1: Preparation of tert-butyl-4-(1-methylpyrazol-4-yl)-3,4-dihydro-1H-isoquinoline-2-carboxylate

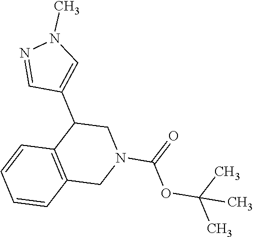 [SMILES: Cn1cc(C2CN(C(=O)OC(C)(C)C)Cc3ccccc32)cn1]

To a suspension of 4-(1-methylpyrazol-4-yl)-1,2,3,4 tetrahydroisoquinoline hydrochloride (prepared as described above in Example P1, step 2, 200 mg, 0.698 mmol) in dichloromethane (3.5 mL) was added, at room temperature, triethylamine (0.196 mL, 1.40 mmol), 4-dimethylaminopyridine (8.6 mg, 0.070 mmol) and di-tert-butyl dicarbonate (0.180 mL, 0.769 mmol). The reaction mixture was stirred at room temperature overnight. The reaction mixture was diluted with an ammonium chloride saturated solution and extracted with dichloromethane (×3). The combined organic layers were dried over sodium sulfate, filtered and concentrated in vacuo. Purification of the crude material by flash chromatography over silica gel (eluting with ethyl acetate in cyclohexane) afforded the title product as a colorless oil.

LC-MS (Method A): 314 [M+H]+; retention time: 1.01 min.

### Step 2: Preparation of tert-butyl-1-methyl-4-(1-methylpyrazol-4-yl)-3,4-dihydro-1H-isoquinoline-2-carboxylate

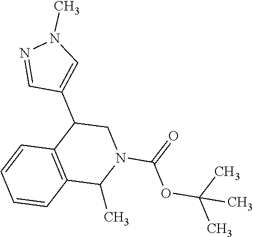 [SMILES: CC1c2ccccc2C(c2cnn(C)c2)CN1C(=O)OC(C)(C)C]

To a solution of tert-butyl-4-(1-methylpyrazol-4-yl)-3,4-dihydro-1H-isoquinoline-2-carboxylate (prepared as described above in step 1, 50 mg, 0.16 mmol) and N,N,N′,N′-tetramethylethylenediamine (0.073 mL, 0.48 mmol) in tetrahydrofurane (1.6 mL) was added dropwise at −78° C. tert-butyl lithium (1.7 M in pentane, 0.19 mL, 0.32 mmol). The reaction mixture was stirred at the same temperature for 30 minutes. After this time, a solution of iodomethane (0.020 mL, 0.32 mmol, 2.0 equiv.) in tetrahydrofuran (0.5 mL) was added dropwise at −78° C. The resulting reaction mixture was stirred for 40 minutes at the same temperature. The reaction mixture was allowed to reach room temperature and diluted with an ammonium chloride saturated solution, and extracted with dichloromethane (×3). The combined organic layers were dried over sodium sulfate, filtered and concentrated in vacuo to afford the desired product that was used without further purification.

LC-MS (Method A): 328 [M+H]+; retention time: 1.05 min.

### Step 3: Preparation of 1-methyl-4-(1-methylpyrazol-4-yl)-1,2,3,4-tetrahydroisoquinolin-2-ium trifluoroacetate Salt

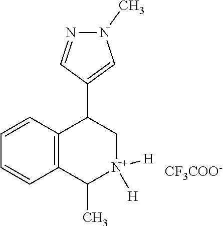

To a solution of tert-butyl-1-methyl-4-(1-methylpyrazol-4-yl)-3,4-dihydro-1H-isoquinoline-2-carboxylate (prepared as described above in step 2, 150 mg, 0.229 mmol) in dichloromethane (1.1 mL) was added at room temperature 2,2,2-trifluoroacetic acid (0.23 mL). The reaction mixture was stirred for 1 hour at room temperature. The reaction mixture was then concentrated under reduced pressure to afford the product, which was used without further purification.

LC-MS (Method A): 228 [M+H]+; retention time: 0.43 min.

### Step 4: Preparation of [5-(2,4-difluorophenyl)isoxazol-3-yl]-[1-methyl-4-(1-methylpyrazol-4-yl)-3,4-dihydro-1H-isoquinolin-2-yl]methanone (Compound P-141, Table T1)

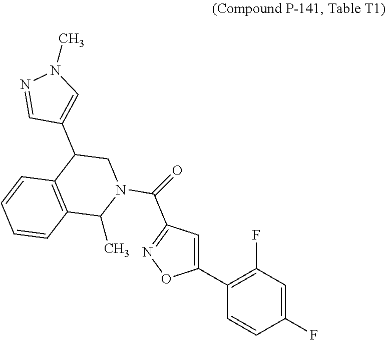 [SMILES: CC1c2ccccc2C(c2cnn(C)c2)CN1C(=O)c1cc(-c2ccc(F)cc2F)on1]

A solution of 1-methyl-4-(1-methylpyrazol-4-yl)-1,2,3,4-tetrahydroisoquinolin-2-ium trifluoroacetate salt (prepared as described above in step 3, 280 mg, 0.6148 mmol) and HATU (313 mg, 0.7993 mmol) in DMF (2 ml) was treated with 5-(2,4-difluorophenyl)isoxazole-3-carboxylic acid (143 mg, 0.6148 mmol) and N,N-diisopropylethylamine (DIPEA) (405 mg, 0.54 ml, 3.074 mmol) and the reaction mixture was allowed to stir over night at room temperature. The reaction mixture was quenched with a NH₄Cl saturated solution and then extracted with dichloromethane (×3). The combined organic layers were dried over Na₂SO₄, filtered and concentrated in vacuo. The crude product was purified by combiflash (24 g SiO₂ cartridge, eluting with a cyclohexane ethyl acetate) to give the title compound as a 3:1 mixture diastereomeric mixture of anti and syn-isomers.

LC-MS (Method A): 435 [M+H]+; retention time: 1.72 min.

¹H NMR (600 MHz, DMSO-d6) δ ppm 1.24 (s, 2H); 1.40 (s, 5H); 1.55 (s, 2H); 1.57 (d, J=6.7 Hz, 10H); 1.63 (d, J=6.9 Hz, 2H); 1.69 (d, J=6.7 Hz, 1H); 1.91 (s, 1H); 2.52-2.55 (m, 5H); 3.58 (br d, J=9.3 Hz, 1H); 3.66 (s, 8H); 3.71-3.74 (m, 3H); 3.80 (s, 2H); 3.85 (s, 1H); 3.90 (dd, J=13.7, 3.5 Hz, 3H); 3.96-4.04 (m, 3H); 4.14 (br s, 4H); 4.20-4.28 (m, 2H); 4.58-4.65 (m, 1H); 5.33 (d, J=6.7 Hz, 1H); 5.76-5.80 (m, 3H); 6.12 (d, J=3.1 Hz, 3H); 6.85 (s, 3H); 6.92 (br d, J=7.7 Hz, 1H); 7.02-7.05 (m, 5H); 7.06 (s, 2H); 7.13-7.17 (m, 4H); 7.17-7.22 (m, 6H); 7.22-7.26 (m, 2H); 7.26-7.30 (m, 4H); 7.30 (d, J=1.1 Hz, 1H); 7.32-7.37 (m, 7H); 7.41 (d, J=7.8 Hz, 3H); 7.44 (s, 1H); 7.56-7.61 (m, 5H); 8.02 (td, J=8.7, 6.4 Hz, 3H); 8.06-8.12 (m, 2H).

### Example P6: Preparation of [5-(2,4-difluorophenyl)isoxazol-3-yl]-[1,4-dimethyl-4-(1-methylpyrazol-4-yl)-1,3-dihydroisoquinolin-2-yl]methanone

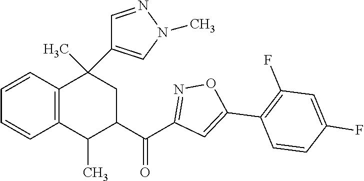 [SMILES: CC1c2ccccc2C(C)(c2cnn(C)c2)CC1C(=O)c1cc(-c2ccc(F)cc2F)on1]

### Step 1: Preparation of methyl 1,4-dimethyl-4-(1-methylpyrazol-4-yl)-1,3-dihydroisoquinoline-2-carboxylate

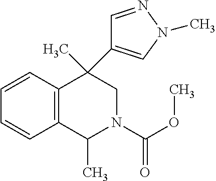 [SMILES: COC(=O)N1CC(C)(c2cnn(C)c2)c2ccccc2C1C]

A 50 mL 3-necked round bottom flask, equipped with a magnetic stirrer bar, was charged with methyl N-[2-(1-methylpyrazol-4-yl)-2-phenyl-propyl]carbamate (prepared as described in Example P2, step 5, 1.00 g, 3.7 mmol), hydrochloric acid (18.3 mL, 223 mmol) and acetaldehyde (0.4 mL, 7.3 mmol). The mixture was stirred at ambient temperature overnight. The reaction mixture was then slowly poured into water (65 mL), neutralized with NaHCO₃ and extracted with ethyl acetate (×3). The combined organic layers were washed with brine, dried over sodium sulfate and concentrated in vacuo to afford compound the title compound as a brown gum. It was used in the next step without further purification.

LC-MS standard: 300 [M+H]+; retention time: 0.91 min.

### Step 2: Preparation of 1,4-dimethyl-4-(1-methylpyrazol-4-yl)-2,3-dihydro-1H-isoquinoline

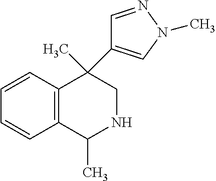 [SMILES: CC1NCC(C)(c2cnn(C)c2)c2ccccc21]

A one necked round bottom flask, equipped with a magnetic stirrer bar, was charged with methyl 1,4-dimethyl-4-(1-methylpyrazol-4-yl)-1,3-dihydroisoquinoline-2-carboxylate (prepared as described above in step 1, 1.1 g, 3.3 mmol), 1,2-dichloroethane (16 mL) and iodotrimethylsilane (1.4 mL, 9.7 mmol).

The mixture was stirred at 60° C. under argon atmosphere for 1 hour. Then the reaction mixture was cooled down to room temperature and slowly poured into sat. NaHCO₃ (30 mL) (gas evolution). The mixture was extracted twice with ethyl acetate and the combined organic layers were washed with brine, dried over sodium sulfate and concentrated in vacuo to give the title compound as a brown gum that was used as such in the next step.

LC-MS standard: 242 [M+H]+; retention time: 0.54 min.

### Step 3: Preparation of 5-(2,4-difluorophenyl)isoxazole-3-carbonyl chloride

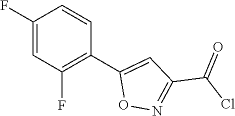 [SMILES: O=C(Cl)c1cc(-c2ccc(F)cc2F)on1]

A suspension of 5-(2,4-difluorophenyl)isoxazole-3-carboxylic acid (1.00 g, 4.3 mmol) in tetrahydrofuran (22 mL) was treated at room temperature with one drop of DMF followed by oxalyl chloride (0.38 mL, 4.3 mmol). The light yellow solution was stirred at ambient temperature under nitrogen atmosphere for 14 hours and then the solvent removed in vacuo to afford the title compound as a yellow solid.

LC-MS (quenched with MeOH) of ester: 240 [M+H]+, retention time: 0.97.

### Step 4: Preparation of [5-(2,4-difluorophenyl)isoxazol-3-yl]-[1,4-dimethyl-4-(1-methylpyrazol-4-yl)-1,3-dihydroisoquinolin-2-yl]methanone

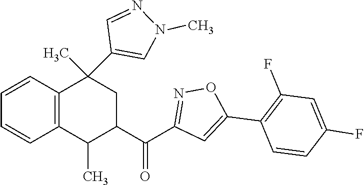 [SMILES: CC1c2ccccc2C(C)(c2cnn(C)c2)CC1C(=O)c1cc(-c2ccc(F)cc2F)on1]

A solution of 1,4-dimethyl-4-(1-methylpyrazol-4-yl)-2,3-dihydro-1H-isoquinoline (prepared as described above in step 2, 0.24 g, 1.0 mmol) in tetrahydrofuran (2.4 mL). was transferred under argon into a supelco vial. To this solution was added 5-(2,4-difluorophenyl)isoxazole-3-carbonyl chloride (0.40 g, 1.0 mmol), and then triethylamine (0.42 mL, 3.0 mmol). The reaction mixture was stirred at room temperature for 17 hours and then poured into an aqueous solution of NaHCO₃ and diluted with ethyl acetate. After separation of the layers, the aqueous phase was back extracted with ethyl acetate, and the combined organic phases washed with water, brine. The organic layer was dried over sodium sulfate, filtered, and concentrated in vacuo. The crude was purified by combiflash (silica gel, gradient: ethyl acetate in cyclohexane) to give the title compound as a brown gum.

LC-MS standard: 449 (M+H)+; retention time: 1.07 min.

NMR analysis showed the compound to be a ca. 1:1 mixture of syn and anti isomers.

A 260 mg sample of [5-(2,4-difluorophenyl)isoxazol-3-yl]-[1,4-dimethyl-4-(1-methylpyrazol-4-yl)-1,3-dihydroisoquinolin-2-yl]methanone (80 mg) was separated into its enantiomers by s chromatography (SFC) chiral HPLC.

### Analytical SFC Method:

SFC:Waters Acquity UPC²/QDa
PDA Detector Waters Acquity UPC²
Column: Daicel SFC CHIRALPAK® IH, 3 μm, 0.46 cm×10 cm, 40° C.
Mobile phase: A: CO2 B: MeOH isocratic: 15% B in 10 min
ABPR: 1800 psi
Flow rate: 2.0 ml/min
Detection: 255 nm
Sample concentration: 1 mg/mL in dichloromethane/acetonitrile
Injection: 1 μL

### Preparative SFC Method:

Sepiatec Prep SFC 100
Column: Daicel CHIRALPAK® IG, 5 μm, 2.0 cm×25 cm
Mobile phase: A: CO2 B: IPA isocratic: 12% B
Backpressure: 150 bar
Flow rate: 90 ml/min
GLS pump: −
Detection: UV 255 nm
Sample concentration: 260 mg in 2 ml dichlorethane/acetonitrile
Injection: 350 μL
   Four Peaks were Isolated:
Peak 1: 46 mg, brown gum; retention time (min) 1.88 min; chemical purity (area % at 255 nm)>93%; enantiomeric excess (%)˜98%.
Peak 2: 38 mg, brown gum; retention time (min) 2.09 min; chemical purity (area % at 255 nm)>96%; enantiomeric excess (%)˜98%.
Peak 3: 43 mg, brown gum; retention time (min) 2.56 min; chemical purity (area % at 255 nm)>98%; enantiomeric excess (%)˜98%.
Peak 4: 46 mg, brown gum; retention time (min) 2.09 min; chemical purity (area % at 255 nm)>99%; enantiomeric excess (%)˜99%.

¹H NMR analysis showed that peaks 1 and 2 had an identical NMR spectrum and have an anti relationship of the methyl to the pyrazole as determined by ROSY 2D NMR, vide infra:

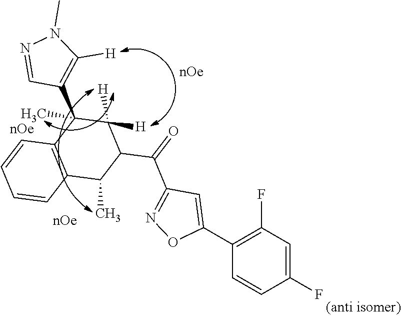

Peaks 3 and 4 also had an identical NMR spectrum but different to the NMR spectrum of peaks 1 and 2. NMR analysis, particularly ROSY 2D NMR, showed nOe's that confirm the methyl and pyrazole groups have a syn relationship, vide infra:

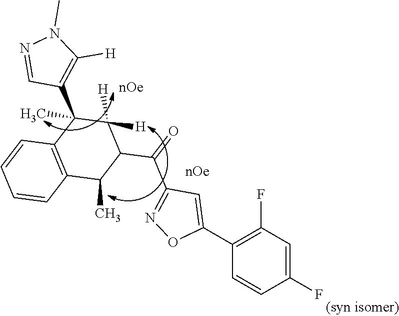

Peak 1 corresponds to [5-(2,4-difluorophenyl)isoxazol-3-yl]-[(1R,4S)-1,4-dimethyl-4-(1-methylpyrazol-4-yl)-1,3-dihydroisoquinolin-2-yl]methanone (compound P9, Table T1).
Peak 2 corresponds to [5-(2,4-difluorophenyl)isoxazol-3-yl]-[(1S,4R)-1,4-dimethyl-4-(1-methylpyrazol-4-yl)-1,3-dihydroisoquinolin-2-yl]methanone (compound P8, Table T1).
Peak 3 corresponds to [5-(2,4-difluorophenyl)isoxazol-3-yl]-[(1R,4R)-1,4-dimethyl-4-(1-methylpyrazol-4-yl)-1,3-dihydroisoquinolin-2-yl]methanone (compound P7, Table T1).
Peak 4 corresponds to [5-(2,4-difluorophenyl)isoxazol-3-yl]-[(1S,S)-1,4-dimethyl-4-(1-methylpyrazol-4-yl)-1,3-dihydroisoquinolin-2-yl]methanone (compound P6, Table T1).

It has been shown that the compound of peak 1 (compound P9, Table T1) is more fungicidally active than the compound of peak 2 (compound P8, Table T1). Also, it has been shown that the compound of peak 4 (compound P6, Table T1) is more fungicidally active than the compound of peak 3 (compound P7, Table T1).

### Example P7: Preparation of rac-(1S,4S)-4-(1,5-dimethylpyrazol-4-yl)-1-methyl-1,2,3,4-tetrahydroisoquinoline

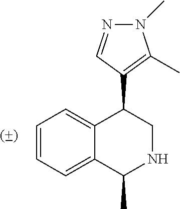 [SMILES: Cc1c([C@H]2CN[C@@H](C)c3ccccc32)cnn1C]

### Step 1: Preparation of methyl N-[2-(1,5-dimethylpyrazol-4-yl)-2-phenyl-ethyl]carbamate

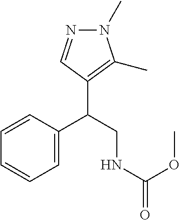 [SMILES: COC(=O)NCC(c1ccccc1)c1cnn(C)c1C]

A three necked flask, equipped with a mechanical stirrer, was charged with 2-(1,5-dimethylpyrazol-4-yl)-2-phenyl-ethanamine (3.5 g, 16 mmol), ethyl acetate (65 mL) and Triethylamine (6.8 mL, 49 mmol). Then methyl chloroformate (1.5 mL, 20 mmol) were added dropwise at 0° C. under argon atmosphere during 30 min and the mixture was stirred at RT for 1 h. The reaction mixture was poured into water (800 mL) and extracted with EtOAc (2×150 mL). The combined organic layers were washed with brine, dried over sodium sulfate and concentrated in vacuo The crude material was purified by FCC (80 g SiO2, EtOAc/Cyclohexane gradient) to afford methyl N-[2-(1,5-dimethylpyrazol-4-yl)-2-phenyl-ethyl]carbamate.

LC-MS (Method A): retention time 0.76 min, 274 (M+H)

1H NMR (400 MHz, CDCl3) δ ppm 2.03-2.13 (m, 3H) 3.62-3.74 (m, 5H) 3.77 (s, 3H) 3.94-4.05 (m, 1H) 4.72 (br s, 1H) 7.21-7.26 (m, 3H) 7.27-7.34 (m, 2H) 7.38 (s, 1H)

### Step 2: Preparation of rac-methyl(1S,4S)-4-(1,5-dimethylpyrazol-4-yl)-1-methyl-3,4-dihydro-1H-isoquinoline-2-carboxylate

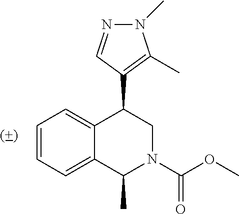 [SMILES: COC(=O)N1C[C@H](c2cnn(C)c2C)c2ccccc2[C@@H]1C]

A one necked round bottom flask, equipped with a magnetic stirrer bar, was charged with methyl N-[2-(1,5-dimethylpyrazol-4-yl)-2-phenyl-ethyl]carbamate (2.0 g, 7.3 mmol), hydrochloric acid (conc. 37 mL, 450 mmol) and acetaldehyde (0.83 mL, 15 mmol). The mixture was stirred at RT for 2 h. The reaction mixture was slowly poured into water (500 mL) neutralized with NaHCO₃ in portions slowly (strong gas evolution) to pH 8. The mixture was extracted with EtOAc (3×50 mL) and the combined organic layers were washed with brine, dried over sodium sulfate and concentrated in vacuo. The crude product was purified by chromatography to afford rac-methyl (1S,4S)-4-(1,5-dimethylpyrazol-4-yl)-1-methyl-3,4-dihydro-1H-isoquinoline-2-carboxylate as a single syn-diastereoisomer by ¹H NMR

LC-MS (Method A): retention time 0.87 min, 300 (M+H)

1H NMR (400 MHz, CDCl3) δ ppm: 1.56 (d, J=6.90 Hz, 3H); 2.18 (br s, 3H); 3.02-3.27 (m, 1H); 3.76 (br s, 3H); 3.83 (s, 3H); 3.97-4.09 (m, 1H); 4.09-4.37 (m, 1H); 5.18-5.45 (m, 1H); 6.86-7.02 (m, 1H); 7.04-7.24 (m, 4H).

### Step 3: Preparation of rac-(1S,4S)-4-(1,5-dimethylpyrazol-4-yl)-1-methyl-1,2,3,4-tetrahydroisoquinoline

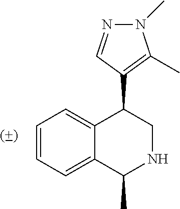 [SMILES: Cc1c([C@H]2CN[C@@H](C)c3ccccc32)cnn1C]

A 100 mL one necked round bottom flask, equipped with a magnetic stirrer bar, was charged with rac-methyl (1S,4S)-4-(1,5-dimethylpyrazol-4-yl)-1-methyl-3,4-dihydro-1H-isoquinoline-2-carboxylate (1.3 g, 4.1 mmol) 1,2-dichloroethane (21 mL) and iodotrimethylsilane (1.7 mL, 12 mmol). The mixture was stirred at 60° C. for 1 h under argon atmosphere. The reaction was cooled to rt then 10% aqueous HCl 22 ml was added to the reaction under ice cooling. The organic solvent was removed in vacuo and the aqeuous residue was adjusted with 10% aqeuous NaOH to pH 8 and then was extracted with tert-dichloromethane. The combined organic layers were dried over magnesium sulfate, filtered and concentrated in vacuo to give rac-(1S,4S)-4-(1,5-dimethylpyrazol-4-yl)-1-methyl-1,2,3,4-tetrahydroisoquinoline which was pure enough to use without further purification.

LC-MS (Method A): retention time 0.35 min, 242 (M+H)

¹H NMR (600 MHz, CHLOROFORM-d) δ ppm 1.86 (d, J=6.9 Hz, 3H) 2.23 (s, 3H) 3.27 (dd, J=12.5, 10.8 Hz, 1H) 3.59 (dd, J=12.8, 5.6 Hz, 1H) 3.83 (s, 3H) 4.58 (dd, J=10.6, 5.4 Hz, 1H) 4.80 (q, J=6.8 Hz, 1H) 7.00 (d, J=7.8 Hz, 1H) 7.11 (s, 1H) 7.16 (d, J=7.6 Hz, 1H) 7.18-7.22 (m, 1H) 7.25-7.28 (m, 1H)

### Example P8: Preparation of 1,4-dimethyl-4-(1-methylpyrazol-4-yl)-2,3-dihydro-1H-isoquinoline

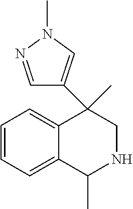 [SMILES: CC1NCC(C)(c2cnn(C)c2)c2ccccc21]

### Step 1: Preparation of tert-butyl N-[2-hydroxy-2-(1-methylpyrazol-4-yl)propyl]-N-(1-phenylethyl)carbamate

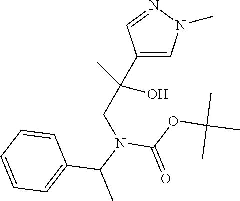 [SMILES: CC(c1ccccc1)N(CC(C)(O)c1cnn(C)c1)C(=O)OC(C)(C)C]

### Method 1: Step 1: Preparation of tert-butyl N-[2-(1-methyl pyrazol-4-yl)-2-oxo-ethyl]-N-(1-phenylethyl)carbamate

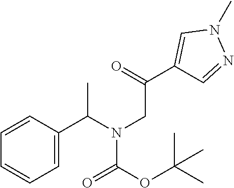 [SMILES: CC(c1ccccc1)N(CC(=O)c1cnn(C)c1)C(=O)OC(C)(C)C]

A three neck flask, equipped with a magnetic stirrer, was charged with 2-bromo-1-(1-methyl-1H-pyrazol-4-yl)ethanone (1 g, 4.72 mmol) and N,N-Dimethylacetamide (23.6 mL). The resulting mixture was cooled down to 0° C. under argon atmosphere and DL-alpha-methylbenzylamine (0.64 mL, 4.72 mmol) was then added slowly (over 4 min). The mixture was stirred for 10 min and triethylamine (0.993 mL, 7.09 mmol) was added at 0° C. under Ar. After 1.5 Hr at 0° C., di-tert-butyl pyrocarbonate (1.14 g, 5.2 mmol) dissolved in N,N-Dimethylacetamide (23.6 mL) was added drop-wise at 0° C. under an argon atmosphere. The reaction mixture was allowed to warm to RT and left to stir overnight. HCl was added to the reaction mixture (to adjust to pH 3) and the aqueous layer was then extracted three times using ethylacetate. The combined organic layers were washed with brine, dried over Na₂SO₄, filtered and concentrated in vacuo To give the crude product which was purified by chromatography over silica gel with a gradient of cyclohexane/ethylacetate to yield the title compound as white solid.

LC-MS (Method A): retention time 0.99 min, 344 (M+H)

¹H NMR (400 MHz, CDCl3) δ ppm 1H NMR (400 MHz, Solvent) δ ppm 7.74-7.99 (m, 2H) 7.35 (br d, J=5.1 Hz, 4H) 7.25-7.31 (m, 1H) 5.29-5.80 (m, 1H) 4.20-4.57 (m, 1H) 3.92 (s, 3H) 3.66-3.90 (m, 1H) 1.31-1.54 (m, 12H)

### Method 1: Step 2: Preparation of tert-butyl N-[2-hydroxy-2-(1-methylpyrazol-4-yl)propyl]-N-(1-phenylethyl)carbamate

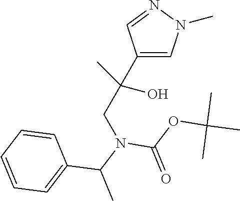 [SMILES: CC(c1ccccc1)N(CC(C)(O)c1cnn(C)c1)C(=O)OC(C)(C)C]

A round bottom flask, equipped with a magnetic stirrer bar, was charged with tert-butyl N-[2-(1-methylpyrazol-4-yl)-2-oxo-ethyl]-N-(1-phenylethyl)carbamate (100 mg, 0.291 mmol) and tetrahydrofuran (1.5 mL) and the resulting mixture was cooled to 0° C. under argon atmosphere. Methylmagnesium bromide solution 3 M (0.24 ml, 0.728 mmol) was then added dropwise under Argon at 0° C. After 1.5 Hr, the reaction mixture was allowed to warm to RT. A further portion of Methylmagnesium bromide solution 3 M (0.24 mL, 0.728 mmol) was added at RT and the mixture stirred for 2.5 hr at RT, and then 21 hours at 60° C. The reaction mixture was cooled, and added to sat. aq. sol. NH₄Cl. The aqueous layer was then extracted with ethyl acetate, and the combined organic layers were washed brine, and dried over Na₂SO₄, filtered and concentrated in vacuo to give crude mixture containing tert-butyl N-[2-hydroxy-2-(1-methylpyrazol-4-yl)propyl]-N-(1-phenylethyl)carbamate (around 70%) and tert-butyl N-[2-(1-methylpyrazol-4-yl)-2-oxo-ethyl]-N-(1-phenylethyl)carbamate (around 30%). The desired product was not isolated using further purification method.

LC-MS (Method A): retention time 1.02 min, 360 (M+H)

### Method 2: Step 1: Preparation of tert-butyl N-acetonyl-N-(1-phenylethyl)carbamate

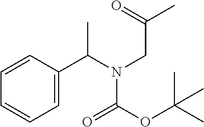 [SMILES: CC(=O)CN(C(=O)OC(C)(C)C)C(C)c1ccccc1]

A three neck flask, equipped with a magnetic stirrer, was charged with 1-chloropropan-2-one (0.86 mL, 10.27 mmol), N,N-Dimethylacetamide (51 mL) and potassium iodide (1.705 g, 10.27 mmol). The resulting mixture was cooled down to 0° C. under argon atmosphere and DL-alpha-methylbenzylamine (1.4 mL, 10.27 mmol) was then added slowly. The mixture was stirred for 20 min and Triethylamine (2.16 mL, 15.40 mmol) was added at 0° C. under Ar. After 3 hours, 1-chloropropan-2-one (0.86 mL, 10.27 mmol) was added again as starting material was still present. The resulting mixture was stirred 3 hours at 0° C. to 10° C. then Di-tert-butyl pyrocarbonate (2.49 g, 11.22 mmol) dissolved in N,N-Dimethylacetamide (51 mL) was added drop-wise at 0° C. under Argon atmosphere. The reaction mixture was warmed to RT and left to stir overnight. HCl was then added to the mixture (to pH 3) and the aqueous layer was then extracted three times using ethylacetate. The combined organic layers were washed brine, dried over Na₂SO₄, filtered and concentrated in vacuo. The crude product was purified by chromatography over silica gel with a gradient of cyclohexane/ethylacetate to give tert-butyl N-acetonyl-N-(1-phenylethyl)carbamate as a orange/brown liquid.

LC-MS (Method A): retention time 1.04 min, 300 (M+Na)

1H NMR (400 MHz, CDCl3) δ ppm 7.28-7.45 (m, 5H) 5.27-5.85 (m, 1H) 3.47 (br s, 2H) 1.89-2.06 (m, 3H) 1.41-1.53 (m, 12H)

### Method 2: Step 2: Preparation of tert-butyl N-[2-hydroxy-2-(1-methylpyrazol-4-yl)propyl]-N-(1-phenylethyl)carbamate

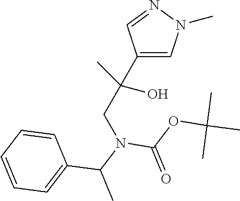 [SMILES: CC(c1ccccc1)N(CC(C)(O)c1cnn(C)c1)C(=O)OC(C)(C)C]

A 100 ml three necked flask was charged with 4-iodo-1-methyl-1H-pyrazole (3.1 g, 14 mmol) and Tetrahydrofuran (12 mL) and the resulting mixture was cooled to 0° C. under argon atmosphere. Isopropylmagnesium chloride lithium chloride complex solution (1.3 mol/L) in THF (12 mL, 16 mmol) was added slowly (over 15 min). The mixture was left to stir at 0° C. After 45 min, tert-butyl N-acetonyl-N-(1-phenylethyl)carbamate (1.6 g, 5.8 mmol) dissolved in Tetrahydrofuran (12 mL) was then added drop-wise under Argon at 0° C. The reaction mixture was brought to room temperature (Precipitate cleared to give a yellow solution) and stirred over night at room temperature. The reaction mixture was neutralised using sat. aq. sol. HCl. The aqueous layer was then extracted three times using ethylacetate and the combined organic layers were washed, dried over Na₂SO₄, filtered and concentrated in vacuo to give the crude product as a yellow sticky oil. The crude mixture was purified by chromatography over silica gel with a gradient of cyclohexane/ethylacetate to give tert-butyl N-[2-hydroxy-2-(1-methylpyrazol-4-yl)propyl]-N-(1-phenylethyl)carbamate.

LC-MS (Method A): retention time 1.01 min, 361 (M+2H)

¹H NMR (400 MHz, CDCl3) δ ppm 7.15-7.42 (m, 7H) 4.79-5.20 (m, 1H) 3.86 (m, Hz, 3H) 3.27-3.49 (m, 3H) 1.30-1.51 (m, 12H)

### Step 2: Preparation of 1,4-dimethyl-4-(1-methylpyrazol-4-yl)-2,3-dihydro-1H-isochinoline

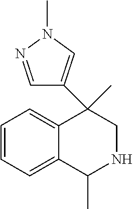 [SMILES: CC1NCC(C)(c2cnn(C)c2)c2ccccc21]

### Method 1:

A 5 mL vial was charged with tert-butyl N-[2-hydroxy-2-(1-methylpyrazol-4-yl)propyl]-N-(1-phenylethyl)carbamate (50 mg, 0.139 mmol) at 0° C. Then, a mixture of Water (0.27 mL) and Sulfuric acid (0.27 mL) was added at 0° C. The reaction mixture was stirred at 40° C. for 30 min then 24 hours at 60° C. The reaction mixture was allowed to cool down to rt. Sulfuric acid (0.2782 mL) was added again and the reaction mixture was heated to 40° C. for 2 hours and 3 days at room temperature and finally 2 hours at 60°. The reaction mixture was carefully poured into a saturated solution of sodium bicarbonate and the mixture extracted with ethyl acetate. The combined organic layers were dried over sodium sulfate, filtered and concentrated in vacuo to yield the crude title compound as a mixture of the diastereoisomers (syn and anti˜1:3)

LC-MS (Method A): retention time 0.51 min, 242 (M+H)

1H NMR (400 MHz, CDCl3) δ ppm) 7.28-7.41 (m, 1H) 7.10-7.26 (m, 6H) 6.87 (s, 1H) 6.96 (s, 1H) 4.15 (dq, J=17.85, 7.01 Hz, 2H) 3.73-3.93 (m, 5H) 2.98-3.13 (m, 3H) 2.11-2.27 (m, 2H) 1.98-2.11 (m, 2H) 1.59-1.66 (m, 4H) 1.44-1.55 (m, 4H) 1.37 (d, J=6.54 Hz, 1H) 1.26 (t, J=7.08 Hz, 2H)

### Method 2:

A 5 mL vial under argon was charged with tert-butyl N-[2-hydroxy-2-(1-methylpyrazol-4-yl)propyl]-N-(1-phenylethyl)carbamate (50 mg, 0.139 mmol), Chlorobenzene (0.4172 mL) and aluminum chloride (0.028 g, 0.208 mmol) at room temperature. The resulting mixture was stirred 1 hour at room temperature, 1 hour at 50° C. and over night at room temperature, and finally 3 hours at 60° C. Further aluminum chloride (0.02782 g, 0.208 mmol) was added to the reaction mixture at RT and the reaction mixture was heated to 60° C. The reaction mixture was allowed to cool down to room temperature, poured into a saturated solution of sodium bicarbonate, and then extracted with ethyl acetate, dried over sodium sulfate, filtered and concentrated in vacuo to get crude title product. The analyses of the crude material were in accordance with the structure of the product (mixture of diastereoisomers anti and syn˜1:1).

LC-MS (Method A): retention time 0.51 min, 242 (M+H)

¹H NMR (400 MHz, CDCl3) δ ppm 7.28-7.41 (m, 1H) 7.10-7.26 (m, 6H) 6.87 (s, 1H) 6.96 (s, 1H) 4.15 (dq, J=17.85, 7.01 Hz, 2H) 3.73-3.93 (m, 5H) 2.98-3.13 (m, 3H) 2.11-2.27 (m, 2H) 1.98-2.11 (m, 2H) 1.59-1.66 (m, 4H) 1.44-1.55 (m, 4H) 1.37 (d, J=6.54 Hz, 1H) 1.26 (t, J=7.08 Hz, 2H)

### Example P10: Preparation of 1-methyl-4-(1-methylpyrazol-4-yl)-1,2,3,4-tetrahydroisoquinoline

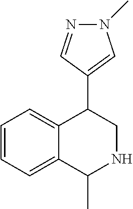 [SMILES: CC1NCC(c2cnn(C)c2)c2ccccc21]

### Step 1: Preparation of tert-butyl N-[2-hydroxy-2-(1-methylpyrazol-4-yl)ethyl]-N-(1-phenylethyl)carbamate

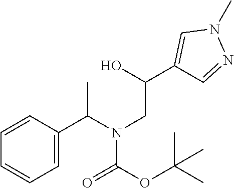 [SMILES: CC(c1ccccc1)N(CC(O)c1cnn(C)c1)C(=O)OC(C)(C)C]

A 25 mL flask was charged with tert-butyl N-[2-(1-methylpyrazol-4-yl)-2-oxo-ethyl]-N-(1-phenylethyl)carbamate (0.52 g, 1.51 mmol), methanol (6 mL), tetrahydrofuran (1.5 mL) and sodium borohydride (0.1146 g, 3.02 mmol) at 0° C. Then, the reaction mixture was allowed to warm up to room temperature for and stirred for 1.5 Hr. The reaction mixture was diluted with a saturated solution of ammonium chloride and ethyl acetate. After separation of the layer, the aqueous layer was extracted once with ethyl acetate. The combined organic layer were dried over sodium sulfate, filtered and concentrated in vacuo to afford tert-butyl N-[2-hydroxy-2-(1-methylpyrazol-4-yl)ethyl]-N-(1-phenylethyl)carbamate.

LC-MS (Method A): retention time 0.98 min, 346 (M+H)

¹H NMR (400 MHz, CDCl3) δ ppm 7.10 (s, 6H) 6.95 (br d, J=0.73 Hz, 7H) 5.20-5.45 (m, 2H) 4.85 (br d, J=8.72 Hz, 1H) 4.16-4.31 (m, 1H) 3.85 (d, J=13.08 Hz, 6H) 3.31-3.65 (m, 2H) 2.91-3.15 (m, 2H) 1.59 (d, J=7.27 Hz, 3H) 1.44-1.51 (m, 12H) 1.59 (d, J=7.27 Hz, 3H)

### Step 2: Preparation of 1-methyl-4-(1-methylpyrazol-4-yl)-1,2,3,4-tetrahydroisoquinoline

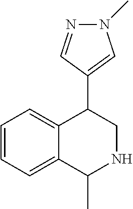 [SMILES: CC1NCC(c2cnn(C)c2)c2ccccc21]

### Method 1:

A 5 mL vial was charged with N-[2-hydroxy-2-(1-methylpyrazol-4-yl)ethyl]-N-(1-phenylethyl)carbamate (50 mg, 0.14 mmol) at 0° C. Then, a mixture of water (0.1448 mL) and sulfuric acid (0.4343 mL) was added at 0° C. The reaction mixture was stirred at rt for 3 hours then 3.5 hr at 40° C. The reaction mixture was quenched carefully with a saturated solution of sodium bicarbonate and diluted with ethyl acetate. After separation of the organic layer, the aqueous layer (pH=8-9) was back extracted twice with ethyl acetate. The combined organic layers were dried over sodium sulfate, filtered and concentrated in vacuo to afford 1-methyl-4-(1-methylpyrazol-4-yl)-1,2,3,4-tetrahydroisoquinoline as a mixture of ianti:syn diastereoisomers in a ratio of 3:1.

LC-MS (Method A): retention time 0.15-0.33 min, 228 (M+H)

1H NMR (400 MHz, CDCl3) δ ppm 7.30 (s, 1H) 7.04-7.25 (m, 6H) 6.99 (s, 1H) 4.08-4.31 (m, 3H) 3.99-4.04 (m, 1H) 3.77-3.90 (m, 4H) 3.45 (dd, J=12.90, 4.90 Hz, 1H) 3.11-3.37 (m, 1H) 3.03 (dd, J=12.90, 7.81 Hz, 1H) 2.79-2.98 (m, 2H) 1.47-1.58 (m, 4H) 2.79-2.98 (m, 2H)

### Method 2: Step 1: Preparation of 5-(1-methylpyrazol-4-yl)-3-(1-phenylethyl)oxazolidin-2-one

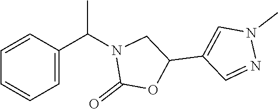 [SMILES: CC(c1ccccc1)N1CC(c2cnn(C)c2)OC1=O]

A 20 mL vial was charged with N-[2-hydroxy-2-(1-methylpyrazol-4-yl)ethyl]-N-(1-phenylethyl)carbamate (550 mg, 1.43 mmol), ethyl acetate (7 mL) and (1S)-(+)-Camphor-10-sulfonic acid (0.509 g, 2.150 mmol) at rt. Then, the resulting mixture was heated at 50° C. for 1 h 30. The reaction mixture was allowed to cool down to rt and carefully poured into a saturated solution of sodium bicarbonate. Then, it was diluted with ethyl acetate. After separation of the organic layer, the aqueous layer (pH=8) was back extracted twice with ethyl acetate. The combined organic layers were dried over sodium sulfate, filtered and concentrated in vacuo. The crude material was purified by chromatography to afford 5-(1-methylpyrazol-4-yl)-3-(1-phenylethyl)oxazolidin-2-one as mixture of diastereoisomers (compound 1: 104 mg, 0.383 mmol and compound 2: 86 mg, 0.388 mmol)

### Compound 1

LC-MS (Method A): retention time 0.77 min, 272 (M+H)

1H NMR (400 MHz, CDCl3) δ ppm 7.37 (d, J=2.18 Hz, 5H) 7.31 (s, 1H) 7.22 (s, 1H) 5.45 (dd, J=8.36, 6.90 Hz, 1H) 5.30 (q, J=7.15 Hz, 1H) 3.87 (s, 3H) 3.76-3.81 (m, 1H) 3.09 (dd, J=8.90, 6.72 Hz, 1H) 2.63-2.90 (m, 1H) 1.63 (d, J=7.27 Hz, 3H)

### Compound 2

LC-MS (Method A): retention time 0.79 min, 272 (M+H)

1H NMR (400 MHz, CDCl3) δ ppm 7.52 (s, 1H) 7.47 (s, 1H) 7.30-7.42 (m, 5H) 5.40 (t, J=7.81 Hz, 2H) 5.21-5.34 (m, 2H) 3.95 (s, 3H) 3.35-3.52 (m, 3H) 2.91-3.13 (m, 1H) 1.60 (d, J=6.90 Hz, 3H)

### Method 2: Step 2: Preparation of 1-methyl-4-(1-methylpyrazol-4-yl)-1,2,3,4-tetrahydroisoquinoline

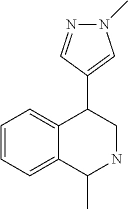 [SMILES: CC1NCC(c2cnn(C)c2)c2ccccc21]

A 5 mL vial under argon was charged with 5-(1-methylpyrazol-4-yl)-3-(1-phenylethyl)oxazolidin-2-one (50 mg, 0.184 mmol), chlorobenzene (0.55 mL) and nitroethane (0.033 mL, 0.460 mmol) at rt. Then, aluminum chloride (0.06144 g, 0.460 mmol) was added at rt. The reaction mixture was heated to 50° C. for 1 hour then at 60° C. over night. The reaction mixture was carefully poured into a saturated solution of sodium bicarbonate and the mixture was diluted with ethyl acetate. After separation of the organic layer, the aqueous layer (pH=9) was back extracted twice with ethyl acetate. The combined organic layers were dried over sodium sulfate, filtered and concentrated in vacuo to afford 1-methyl-4-(1-methylpyrazol-4-yl)-1,2,3,4-tetrahydroisoquinoline as a mixture of diastereoisomers anti/syn 3:2.

LC-MS (Method A): retention time 0.34 min, 228 (M+H)

Further examples of synthesized compounds of formula (I) are shown in Table T1

TABLE T1
Synthesised compounds and Spectral and Physical Chemical Data.

| | | | RT | [M + H] | | MP in |
|---|---|---|---|---|---|---|
| Entry | IUPAC name | Structure | (min) | (measured) | Method | [° C.] |
| P-1 | [5-(2,4-difluorophenyl)- 1,3,4-thiadiazol-2-yl]-[4- (1-methylpyrazol-4-yl)- 3,4-dihydro-1H-isoquin- olin-2-yl]methanone | 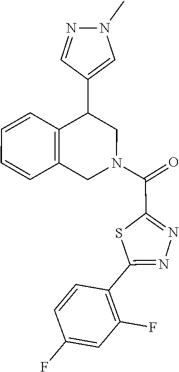 [SMILES: Cn1cc(C2CN(C(=O)c3nnc(-c4ccc(F)cc4F)s3)Cc3ccccc32)cn1] | 1.18 | 436 | C | 158- 160 |
| P-2 | [(1S,4S)-1,4-dimethyl-4- (1-methylpyrazol-4-yl)- 1,3-dihydroisoquinolin-2- yl]-(5-phenylisoxazol-3- yl)methanone | 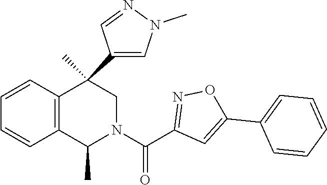 [SMILES: C[C@H]1c2ccccc2[C@@](C)(c2cnn(C)c2)CN1C(=O)c1cc(-c2ccccc2)on1] | 1.07 | 413 | A | |
| P-3 | [(1S,4S)-1,4-dimethyl-4- (1-methylpyrazol-4-yl)- 1,3-dihydroisoquinolin-2- yl]-(5-phenylisoxazol-3- yl)methanone | 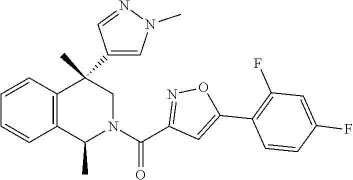 [SMILES: C[C@H]1c2ccccc2[C@](C)(c2cnn(C)c2)CN1C(=O)c1cc(-c2ccc(F)cc2F)on1] | 1.07 | 413 | A | |
| P-4 | [(1R,4S)-1,4-dimethyl-4- (1-methylpyrazol-4-yl)- 1,3-dihydroisoquinolin-2- yl]-(5-phenylisoxazol-3- yl)methanone | 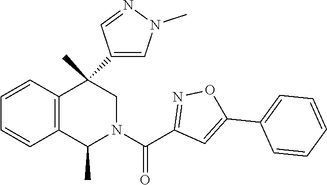 [SMILES: C[C@H]1c2ccccc2[C@](C)(c2cnn(C)c2)CN1C(=O)c1cc(-c2ccccc2)on1] | 1.07 | 413 | A | |
| P-5 | [(1S,4R)-1,4-dimethyl-4- (1-methylpyrazol-4-yl)- 1,3-dihydroisoquinolin-2- yl]-(5-phenylisoxazol-3- yl)methanone | 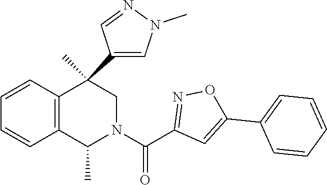 [SMILES: C[C@@H]1c2ccccc2[C@@](C)(c2cnn(C)c2)CN1C(=O)c1cc(-c2ccccc2)on1] | 1.07 | 413 | A | |
| P-6 | [5-(2,4-difluorophenyl)- isoxazol-3-yl]-[(1S,4S)- 1,4-dimethyl-4-(1-meth- ylpyrazol-4-yl)-1,3- dihydroisoquinolin-2-yl] methanone | 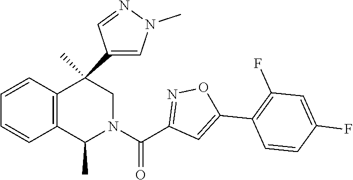 [SMILES: C[C@H]1c2ccccc2[C@@](C)(c2cnn(C)c2)CN1C(=O)c1cc(-c2ccc(F)cc2F)on1] | 1.07 | 449 | A | |
| P-7 | [5-(2,4-difluorophenyl)- isoxazol-3-yl]-[(1R,4R)- 1,4-dimethyl-4-(1-methyl- pyrazol-4-yl)-1,3- dihydroisoquinolin-2-yl] methanone | 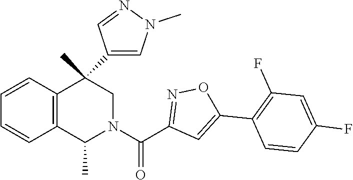 [SMILES: C[C@@H]1c2ccccc2[C@](C)(c2cnn(C)c2)CN1C(=O)c1cc(-c2ccc(F)cc2F)on1] | 1.07 | 449 | A | |
| P-8 | [5-(2,4-difluorophenyl)- isoxazol-3-yl]-[(1S,4R)- 1,4-dimethyl-4-(1-meth- ylpyrazol-4-yl)-1,3- dihydroisoquinolin-2-yl] methanone | 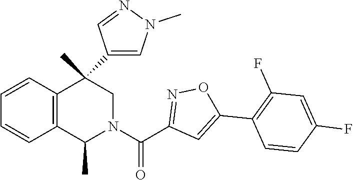 [SMILES: C[C@H]1c2ccccc2[C@](C)(c2cnn(C)c2)CN1C(=O)c1cc(-c2ccc(F)cc2F)on1] | 1.07 | 449 | A | |
| P-9 | [5-(2,4-difluorophenyl)- isoxazol-3-yl]-[(1R,4S)- 1,4-dimethyl-4-(1-meth- ylpyrazol-4-yl)-1,3- dihydroisoquinolin-2-yl] methanone | 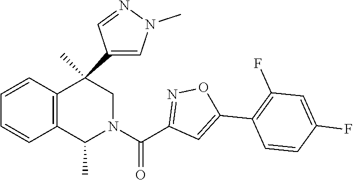 [SMILES: C[C@@H]1c2ccccc2[C@@](C)(c2cnn(C)c2)CN1C(=O)c1cc(-c2ccc(F)cc2F)on1] | 1.07 | 449 | A | |
| P-10 | [5-(2,4-difluorophenyl)- isoxazol-3-yl]-[1-(N- methoxy-C-methyl- carbonimidoyl)-4-(1- methylpyrazol-4-yl)-3,4- dihydro-1H-isoquinolin- 2-yl]methanone | 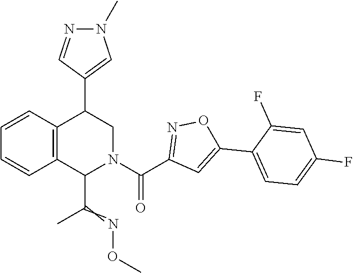 [SMILES: CON=C(C)C1c2ccccc2C(c2cnn(C)c2)CN1C(=O)c1cc(-c2ccc(F)cc2F)on1] | 1.09 | 492 | A | |
| P-11 | [5-(2,4-difluorophenyl)- isoxazol-3-yl]-[4-(5- fluoro-1-methyl-pyrazol- 4-yl)-1,4-dimethyl-1,3- dihydroisoquinolin-2-yl] methanone | 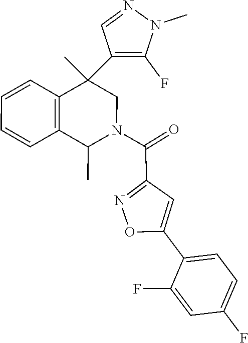 [SMILES: CC1c2ccccc2C(C)(c2cnn(C)c2F)CN1C(=O)c1cc(-c2ccc(F)cc2F)on1] | 1.13 | 467 | A | |
| P-12 | ethyl 4-[2-[5-(2,4- difluorophenyl)isoxazole- 3-carbon-yl]-3,4-dihydro- 1H-isoquinolin-4-yl]-2- methyl-pyrazole-3- carboxylate | 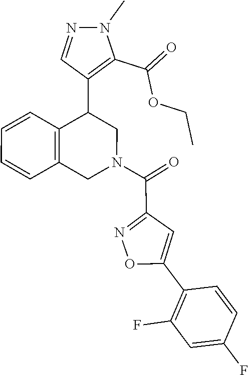 [SMILES: CCOC(=O)c1c(C2CN(C(=O)c3cc(-c4ccc(F)cc4F)on3)Cc3ccccc32)cnn1C] | 1.03 | 493 | A | |
| P-13 | [5-(2,4-difluorophenyl)- isoxazol-3-yl]-[4-[5-(N- methoxy-C-methyl- carbonimidoyl)-1-meth- yl-pyrazol-4-yl]-3,4- dihydro-1H-isoquinolin- 2-yl]methanone | 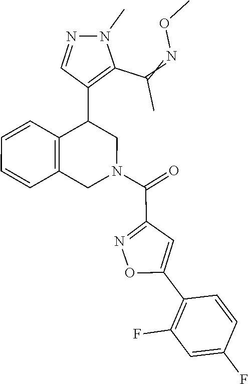 [SMILES: CON=C(C)c1c(C2CN(C(=O)c3cc(-c4ccc(F)cc4F)on3)Cc3ccccc32)cnn1C] | 1.06 and 1.08 | 492 | A | |
| P-14 | 1-[4-[2-[5-(2,4-difluoro- phenyl)isoxazole-3- carbonyl]-3,4-dihydro- 1H-isoquinolin-4-yl]-2- methyl-pyrazol-3-yl] ethanone | 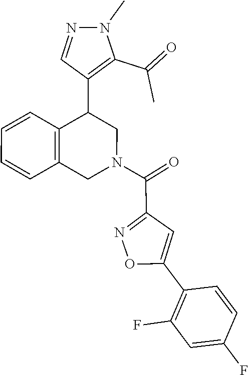 [SMILES: CC(=O)c1c(C2CN(C(=O)c3cc(-c4ccc(F)cc4F)on3)Cc3ccccc32)cnn1C] | 1.06 | 463 | A | |
| P-15 | [4-(1,5-dimethylpyrazol- 4-yl)-1,4-dimethyl-1,3- dihydroisoquinolin-2-yl]- (5-phenylisoxazol-3-yl)- methanone | 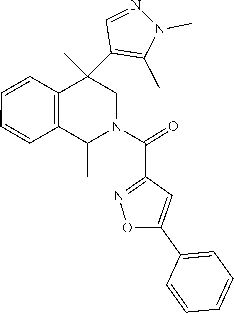 [SMILES: Cc1c(C2(C)CN(C(=O)c3cc(-c4ccccc4)on3)C(C)c3ccccc32)cnn1C] | 1.08 | 427 | A | |
| P-16 | [5-(2,4-difluorophenyl)- isoxazol-3-yl]-[4-(1,5- dimethylpyrazol-4-yl)- 1,4-dimethyl-1,3- dihydroisoquinolin-2- yl]methanone | 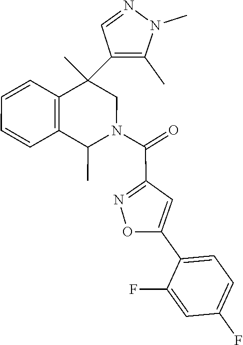 [SMILES: Cc1c(C2(C)CN(C(=O)c3cc(-c4ccc(F)cc4F)on3)C(C)c3ccccc32)cnn1C] | 1.11 | 463 | A | |
| P-17 | 1-[2-[5-(2,4-difluorophen- yl)isoxazole-3-carbonyl]- 4-(1-methylpyrazol-4-yl)- 3,4-dihydro-1H-isoquin- olin-1-yl]ethanone | 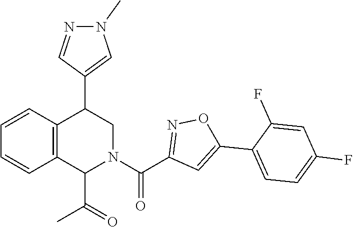 [SMILES: CC(=O)C1c2ccccc2C(c2cnn(C)c2)CN1C(=O)c1cc(-c2ccc(F)cc2F)on1] | 1.04 | 463 | A | |
| P-18 | [5-(2,4-difluorophenyl)- isoxazol-3-yl]-[7-meth- oxy-1,4-dimethyl-4-(1- methylpyrazol-4-yl)-1,3- dihydroisoquinolin-2- yl]methanone | 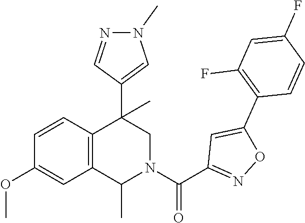 [SMILES: COc1ccc2c(c1)C(C)N(C(=O)c1cc(-c3ccc(F)cc3F)on1)CC2(C)c1cnn(C)c1] | 1.08 | 479 | A | |
| P-19 | 2-[5-(2,4-difluorophenyl)- isoxazole-3-carbonyl]-N- methoxy-N-methyl-4-(1- methylpyrazol-4-yl)-3,4- dihydro-1H-isoquinoline- 1-carboxamide | 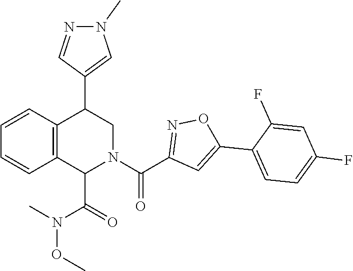 [SMILES: CON(C)C(=O)C1c2ccccc2C(c2cnn(C)c2)CN1C(=O)c1cc(-c2ccc(F)cc2F)on1] | 1.05 | 508 | A | |
| P-20 | [5-(2-fluoro-4-methyl- sulfonyl-phenyl)isoxazol- 3-yl]-[4-(1-methylpyr- azol-4-yl)-3,4-dihydro- 1H-isoquinolin-2-yl] methanone | 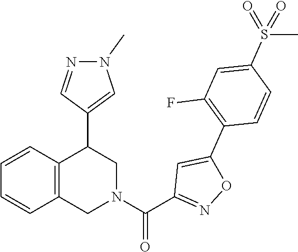 [SMILES: Cn1cc(C2CN(C(=O)c3cc(-c4ccc(S(C)(=O)=O)cc4F)on3)Cc3ccccc32)cn1] | 0.91 | 481 | A | |
| P-21 | [5-(2,4-difluorophenyl)- isoxazol-3-yl]-[4-(1- methylpyrazol-4-yl)-7- phenyl-3,4-dihydro-1H- isoquinolin-2-yl] methanone | 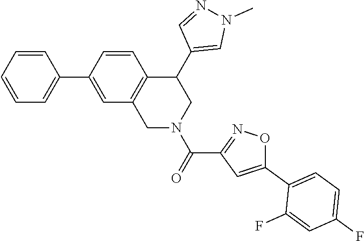 [SMILES: Cn1cc(C2CN(C(=O)c3cc(-c4ccc(F)cc4F)on3)Cc3cc(-c4ccccc4)ccc32)cn1] | 1.14 | 497 | A | |
| P-22 | N,N-dimethyl-4-(1- methylpyrazol-4-yl)-2- (5-phenylisoxazole-3- carbonyl)-3,4-dihydro- 1H-isoquinoline-1- carboxamide | 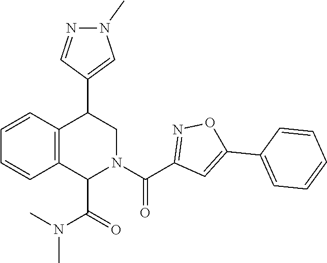 [SMILES: CN(C)C(=O)C1c2ccccc2C(c2cnn(C)c2)CN1C(=O)c1cc(-c2ccccc2)on1] | 0.97 | 456 | A | |
| P-23 | 2-[5-(2,4-difluorophen- yl)isoxazole-3-carbonyl]- N,N-dimethyl-4-(1- methylpyrazol-4-yl)-3,4- dihydro-1H-isoquinoline- 1-carboxamide | 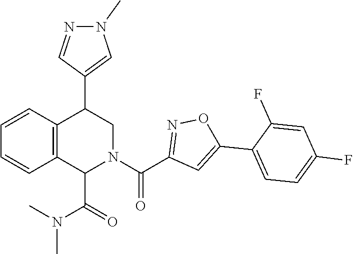 [SMILES: CN(C)C(=O)C1c2ccccc2C(c2cnn(C)c2)CN1C(=O)c1cc(-c2ccc(F)cc2F)on1] | 1.00 | 492 | A | |
| P-24 | N-methyl-4-(1-methyl- pyrazol-4-yl)-2-(5-phen- ylisoxazole-3-carbonyl)- 3,4-dihydro-1H-isoquin- oline-1-carboxamide | 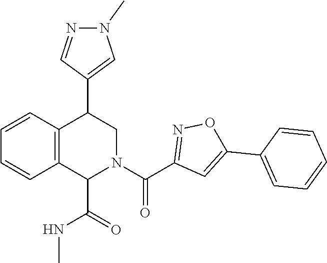 [SMILES: CNC(=O)C1c2ccccc2C(c2cnn(C)c2)CN1C(=O)c1cc(-c2ccccc2)on1] | 0.93 | 442 | A | |
| P-25 | 2-[5-(2,4-difluorophenyl)- isoxazole-3-carbonyl]- N-methyl-4-(1-methyl- pyrazol-4-yl)-3,4- dihydro-1H-isoquinoline- 1-carboxamide | 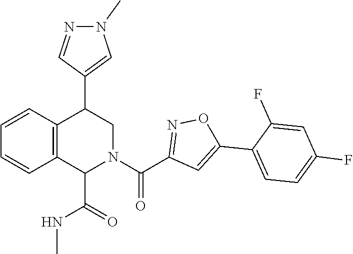 [SMILES: CNC(=O)C1c2ccccc2C(c2cnn(C)c2)CN1C(=O)c1cc(-c2ccc(F)cc2F)on1] | 0.95 | 478 | A | |
| P-26 | [6-(2,2-difluoroethoxy)- 1,4-dimethyl-4-(1- methylpyrazol-4-yl)-1,3- dihydroisoquinolin-2-yl]- (5-phenylisoxazol-3-yl)- methanone | 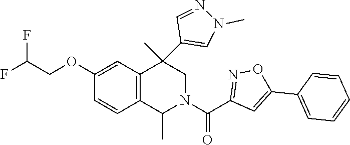 [SMILES: CC1c2ccc(OCC(F)F)cc2C(C)(c2cnn(C)c2)CN1C(=O)c1cc(-c2ccccc2)on1] | 1.08 | 493 | A | |
| P-27 | [1,4-dimethyl-4-(1-meth- ylpyrazol-4-yl)-6-(2,2,2- trifluoroethoxy)-1,3- dihydroisoquinolin-2-yl]- (5-phenylisoxazol-3-yl)- methanone | 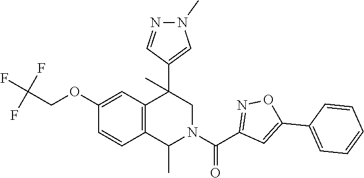 [SMILES: CC1c2ccc(OCC(F)(F)F)cc2C(C)(c2cnn(C)c2)CN1C(=O)c1cc(-c2ccccc2)on1] | 1.13 | 511 | A | |
| P-28 | [6-(difluoromethoxy)- 1,4-dimethyl-4-(1-meth- ylpyrazol-4-yl)-1,3- dihydroisoquinolin-2-yl]- (5-phenylisoxazol-3-yl)- methanone | 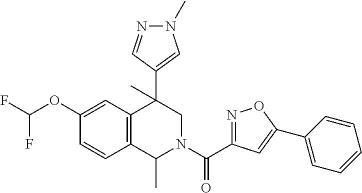 [SMILES: CC1c2ccc(OC(F)F)cc2C(C)(c2cnn(C)c2)CN1C(=O)c1cc(-c2ccccc2)on1] | 1.09 | 479 | A | |
| P-29 | [6-hydroxy-1,4-dimethyl- 4-(1-methylpyrazol-4-yl)- 1,3-dihydroisoquinolin-2- yl]-(5-phenylisoxazol-3- yl)methanone | 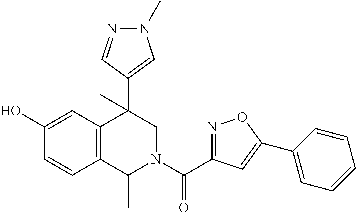 [SMILES: CC1c2ccc(O)cc2C(C)(c2cnn(C)c2)CN1C(=O)c1cc(-c2ccccc2)on1] | 0.94 | 429 | A | |
| P-30 | [6-(difluoromethoxy)-1,4- dimethyl-4-(1-methyl- pyrazol-4-yl)-1,3-dihydro- isoquinolin-2-yl]-[5-(2,4- difluorophenyl)isoxazol- 3-yl]methanone | 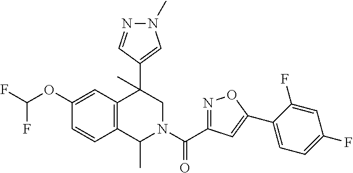 [SMILES: CC1c2ccc(OC(F)F)cc2C(C)(c2cnn(C)c2)CN1C(=O)c1cc(-c2ccc(F)cc2F)on1] | 1.10 | 515 | A | |
| P-31 | [5-(4-ethynyl-2-fluoro- phenyl)isoxazol-3-yl]-[4- (1-methylpyrazol-4-yl)- 3,4-dihydro-1H-isoquin- olin-2-yl]methanone | 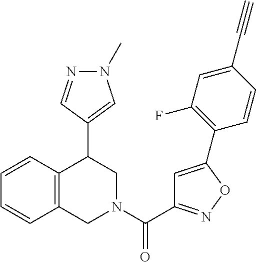 [SMILES: C#Cc1ccc(-c2cc(C(=O)N3Cc4ccccc4C(c4cnn(C)c4)C3)no2)c(F)c1] | 1.05 | 427 | A | |
| P-32 | [5-(4-bromo-2-fluoro- phenyl)isoxazol-3-yl]- [4-(1-methylpyrazol-4- yl)-3,4-dihydro-1H-iso- quinolin-2-yl]methanone | 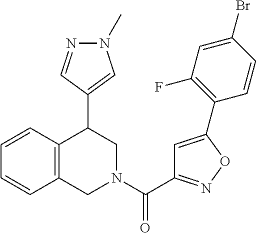 [SMILES: Cn1cc(C2CN(C(=O)c3cc(-c4ccc(Br)cc4F)on3)Cc3ccccc32)cn1] | 1.11 | 481/483 | A | |
| P-33 | [6-(2,2-difluoroethoxy)- 1,4-dimethyl-4-(1-meth- ylpyrazol-4-yl)-1,3- dihydroisoquinolin-2-yl]- [5-(2,4-difluorophenyl)- isoxazol-3-yl]methanone | 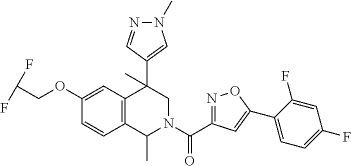 [SMILES: CC1c2ccc(OCC(F)F)cc2C(C)(c2cnn(C)c2)CN1C(=O)c1cc(-c2ccc(F)cc2F)on1] | 1.10 | 529 | A | |
| P-34 | [5-(2,4-difluorophenyl)- isoxazol-3-yl]-[1,4- dimethyl-4-(1-methyl- pyrazol-4-yl)-6-(2,2,2- trifluoroethoxy)-1,3- dihydroisoquinolin-2- yl]methanone | 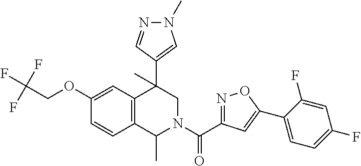 [SMILES: CC1c2ccc(OCC(F)(F)F)cc2C(C)(c2cnn(C)c2)CN1C(=O)c1cc(-c2ccc(F)cc2F)on1] | 1.14 | 547 | A | |
| P-35 | [5-(2,4-difluorophenyl)- isoxazol-3-yl]-[6- hydroxy-1,4-dimethyl-4- (1-methylpyrazol-4-yl)- 1,3-dihydroisoquinolin- 2-yl]methanone | 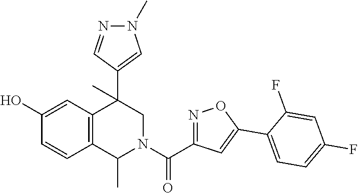 [SMILES: CC1c2ccc(O)cc2C(C)(c2cnn(C)c2)CN1C(=O)c1cc(-c2ccc(F)cc2F)on1] | 0.96 and 0.97 | 465 | A | |
| P-36 | [5-(2,4-difluorophenyl)- isoxazol-3-yl]-[1-ethyl-4- (1-methylpyrazol-4-yl)- 3,4-dihydro-1H-isoquin- olin-2-yl]methanone | 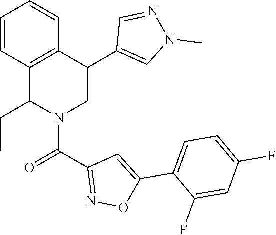 [SMILES: CCC1c2ccccc2C(c2cnn(C)c2)CN1C(=O)c1cc(-c2ccc(F)cc2F)on1] | 1.09 and 1.11 | 449 | A | |
| P-37 | [1-ethyl-4-(1-methylpyr- azol-4-yl)-3,4-dihydro- 1H-isoquinolin-2-yl]-(5- phenylisoxazol-3-yl)- methanone | 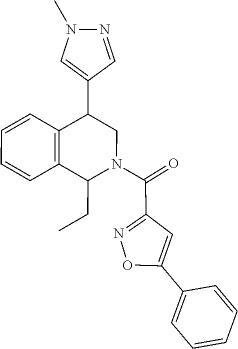 [SMILES: CCC1c2ccccc2C(c2cnn(C)c2)CN1C(=O)c1cc(-c2ccccc2)on1] | 1.07 min and 1.12 | 413 | A | |
| P-38 | [4-(5-chloro-1-methyl- pyrazol-4-yl)-3,4-dihydro- 1H-isoquinolin-2-yl]-[5- (2,4-difluorophenyl)isox- azol-3-yl]methanone | 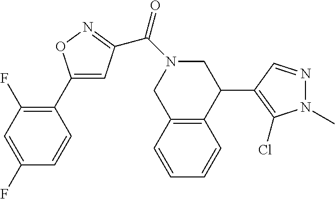 [SMILES: Cn1ncc(C2CN(C(=O)c3cc(-c4ccc(F)cc4F)on3)Cc3ccccc32)c1Cl] | 1.15 | 455/457 | A | |
| P-39 | [6-methoxy-1,4-dimethyl- 4-(1-methylpyrazol-4-yl)- 1,3-dihydroisoquinolin-2- yl]-(5-phenylisoxazol-3- yl)methanone | 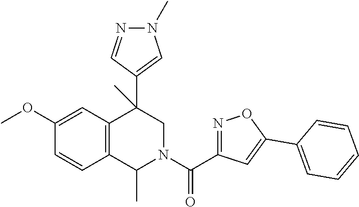 [SMILES: COc1ccc2c(c1)C(C)(c1cnn(C)c1)CN(C(=O)c1cc(-c3ccccc3)on1)C2C] | 1.05 and 1.08 | 443 | A | |
| P-40 | [5-(2,4-difluorophenyl)- isoxazol-3-yl]-[6-meth- oxy-1,4-dimethyl-4-(1- methylpyrazol-4-yl)-1,3- dihydroisoquinolin-2- yl]methanone | 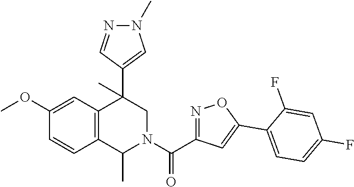 [SMILES: COc1ccc2c(c1)C(C)(c1cnn(C)c1)CN(C(=O)c1cc(-c3ccc(F)cc3F)on1)C2C] | 1.07 and 1.10 | 479 | A | |
| P-41 | methyl 4-(1-methylpyr- azol-4-yl)-2-(5-phenyl- isoxazole-3-carbonyl)- 3,4-dihydro-1H-isoquin- oline-1-carboxylate | 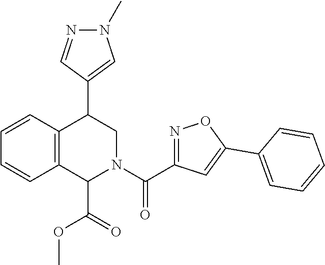 [SMILES: COC(=O)C1c2ccccc2C(c2cnn(C)c2)CN1C(=O)c1cc(-c2ccccc2)on1] | 1.01 and 1.04 | 443 | A | |
| P-42 | methyl 2-[5-(2,4- difluorophenyl)isox- azole-3-carbonyl]-4-(1- methylpyrazol-4-yl)- 3,4-dihydro-1H-isoquin- oline-1-carboxylate | 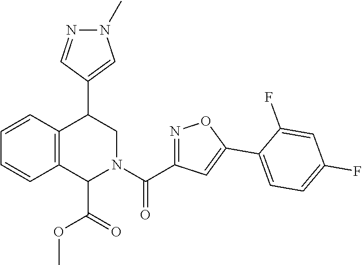 [SMILES: COC(=O)C1c2ccccc2C(c2cnn(C)c2)CN1C(=O)c1cc(-c2ccc(F)cc2F)on1] | 1.04 and 1.06 | 479 | A | |
| P-43 | [5-(2,4-difluorophenyl)- isoxazol-3-yl]-[4-(1-meth- ylpyrazol-4-yl)-1-phenyl- 3,4-dihydro-1H-isoquin- olin-2-yl]methanone | 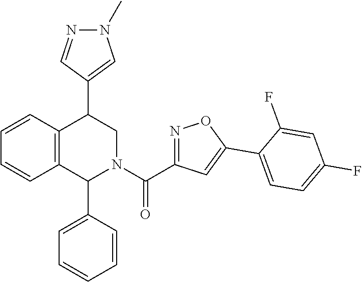 [SMILES: Cn1cc(C2CN(C(=O)c3cc(-c4ccc(F)cc4F)on3)C(c3ccccc3)c3ccccc32)cn1] | 1.16 and 1.18 | 497 | A | |
| P-44 | [5-(2,4-difluorophenyl)- isoxazol-3-yl]-[1-isoprop- yl-4-(1-methylpyrazol-4- yl)-3,4-dihydro-1H-iso- quinolin-2-yl]methanone | 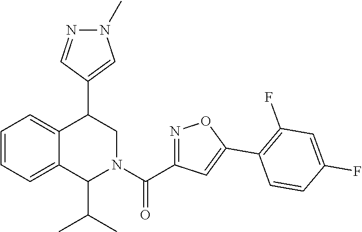 [SMILES: CC(C)C1c2ccccc2C(c2cnn(C)c2)CN1C(=O)c1cc(-c2ccc(F)cc2F)on1] | 1.17 | 463 | A | |
| P-45 | [4-(5-bromo-1-methyl- pyrazol-4-yl)-3,4- dihydro-1H-isoquinolin- 2-yl]-[5-(2,4-difluoro- phenyl)isoxazol-3-yl] methanone | 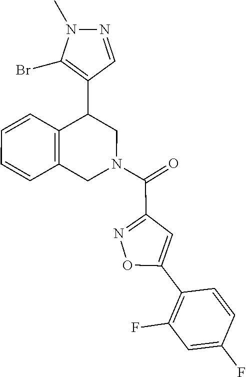 [SMILES: Cn1ncc(C2CN(C(=O)c3cc(-c4ccc(F)cc4F)on3)Cc3ccccc32)c1Br] | 1.12 | 499/501 | A | |
| P-46 | [4-(1,5-dimethylpyrazol- 4-yl)-3,4-dihydro-1H- isoquinolin-2-yl]-[5- (4-fluoro-2-methoxy- phenyl)isoxazol-3-yl] methanone | 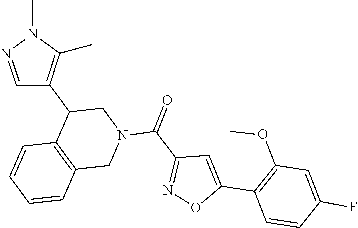 [SMILES: COc1cc(F)ccc1-c1cc(C(=O)N2Cc3ccccc3C(c3cnn(C)c3C)C2)no1] | 1.41 | 447 | E | 75-77 |
| P-47 | [5-(2,4-difluorophenyl)- isoxazol-3-yl]-[6-meth- oxy-1-methyl-4-(1-meth- ylpyrazol-4-yl)-3,4- dihydro-1H-isoquinolin- 2-yl]methanone | 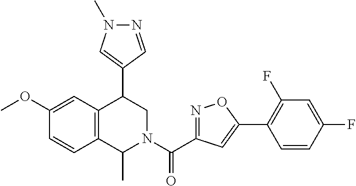 [SMILES: COc1ccc2c(c1)C(c1cnn(C)c1)CN(C(=O)c1cc(-c3ccc(F)cc3F)on1)C2C] | 1.06 | 465 | A | |
| P-48 | [5-(2,4-difluorophenyl)- isoxazol-3-yl]-[(4S)-4- methyl-4-(1-methyl- pyrazol-4-yl)-1,3- dihydro-isoquinolin-2- yl]methanone | 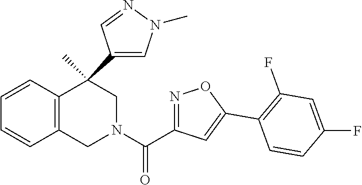 [SMILES: Cn1cc([C@@]2(C)CN(C(=O)c3cc(-c4ccc(F)cc4F)on3)Cc3ccccc32)cn1] | 1.05 | 435 | A | |
| P-49 | [5-(2,4-difluorophenyl)- isoxazol-3-yl]-[(4R)- 4-methyl-4-(1-methyl- pyrazol-4-yl)-1,3- dihydroisoquinolin-2- yl]methanone | 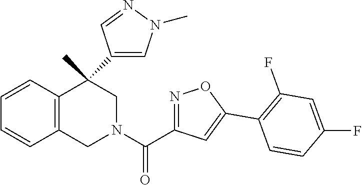 [SMILES: Cn1cc([C@]2(C)CN(C(=O)c3cc(-c4ccc(F)cc4F)on3)Cc3ccccc32)cn1] | 1.05 | 435 | A | |
| P-50 | [7-methoxy-4-(1-methyl- pyrazol-4-yl)-3,4-dihydro- 1H-isoquinolin-2-yl]-(5- phenylisoxazol-3-yl)- methanone | 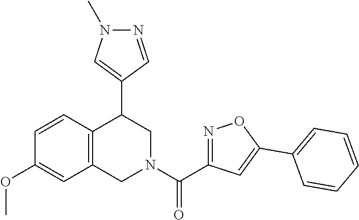 [SMILES: COc1ccc2c(c1)CN(C(=O)c1cc(-c3ccccc3)on1)CC2c1cnn(C)c1] | 0.99 | 415 | A | |
| P-51 | [5-(2,4-difluorophenyl)- isoxazol-3-yl]-[7-meth- oxy-4-(1-methylpyr- azol-4-yl)-3,4-dihydro- 1H-isoquinolin-2-yl] methanone | 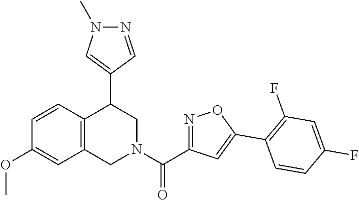 [SMILES: COc1ccc2c(c1)CN(C(=O)c1cc(-c3ccc(F)cc3F)on1)CC2c1cnn(C)c1] | 1.01 | 451 | A | 126- 127 |
| P-52 | [5-(2,4-difluorophenyl)- isoxazol-3-yl]-[4-(5- fluoro-1-methyl-pyrazol- 4-yl)-3,4-dihydro-1H-iso- quinolin-2-yl]methanone | 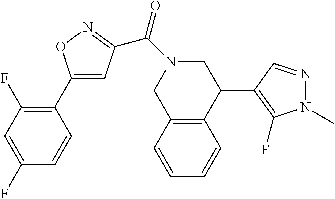 [SMILES: Cn1ncc(C2CN(C(=O)c3cc(-c4ccc(F)cc4F)on3)Cc3ccccc32)c1F] | 1.06 | 439 | A | |
| P-53 | [5-(2,4-difluorophenyl)- isoxazol-3-yl]-[4-methyl- 4-(1-methylpyrazol-4-yl)- 1,3-dihydroisoquinolin- 2-yl]methanone | 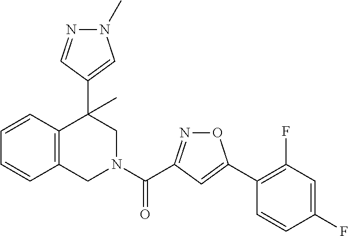 [SMILES: Cn1cc(C2(C)CN(C(=O)c3cc(-c4ccc(F)cc4F)on3)Cc3ccccc32)cn1] | 1.05 | 435 | A | |
| P-54 | [5-(2-fluorophenyl)- 1,3,4-oxadiazol-2-yl]-[4- (1-methylpyrazol-4-yl)- 3,4-dihydro-1H-isoquin- olin-2-yl]methanone | 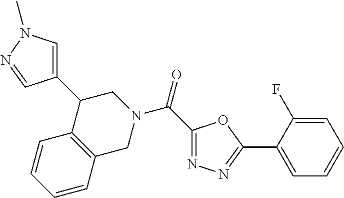 [SMILES: Cn1cc(C2CN(C(=O)c3nnc(-c4ccccc4F)o3)Cc3ccccc32)cn1] | 1.07 | 404 | C | 122- 123 |
| P-55 | [5-(2,4-difluorophenyl)- 1,3,4-oxadiazol-2-yl]-[4- (1-methylpyrazol-4-yl)- 3,4-dihydro-1H-isoquin- olin-2-yl]methanone | 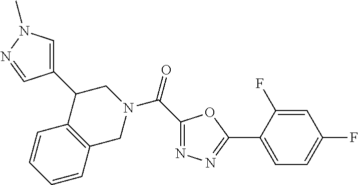 [SMILES: Cn1cc(C2CN(C(=O)c3nnc(-c4ccc(F)cc4F)o3)Cc3ccccc32)cn1] | 1.09 | 422 | C | 124- 125 |
| P-56 | [5-(4-fluorophenyl)-1,3,4- oxadiazol-2-yl]-[4-(1- methylpyrazol-4-yl)-3,4- dihydro-1H-isoquinolin- 2-yl]methanone | 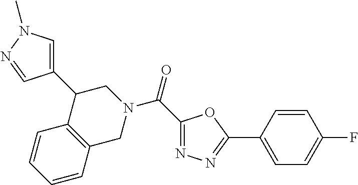 [SMILES: Cn1cc(C2CN(C(=O)c3nnc(-c4ccc(F)cc4)o3)Cc3ccccc32)cn1] | 1.10 | 404 | C | 146- 148 |
| P-57 | [5-(2,4-difluorophenyl)- isoxazol-3-yl]-[4-(1- methylpyrazol-4-yl)-6- methylsulfonyl-3,4- dihydro-1H-isoquinolin- 2-yl]methanone | 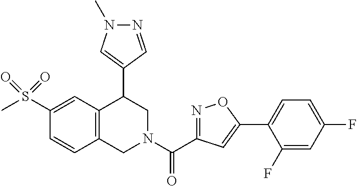 [SMILES: Cn1cc(C2CN(C(=O)c3cc(-c4ccc(F)cc4F)on3)Cc3ccc(S(C)(=O)=O)cc32)cn1] | 0.91 | 499 | A | |
| P-58 | [6-chloro-4-(1-methyl- pyrazol-4-yl)-3,4- dihydro-1H-isoquinolin- 2-yl]-[5-(2,4-difluoro- phenyl)isoxazol-3-yl] methanone | 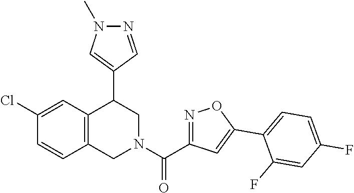 [SMILES: Cn1cc(C2CN(C(=O)c3cc(-c4ccc(F)cc4F)on3)Cc3ccc(Cl)cc32)cn1] | 1.09 | 455 | A | |
| P-59 | [5-(2,4-difluorophenyl)- isoxazol-3-yl]-[4-(1- methylpyrazol-4-yl)-1- propyl-3,4-dihydro- 1H-isoquinolin-2-yl] methanone | 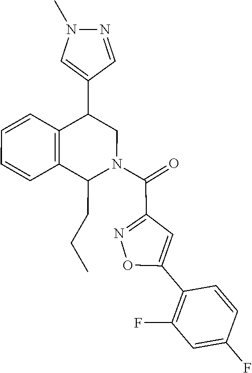 [SMILES: CCCC1c2ccccc2C(c2cnn(C)c2)CN1C(=O)c1cc(-c2ccc(F)cc2F)on1] | 1.13 | 463 | A | |
| P-60 | [6-methoxy-4-(1-methyl- pyrazol-4-yl)-3,4- dihydro-1H-isoquinolin- 2-yl]-(5-phenylisoxazol- 3-yl)methanone | 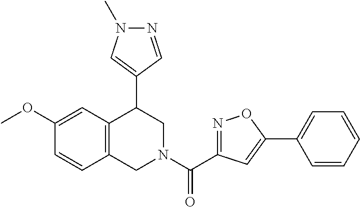 [SMILES: COc1ccc2c(c1)C(c1cnn(C)c1)CN(C(=O)c1cc(-c3ccccc3)on1)C2] | 0-98 | 415 | A | |
| P-61 | methyl 2-[5-(2,4-difluoro- phenyl)isoxazole-3-car- bonyl]-4-(1-methylpyr- azol-4-yl)-3,4-dihydro- 1H-isoquinoline-6- carboxylate | 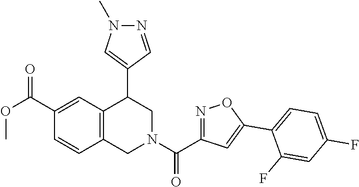 [SMILES: COC(=O)c1ccc2c(c1)C(c1cnn(C)c1)CN(C(=O)c1cc(-c3ccc(F)cc3F)on1)C2] | 1.00 | 479 | A | |
| P-62 | 2-[5-(2,4-difluorophenyl)- isoxazole-3-carbonyl]-4- (1-methylpyrazol-4-yl)- 3,4-dihydro-1H-isoquin- oline-6-carbonitrile | 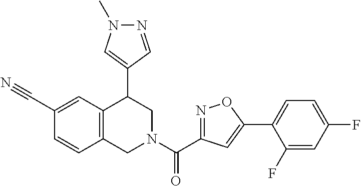 [SMILES: Cn1cc(C2CN(C(=O)c3cc(-c4ccc(F)cc4F)on3)Cc3ccc(C#N)cc32)cn1] | 0.98 | 446 | A | |
| P-63 | [4-(1,5-dimethylpyrazol- 4-yl)-3,4-dihydro-1H- isoquinolin-2-yl]-[5- (4-fluorophenyl)-1,3,4- thiadiazol-2-yl]methanone | 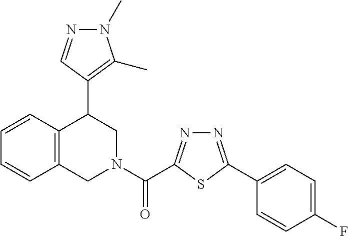 [SMILES: Cc1c(C2CN(C(=O)c3nnc(-c4ccc(F)cc4)s3)Cc3ccccc32)cnn1C] | 1.14 | 434 | D | 165- 168 |
| P-64 | [4-(1,5-dimethylpyrazol- 4-yl)-3,4-dihydro-1H- isoquinolin-2-yl]-[5-(2- fluorophenyl)-1,3,4- thiadiazol-2-yl]methanone | 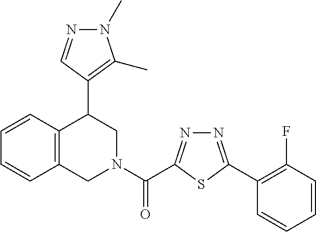 [SMILES: Cc1c(C2CN(C(=O)c3nnc(-c4ccccc4F)s3)Cc3ccccc32)cnn1C] | 1.14 | 434 | D | 58-62 |
| P-65 | [5-(2,4-difluorophenyl)- 1,3,4-thiadiazol-2-yl]-[4- (1,5-dimethylpyrazol-4- yl)-3,4-dihydro-1H-iso- quinolin-2-yl]methanone | 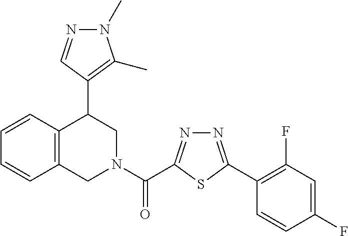 [SMILES: Cc1c(C2CN(C(=O)c3nnc(-c4ccc(F)cc4F)s3)Cc3ccccc32)cnn1C] | 1.15 | 452 | D | 150- 152 |
| P-66 | [5-(2,4-difluorophenyl)- isoxazol-3-yl]-[6-meth- oxy-4-(1-methylpyrazol- 4-yl)-3,4-dihydro-1H-iso- quinolin-2-yl]methanone | 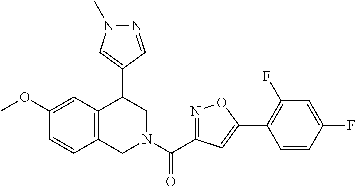 [SMILES: COc1ccc2c(c1)C(c1cnn(C)c1)CN(C(=O)c1cc(-c3ccc(F)cc3F)on1)C2] | 1.01 | 451 | A | |
| P-67 | [4-(1-methylpyrazol-4- yl)-3,4-dihydro-1H- isoquinolin-2-yl]-(5- phenyl-4H-1,2,4-triazol- 3-yl)methanone | 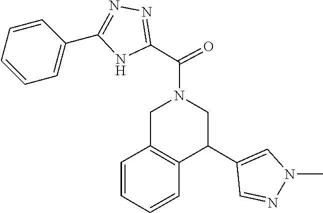 [SMILES: Cn1cc(C2CN(C(=O)c3nnc(-c4ccccc4)[nH]3)Cc3ccccc32)cn1] | 0.93 | 385 | B | |
| P-68 | (5-cyclopropyloxazol- 2-yl)-[4-(1-methylpyr- azol-4-yl)-3,4-dihydro- 1H-isoquinolin-2-yl] methanone | 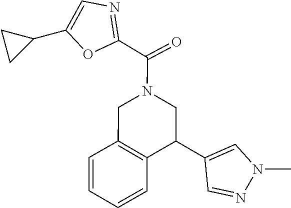 [SMILES: Cn1cc(C2CN(C(=O)c3ncc(C4CC4)o3)Cc3ccccc32)cn1] | 0.95 | 349 | B | |
| P-69 | [2-(4-fluorophenyl)- oxazol-4-yl]-[4-(1-meth- ylpyrazol-4-yl)-3,4- dihydro-1H-isoquinolin- 2-yl]methanone | 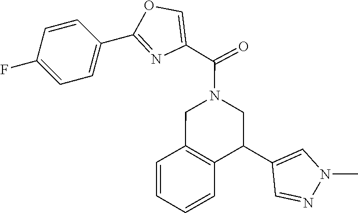 [SMILES: Cn1cc(C2CN(C(=O)c3coc(-c4ccc(F)cc4)n3)Cc3ccccc32)cn1] | 1.05 | 403 | B | |
| P-70 | (5-ethyl-4-methyl- isoxazol-3-yl)-[4-(1- methylpyrazol-4-yl)-3,4- dihydro-1H-isoquinolin- 2-yl]methanone | 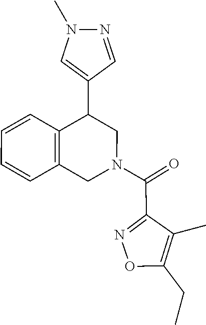 [SMILES: CCc1onc(C(=O)N2Cc3ccccc3C(c3cnn(C)c3)C2)c1C] | 0.98 | 351 | B | |
| P-71 | [2-(4-fluorophenyl)-4- methyl-thiazol-5-yl]-[4- (1-methylpyrazol-4-yl)- 3,4-dihydro-1H-isoquin- olin-2-yl]methanone | 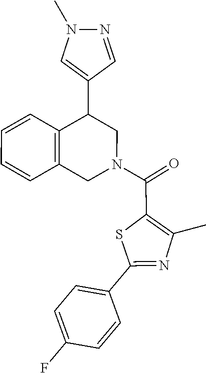 [SMILES: Cc1nc(-c2ccc(F)cc2)sc1C(=O)N1Cc2ccccc2C(c2cnn(C)c2)C1] | 1.07 | 433 | B | |
| P-72 | (5-cyclopropyl-1-ethyl- pyrazol-3-yl)-[4-(1- methylpyrazol-4-yl)-3,4- dihydro-1H-isoquinolin- 2-yl]methanone | 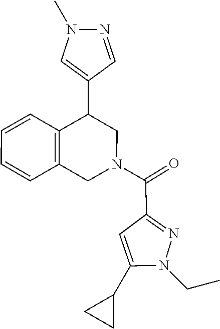 [SMILES: CCn1nc(C(=O)N2Cc3ccccc3C(c3cnn(C)c3)C2)cc1C1CC1] | 0.98 | 376 | B | |
| P-73 | (5-isopropyl-1,2,4- oxadiazol-3-yl)-[4-(1- methylpyrazol-4-yl)-3,4- dihydro-1H-isoquinolin- 2-yl]methanone | 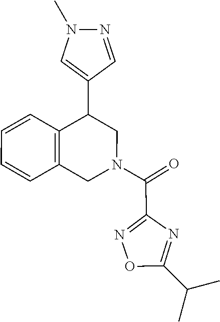 [SMILES: CC(C)c1nc(C(=O)N2Cc3ccccc3C(c3cnn(C)c3)C2)no1] | 0.97 | 352 | B | |
| P-74 | [5-(2-fluoro-4-methoxy- phenyl)isoxazol-3-yl]-[4- (1-methylpyrazol-4-yl)- 3,4-dihydro-1H-isoquin- olin-2-yl]methanone | 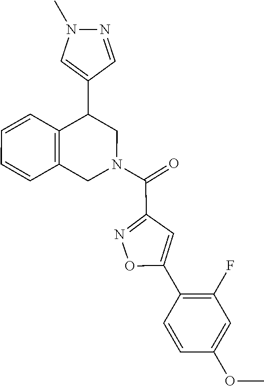 [SMILES: COc1ccc(-c2cc(C(=O)N3Cc4ccccc4C(c4cnn(C)c4)C3)no2)c(F)c1] | 1.10 | 433 | B | |
| P-75 | (4-cyclopropylthiazol- 2-yl)-[4-(1-methylpyr- azol-4-yl)-3,4-dihydro- 1H-isoquinolin-2-yl] methanone | 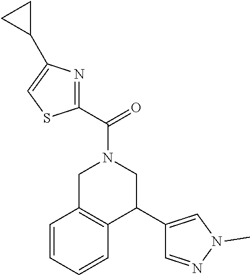 [SMILES: Cn1cc(C2CN(C(=O)c3nc(C4CC4)cs3)Cc3ccccc32)cn1] | 1.06 | 365 | B | |
| P-76 | [4-(1-methylpyrazol-4- yl)-3,4-dihydro-1H- isoquinolin-2-yl]-[5-[4- (trifluoromethyl)phenyl]- 2-furyl]methanone | 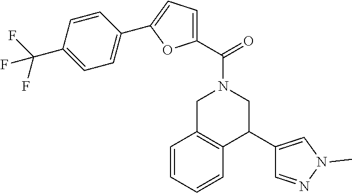 [SMILES: Cn1cc(C2CN(C(=O)c3ccc(-c4ccc(C(F)(F)F)cc4)o3)Cc3ccccc32)cn1] | 1.18 | 452 | B | |
| P-77 | [3-(2-fluorophenyl)-1H- pyrazol-5-yl]-[4-(1-meth- ylpyrazol-4-yl)-3,4- dihydro-1H-isoquinolin- 2-yl]methanone | 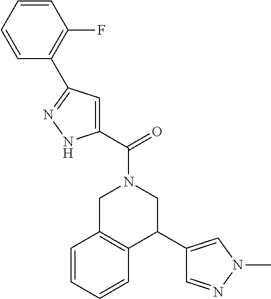 [SMILES: Cn1cc(C2CN(C(=O)c3cc(-c4ccccc4F)n[nH]3)Cc3ccccc32)cn1] | 0.96 | 402 | B | |
| P-78 | (2-cyclopropylthiazol- 4-yl)-[4-(1-methylpyr- azol-4-yl)-3,4-dihydro- 1H-isoquinolin-2-yl] methanone | 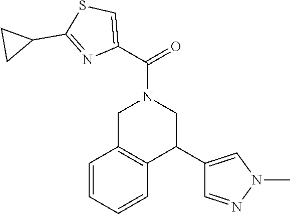 [SMILES: Cn1cc(C2CN(C(=O)c3csc(C4CC4)n3)Cc3ccccc32)cn1] | 0.98 | 365 | B | |
| P-79 | (2-methyl-5-phenyl- pyrazol-3-yl)-[4-(1- methylpyrazol-4-yl)-3,4- dihydro-1H-isoquinolin- 2-yl]methanone | 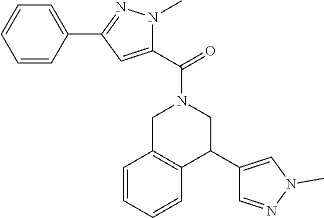 [SMILES: Cn1cc(C2CN(C(=O)c3cc(-c4ccccc4)nn3C)Cc3ccccc32)cn1] | 1.04 | 398 | B | |
| P-80 | [4-(1-methylpyrazol-4- yl)-3,4-dihydro-1H-iso- quinolin-2-yl]-(3-phenyl- 1,2,4-thiadiazol-5-yl)- methanone | 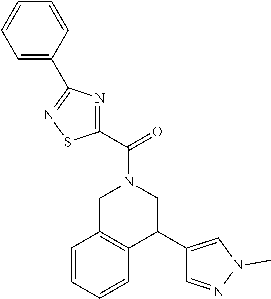 [SMILES: Cn1cc(C2CN(C(=O)c3nc(-c4ccccc4)ns3)Cc3ccccc32)cn1] | 1.16 | 402 | B | |
| P-81 | (1-methyl-5-phenyl- imidazol-2-yl)-[4-(1- methylpyrazol-4-yl)-3,4- dihydro-1H-isoquinolin- 2-yl]methanone | 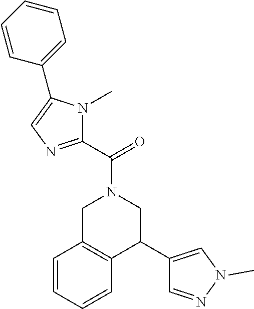 [SMILES: Cn1cc(C2CN(C(=O)c3ncc(-c4ccccc4)n3C)Cc3ccccc32)cn1] | 0.97 | 398 | B | |
| P-82 | [4-(1-methylpyrazol-4- yl)-3,4-dihydro-1H-iso- quinolin-2-yl]-(2-phen- ylthiazol-4-yl)methanone | 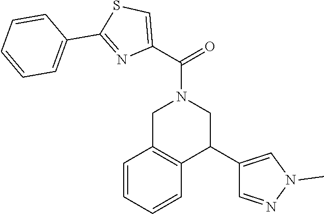 [SMILES: Cn1cc(C2CN(C(=O)c3csc(-c4ccccc4)n3)Cc3ccccc32)cn1] | 1.08 | 401 | B | |
| P-83 | (3-tert-butylisoxazol-5- yl)-[4-(1-methylpyrazol- 4-yl)-3,4-dihydro-1H-iso- quinolin-2-yl]methanone | 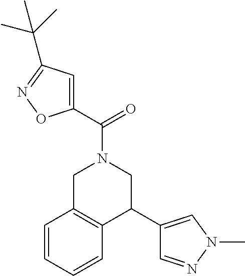 [SMILES: Cn1cc(C2CN(C(=O)c3cc(C(C)(C)C)no3)Cc3ccccc32)cn1] | 1.04 | 365 | B | |
| P-84 | [4-(1-methylpyrazol-4- yl)-3,4-dihydro-1H-iso- quinolin-2-yl]-(5-propyl- isoxazol-3-yl)methanone | 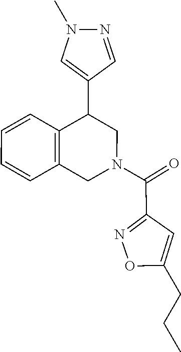 [SMILES: CCCc1cc(C(=O)N2Cc3ccccc3C(c3cnn(C)c3)C2)no1] | 1.03 | 351 | B | |
| P-85 | [4-(1-methylpyrazol-4- yl)-3,4-dihydro-1H-iso- quinolin-2-yl]-(2-phenyl- oxazol-5-yl)methanone | 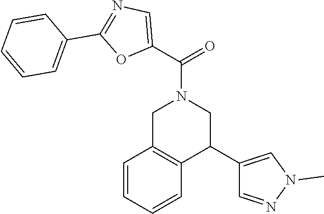 [SMILES: Cn1cc(C2CN(C(=O)c3cnc(-c4ccccc4)o3)Cc3ccccc32)cn1] | 1.00 | 385 | B | |
| P-86 | [4-(1-methylpyrazol-4- yl)-3,4-dihydro-1H- isoquinolin-2-yl]-(5- phenyl-1H-pyrazol-3-yl)- methanone | 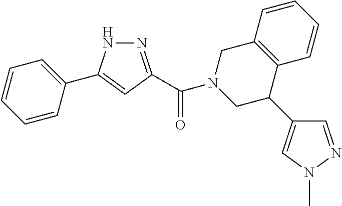 [SMILES: Cn1cc(C2CN(C(=O)c3cc(-c4ccccc4)[nH]n3)Cc3ccccc32)cn1] | 0.95 | 384 | B | |
| P-87 | [4-(4-fluorophenyl)-2- thienyl]-[4-(1-methyl- pyrazol-4-yl)-3,4- dihydro-1H-isoquinolin- 2-yl]methanone | 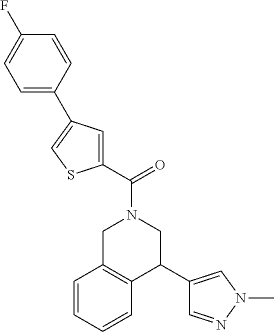 [SMILES: Cn1cc(C2CN(C(=O)c3cc(-c4ccc(F)cc4)cs3)Cc3ccccc32)cn1] | 1.11 | 418 | B | |
| P-88 | [4-(1-methylpyrazol-4- yl)-3,4-dihydro-1H- isoquinolin-2-yl]-(3- sec-butylisoxazol-5-yl)- methanone | 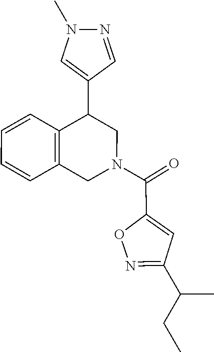 [SMILES: CCC(C)c1cc(C(=O)N2Cc3ccccc3C(c3cnn(C)c3)C2)on1] | 1.05 | 365 | B | |
| P-89 | (5-cyclopropyl-1-methyl- pyrazol-3-yl)-[4-(1-meth- ylpyrazol-4-yl)-3,4- dihydro-1H-isoquinolin- 2-yl]methanone | 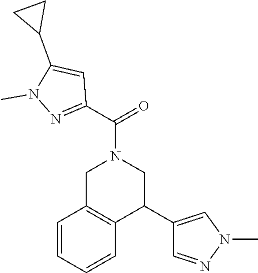 | 0.93 | 362 | B | |
| P-90 | [5-(2,5-difluorophenyl)- 1,2,4-oxadiazol-3-yl]-[4- (1-methylpyrazol-4-yl)- 3,4-dihydro-1H-isoquin- olin-2-yl]methanone | 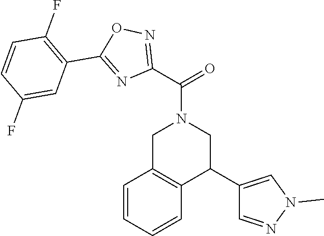 [SMILES: Cn1cc(C2CN(C(=O)c3noc(-c4cc(F)ccc4F)n3)Cc3ccccc32)cn1] | 1.05 | 422 | B | |
| P-91 | (4-methyl-5-phenyl- isoxazol-3-yl)-[4-(1- methylpyrazol-4-yl)-3,4- dihydro-1H-isoquinolin- 2-yl]methanone | 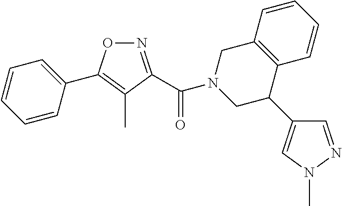 [SMILES: Cc1c(C(=O)N2Cc3ccccc3C(c3cnn(C)c3)C2)noc1-c1ccccc1] | 1.10 | 399 | B | |
| P-92 | [5-(3-fluorophenyl)-1,2,4- oxadiazol-3-yl]-[4-(1- methylpyrazol-4-yl)-3,4- dihydro-1H-isoquinolin- 2-yl]methanone | 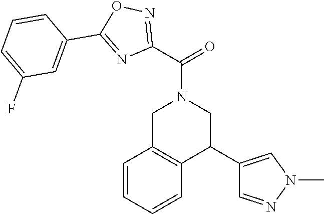 [SMILES: Cn1cc(C2CN(C(=O)c3noc(-c4cccc(F)c4)n3)Cc3ccccc32)cn1] | 1.06 | 404 | B | |
| P-93 | [5-(4-fluorophenyl)-2- methyl-pyrazol-3-yl]- [4-(1-methylpyrazol-4- yl)-3,4-dihydro-1H-iso- quinolin-2-yl]methanone | 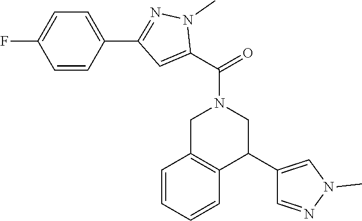 [SMILES: Cn1cc(C2CN(C(=O)c3cc(-c4ccc(F)cc4)nn3C)Cc3ccccc32)cn1] | 1.06 | 416 | B | |
| P-94 | [2-(4-fluorophenyl)- thiazol-4-yl]-[4-(1- methylpyrazol-4-yl)-3,4- dihydro-1H-isoquinolin- 2-yl]methanone | 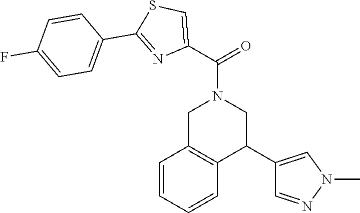 [SMILES: Cn1cc(C2CN(C(=O)c3csc(-c4ccc(F)cc4)n3)Cc3ccccc32)cn1] | 1.09 | 419 | B | |
| P-95 | [4-(1-methylpyrazol-4- yl)-3,4-dihydro-1H- isoquinolin-2-yl]-(2- phenyloxazol-4-yl)- methanone | 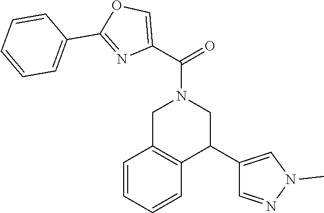 [SMILES: Cn1cc(C2CN(C(=O)c3coc(-c4ccccc4)n3)Cc3ccccc32)cn1] | 1.05 | 385 | B | |
| P-96 | [5-(4-fluorophenyl)-1H- pyrazol-3-yl]-[4-(1- methylpyrazol-4-yl)-3,4- dihydro-1H-isoquinolin- 2-yl]methanone | 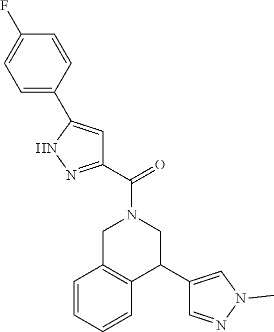 [SMILES: Cn1cc(C2CN(C(=O)c3cc(-c4ccc(F)cc4)[nH]n3)Cc3ccccc32)cn1] | 0.97 | 402 | B | |
| P-97 | [5-(4-fluorophenyl)-2- furyl]-[4-(1-methylpyr- azol-4-yl)-3,4-dihydro- 1H-isoquinolin-2-yl] methanone | 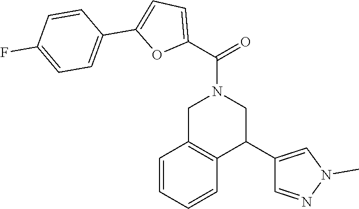 [SMILES: Cn1cc(C2CN(C(=O)c3ccc(-c4ccc(F)cc4)o3)Cc3ccccc32)cn1] | 1.09 | 402 | B | |
| P-98 | [5-(4-fluorophenyl)-1- methyl-pyrazol-3-yl]-[4- (1-methylpyrazol-4-yl)- 3,4-dihydro-1H-isoquin- olin-2-yl]methanone | 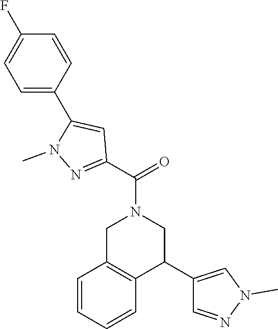 [SMILES: Cn1cc(C2CN(C(=O)c3cc(-c4ccc(F)cc4)n(C)n3)Cc3ccccc32)cn1] | 1.03 | 416 | B | |
| P-99 | (3-isopropylisoxazol-5- yl)-[4-(1-methylpyrazol- 4-yl)-3,4-dihydro-1H-iso- quinolin-2-yl]methanone | 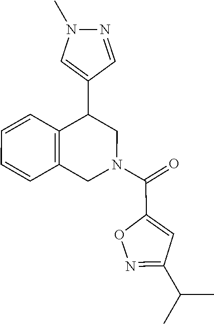 [SMILES: CC(C)c1cc(C(=O)N2Cc3ccccc3C(c3cnn(C)c3)C2)on1] | 0.99 | 351 | B | |
| P-100 | [5-(2,4-difluorophenyl)- isoxazol-3-yl]-[4-(1,5- dimethylpyrazol-4-yl)- 3,3-dimethyl-1,4- dihydroisoquinolin-2-yl] methanone | 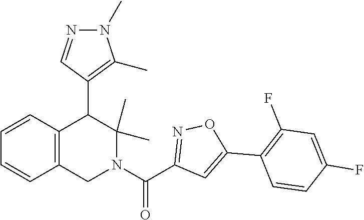 [SMILES: Cc1c(C2c3ccccc3CN(C(=O)c3cc(-c4ccc(F)cc4F)on3)C2(C)C)cnn1C] | 1.10 | 463 | A | |
| P-101 | 3-[3-[4-(1-methylpyr- azol-4-yl)-3,4-dihydro- 1H-isoquinoline-2-car- bonyl]isoxazol-5-yl]- 1H-pyridin-2-one | 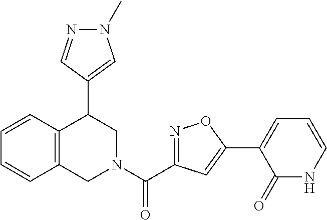 [SMILES: Cn1cc(C2CN(C(=O)c3cc(-c4ccc[nH]c4=O)on3)Cc3ccccc32)cn1] | | | | |
| P-102 | 5-[3-[4-(1-methylpyrazol- 4-yl)-3,4-dihydro-1H- isoquinoline-2-carbon- yl]isoxazol-5-yl]-1H- pyridin-2-one | 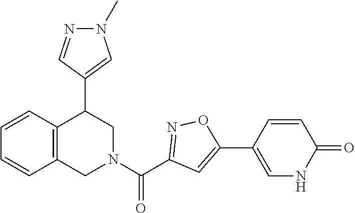 [SMILES: Cn1cc(C2CN(C(=O)c3cc(-c4ccc(=O)[nH]c4)on3)Cc3ccccc32)cn1] | | | | |
| P-103 | [4-(1-methylpyrazol-4- yl)-7-(trifluoromethyl)- 3,4-dihydro-1H-isoquin- olin-2-yl]-(5-phenyl- isoxazol-3-yl)methanone | 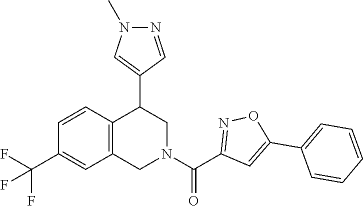 [SMILES: Cn1cc(C2CN(C(=O)c3cc(-c4ccccc4)on3)Cc3cc(C(F)(F)F)ccc32)cn1] | 1.08 | 453 | A | 181- 183 |
| P-104 | [4-(1-methylpyrazol-4-yl)- 6-(trifluoromethyl)-3,4- dihydro-1H-isoquinolin- 2-yl]-(5-phenylisoxazol- 3-yl)methanone | 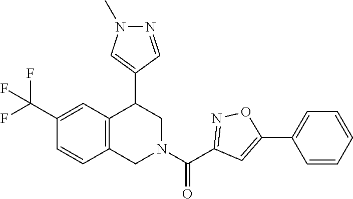 [SMILES: Cn1cc(C2CN(C(=O)c3cc(-c4ccccc4)on3)Cc3ccc(C(F)(F)F)cc32)cn1] | 1.08 | 453 | A | 63-65 |
| P-105 | [5-(2,4-difluorophenyl)- isoxazol-3-yl]-[3,3- dimethyl-4-(1-methyl- pyrazol-4-yl)-1,4- dihydroisoquinolin-2- yl]methanone | 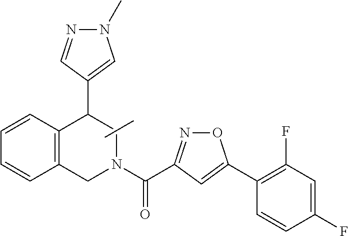 | 1.01 | 449 | A | |
| P-106 | [5-(2-methoxy-3-pyrid- yl)isoxazol-3-yl]-[4-(1- methylpyrazol-4-yl)-3,4- dihydro-1H-isoquinolin- 2-yl]methanone | 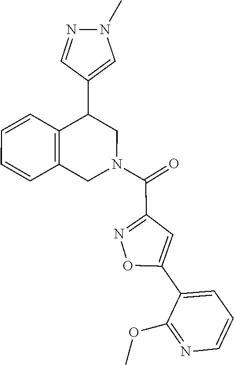 [SMILES: COc1ncccc1-c1cc(C(=O)N2Cc3ccccc3C(c3cnn(C)c3)C2)no1] | 0.98 | 416 | A | |
| P-107 | [5-(6-methoxy-3-pyridyl)- isoxazol-3-yl]-[4-(1- methylpyrazol-4-yl)-3,4- dihydro-1H-isoquinolin- 2-yl]methanone | 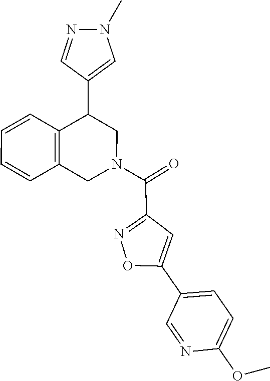 [SMILES: COc1ccc(-c2cc(C(=O)N3Cc4ccccc4C(c4cnn(C)c4)C3)no2)cn1] | 0.95 | 416 | A | |
| P-108 | [3-(2,4-difluorophenyl)- isoxazol-5-yl]-[4-(1,5- dimethylpyrazol-4-yl)- 3,4-dihydro-1H-isoquin- olin-2-yl]methanone | 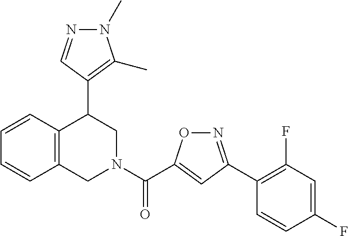 [SMILES: Cc1c(C2CN(C(=O)c3cc(-c4ccc(F)cc4F)no3)Cc3ccccc32)cnn1C] | 1.01 | 435 | A | |
| P-109 | [4-(1,5-dimethylpyrazol- 4-yl)-3,4-dihydro-1H- isoquinolin-2-yl]-[3-(4- fluorophenyl)isoxazol-5- yl]methanone | 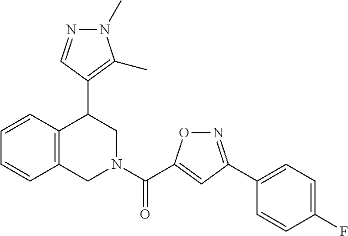 [SMILES: Cc1c(C2CN(C(=O)c3cc(-c4ccc(F)cc4)no3)Cc3ccccc32)cnn1C] | 1.01 | 417 | A | |
| P-110 | [4-(1,5-dimethylpyrazol- 4-yl)-3,4-dihydro-1H- isoquinolin-2-yl]-[3-(2- fluorophenyl)isoxazol-5- yl]methanone | 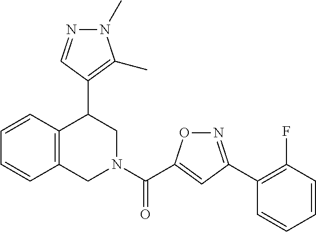 [SMILES: Cc1c(C2CN(C(=O)c3cc(-c4ccccc4F)no3)Cc3ccccc32)cnn1C] | 0.99 | 417 | A | |
| P-111 | [5-(2,4-difluorophenyl)- isoxazol-3-yl]-[4-(1- methylpyrazol-4-yl)- 7-(trifluoromethyl)-3,4- dihydro-1H-isoquinolin- 2-yl]methanone | 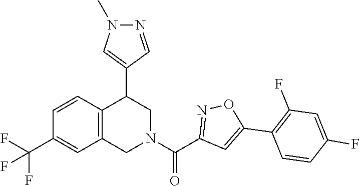 [SMILES: Cn1cc(C2CN(C(=O)c3cc(-c4ccc(F)cc4F)on3)Cc3cc(C(F)(F)F)ccc32)cn1] | 1.09 | 489 | A | |
| P-112 | [5-(2,4-difluorophenyl)- isoxazol-3-yl]-[4-(1- methylpyrazol-4-yl)- 6-(trifluoromethyl)-3,4- dihydro-1H-isoquinolin- 2-yl]methanone | 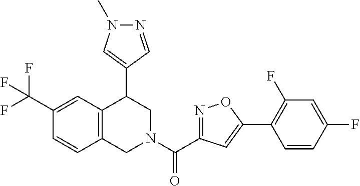 [SMILES: Cn1cc(C2CN(C(=O)c3cc(-c4ccc(F)cc4F)on3)Cc3ccc(C(F)(F)F)cc32)cn1] | 1.08 | 489 | A | |
| P-113 | [5-(2,4-difluorophenyl)- isoxazol-3-yl]-[3-methyl- 4-(1,3,5-trimethylpyrazol- 4-yl)-3,4-dihydro-1H-iso- quinolin-2-yl]methanone | 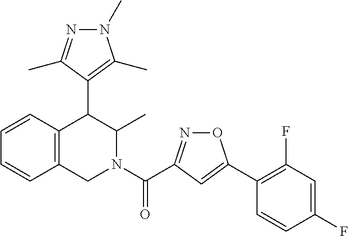 [SMILES: Cc1nn(C)c(C)c1C1c2ccccc2CN(C(=O)c2cc(-c3ccc(F)cc3F)on2)C1C] | 1.18 | 463 | B | 80-85 |
| P-114 | [5-(2,4-difluorophenyl)- isoxazol-3-yl]-[8-methyl- 4-(1,3,5-trimethylpyrazol- 4-yl)-3,4-dihydro-1H-iso- quinolin-2-yl]methanone | 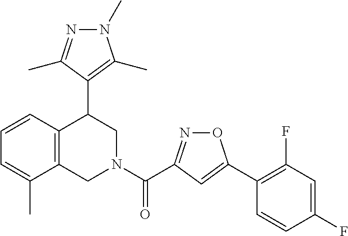 [SMILES: Cc1cccc2c1CN(C(=O)c1cc(-c3ccc(F)cc3F)on1)CC2c1c(C)nn(C)c1C] | 1.18 | 463 | B | 170- 175 |
| P-115 | [5-(2,4-difluorophenyl)- isothiazol-3-yl]-[4-(1,3,5- trimethylpyrazol-4-yl)- 3,4-dihydro-1H-isoquin- olin-2-yl]methanone | 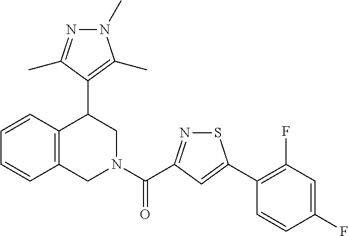 [SMILES: Cc1nn(C)c(C)c1C1CN(C(=O)c2cc(-c3ccc(F)cc3F)sn2)Cc2ccccc21] | 1.16 | 465 | B | 110- 115 |
| P-116 | [5-(2,4-difluorophenyl)- isoxazol-3-yl]-[4-(1-ethyl- 3,5-dimethyl-pyrazol-4- yl)-3,4-dihydro-1H-iso- quinolin-2-yl]methanone | 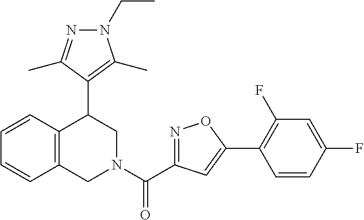 [SMILES: CCn1nc(C)c(C2CN(C(=O)c3cc(-c4ccc(F)cc4F)on3)Cc3ccccc32)c1C] | 1.09 | 463 | A | |
| P-117 | [5-(2-fluorophenyl)isox- azol-3-yl]-[4-(1,3,5- trimethylpyrazol-4-yl)- 3,4-dihydro-1H-isoquin- olin-2-yl]methanone | 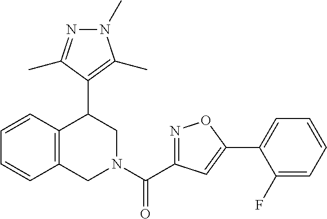 [SMILES: Cc1nn(C)c(C)c1C1CN(C(=O)c2cc(-c3ccccc3F)on2)Cc2ccccc21] | 1.03 | 431 | A | |
| P-118 | [5-(4-fluorophenyl)isox- azol-3-yl]-[4-(1,3,5- trimethylpyrazol-4-yl)- 3,4-dihydro-1H-isoquin- olin-2-yl]methanone | 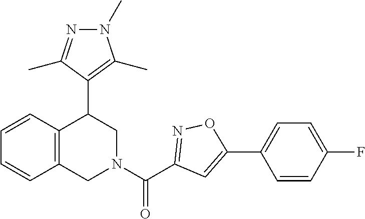 [SMILES: Cc1nn(C)c(C)c1C1CN(C(=O)c2cc(-c3ccc(F)cc3)on2)Cc2ccccc21] | 1.03 | 431 | A | 173- 178 |
| P-119 | [4-(1,5-dimethylpyrazol- 4-yl)-3,4-dihydro-1H- isoquinolin-2-yl]-[5-(2- fluorophenyl)isoxazol-3- yl]methanone | 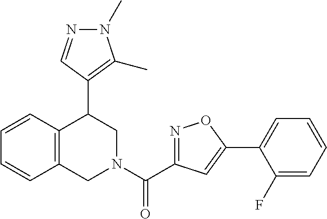 [SMILES: Cc1c(C2CN(C(=O)c3cc(-c4ccccc4F)on3)Cc3ccccc32)cnn1C] | 1.03 | 417 | A | |
| P-120 | [4-(1,5-dimethylpyrazol- 4-yl)-3,4-dihydro-1H- isoquinolin-2-yl]-[5-(4- fluorophenyl)isoxazol-3- yl]methanone | 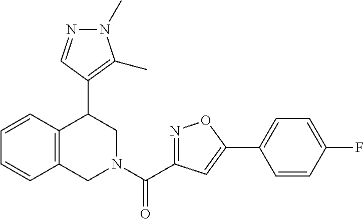 [SMILES: Cc1c(C2CN(C(=O)c3cc(-c4ccc(F)cc4)on3)Cc3ccccc32)cnn1C] | 1.03 | 417 | A | 159- 160 |
| P-121 | [5-(2,4-difluorophenyl)- isoxazol-3-yl]-[4-(1,3- dimethylpyrazol-4-yl)- 3,4-dihydro-1H-isoquin- olin-2-yl]methanone | 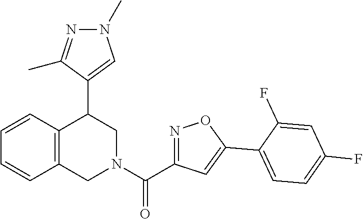 [SMILES: Cc1nn(C)cc1C1CN(C(=O)c2cc(-c3ccc(F)cc3F)on2)Cc2ccccc21] | 1.05 | 435 | A | |
| P-122 | [5-(2,4-difluorophenyl)- isoxazol-3-yl]-[8-fluoro- 4-(1,3,5-trimethylpyr- azol-4-yl)-3,4-dihydro- 1H-isoquinolin-2-yl] methanone | 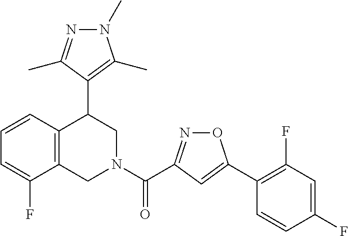 [SMILES: Cc1nn(C)c(C)c1C1CN(C(=O)c2cc(-c3ccc(F)cc3F)on2)Cc2c(F)cccc21] | 1.16 | 467 | B | |
| P-123 | [5-(2,4-difluorophenyl)- isoxazol-3-yl]-[5-fluoro- 4-(1,3,5-trimethylpyr- azol-4-yl)-3,4-dihydro- 1H-isoquinolin-2-yl] methanone | 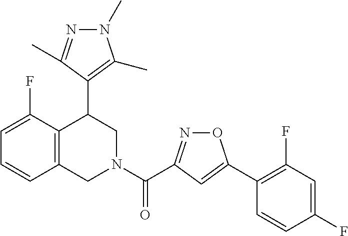 [SMILES: Cc1nn(C)c(C)c1C1CN(C(=O)c2cc(-c3ccc(F)cc3F)on2)Cc2cccc(F)c21] | 1.11 | 467 | B | 140- 145 |
| P-124 | [5-(2,4-difluorophenyl)- isoxazol-3-yl]-[5-(1,3,5- trimethylpyrazol-4-yl)- 6,8-dihydro-5H-1,7-naph- thyridin-7-yl]methanone | 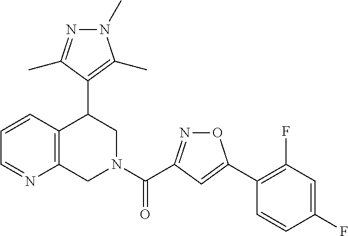 [SMILES: Cc1nn(C)c(C)c1C1CN(C(=O)c2cc(-c3ccc(F)cc3F)on2)Cc2ncccc21] | 0.97 | 450 | B | 60-65 |
| P-125 | [5-(4-chloro-2-fluoro- phenyl)isoxazol-3-yl]- [4-(1,3,5-trimethylpyr- azol-4-yl)-3,4-dihydro- 1H-isoquinolin-2-yl] methanone | 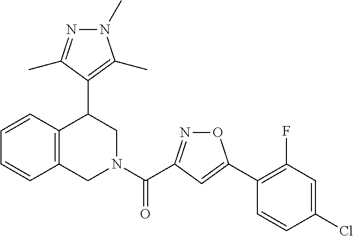 [SMILES: Cc1nn(C)c(C)c1C1CN(C(=O)c2cc(-c3ccc(Cl)cc3F)on2)Cc2ccccc21] | 1.19 | 465 | B | 90- 100 |
| P-126 | [5-(2,4-difluorophenyl)- isoxazol-3-yl]-[4-(1,3,5- trimethylpyrazol-4-yl)- 3,4-dihydro-1H-isoquin- olin-2-yl]methanone | 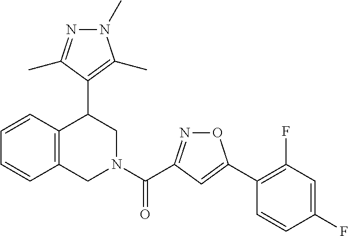 [SMILES: Cc1nn(C)c(C)c1C1CN(C(=O)c2cc(-c3ccc(F)cc3F)on2)Cc2ccccc21] | | | | 80-85 |
| P-127 | [3-(2,4-difluorophenyl)- 1,2,4-oxadiazol-5-yl]-[4- (1-methylpyrazol-4-yl)- 3,4-dihydro-1H-isoquin- olin-2-yl]methanone | 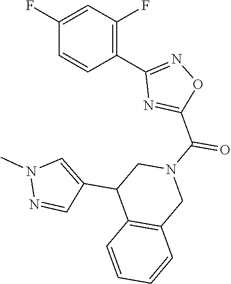 [SMILES: Cn1cc(C2CN(C(=O)c3nc(-c4ccc(F)cc4F)no3)Cc3ccccc32)cn1] | 1.03 | 422 | A | |
| P-128 | (5-phenylisoxazol-3-yl)- [4-(1-prop-2-ynylpyr- azol-4-yl)-3,4-dihydro- 1H-isoquinolin-2-yl] methanone | 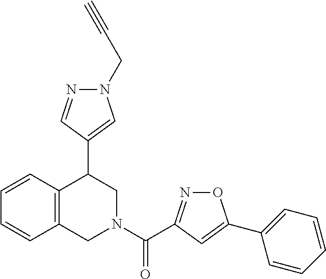 [SMILES: C#CCn1cc(C2CN(C(=O)c3cc(-c4ccccc4)on3)Cc3ccccc32)cn1] | 1.04 | 409 | A | |
| P-129 | [4-(1-methylpyrazol-4- yl)-3,4-dihydro-1H- isoquinolin-2-yl]-[3- (2,4,6-trifluorophenyl)- isoxazol-5-yl]methanone | 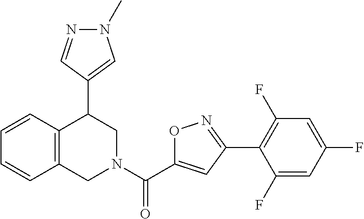 [SMILES: Cn1cc(C2CN(C(=O)c3cc(-c4c(F)cc(F)cc4F)no3)Cc3ccccc32)cn1] | 1.01 | 439 | A | |
| P-130 | (5-phenylisoxazol-3-yl)- [4-(1H-pyrazol-4-yl)-3,4- dihydro-1H-isoquinolin- 2-yl]methanone | 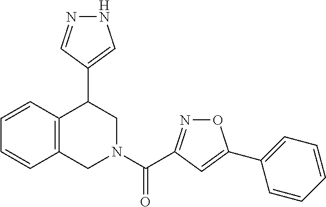 [SMILES: O=C(c1cc(-c2ccccc2)on1)N1Cc2ccccc2C(c2cn[nH]c2)C1] | 0.96 | 371 | A | |
| P-131 | [5-(2,4-difluorophenyl)- isoxazol-3-yl]-[4-(1- ethylpyrazol-4-yl)-3,4- dihydro-1H-isoquinolin- 2-yl]methanone | 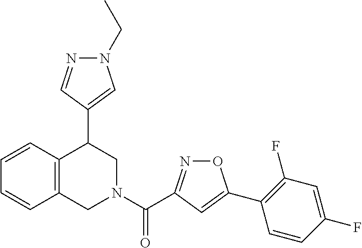 [SMILES: CCn1cc(C2CN(C(=O)c3cc(-c4ccc(F)cc4F)on3)Cc3ccccc32)cn1] | 1.07 | 435 | A | |
| P-132 | [5-(2,4-difluorophenyl)- isoxazol-3-yl]-[4-(1H- pyrazol-4-yl)-3,4- dihydro-1H-isoquin- olin-2-yl]methanone | 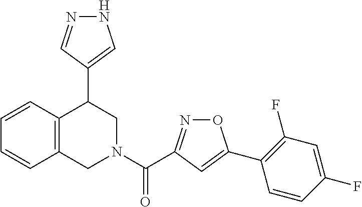 [SMILES: O=C(c1cc(-c2ccc(F)cc2F)on1)N1Cc2ccccc2C(c2cn[nH]c2)C1] | 0.99 | 407 | A | |
| P-133 | [5-(2,4-difluorophenyl)- isoxazol-3-yl]-[6-methyl- 4-(1-methylpyrazol-4- yl)-3,4-dihydro-1H-iso- quinolin-2-yl]methanone | 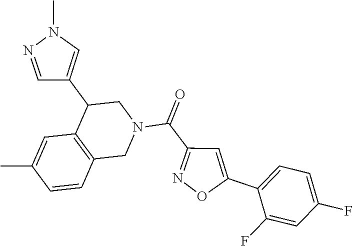 [SMILES: Cc1ccc2c(c1)C(c1cnn(C)c1)CN(C(=O)c1cc(-c3ccc(F)cc3F)on1)C2] | 1.09 | 435 | A | |
| P-134 | [5-(2,4-difluorophenyl)- isoxazol-3-yl]-[7-fluoro- 4-(1-methylpyrazol-4-yl)- 3,4-dihydro-1H-isoquin- olin-2-yl]methanone | 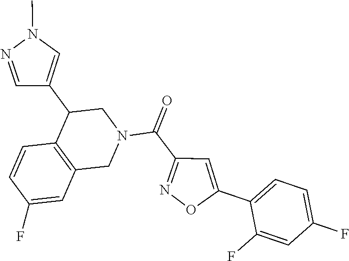 [SMILES: Cn1cc(C2CN(C(=O)c3cc(-c4ccc(F)cc4F)on3)Cc3cc(F)ccc32)cn1] | 1.05 | 439 | A | |
| P-135 | [5-(2,4-difluorophenyl)- isoxazol-3-yl]-[6-fluoro- 4-(1-methylpyrazol-4-yl)- 3,4-dihydro-1H-isoquin- olin-2-yl]methanone | 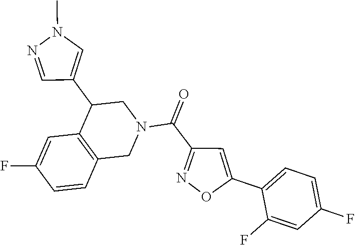 [SMILES: Cn1cc(C2CN(C(=O)c3cc(-c4ccc(F)cc4F)on3)Cc3ccc(F)cc32)cn1] | 1.05 | 439 | A | |
| P-136 | [4-(1-methylpyrazol-4- yl)-3,4-dihydro-1H-iso- quinolin-2-yl]-[5-(2,4,6- trifluorophenyl)isoxazol- 3-yl]methanone | 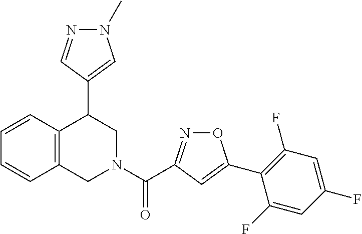 [SMILES: Cn1cc(C2CN(C(=O)c3cc(-c4c(F)cc(F)cc4F)on3)Cc3ccccc32)cn1] | 1.04 | 439 | A | |
| P-137 | [6-methyl-4-(1-methyl- pyrazol-4-yl)-3,4- dihydro-1H-isoquinolin- 2-yl]-(5-phenylisoxazol- 3-yl)methanone | 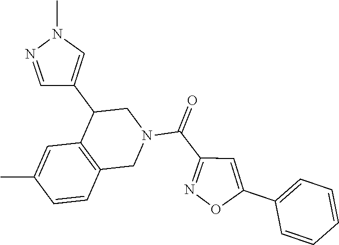 [SMILES: Cc1ccc2c(c1)C(c1cnn(C)c1)CN(C(=O)c1cc(-c3ccccc3)on1)C2] | 1.07 | 399 | A | |
| P-138 | [7-fluoro-4-(1-methyl- pyrazol-4-yl)-3,4- dihydro-1H-isoquinolin- 2-yl]-(5-phenylisoxazol- 3-yl)methanone | 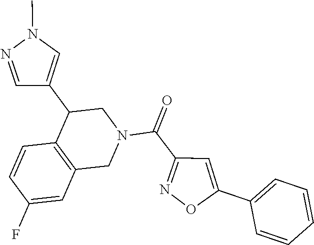 [SMILES: Cn1cc(C2CN(C(=O)c3cc(-c4ccccc4)on3)Cc3cc(F)ccc32)cn1] | 1.03 | 403 | A | |
| P-139 | [6-fluoro-4-(1-methyl- pyrazol-4-yl)-3,4- dihydro-1H-isoquinolin- 2-yl]-(5-phenylisoxazol- 3-yl)methanone | 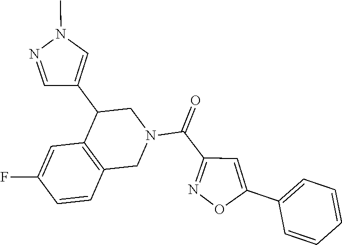 [SMILES: Cn1cc(C2CN(C(=O)c3cc(-c4ccccc4)on3)Cc3ccc(F)cc32)cn1] | 1.03 | 403 | A | |
| P-140 | [5-(2,4-difluorophenyl)- isoxazol-3-yl]-[4-(1,5- dimethylpyrazol-4-yl)- 3,4-dihydro-1H-iso- quinolin-2-yl]methanone | 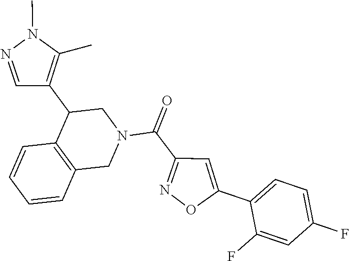 [SMILES: Cc1c(C2CN(C(=O)c3cc(-c4ccc(F)cc4F)on3)Cc3ccccc32)cnn1C] | 1.69 | 435 | C | 144- 146 |
| P-141 | [5-(2,4-difluorophenyl)- isoxazol-3-yl]-[1-methyl- 4-(1-methylpyrazol-4-yl)- 3,4-dihydro-1H-isoquin- olin-2-yl]methanone | 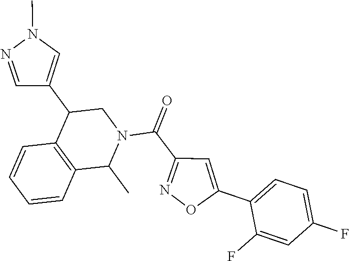 [SMILES: CC1c2ccccc2C(c2cnn(C)c2)CN1C(=O)c1cc(-c2ccc(F)cc2F)on1] | 1.72 | 435 | A | |
| P-142 | [5-(2,4-difluorophenyl)- isoxazol-3-yl]-[4-(1- methylpyrazol-4-yl)- 3,4-dihydro-1H-isoquin- olin-2-yl]methanone (Isomer A) | 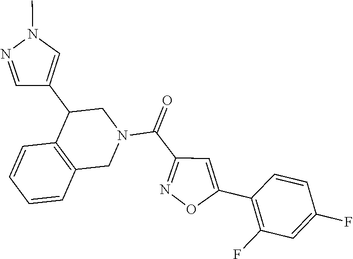 [SMILES: Cn1cc(C2CN(C(=O)c3cc(-c4ccc(F)cc4F)on3)Cc3ccccc32)cn1] | 1.04 | 421 | A | |
| P-143 | [5-(2,4-difluorophenyl)- isoxazol-3-yl]-[4-(1- methylpyrazol-4-yl)- 3,4-dihydro-1H-isoquin- olin-2-yl]methanone (Isomer B) | 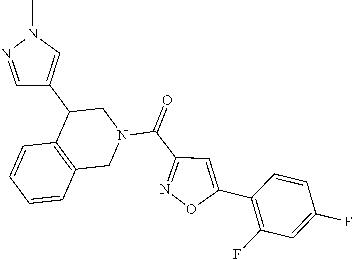 [SMILES: Cn1cc(C2CN(C(=O)c3cc(-c4ccc(F)cc4F)on3)Cc3ccccc32)cn1] | 1.04 | 421 | A | |
| P-144 | [4-(1-methylpyrazol-4- yl)-3,4-dihydro-1H- isoquinolin-2-yl]-[5- (2,3,4-trifluorophenyl)- isoxazol-3-yl]methanone | 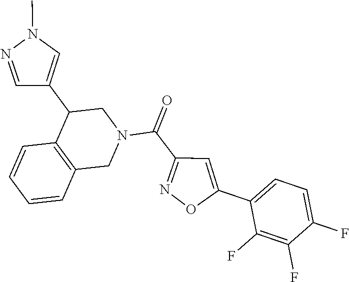 [SMILES: Cn1cc(C2CN(C(=O)c3cc(-c4ccc(F)c(F)c4F)on3)Cc3ccccc32)cn1] | 1.06 | 439 | A | |
| P-145 | [3-(2,6-difluorophenyl)- isoxazol-5-yl]-[4-(1- methylpyrazol-4-yl)-3,4- dihydro-1H-isoquinolin- 2-yl]methanone | 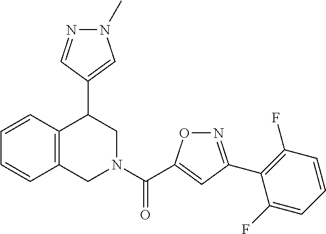 [SMILES: Cn1cc(C2CN(C(=O)c3cc(-c4c(F)cccc4F)no3)Cc3ccccc32)cn1] | 0.99 | 421 | A | |
| P-146 | [4-(1-methylpyrazol-4- yl)-3,4-dihydro-1H-iso- quinolin-2-yl]-(5-phenyl- 1,2,4-oxadiazol-3-yl)- methanone | 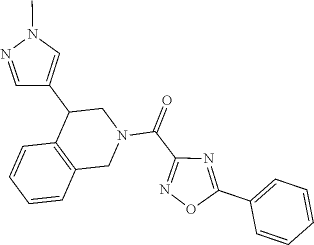 [SMILES: Cn1cc(C2CN(C(=O)c3noc(-c4ccccc4)n3)Cc3ccccc32)cn1] | 0.99 | 386 | A | |
| P-147 | [4-(1-methylpyrazol-4- yl)-3,4-dihydro-1H-iso- quinolin-2-yl]-(5-phen- yl-3-furyl)methanone | 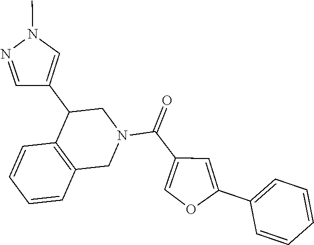 [SMILES: Cn1cc(C2CN(C(=O)c3coc(-c4ccccc4)c3)Cc3ccccc32)cn1] | 1.02 | 384 | A | |
| P-148 | (5-phenylisoxazol-3-yl)- [4-(1,3,5-trimethylpyr- azol-4-yl)-3,4-dihydro- 1H-isoquinolin-2-yl] methanone | 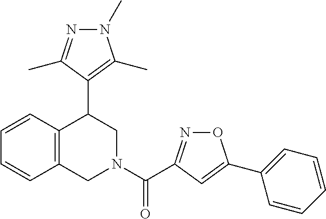 [SMILES: Cc1nn(C)c(C)c1C1CN(C(=O)c2cc(-c3ccccc3)on2)Cc2ccccc21] | 1.05 | 413 | A | |
| P-149 | [5-(2,5-difluorophenyl)- isoxazol-3-yl]-[4-(1- methylpyrazol-4-yl)-3,4- dihydro-1H-isoquinolin- 2-yl]methanone | 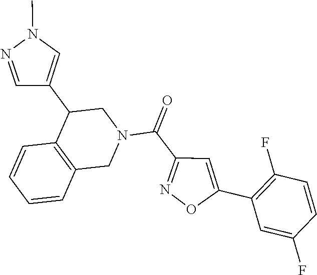 [SMILES: Cn1cc(C2CN(C(=O)c3cc(-c4cc(F)ccc4F)on3)Cc3ccccc32)cn1] | 1.04 | 421 | A | |
| P-150 | [5-(2,3-difluorophenyl)- isoxazol-3-yl]-[4-(1- methylpyrazol-4-yl)-3,4- dihydro-1H-isoquinolin- 2-yl]methanone | 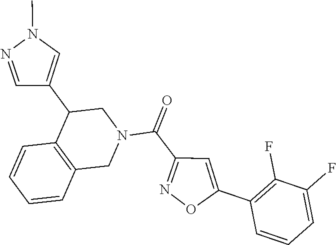 [SMILES: Cn1cc(C2CN(C(=O)c3cc(-c4cccc(F)c4F)on3)Cc3ccccc32)cn1] | 1.04 | 421 | A | |
| P-151 | [3-(2,4-difluorophenyl)- isoxazol-5-yl]-[4-(1- methylpyrazol-4-yl)-3,4- dihydro-1H-isoquinolin- 2-yl]methanone | 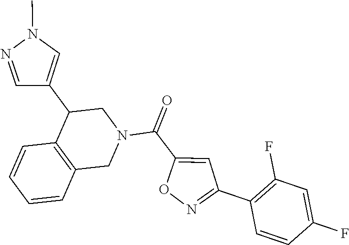 [SMILES: Cn1cc(C2CN(C(=O)c3cc(-c4ccc(F)cc4F)no3)Cc3ccccc32)cn1] | 1.03 | 421 | A | |
| P-152 | [4-(1-methylpyrazol-4- yl)-3,4-dihydro-1H- isoquinolin-2-yl]-(3- phenyl-1,2,4-oxadiazol- 5-yl)methanone | 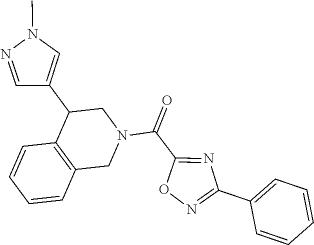 [SMILES: Cn1cc(C2CN(C(=O)c3nc(-c4ccccc4)no3)Cc3ccccc32)cn1] | 1.03 | 386 | A | |
| P-153 | [4-(1-methylpyrazol-4- yl)-3,4-dihydro-1H-iso- quinolin-2-yl]-(5-phenyl- 2-thienyl)methanone | 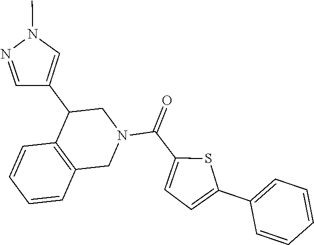 [SMILES: Cn1cc(C2CN(C(=O)c3ccc(-c4ccccc4)s3)Cc3ccccc32)cn1] | 1.06 | 400 | A | |
| P-154 | [3-(2-fluorophenyl)isox- azol-5-yl]-[4-(1-methyl- pyrazol-4-yl)-3,4- dihydro-1H-isoquinolin- 2-yl]methanone | 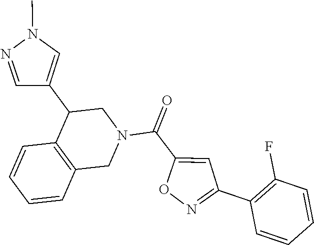 [SMILES: Cn1cc(C2CN(C(=O)c3cc(-c4ccccc4F)no3)Cc3ccccc32)cn1] | 1.02 | 403 | A | |
| P-155 | (3-cyclohexylisoxazol-5- yl)-[4-(1-methylpyrazol- 4-yl)-3,4-dihydro-1H-iso- quinolin-2-yl]methanone | 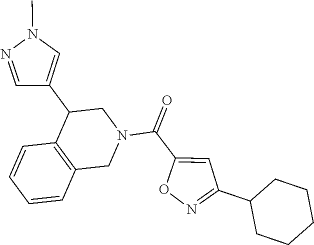 [SMILES: Cn1cc(C2CN(C(=O)c3cc(C4CCCCC4)no3)Cc3ccccc32)cn1] | 1.07 | 391 | A | |
| P-156 | [4-(1-methylpyrazol-4- yl)-3,4-dihydro-1H-iso- quinolin-2-yl]-(5-phenyl- thiazol-2-yl)methanone | 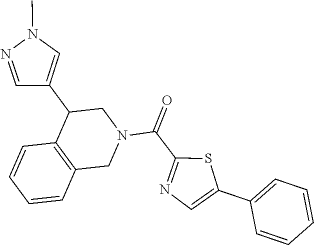 [SMILES: Cn1cc(C2CN(C(=O)c3ncc(-c4ccccc4)s3)Cc3ccccc32)cn1] | 1.08 | 401 | A | |
| P-157 | [4-(1-methylpyrazol-4- yl)-3,4-dihydro-1H-iso- quinolin-2-yl]-(5-phenyl- 2-furyl)methanone | 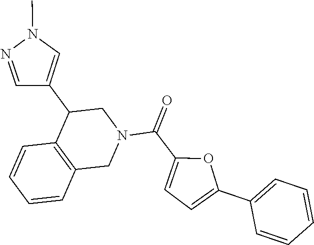 [SMILES: Cn1cc(C2CN(C(=O)c3ccc(-c4ccccc4)o3)Cc3ccccc32)cn1] | 1.02 | 384 | A | |
| P-158 | [4-(1-methylpyrazol-4- yl)-3,4-dihydro-1H-iso- quinolin-2-yl]-(3-phenyl- isoxazol-5-yl)methanone | 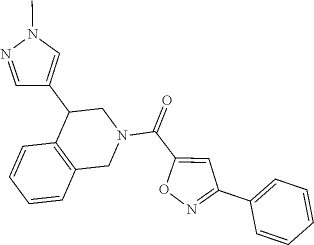 [SMILES: Cn1cc(C2CN(C(=O)c3cc(-c4ccccc4)no3)Cc3ccccc32)cn1] | 1.00 | 385 | A | |
| P-159 | [3-(3-fluorophenyl)isox- azol-5-yl]-[4-(1-methyl- pyrazol-4-yl)-3,4- dihydro-1H-isoquinolin- 2-yl]methanone | 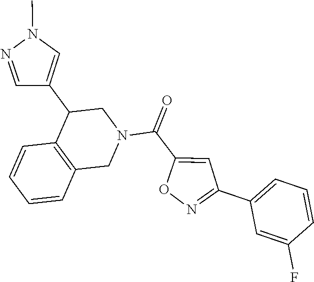 [SMILES: Cn1cc(C2CN(C(=O)c3cc(-c4cccc(F)c4)no3)Cc3ccccc32)cn1] | 1.02 | 403 | A | |
| P-160 | [3-(4-fluorophenyl)isox- azol-5-yl]-[4-(1-methyl- pyrazol-4-yl)-3,4- dihydro-1H-isoquinolin- 2-yl]methanone | 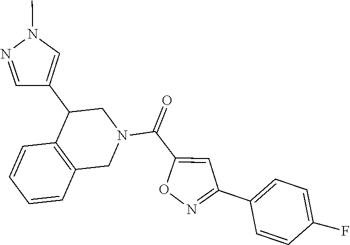 [SMILES: Cn1cc(C2CN(C(=O)c3cc(-c4ccc(F)cc4)no3)Cc3ccccc32)cn1] | 1.01 | 403 | A | |
| P-161 | [4-(1-methylpyrazol-4- yl)-3,4-dihydro-1H-iso- quinolin-2-yl]-(3-phenyl- isothiazol-5-yl)methanone | 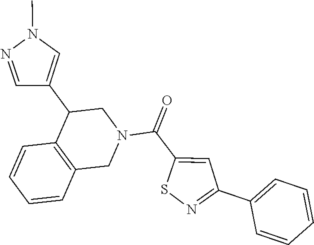 [SMILES: Cn1cc(C2CN(C(=O)c3cc(-c4ccccc4)ns3)Cc3ccccc32)cn1] | 1.03 | 401 | A | |
| P-162 | [4-(1-methylpyrazol-4- yl)-3,4-dihydro-1H-iso- quinolin-2-yl]-(5-phenyl- 1,3,4-thiadiazol-2-yl)- methanone | 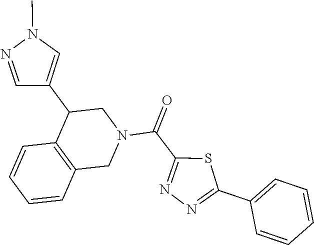 [SMILES: Cn1cc(C2CN(C(=O)c3nnc(-c4ccccc4)s3)Cc3ccccc32)cn1] | 1.01 | 402 | A | |
| P-163 | [4-(1-methylpyrazol-4- yl)-3,4-dihydro-1H-iso- quinolin-2-yl]-(5-phenyl- 1,3,4-oxadiazol-2-yl)- methanone | 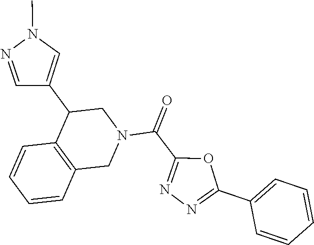 [SMILES: Cn1cc(C2CN(C(=O)c3nnc(-c4ccccc4)o3)Cc3ccccc32)cn1] | 0.95 | 386 | A | |
| P-164 | [4-(1-methylpyrazol-4- yl)-3,4-dihydro-1H-iso- quinolin-2-yl]-(2-phenyl- thiazol-5-yl)methanone | 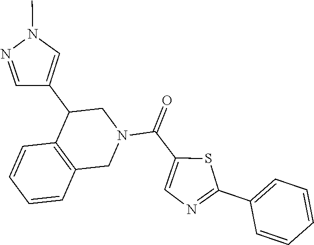 [SMILES: Cn1cc(C2CN(C(=O)c3cnc(-c4ccccc4)s3)Cc3ccccc32)cn1] | 1.00 | 401 | A | |
| P-165 | [(4S)-4-(1-methylpyrazol- 4-yl)-3,4-dihydro-1H- isoquinolin-2-yl]-(5- phenylisoxazol-3-yl)- methanone (Isomer A) | 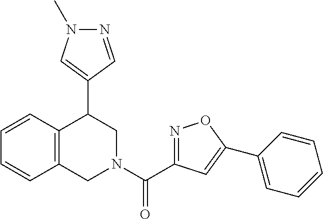 [SMILES: Cn1cc(C2CN(C(=O)c3cc(-c4ccccc4)on3)Cc3ccccc32)cn1] | 1.02 | 385 | A | |
| P-166 | [(4S)-4-(1-methylpyrazol- 4-yl)-3,4-dihydro-1H- isoquinolin-2-yl]-(5- phenylisoxazol-3-yl)- methanone (Isomer B) | 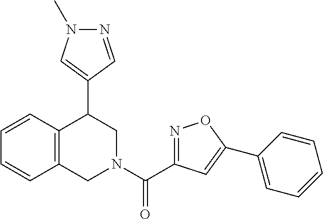 [SMILES: Cn1cc(C2CN(C(=O)c3cc(-c4ccccc4)on3)Cc3ccccc32)cn1] | 1.02 | 385 | A | |
| P-167 | [5-(3-methoxyphenyl)- isoxazol-3-yl]-[4-(1- methylpyrazol-4-yl)-3,4- dihydro-1H-isoquinolin- 2-yl]methanone | 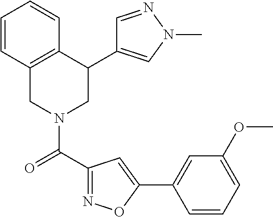 [SMILES: COc1cccc(-c2cc(C(=O)N3Cc4ccccc4C(c4cnn(C)c4)C3)no2)c1] | 1.59 | 415 | B | |
| P-168 | [5-(3-fluorophenyl)isox- azol-3-yl]-[4-(1-methyl- pyrazol-4-yl)-3,4- dihydro-1H-isoquinolin- 2-yl]methanone | 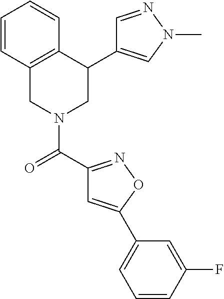 [SMILES: Cn1cc(C2CN(C(=O)c3cc(-c4cccc(F)c4)on3)Cc3ccccc32)cn1] | 1.59 | 403 | B | |
| P-169 | [4-(1-methylpyrazol-4- yl)-3,4-dihydro-1H- isoquinolin-2-yl]-[5- (o-tolyl)isoxazol-3-yl] methanone | 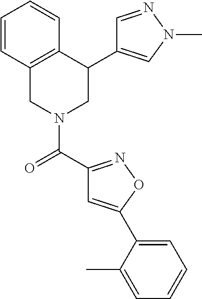 [SMILES: Cc1ccccc1-c1cc(C(=O)N2Cc3ccccc3C(c3cnn(C)c3)C2)no1] | 1.05 | 399 | A | |
| P-170 | [5-(3-fluoro-4-methoxy- phenyl)isoxazol-3-yl]- [4-(1-methylpyrazol-4- yl)-3,4-dihydro-1H-iso- quinolin-2-yl]methanone | 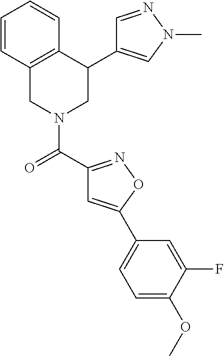 [SMILES: COc1ccc(-c2cc(C(=O)N3Cc4ccccc4C(c4cnn(C)c4)C3)no2)cc1F] | 1.58 | 433 | B | |
| P-171 | [5-(4-ethylphenyl)isox- azol-3-yl]-[4-(1-methyl- pyrazol-4-yl)-3,4- dihydro-1H-isoquinolin- 2-yl]methanone | 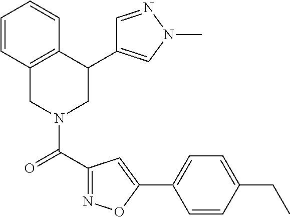 [SMILES: CCc1ccc(-c2cc(C(=O)N3Cc4ccccc4C(c4cnn(C)c4)C3)no2)cc1] | 1.78 | 413 | B | |
| P-172 | [5-(3,4-difluorophenyl)- isoxazol-3-yl]-[4-(1- methylpyrazol-4-yl)-3,4- dihydro-1H-isoquinolin- 2-yl]methanone | 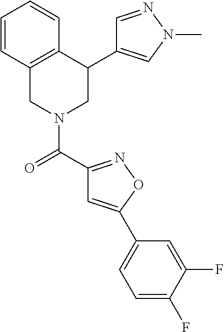 [SMILES: Cn1cc(C2CN(C(=O)c3cc(-c4ccc(F)c(F)c4)on3)Cc3ccccc32)cn1] | 1.63 | 421 | B | |
| P-173 | [5-(3,5-difluorophenyl)- isoxazol-3-yl]-[4-(1- methylpyrazol-4-yl)-3,4- dihydro-1H-isoquinolin- 2-yl]methanone | 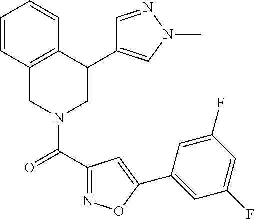 [SMILES: Cn1cc(C2CN(C(=O)c3cc(-c4cc(F)cc(F)c4)on3)Cc3ccccc32)cn1] | 1.65 | 421 | B | |
| P-174 | [5-(3,4-dimethylphenyl)- isoxazol-3-yl]-[4-(1- methylpyrazol-4-yl)-3,4- dihydro-1H-isoquinolin- 2-yl]methanone | 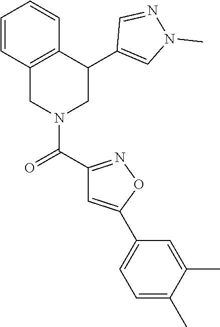 [SMILES: Cc1ccc(-c2cc(C(=O)N3Cc4ccccc4C(c4cnn(C)c4)C3)no2)cc1C] | 1.76 | 413 | B | |
| P-175 | [5-(4-isopropylphenyl)- isoxazol-3-yl]-[4-(1- methylpyrazol-4-yl)-3,4- dihydro-1H-isoquinolin- 2-yl]methanone | 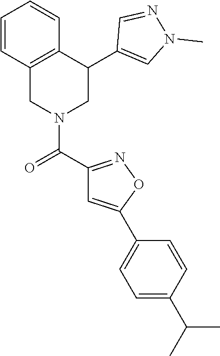 [SMILES: CC(C)c1ccc(-c2cc(C(=O)N3Cc4ccccc4C(c4cnn(C)c4)C3)no2)cc1] | 1.87 | 487 | B | |
| P-176 | (5-cyclopentylisoxazol- 3-yl)-[4-(1-methylpyr- azol-4-yl)-3,4-dihydro- 1H-isoquinolin-2-yl] methanone | 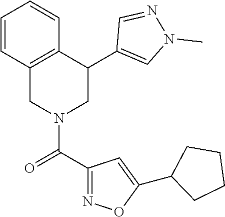 [SMILES: Cn1cc(C2CN(C(=O)c3cc(C4CCCC4)on3)Cc3ccccc32)cn1] | 1.59 | 377 | B | |
| P-177 | (5-cyclohexylisoxazol- 3-yl)-[4-(1-methylpyr- azol-4-yl)-3,4-dihydro- 1H-isoquinolin-2-yl] methanone | 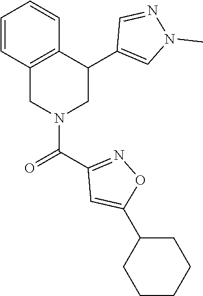 [SMILES: Cn1cc(C2CN(C(=O)c3cc(C4CCCCC4)on3)Cc3ccccc32)cn1] | 1.70 | 391 | B | |
| P-178 | [4-(1-methylpyrazol-4- yl)-3,4-dihydro-1H- isoquinolin-2-yl]-[5-(2- pyridyl)isoxazol-3-yl] methanone | 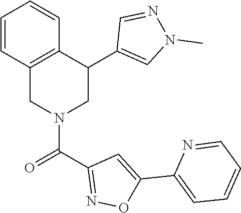 [SMILES: Cn1cc(C2CN(C(=O)c3cc(-c4ccccn4)on3)Cc3ccccc32)cn1] | 1.30 | 386 | B | |
| P-179 | [4-(1-methylpyrazol-4- yl)-3,4-dihydro-1H-iso- quinolin-2-yl]-[5-(1- methylpyrrol-2-yl)isox- azol-3-yl]methanone | 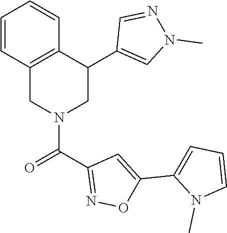 [SMILES: Cn1cc(C2CN(C(=O)c3cc(-c4cccn4C)on3)Cc3ccccc32)cn1] | 1.46 | 387 | B | |
| P-180 | [5-(2,4-difluorophenyl)- isoxazol-3-yl]-[4-(1- methylpyrazol-4-yl)-3,4- dihydro-1H-isoquinolin- 2-yl]methanone | 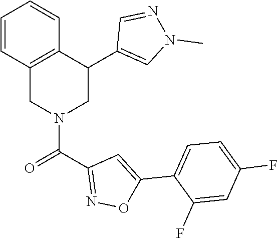 [SMILES: Cn1cc(C2CN(C(=O)c3cc(-c4ccc(F)cc4F)on3)Cc3ccccc32)cn1] | 1.04 | 421 | A | 136- 139 |
| P-181 | [4-(1-methylpyrazol-4- yl)-3,4-dihydro-1H- isoquinolin-2-yl]-[5-(3- thienyl)isoxazol-3-yl] methanone | 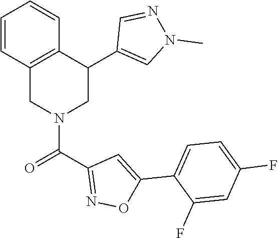 [SMILES: Cn1cc(C2CN(C(=O)c3cc(-c4ccc(F)cc4F)on3)Cc3ccccc32)cn1] | 1.49 | 391 | B | |
| P-182 | (5-cyclobutylisoxazol- 3-yl)-[4-(1-methylpyr- azol-4-yl)-3,4-dihydro- 1H-isoquinolin-2-yl] methanone | 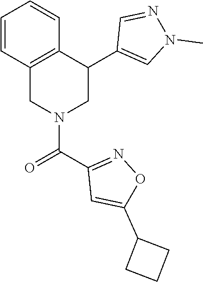 [SMILES: Cn1cc(C2CN(C(=O)c3cc(C4CCC4)on3)Cc3ccccc32)cn1] | 1.50 | 363 | B | |
| P-183 | [5-(2,4-dichlorophenyl)- isoxazol-3-yl]-[4-(1- methylpyrazol-4-yl)-3,4- dihydro-1H-isoquinolin- 2-yl]methanone | 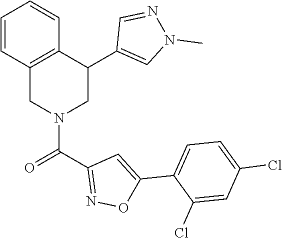 [SMILES: Cn1cc(C2CN(C(=O)c3cc(-c4ccc(Cl)cc4Cl)on3)Cc3ccccc32)cn1] | 1.81 | 453 | B | |
| P-184 | [5-(1-methylcycloprop- yl)isoxazol-3-yl]-[4-(1- methylpyrazol-4-yl)-3,4- dihydro-1H-isoquinolin- 2-yl]methanone | 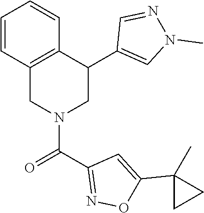 [SMILES: Cn1cc(C2CN(C(=O)c3cc(C4(C)CC4)on3)Cc3ccccc32)cn1] | 1.47 | 363 | B | |
| P-185 | [4-(1-methylpyrazol-4- yl)-3,4-dihydro-1H- isoquinolin-2-yl]-[5-(1- methylpyrazol-3-yl)isox- azol-3-yl]methanone | 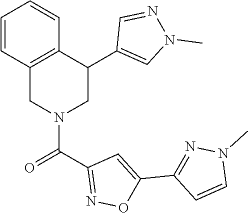 [SMILES: Cn1cc(C2CN(C(=O)c3cc(-c4ccn(C)n4)on3)Cc3ccccc32)cn1] | 1.25 | 389 | B | |
| P-186 | [5-(3,4-dimethoxyphen- yl)isoxazol-3-yl]-[4-(1- methylpyrazol-4-yl)-3,4- dihydro-1H-isoquinolin- 2-yl]methanone | 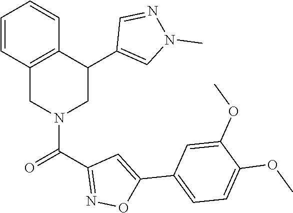 [SMILES: COc1ccc(-c2cc(C(=O)N3Cc4ccccc4C(c4cnn(C)c4)C3)no2)cc1OC] | 1.49 | 445 | B | |
| P-187 | [4-(1-methylpyrazol-4- yl)-3,4-dihydro-1H- isoquinolin-2-yl]-[5- (m-tolyl)isoxazol-3-yl] methanone | 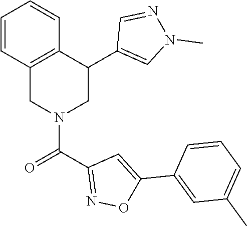 [SMILES: Cc1cccc(-c2cc(C(=O)N3Cc4ccccc4C(c4cnn(C)c4)C3)no2)c1] | 1.67 | 399 | B | |
| P-188 | [5-(2-furyl)isoxazol-3- yl]-[4-(1-methylpyr- azol-4-yl)-3,4-dihydro- 1H-isoquinolin-2-yl] methanone | 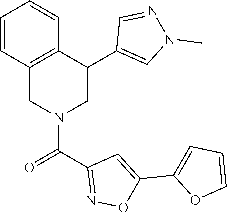 [SMILES: Cn1cc(C2CN(C(=O)c3cc(-c4ccco4)on3)Cc3ccccc32)cn1] | 1.43 | 375 | B | |
| P-189 | [5-(2-methoxyphenyl)- isoxazol-3-yl]-[4-(1- methylpyrazol-4-yl)-3,4- dihydro-1H-isoquinolin- 2-yl]methanone | 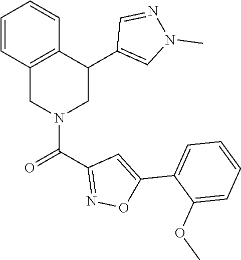 [SMILES: COc1ccccc1-c1cc(C(=O)N2Cc3ccccc3C(c3cnn(C)c3)C2)no1] | 1.60 | 415 | B | |
| P-190 | [5-(3-chlorophenyl)-isox- azol-3-yl]-[4-(1-methyl- pyrazol-4-yl)-3,4- dihydro-1H-isoquinolin- 2-yl]methanone | 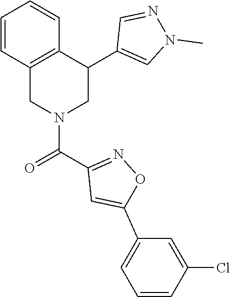 [SMILES: Cn1cc(C2CN(C(=O)c3cc(-c4cccc(Cl)c4)on3)Cc3ccccc32)cn1] | 1.70 | 419 | B | |
| P-191 | [5-(2-fluorophenyl)isox- azol-3-yl]-[4-(1-methyl- pyrazol-4-yl)-3,4- dihydro-1H-isoquinolin- 2-yl]methanone | 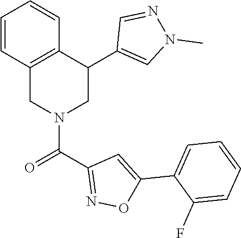 [SMILES: Cn1cc(C2CN(C(=O)c3cc(-c4ccccc4F)on3)Cc3ccccc32)cn1] | 1.02 | 403 | A | |
| P-192 | [5-(4-fluorophenyl)isox- azol-3-yl]-[4-(1-methyl- pyrazol-4-yl)-3,4- dihydro-1H-isoquinolin- 2-yl]methanone | 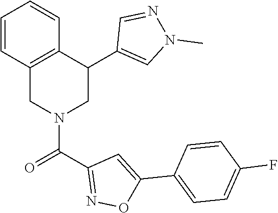 [SMILES: Cn1cc(C2CN(C(=O)c3cc(-c4ccc(F)cc4)on3)Cc3ccccc32)cn1] | 1.02 | 403 | A | |
| P-193 | [5-(4-chlorophenyl)isox- azol-3-yl]-[4-(1-methyl- pyrazol-4-yl)-3,4- dihydro-1H-isoquinolin- 2-yl]methanone | 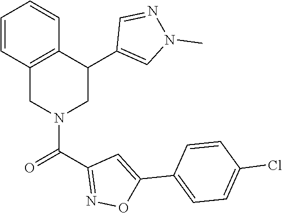 [SMILES: Cn1cc(C2CN(C(=O)c3cc(-c4ccc(Cl)cc4)on3)Cc3ccccc32)cn1] | 1.70 | 419 | B | |
| P-194 | (5-methylisoxazol-3-yl)- [4-(1-methylpyrazol-4- yl)-3,4-dihydro-1H-iso- quinolin-2-yl]methanone | 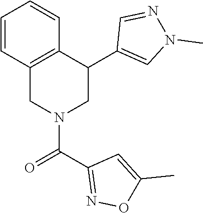 [SMILES: Cc1cc(C(=O)N2Cc3ccccc3C(c3cnn(C)c3)C2)no1] | 1.20 | 323 | B | |
| P-195 | (5-tert-butylisoxazol-3- yl)-[4-(1-methylpyr- azol-4-yl)-3,4-dihydro- 1H-isoquinolin-2-yl] methanone | 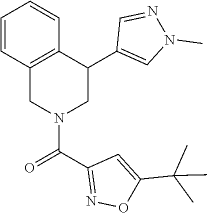 [SMILES: Cn1cc(C2CN(C(=O)c3cc(C(C)(C)C)on3)Cc3ccccc32)cn1] | 1.40 | 402 | B | |
| P-196 | [5-(4-methoxyphenyl)- isoxazol-3-yl]-[4-(1- methylpyrazol-4-yl)- 3,4-dihydro-1H-isoquin- olin-2-yl]methanone | 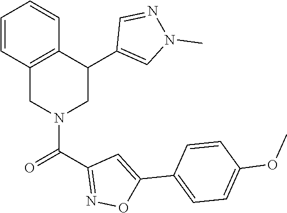 [SMILES: COc1ccc(-c2cc(C(=O)N3Cc4ccccc4C(c4cnn(C)c4)C3)no2)cc1] | 1.57 | 415 | B | |
| P-197 | [5-(2-chlorophenyl)- isoxazol-3-yl]-[4-(1- methylpyrazol-4-yl)-3,4- dihydro-1H-isoquinolin- 2-yl]methanone | 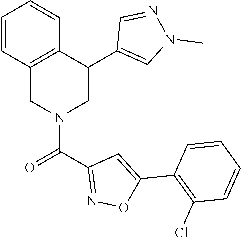 [SMILES: Cn1cc(C2CN(C(=O)c3cc(-c4ccccc4Cl)on3)Cc3ccccc32)cn1] | 1.65 | 419 | B | |
| P-198 | 2-[3-[4-(1-methylpyrazol- 4-yl)-3,4-dihydro-1H- isoquinoline-2-carbon- yl]isoxazol-5-yl] benzonitrile | 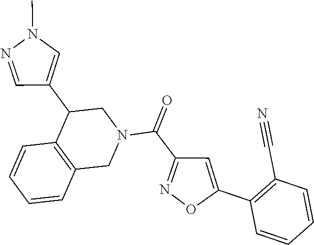 [SMILES: Cn1cc(C2CN(C(=O)c3cc(-c4ccccc4C#N)on3)Cc3ccccc32)cn1] | 0.98 | 410 | A | |
| P-199 | [4-(1-methylpyrazol-4- yl)-3,4-dihydro-1H-iso- quinolin-2-yl]-[5-(3- pyridyl)isoxazol-3-yl] methanone | 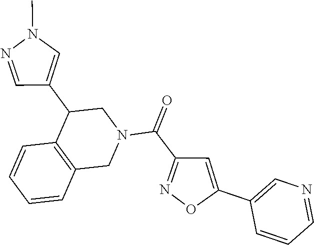 [SMILES: Cn1cc(C2CN(C(=O)c3cc(-c4cccnc4)on3)Cc3ccccc32)cn1] | 0.86 | 386 | A | |
| P-200 | 4-[3-[4-(1-methylpyrazol- 4-yl)-3,4-dihydro-1H- isoquinoline-2-carbon- yl]isoxazol-5-yl] benzonitrile | 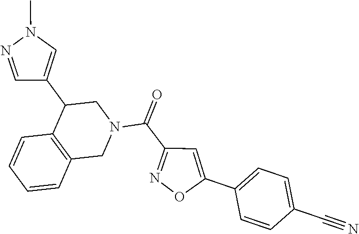 [SMILES: Cn1cc(C2CN(C(=O)c3cc(-c4ccc(C#N)cc4)on3)Cc3ccccc32)cn1] | 0.98 | 410 | A | |
| P-201 | [4-(1-methylpyrazol-4- yl)-3,4-dihydro-1H- isoquinolin-2-yl]-[5-(4- pyridyl)isoxazol-3-yl] methanone | 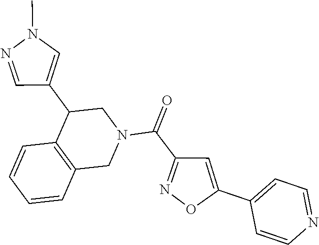 [SMILES: Cn1cc(C2CN(C(=O)c3cc(-c4ccncc4)on3)Cc3ccccc32)cn1] | 0.84 | 386 | A | |
| P-202 | (5-cyclopropylisoxazol- 3-yl)-[4-(1-methylpyr- azol-4-yl)-3,4-dihydro- 1H-isoquinolin-2-yl] methanone | 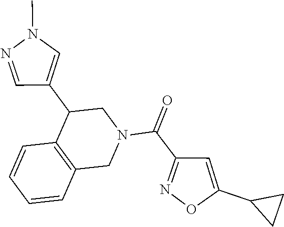 [SMILES: Cn1cc(C2CN(C(=O)c3cc(C4CC4)on3)Cc3ccccc32)cn1] | 0.92 | 349 | A | |
| P-203 | [4-(1-methylpyrazol-4- yl)-3,4-dihydro-1H-iso- quinolin-2-yl]-[5-(2- methylthiazol-4-yl)isox- azol-3-yl]methanone | 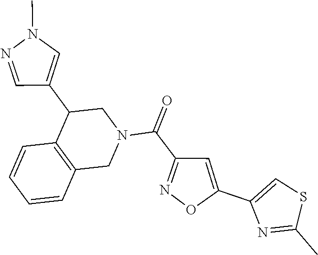 [SMILES: Cc1nc(-c2cc(C(=O)N3Cc4ccccc4C(c4cnn(C)c4)C3)no2)cs1] | 0.89 | 406 | A | |
| P-204 | [4-(1-methylpyrazol-4- yl)-3,4-dihydro-1H-iso- quinolin-2-yl]-[5-(p- tolyl)isoxazol-3-yl] methanone | 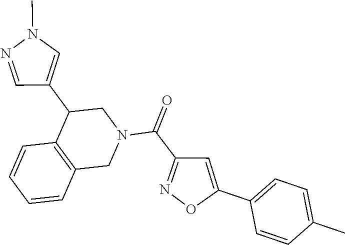 [SMILES: Cc1ccc(-c2cc(C(=O)N3Cc4ccccc4C(c4cnn(C)c4)C3)no2)cc1] | 1.06 | 399 | A | |
| P-205 | [4-(1-methylpyrazol-4- yl)-3,4-dihydro-1H- isoquinolin-2-yl]-(5- phenylisoxazol-3-yl)- methanone | 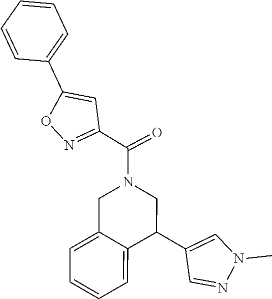 [SMILES: Cn1cc(C2CN(C(=O)c3cc(-c4ccccc4)on3)Cc3ccccc32)cn1] | 1.02 | 385 | A | |
| P-206 | [4-(1-methylpyrazol-4- yl)-3,4-dihydro-1H-iso- quinolin-2-yl]-[5-(2- thienyl)isoxazol-3-yl] methanone | 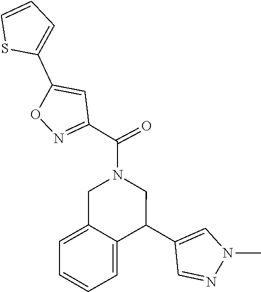 [SMILES: Cn1cc(C2CN(C(=O)c3cc(-c4cccs4)on3)Cc3ccccc32)cn1] | 1.00 | 391 | A | |
| P-207 | [5-(2,4-difluorophenyl)- 1,3,4-thiadiazol-2-yl]- [4-methyl-4-(1-methyl- pyrazol-4-yl)-1,3- dihydroisoquinolin-2- yl]methanone | 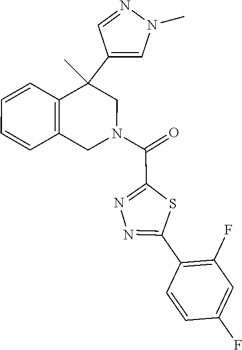 [SMILES: Cn1cc(C2(C)CN(C(=O)c3nnc(-c4ccc(F)cc4F)s3)Cc3ccccc32)cn1] | 1.07 | 452 | A | |
| P-208 | [5-(2,4-difluorophenyl)- 1,3,4-thiadiazol-2-yl]- [(4R)-4-methyl-4-(1- methylpyrazol-4-yl)-1,3- dihydroisoquinolin-2- yl]methanone | 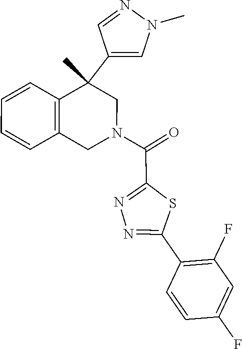 [SMILES: Cn1cc([C@]2(C)CN(C(=O)c3nnc(-c4ccc(F)cc4F)s3)Cc3ccccc32)cn1] | 1.07 | 452 | A | |
| P-209 | [5-(2,4-difluorophenyl)- 1,3,4-thiadiazol-2-yl]- [(4S)-4-methyl-4-(1- methylpyrazol-4-yl)-1,3- dihydroisoquinolin-2- yl]methanone | 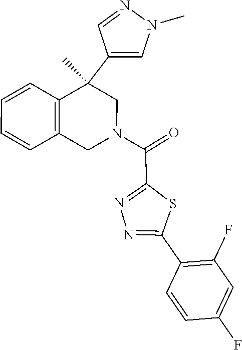 [SMILES: Cn1cc([C@@]2(C)CN(C(=O)c3nnc(-c4ccc(F)cc4F)s3)Cc3ccccc32)cn1] | 1.07 | 452 | A | |
| P-210 | [(4R)-4-(5-chloro-1- methyl-pyrazol-4-yl)-3,4- dihydro-1H-isoquinolin- 2-yl]-[5-(2,4-difluoro- phenyl)isoxazol-3-yl] methanone (Isomer A) | 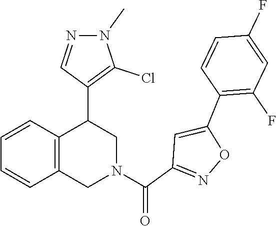 | 1.10 | 455 | A | |
| P-211 | [(4S)-4-(5-chloro-1- methyl-pyrazol-4-yl)-3,4- dihydro-1H-isoquinolin- 2-yl]-[5-(2,4-difluoro- phenyl)isoxazol-3-yl] methanone (Isomer B) | 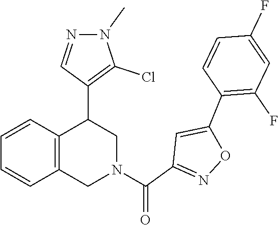 | 1.10 | 455 | A | |
| P-212 | [4-(5-chloro-1-methyl- pyrazol-4-yl)-3,4- dihydro-1H-isoquinolin- 2-yl]-[5-(2,4-difluoro- phenyl)-1,3,4-thiadiazol- 2-yl]methanone | 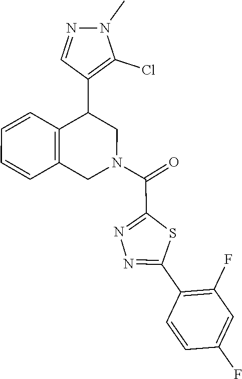 [SMILES: Cn1ncc(C2CN(C(=O)c3nnc(-c4ccc(F)cc4F)s3)Cc3ccccc32)c1Cl] | 1.11 | 472 | A | 139- 140 |
| P-213 | [(4R)-4-(5-chloro-1- methyl-pyrazol-4-yl)- 3,4-dihydro-1H-isoquin- olin-2-yl]-[5-(2,4- difluorophenyl)-1,3,4- thiadiazol-2-yl]meth- anone (Isomer A) | 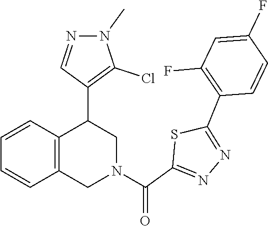 | 1.11 | 472 | A | |
| P-214 | [(4S)-4-(5-chloro-1- methyl-pyrazol-4-yl)- 3,4-dihydro-1H-isoquin- olin-2-yl]-[5-(2,4- difluorophenyl)-1,3,4- thiadiazol-2-yl]meth- anone (Isomer B) | 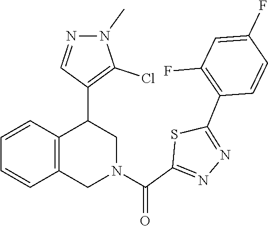 | 1.11 | 472 | A | |
| P-215 | [6-bromo-1,4-dimethyl- 4-(1-methylpyrazol-4- yl)-1,3-dihydroisoquin- olin-2-yl]-[5-(2,4- difluorophenyl)isoxazol- 3-yl]methanone | 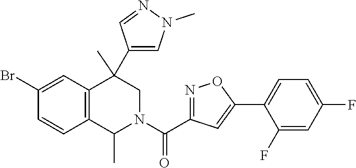 [SMILES: CC1c2ccc(Br)cc2C(C)(c2cnn(C)c2)CN1C(=O)c1cc(-c2ccc(F)cc2F)on1] | 1.15 | 527/529 | A | |
| P-216 | [5-(2,4-difluorophenyl)- isoxazol-3-yl]-[1,4,6- trimethyl-4-(1-methyl- pyrazol-4-yl)-1,3- dihydroisoquinolin-2- yl]methanone | 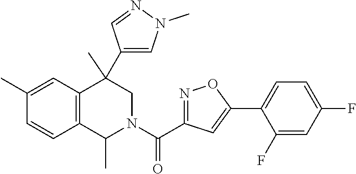 [SMILES: Cc1ccc2c(c1)C(C)(c1cnn(C)c1)CN(C(=O)c1cc(-c3ccc(F)cc3F)on1)C2C] | 1.13 | 463 | A | |
| P-217 | [5-(2,4-difluorophenyl)- isoxazol-3-yl]-[rac- (1R,4S)-6-methoxy-1- methyl-4-(1-methyl- pyrazol-4-yl)-3,4- dihydro-1H-isoquinolin- 2-yl]methanone | 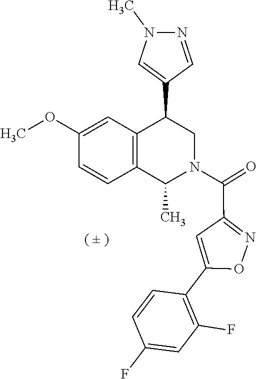 | 1.03 | 465 | A | 135- 137 |
| P-218 | [5-(2,4-difluorophenyl)- isoxazol-3-yl]-[rac-(1S, 4S)-6-methoxy-1-methyl- 4-(1-methylpyrazol-4- yl)-3,4-dihydro-1H-iso- quinolin-2-yl]methanone | 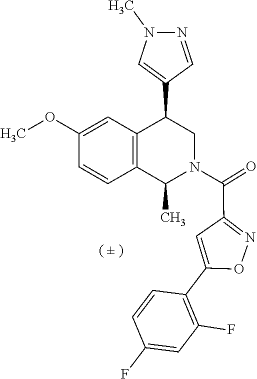 | 1.03 | 465 | A | 120- 122 |
| P-219 | [5-(2,4-difluorophenyl)- isoxazol-3-yl]-[4- isopropyl-4-(1-methyl- pyrazol-4-yl)-1,3- dihydroisoquinolin-2- yl]methanone | 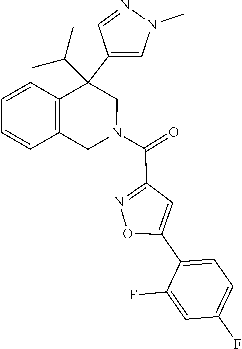 [SMILES: CC(C)C1(c2cnn(C)c2)CN(C(=O)c2cc(-c3ccc(F)cc3F)on2)Cc2ccccc21] | 1.13 | 462 | A | |
| P-220 | [5-(2,4-difluorophenyl)- isoxazol-3-yl]-[4-iso- propyl-1-methyl-4-(1- methylpyrazol-4-yl)-1,3- dihydroisoquinolin-2- yl]methanone | 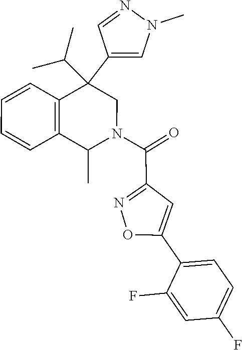 [SMILES: CC1c2ccccc2C(c2cnn(C)c2)(C(C)C)CN1C(=O)c1cc(-c2ccc(F)cc2F)on1] | 1.15 | 477 | A | |
| P-221 | [5-(2,4-difluorophenyl)- isoxazol-3-yl]-[4-ethyl-4- (1-methylpyrazol-4-yl)- 1,3-dihydroisoquinolin- 2-yl]methanone | 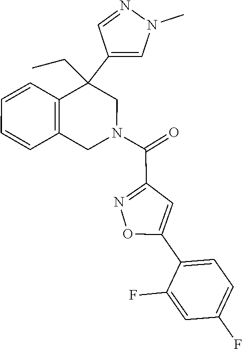 [SMILES: CCC1(c2cnn(C)c2)CN(C(=O)c2cc(-c3ccc(F)cc3F)on2)Cc2ccccc21] | 1.09 | 449 | A | |
| P-222 | [5-(2,4-difluorophenyl)- isoxazol-3-yl]-[4-ethyl- 1-methyl-4-(1-methyl- pyrazol-4-yl)-1,3- dihydroisoquinolin-2- yl]methanone | 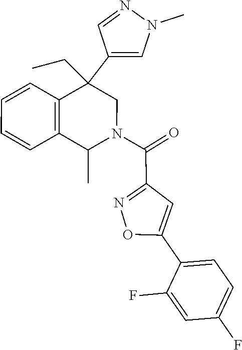 [SMILES: CCC1(c2cnn(C)c2)CN(C(=O)c2cc(-c3ccc(F)cc3F)on2)C(C)c2ccccc21] | 1.12 | 463 | A | |
| P-223 | [5-(2,4-difluorophenyl)- isoxazol-3-yl]-[4-(1,5- dimethylpyrazol-4-yl)- 3,4-dihydro-1H-isoquin- olin-2-yl]methanone (Isomer B) | 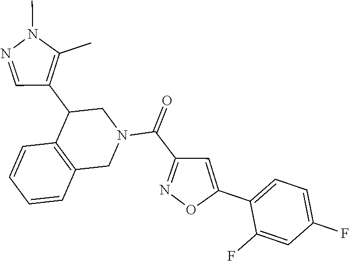 [SMILES: Cc1c(C2CN(C(=O)c3cc(-c4ccc(F)cc4F)on3)Cc3ccccc32)cnn1C] | 1.06 | 435 | C | 65-67 |
| P-224 | [5-(2,4-difluorophenyl)- isoxazol-3-yl]-[4-(1,5- dimethylpyrazol-4-yl)- 3,4-dihydro-1H-isoquin- olin-2-yl]methanone (Isomer A) | 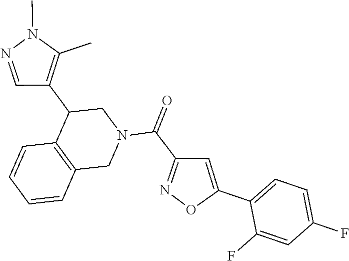 [SMILES: Cc1c(C2CN(C(=O)c3cc(-c4ccc(F)cc4F)on3)Cc3ccccc32)cnn1C] | 1.06 | 435 | C | 66-68 |
| P-225 | 5-(2,4-difluorophenyl)- 1,3,4-oxadiazol-2-yl]-[4- (1,5-dimethylpyrazol-4- yl)-3,4-dihydro-1H-iso- quinolin-2-yl]methanone | 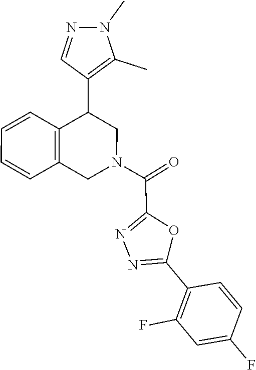 [SMILES: Cc1c(C2CN(C(=O)c3nnc(-c4ccc(F)cc4F)o3)Cc3ccccc32)cnn1C] | 1.09 | 436 | C | |
| P-226 | 4-(1,5-dimethylpyrazol- 4-yl)-3,4-dihydro-1H- isoquinolin-2-yl]-[5-(2,5- dimethylpyrazol-3-yl)- isoxazol-3-yl]methanone | 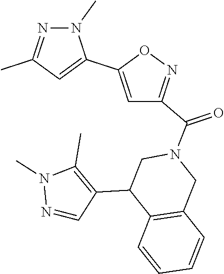 [SMILES: Cc1cc(-c2cc(C(=O)N3Cc4ccccc4C(c4cnn(C)c4C)C3)no2)n(C)n1] | 1.41 | 417 | E | |
| P-227 | [3-(2,4-difluorophenyl)- 1,2,4-oxadiazol-5-yl]-[4- (1,5-dimethylpyrazol-4- yl)-3,4-dihydro-1H-iso- quinolin-2-yl]methanone | 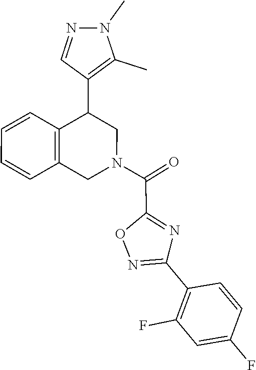 [SMILES: Cc1c(C2CN(C(=O)c3nc(-c4ccc(F)cc4F)no3)Cc3ccccc32)cnn1C] | 1.51 | 436 | C | |
| P-228 | [5-(2,4-difluorophenyl)- 1,2,4-oxadiazol-3-yl]-[4- (1,5-dimethylpyrazol-4- yl)-3,4-dihydro-1H-iso- quinolin-2-yl]methanone | 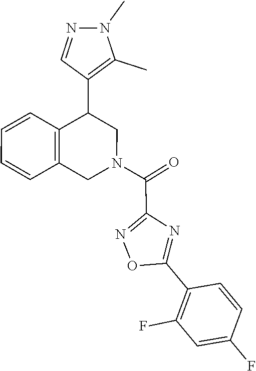 [SMILES: Cc1c(C2CN(C(=O)c3noc(-c4ccc(F)cc4F)n3)Cc3ccccc32)cnn1C] | 1.47 | 436 | E | |
| P-229 | [4-(1,5-dimethylpyrazol- 4-yl)-3,4-dihydro-1H- isoquinolin-2-yl]-(5- propyl-1,3,4-thiadiazol- 2-yl)methanone | 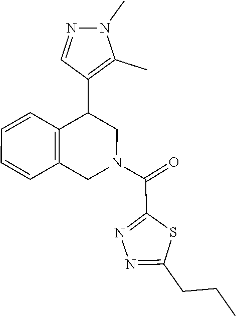 [SMILES: CCCc1nnc(C(=O)N2Cc3ccccc3C(c3cnn(C)c3C)C2)s1] | 1.06 | 382 | C | |
| P-230 | [4-(1,5-dimethylpyrazol- 4-yl)-3,4-dihydro-1H- isoquinolin-2-yl]-(5- methyl-1,3,4-thiadiazol- 2-yl)methanone | 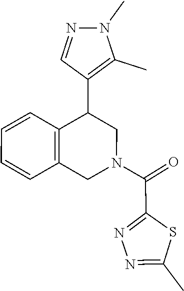 [SMILES: Cc1nnc(C(=O)N2Cc3ccccc3C(c3cnn(C)c3C)C2)s1] | 1.00 | 354 | C | |
| P-231 | [4-(1,5-dimethylpyrazol- 4-yl)-3,4-dihydro-1H- isoquinolin-2-yl]-[5-(1- methylpyrrol-2-yl)isox- azol-3-yl]methanone | 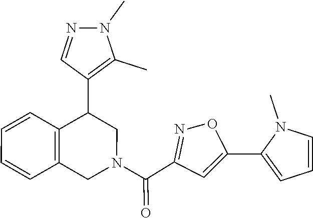 [SMILES: Cc1c(C2CN(C(=O)c3cc(-c4cccn4C)on3)Cc3ccccc32)cnn1C] | 1.12 | 402 | C | |
| P-232 | (5-cyclobutyl-1,3,4- thiadiazol-2-yl)-[4-(1,5- dimethylpyrazol-4-yl)- 3,4-dihydro-1H-isoquin- olin-2-yl]methanone | 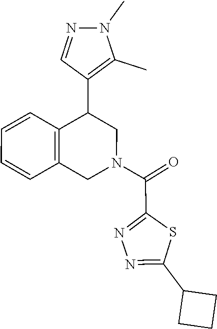 [SMILES: Cc1c(C2CN(C(=O)c3nnc(C4CCC4)s3)Cc3ccccc32)cnn1C] | 1.05- 1.10 | 394 | GC | 148- 150 |
| P-233 | (5-cyclopropyl-1,3,4- thiadiazol-2-yl)-[4-(1,5- dimethylpyrazol-4-yl)- 3,4-dihydro-1H-isoquin- olin-2-yl]methanone | 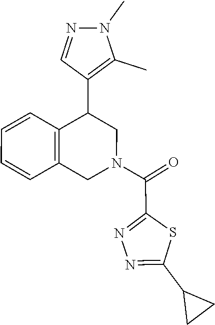 [SMILES: Cc1c(C2CN(C(=O)c3nnc(C4CC4)s3)Cc3ccccc32)cnn1C] | 1.08 | 380 | E | |
| P-234 | (5-cyclohexyl-1,3,4- thiadiazol-2-yl)-[4-(1,5- dimethylpyrazol-4-yl)- 3,4-dihydro-1H-isoquin- olin-2-yl]methanone | 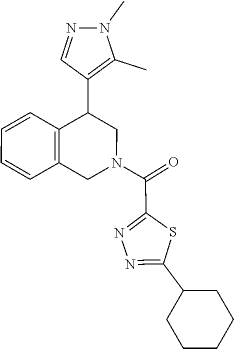 [SMILES: Cc1c(C2CN(C(=O)c3nnc(C4CCCCC4)s3)Cc3ccccc32)cnn1C] | 1.16 | 422 | C | |
| P-235 | (5-cyclopentylisoxazol- 3-yl)-[4-(1,5-dimethyl- pyrazol-4-yl)-3,4- dihydro-1H-isoquinolin- 2-yl]methanone | 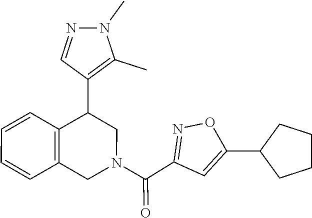 [SMILES: Cc1c(C2CN(C(=O)c3cc(C4CCCC4)on3)Cc3ccccc32)cnn1C] | 1.11 | 391 | D | 126- 128 |
| P-236 | (5-cyclohexylisoxazol- 3-yl)-[4-(1,5-dimethyl- pyrazol-4-yl)-3,4- dihydro-1H-isoquinolin- 2-yl]methanone | 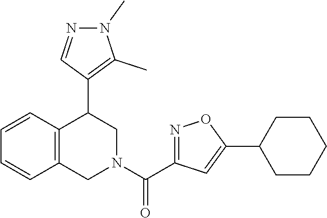 [SMILES: Cc1c(C2CN(C(=O)c3cc(C4CCCCC4)on3)Cc3ccccc32)cnn1C] | 1.13 | 405 | D | |
| P-237 | (5-cyclopropylisoxazol- 3-yl)-[4-(1,5-dimethyl- pyrazol-4-yl)-3,4- dihydro-1H-isoquinolin- 2-yl]methanone | 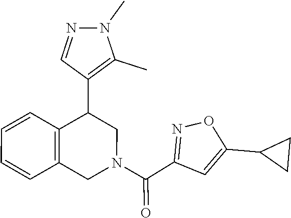 [SMILES: Cc1c(C2CN(C(=O)c3cc(C4CC4)on3)Cc3ccccc32)cnn1C] | 1.10 | 363 | C | |
| P-238 | [4-(1,5-dimethylpyrazol- 4-yl)-3,4-dihydro-1H- isoquinolin-2-yl]-(5- methylisoxazol-3-yl)- methanone | 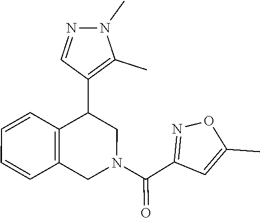 [SMILES: Cc1cc(C(=O)N2Cc3ccccc3C(c3cnn(C)c3C)C2)no1] | 1.06 | 337 | C | |
| P-239 | [5-(2,4-difluorophenyl)- isoxazol-3-yl]-[(1S,4R)- 4-isopropyl-1-methyl-4- (1-methylpyrazol-4-yl)- 1,3-dihydroisoquinolin- 2-yl]methanone | 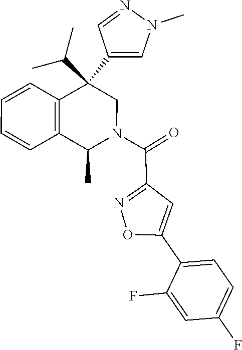 [SMILES: CC(C)[C@@]1(c2cnn(C)c2)CN(C(=O)c2cc(-c3ccc(F)cc3F)on2)[C@@H](C)c2ccccc21] | 1.15 | 477 | A | |
| P-240 | [5-(2,4-difluorophenyl)- isoxazol-3-yl]-[(1R,4S)- 4-isopropyl-1-methyl-4- (1-methylpyrazol-4-yl)- 1,3-dihydroisoquinolin- 2-yl]methanone | 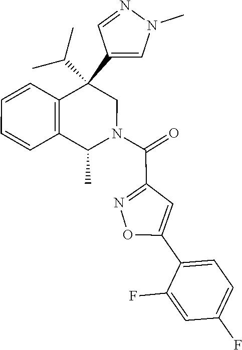 [SMILES: CC(C)[C@]1(c2cnn(C)c2)CN(C(=O)c2cc(-c3ccc(F)cc3F)on2)[C@H](C)c2ccccc21] | 1.17 | 477 | A | |
| P-241 | [5-(2,4-difluorophenyl)- isoxazol-3-yl]-[(1S,4S)- 4-isopropyl-1-methyl-4- (1-methylpyrazol-4-yl)- 1,3-dihydroisoquinolin- 2-yl]methanone | 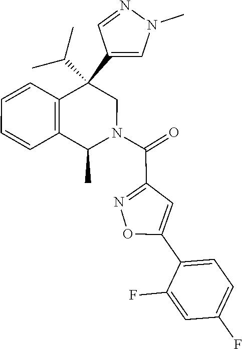 [SMILES: CC(C)[C@]1(c2cnn(C)c2)CN(C(=O)c2cc(-c3ccc(F)cc3F)on2)[C@@H](C)c2ccccc21] | 1.15 | 477 | A | |
| 242 | [5-(2,4-difluorophenyl)- isoxazol-3-yl]-[rac-(1S, 4S)-4-(1,5-dimethylpyr- azol-4-yl)-1-methyl-3,4- dihydro-1H-isoquinolin- 2-yl]methanone | 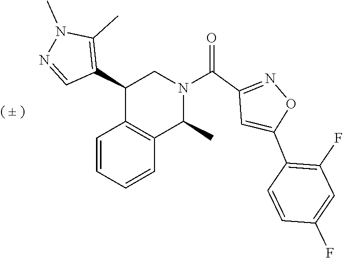 | 1.05 | 449 | A | 78-80 |
| P-243 | [5-(2,4-difluorophenyl)- isoxazol-3-yl]-[rac-(1R, 4S)-6-hydroxy-1-methyl- 4-(1-methylpyrazol-4-yl)- 3,4-dihydro-1H-isoquin- olin-2-yl]methanone | 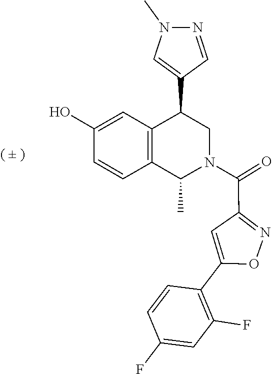 | 0.91 | 451 | A | |
| P-244 | [4-(1,5-dimethylpyrazol- 4-yl)-3,4-dihydro-1H- isoquinolin-2-yl]-(5- ethyl-1,3,4-thiadiazol-2- yl)methanone | 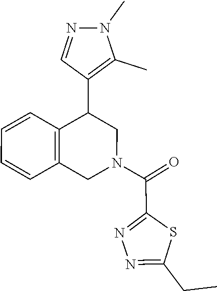 [SMILES: CCc1nnc(C(=O)N2Cc3ccccc3C(c3cnn(C)c3C)C2)s1] | 0.89 | 368 | A | |
| P-245 | [5-(2,4-difluorophenyl)- isoxazol-3-yl]-[rac-(1S, 4S)-6-hydroxy-1-methyl- 4-(1-methylpyrazol-4- yl)-3,4-dihydro-1H-iso- quinolin-2-yl]methanone | 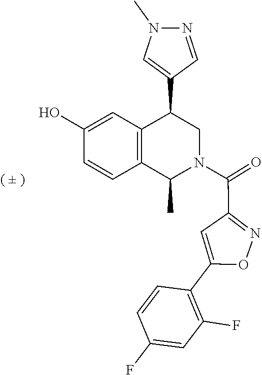 | 0.94 | 451 | A | |
| P-246 | [5-(2,4-difluorophenyl)- isoxazol-3-yl]-[4-isoprop- yl-4-(1-methylpyrazol-4- yl)-1,3-dihydroisoquin- olin-2-yl]methanone | 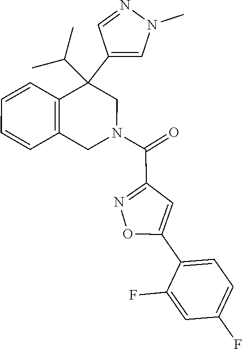 [SMILES: CC(C)C1(c2cnn(C)c2)CN(C(=O)c2cc(-c3ccc(F)cc3F)on2)Cc2ccccc21] | 1.13 | 463 | A | |
| P-247 | [5-(2,4-difluorophenyl)- isoxazol-3-yl]-[4-isoprop- | 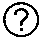 | 1.15 | 477 | A | |
| | yl-1-methyl-4-(1-meth- | | | | | |
| | ylpyrazol-4-yl)-1,3- | | | | | |
| | dihydroisoquinolin-2- | | | | | |
| | yl]methanone | | | | | |
| P-248 | [5-(2,4-difluorophenyl)- isoxazol-3-yl]-[4-ethyl-4- (1-methylpyrazol-4-yl)- 1,3-dihydroisoquinolin- 2-yl]methanone | 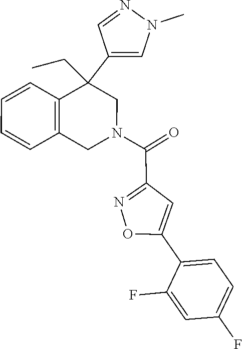 [SMILES: CCC1(c2cnn(C)c2)CN(C(=O)c2cc(-c3ccc(F)cc3F)on2)Cc2ccccc21] | 1.10 | 449 | A | |
| P-249 | [6-bromo-4-methyl-4-(1- methylpyrazol-4-yl)-1,3- dihydroisoquinolin-2-yl]- [5-(2,4-difluorophenyl)- isoxazol-3-yl]methanone | 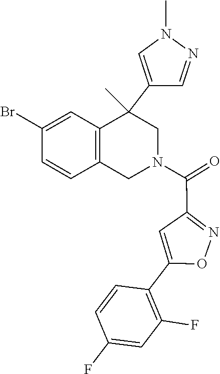 [SMILES: Cn1cc(C2(C)CN(C(=O)c3cc(-c4ccc(F)cc4F)on3)Cc3ccc(Br)cc32)cn1] | 1.12 | 513 | A | |
| P-250 | [8-bromo-4-methyl-4-(1- methylpyrazol-4-yl)-1,3- dihydroisoquinolin-2-yl]- [5-(2,4-difluorophenyl)- isoxazol-3-yl]methanone | 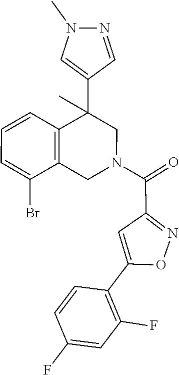 [SMILES: Cn1cc(C2(C)CN(C(=O)c3cc(-c4ccc(F)cc4F)on3)Cc3c(Br)cccc32)cn1] | 1.13 | 513 | A | |
| P-251 | 2-[5-(2,4-difluorophenyl)- isoxazole-3-carbonyl]-4- methyl-4-(1-methylpyr- azol-4-yl)-1,3-dihydro- isoquinoline-8-carbonitrile | 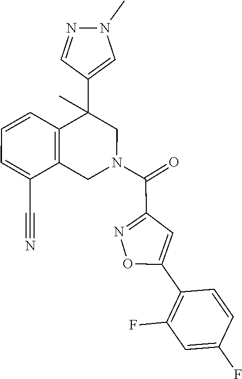 [SMILES: Cn1cc(C2(C)CN(C(=O)c3cc(-c4ccc(F)cc4F)on3)Cc3c(C#N)cccc32)cn1] | 1.01 | 460 | A | |
| P-252 | 2-[5-(2,4-difluorophen- yl)isoxazole-3-carbonyl]- 4-methyl-4-(1-methylpyr- azol-4-yl)-1,3-dihydro- isoquinoline-6-carbonitrile | 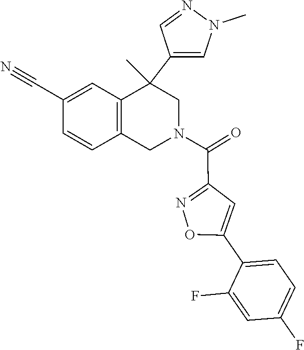 [SMILES: Cn1cc(C2(C)CN(C(=O)c3cc(-c4ccc(F)cc4F)on3)Cc3ccc(C#N)cc32)cn1] | 1.00 | 460 | A | |

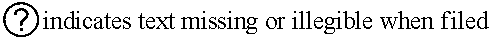

### BIOLOGICAL EXAMPLES

### Example B1: Alternaria solani/Tomato/Leaf Disc (Early Blight)

Tomato leaf disks cv. Baby are placed on agar in multiwell plates (24-well format) and sprayed with the formulated test compound diluted in water. The leaf disks are inoculated with a spore suspension of the fungus 2 days after application. The inoculated leaf disks are incubated at 23° C./21° C. (day/night) and 80% relative humidity under a light regime of 12 h/12 h (light/dark) in a climate cabinet and the activity of a compound is assessed as percent disease control compared to untreated when an appropriate level of disease damage appears on untreated check disk leaf disks (5-7 days after application).

The following compounds gave at least 80% control of Alternaria solani at 200 ppm when compared to untreated control under the same conditions, which showed extensive disease development: P-2, P-6, P-10, P-11, P-13, P-18, P-38, P-42, P-45, P-46, P-48, P-51, P-52, P-53, P-55, P-56, P-63, P-64, P-65, P-92, P-100, P-104, P-105, P-108, P-109, P-110, P-115, P-118, P-119, P-120, P-124, P-126, P-127, P-131, P-133, P-134, P-136, P-140, P-141, P-142, P-143, P-146, P-148, P-151, P-158, P-163, P-165, P-168, P-180, P-181, P-182, P-191, P-207, P-208, P-225, and P-228

### Example B2: Botryotinia Fuckeliana (Botrytis cinerea)/Liquid Culture (Gray Mould)

Conidia of the fungus from cryogenic storage are directly mixed into a nutrient broth (Vogels broth). After placing a DMSO solution of test compound into a microtiter plate (96-well format), the nutrient broth containing the fungal spores is added. The test plates are incubated at 24° C. and the inhibition of growth is determined photometrically 3-4 days after application.

The following compounds gave at least 80% control of Botryotinia fuckeliana at 20 ppm when compared to untreated control under the same conditions, which showed extensive disease development:

P-1, P-6, P-11, P-16, P-38, P-42, P-45, P-47, P-48, P-49, P-52, P-53, P-63, P-64, P-65, P-100, P-105, P-108, P-109, P-110, P-119, P-120, P-135, P-140, P-141, P-142, P-143, P-146, P-151, P-152, P-154, P-158, P-159, P-162, P-163, P-180, P-192, P-207, P-208, P-209, P-212, P-215, P-216, P-218, P-223, P-224, and P-227

### Example B3: Cercospora kikuchii (Leaf Blight of Soybean)

Conidia of the fungus from cryogenic storage were directly mixed into a nutrient broth (potato dextrose broth). A DMSO solution of the test compounds was placed into a microtiter plate (96-well format) and the nutrient broth containing the fungal spores was added to it. The test plates were incubated at 24° C. and the inhibition of growth was determined photometrically after 3-4 days at 620 nm.

The following compounds gave at least 80% control of Cercospora kikuchii at 20 ppm when compared to untreated control under the same conditions, which showed extensive disease development: P-38, P-53, P-65, P-140, and P-180.

### Example B4: Cercospora Sojina (Frogeye Leaf Spot of Soybean)

Conidia of the fungus from cryogenic storage were directly mixed into a nutrient broth (potato dextrose broth). A DMSO solution of the test compounds was placed into a microtiter plate (96-well format) and the nutrient broth containing the fungal spores was added to it. The test plates were incubated at 24° C. and the inhibition of growth was determined photometrically after 3-4 days at 620 nm. The following compounds gave at least 80% control of Cercospora sojina at 20 ppm when compared to untreated control under the same conditions, which showed extensive disease development: P-38, P-53, P-65, P-140, and P-180.

### Example B5: Glomerella Lagenarium (Colletotrichum Lagenarium)/Liquid Culture (Anthracnose)

Conidia of the fungus from cryogenic storage are directly mixed into a nutrient broth (potato dextrose broth). After placing a DMSO solution of test compound into a microtiter plate (96-well format), the nutrient broth containing the fungal spores is added. The test plates are incubated at 24° C. and the inhibition of growth is measured photometrically 3-4 days after application.

The following compounds gave at least 80% control of Glomerella lagenarium at 20 ppm when compared to untreated control under the same conditions, which showed extensive disease development:

P-1, P-6, P-10, P-11, P-14, P-16, P-18, P-38, P-45, P-47, P-48, P-49, P-50, P-51, P-52, P-53, P-56, P-58, P-62, P-63, P-64, P-65, P-76, P-92, P-97, P-100, P-104, P-105, P-108, P-109, P-110, P-111, P-117, P-119, P-120, P-121, P-124, P-126, P-138, P-139, P-140, P-141, P-142, P-143, P-149, P-151, P-152, P-154, P-155, P-158, P-162, P-163, P-165, P-166, P-169, P-180, P-181, P-187, P-188, P-190, P-191, P-192, P-194, P-205, P-206, P-207, P-208, P-209, P-212, P-215, P-216, P-223, P-224, P-225, P-226, P-227, P-228, P-229, P-231, P-232, and P-234

### Example B6: Corynespora cassiicola (target leaf spot of tomato)

Conidia of the fungus from cryogenic storage were directly mixed into a nutrient broth (potato dextrose broth). A DMSO solution of the test compounds was placed into a microtiter plate (96-well format) and the nutrient broth containing the fungal spores was added to it. The test plates were incubated at 24° C. and the inhibition of growth was determined photometrically after 3-4 days at 620 nm. The following compounds gave at least 80% control of Corynespora cassiicola at 20 ppm when compared to untreated control under the same conditions, which showed extensive disease development: P-38, P-53, P-65, P-140, and P-180.

### Example B7: Blumeria graminis f. Sp. Tritici (Erysiphe graminis f. Sp. Tritici)/Wheat/Leaf Disc Preventative (Powdery Mildew on Wheat)

Wheat leaf segments cv. Kanzler are placed on agar in a multiwell plate (24-well format) and sprayed with the formulated test compound diluted in water. The leaf disks are inoculated by shaking powdery mildew infected plants above the test plates 1 day after application. The inoculated leaf disks are incubated at 20° C. and 60% relative humidity under a light regime of 24 h darkness followed by 12 h light/12 h darkness in a climate chamber and the activity of a compound is assessed as percent disease control compared to untreated when an appropriate level of disease damage appears on untreated check leaf segments (6-8 days after application).

The following compounds gave at least 80% control of Blumeria graminis f. sp. tritici at 200 ppm when compared to untreated control under the same conditions, which showed extensive disease development:

P-11, P-63, P-65, P-104, P-108, P-109, P-140, P-141, P-151, P-207, and P-208.

### Example B8: Fusarium culmorum/Liquid Culture (Head Blight)

Conidia of the fungus from cryogenic storage are directly mixed into a nutrient broth (potato dextrose broth). After placing a DMSO solution of test compound into a microtiter plate (96-well format), the nutrient broth containing the fungal spores is added. The test plates are incubated at 24° C. and the inhibition of growth is determined photometrically 3-4 days after application.

The following compounds gave at least 80% control of Fusarium culmorum at 20 ppm when compared to untreated control under the same conditions, which showed extensive disease development:

P-38, P-52, P-64, P-65, P-108, P-119, P-140, P-212, and P-223.

### Example B9: Phaeosphaeria nodorum (Septoria nodorum)/Wheat/Leaf Disc Preventative (Glume Blotch)

Wheat leaf segments cv. Kanzler are placed on agar in a multiwell plate (24-well format) and sprayed with the formulated test compound diluted in water. The leaf disks are inoculated with a spore suspension of the fungus 2 days after application. The inoculated test leaf disks are incubated at 20° C. and 75% relative humidity under a light regime of 12 h light/12 h darkness in a climate cabinet and the activity of a compound is assessed as percent disease control compared to untreated when an appropriate level of disease damage appears in untreated check leaf disks (5-7 days after application).

The following compounds gave at least 80% control of Phaeosphaeria nodorum at 200 ppm when compared to untreated control under the same conditions, which showed extensive disease development:

P-2, P-6, P-10, P-11, P-13, P-38, P-40, P-42, P-45, P-46, P-48, P-49, P-51, P-52, P-53, P-56, P-62, P-63, P-64, P-65, P-100, P-104, P-105, P-108, P-109, P-110, P-117, P-119, P-120, P-136, P-140, P-141, P-142, P-143, P-146, P-151, P-180, P-204, P-208, P-221 and P-248

### Example B10: Monographella nivalis (Microdochium nivale)/Liquid Culture (Foot Rot Cereals)

Conidia of the fungus from cryogenic storage are directly mixed into a nutrient broth (potato dextrose broth). After placing a DMSO solution of test compound into a microtiter plate (96-well format), the nutrient broth containing the fungal spores is added. The test plates are incubated at 24° C. and the inhibition of growth is determined photometrically 4-5 days after application.

The following compounds gave at least 80% control of Monographella nivalis at 20 ppm when compared to untreated control under the same conditions, which showed extensive disease development:

P-1, P-2, P-6, P-9, P-11, P-13, P-14, P-16, P-17, P-18, P-30, P-33, P-34, P-36, P-38, P-39, P-40, P-42, P-45, P-46, P-47, P-48, P-49, P-51, P-52, P-53, P-56, P-58, P-62, P-63, P-64, P-65, P-74, P-92, P-100, P-104, P-105, P-108, P-109, P-110, P-111, P-117, P-119, P-120, P-121, P-124, P-125, P-126, P-127, P-133, P-134, P-135, P-136, P-138, P-140, P-141, P-142, P-143, P-146, P-149, P-151, P-152, P-154, P-155, P-156, P-158, P-159, P-160, P-162, P-163, P-164, P-165, P-166, P-169, P-177, P-179, P-180, P-190, P-191, P-194, P-197, P-205, P-206, P-207, P-208, P-209, P-212, P-215, P-216, P-218, P-219, P-221, P-222, P-223, P-224, P-225, P-227, P-228, P-234, P-236, P-246, and P-248

### Example B11: Mycosphaerella arachidis (Cercospora arachidicola)/Liquid Culture (Early Leaf Spot)

Conidia of the fungus from cryogenic storage are directly mixed into a nutrient broth (potato dextrose broth). After placing a DMSO solution of test compound into a microtiter plate (96-well format), the nutrient broth containing the fungal spores is added. The test plates are incubated at 24° C. and the inhibition of growth is determined photometrically 4-5 days after application.

The following compounds gave at least 80% control of Mycosphaerella arachidis at 20 ppm when compared to untreated control under the same conditions, which showed extensive disease development:

P-1, P-2, P-6, P-11, P-14, P-16, P-18, P-26, P-30, P-33, P-34, P-35, P-38, P-39, P-40, P-42, P-45, P-46, P-47, P-48, P-50, P-51, P-52, P-53, P-63, P-64, P-65, P-92, P-100, P-104, P-105, P-108, P-109, P-110, P-114, P-117, P-118, P-119, P-120, P-121, P-123, P-124, P-125, P-126, P-133, P-134, P-135, P-137, P-138, P-139, P-140, P-141, P-142, P-143, P-146, P-148, P-151, P-152, P-154, P-155, P-158, P-159, P-162, P-163, P-165, P-166, P-172, P-176, P-179, P-180, P-181, P-182, P-188, P-191, P-194, P-205, P-207, P-208, P-209, P-212, P-215, P-218, P-220, P-221, P-222, P-223, P-224, P-227, P-228, P-229, P-231, P-232, P-234, P-236, P-239, P-241, P-247, and P-248

### Example B12: Plasmopara Viticola/Grape/Leaf Disc Preventative (Late Blight)

Grape vine leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 19° C. and 80% relative humidity under a light regime of 12 h light/12 h darkness in a climate cabinet and the activity of a compound is assessed as percent disease control compared to untreated when an appropriate level of disease damage appears in untreated check leaf disks (6-8 days after application).

The following compounds gave at least 80% control of Plasmopara viticola at 200 ppm when compared to untreated control under the same conditions, which showed extensive disease development:

P-146, and P-147.

### Example B13: Puccinia recondita f. Sp. Tritici/Wheat/Leaf Disc Curative (Brown Rust)

Wheat leaf segments cv. Kanzler are placed on agar in multiwell plates (24-well format). The leaf segments are inoculated with a spore suspension of the fungus. Plates are stored in darkness at 19° C. and 75% relative humidity. The formulated test compound diluted in water is applied 1 day after inoculation. The leaf segments are incubated at 19° C. and 75% relative humidity under a light regime of 12 h light/12 h darkness in a climate cabinet and the activity of a compound is assessed as percent disease control compared to untreated when an appropriate level of disease damage appears in untreated check leaf segments (6-8 days after application).

The following compounds gave at least 80% control of Puccinia recondita f. sp. tritici at 200 ppm when compared to untreated control under the same conditions, which showed extensive disease development:

P-47, P-104, P-140, and P-234

### Example B14: Puccinia recondita f. Sp. Tritici/Wheat/Leaf Disc Preventative (Brown Rust)

Wheat leaf segments cv. Kanzler are placed on agar in multiwell plates (24-well format) and sprayed with the formulated test compound diluted in water. The leaf disks are inoculated with a spore suspension of the fungus 1 day after application. The inoculated leaf segments are incubated at 19° C. and 75% relative humidity under a light regime of 12 h light/12 h darkness in a climate cabinet and the activity of a compound is assessed as percent disease control compared to untreated when an appropriate level of disease damage appears in untreated check leaf segments (7-9 days after application).

The following compounds gave at least 80% control of Puccinia recondita f. sp. tritici at 200 ppm when compared to untreated control under the same conditions, which showed extensive disease development:

P-31, P-34, P-104, P-109, P-133, P-171, P-194, and P-206.

### Example B15: Magnaporthe grisea (Pyricularia oryzae)/Rice/Leaf Disc Preventative (Rice Blast)

Rice leaf segments cv. Ballila are placed on agar in a multiwell plate (24-well format) and sprayed with the formulated test compound diluted in water. The leaf segments are inoculated with a spore suspension of the fungus 2 days after application. The inoculated leaf segments are incubated at 22° C. and 80% relative humidity under a light regime of 24 h darkness followed by 12 h light/12 h darkness in a climate cabinet and the activity of a compound is assessed as percent disease control compared to untreated when an appropriate level of disease damage appears in untreated check leaf segments (5-7 days after application).

The following compounds gave at least 80% control of Magnaporthe grisea at 200 ppm when compared to untreated control under the same conditions, which showed extensive disease development:

P-81, P-108, P-110, P-117, P-154, and P-194.

### Example B16: Pyrenophora teres/Barley/Leaf Disc Preventative (Net Blotch)

Barley leaf segments cv. Hasso are placed on agar in a multiwell plate (24-well format) and sprayed with the formulated test compound diluted in water. The leaf segments are inoculated with a spore suspension of the fungus 2 days after application. The inoculated leaf segments are incubated at 20° C. and 65% relative humidity under a light regime of 12 h light/12 h darkness in a climate cabinet and the activity of a compound is assessed as disease control compared to untreated when an appropriate level of disease damage appears in untreated check leaf segments (5-7 days after application).

The following compounds gave at least 80% control of Pyrenophora teres at 200 ppm when compared to untreated control under the same conditions, which showed extensive disease development:

P-2, P-6, P-14, P-18, P-38, P-45, P-48, P-51, P-52, P-53, P-54, P-56, P-63, P-64, P-65, P-100, P-104, P-105, P-108, P-109, P-110, P-117, P-119, P-120, P-124, P-126, P-136, P-140, P-141, P-143, P-151, P-180, P-208, P-216, P-225, and P-228

### Example B17: Thanatephorus cucumeris (Rhizoctonia solani)/Liquid Culture (Foot Rot, Damping-Off)

Mycelia fragments of a newly grown liquid culture of the fungus are directly mixed into a nutrient broth (potato dextrose broth). After placing a DMSO solution of the test compounds into a microtiter plate (96-well format), the nutrient broth containing the fungal material is added. The test plates are incubated at 24° C. and the inhibition of growth is determined photometrically 3-4 days after application.

The following compounds gave at least 80% control of Thanatephorus cucumeris at 20 ppm when compared to untreated control under the same conditions, which showed extensive disease development:

P-109, and P-140.

### Example B18: Sclerotinia sclerotiorum/Liquid Culture (Cottony Rot)

Mycelia fragments of a newly grown liquid culture of the fungus are directly mixed into a nutrient broth (potato dextrose broth). After placing a DMSO solution of test compound into a microtiter plate (96-well format) the nutrient broth containing the fungal material is added. The test plates are incubated at 24° C. and the inhibition of growth is determined photometrically 3-4 days after application.

The following compounds gave at least 80% control of Sclerotinia sclerotiorum at 20 ppm when compared to untreated control under the same conditions, which showed extensive disease development:

P-48, P-65, P-100, P-109, P-140, P-143, P-180, P-207, P-208, P-212, and P-223

### Example B19: Mycosphaerella graminicola (Septoria tritici)/Liquid Culture (Septoria Blotch)

Conidia of the fungus from cryogenic storage are directly mixed into a nutrient broth (potato dextrose broth). After placing a DMSO solution of test compound into a microtiter plate (96-well format), the nutrient broth containing the fungal spores is added. The test plates are incubated at 24° C. and the inhibition of growth is determined photometrically 4-5 days after application.

The following compounds gave at least 80% control of Mycosphaerella graminicola at 20 ppm when compared to untreated control under the same conditions, which showed extensive disease development:

P-1, P-2, P-6, P-7, P-9, P-10, P-11, P-13, P-14, P-16, P-17, P-18, P-20, P-21, P-26, P-27, P-28, P-29, P-30, P-33, P-34, P-35, P-36, P-37, P-38, P-39, P-40, P-41, P-42, P-43, P-45, P-46, P-47, P-48, P-49, P-50, P-51, P-52, P-53, P-54, P-55, P-56, P-58, P-59, P-61, P-62, P-63, P-64, P-65, P-66, P-68, P-69, P-83, P-84, P-88, P-90, P-92, P-100, P-103, P-104, P-105, P-108, P-109, P-110, P-111, P-112, P-113, P-114, P-115, P-116, P-117, P-118, P-119, P-120, P-121, P-123, P-124, P-125, P-126, P-127, P-128, P-129, P-131, P-133, P-134, P-135, P-136, P-137, P-138, P-139, P-140, P-141, P-142, P-143, P-144, P-146, P-147, P-148, P-149, P-150, P-151, P-152, P-154, P-155, P-156, P-158, P-159, P-160, P-161, P-162, P-163, P-164, P-165, P-166, P-167, P-168, P-169, P-172, P-176, P-177, P-179, P-180, P-181, P-182, P-184, P-187, P-188, P-190, P-191, P-192, P-194, P-195, P-197, P-201, P-202, P-204, P-205, P-206, P-207, P-208, P-209, P-212, P-215, P-216, P-217, P-218, P-219, P-220, P-221, P-222, P-223, P-224, P-225, P-226, P-227, P-228, P-229, P-231, P-232, P-233, P-234, P-235, P-236, P-237, P-239, P-241, P-242, P-244, P-245, P-246, P-247, and P-248

1. Use as a fungicide of a compound of formula (I): 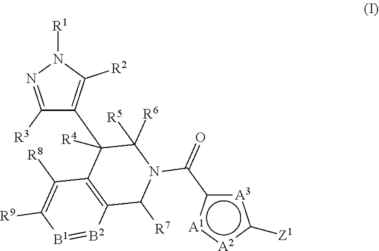
wherein
R¹ is selected from the group consisting of hydrogen, C₁-C₄ alkyl, C₂-C₄ alkenyl, C₂-C₄ alkynyl and C₃-C₆ cycloalkyl;
R² is selected from the group consisting of hydrogen, halogen, C₁-C₄ alkyl, C₂-C₄ alkenyl, C₂-C₄ alkynyl, C₁-C₄ haloalkyl, C₃-C₆ cycloalkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl and C₁-C₄ alkoxycarbonyl;
R³ and R⁴ are independently selected from the group consisting of hydrogen, halogen and C₁-C₄ alkyl;
R⁵ and R⁶ are independently selected from the group consisting of hydrogen and C₁-C₄ alkyl;
R⁷ is selected from the group consisting of hydrogen, C₁-C₄ alkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, C₁-C₄ alkoxycarbonyl, N-methoxy-N-methyl-carbonyl, C₁-C₄ alkylaminocarbonyl, di(C₁-C₄ alkylamino)carbonyl, phenyl, 5- or 6-membered heteroaryl and C₃-C₆ cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl or C₁-C₄ alkoxy;
B¹ is CR¹⁰ or N;
B² is CR¹¹ or N;
R⁸, R⁹, R¹⁰ and R¹¹ are independently selected from the group consisting of hydrogen, halogen, C₁-C₄ alkyl, C₁-C₄haloalkyl, C₁-C₄ alkoxy, C₁-C₄haloalkoxy, C₂-C₄ alkenyloxy, C₂-C₄ alkynyloxy, C₁-C₄ alkylsulfanyl, C₁-C₄ alkylsulfinyl, C₁-C₄ alkylsulfonyl, C₁-C₄ alkoxy-C₁-C₄ alkyl, C₁-C₄ alkoxycarbonyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, hydroxy,
trifluoromethylsulfonyloxy, cyano, carboxy, phenyl, 5- or 6-membered heteroaryl and C₃-C₆ cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl or C₁-C₄ alkoxy;
A¹, A² and A³ are independently selected from the group consisting of CR¹², N, NR¹³, O and S, with the proviso that at least one of A¹, A² and A³ is selected from N, O and S, and that no more than one of A¹, A² and A³ is O or S;
R¹² is selected from the group consisting of hydrogen, C₁-C₄ alkyl, C₂-C₄ alkenyl and C₂-C₄ alkynyl;
R¹³ is selected from the group consisting of hydrogen, C₁-C₄ alkyl, C₂-C₄ alkenyl and C₂-C₄ alkynyl; and
Z¹ is selected from the group consisting of C₁-C₄ alkyl, phenyl, 5- or 6-membered heteroaryl and C₃-C₆-cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl, C₁-C₄ alkoxy, C₁-C₄ haloalkoxy, C₁-C₄ alkylsulfanyl, C₁-C₄ alkylsulfinyl, C₁-C₄ alkylsulfonyl or C₂-C₄ alkynyl;
or an agrochemically acceptable salt, stereoisomer, enantiomer, tautomer or N-oxide thereof.

2. Use according to claim 1, wherein in the compound of formula (I) R¹ is hydrogen or C₁-C₄ alkyl.

3. Use according to claim 1, wherein in the compound of formula (I) R² is selected from the group consisting of hydrogen, halogen, C₁-C₄ alkyl, C₃-C₆cycloalkyl, C₁-C₄ alkylcarbonyl, N—C₁-C₄ alkoxy-C—C₁-C₄ alkyl-carbonimidoyl and N-hydroxy-C—C₁-C₄ alkyl-carbonimidoyl.

4. Use according to claim 1, wherein in the compound of formula (I) R³ and R⁴ are independently selected from the group consisting of hydrogen, halogen and C₁-C₄ alkyl.

5. Use according to claim 1, wherein in the compound of formula (I) R⁵ and R⁶ are independently selected from the group consisting of hydrogen, methyl and ethyl.

6. Use according to claim 1, wherein in the compound of formula (I) R⁷ is selected from the group consisting of hydrogen, C₁-C₄ alkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, C₁-C₄ alkoxycarbonyl, N-methoxy-N-methyl-carbonyl, phenyl, 4-cyanophenyl, cyclopropyl and 1-cyanocyclopropyl.

7. Use according to claim 1, wherein in the compound of formula (I) B¹ is CR¹⁰ and B² is CR¹¹ or B¹ is N and B² is CR¹¹ or B¹ is CR¹⁰ and B² is N.

8. Use according to claim 1, wherein in the compound of formula (I) R⁸ and R¹¹ are independently selected from the group consisting of hydrogen, halogen and C₁-C₄ alkyl.

9. Use according to claim 1, wherein in the compound of formula (I) R⁹ and R¹⁰ are independently selected from the group consisting of hydrogen, chloro, fluoro, methyl, trifluoromethyl, difluoromethoxy, trifluoromethoxy, 2,2-difluoroethoxy, 2,2,2-trifluoroethoxy, methoxy, propoxy, allyloxy, prop-2-ynoxy, methylsulfanyl, methylsulfinyl, methylsulfonyl, methoxymethyl, 2-methoxyethoxymethyl, methoxycarbonyl, acetyl, propanoyl, —C(CH₃)═NOCH₃, —C(CH₃)═NOCH₂CH₃, —C(CH₃)═NOH, methylaminocarbonyl, di(methylamino)carbonyl, trifluoromethylsulfonyloxy, cyano, carboxy, phenyl, 2-cyanophenyl, 3-cyanophenyl, 4-cyanophenyl, [4-(trifluoromethyl)pyrazol-1-yl], [3-(trifluoromethyl)pyrazol-1-yl], (3-cyanopyrazol-1-yl), (4-cyanopyrazol-1-yl), (5-chloropyrazol-1-yl), (4-chloropyrazol-1-yl), (3-chloropyrazol-1-yl), (5-fluoropyrazol-1-yl), (4-fluoropyrazol-1-yl), (3-fluoropyrazol-1-yl), (3,5-dimethylpyrazol-1-yl), (5-methylpyrazol-1-yl), (4-methylpyrazol-1-yl), (3-methylpyrazol-1-yl), pyrazol-1-yl, cyclopropyl and 1-cyanocyclopropyl.

10. Use according to claim 1, wherein in the compound of formula (I) A¹ and A² are independently selected from the group consisting of CR¹², N and O and A³ is CR¹², N, O or S, with the proviso that at least one of A¹, A² and A³ is N or O and that no more than one of A¹, A² and A³ is O.

11. Use according to claim 1, wherein in the compound of formula (I) R¹² is hydrogen or C₁-C₄ alkyl, preferably hydrogen or methyl.

12. Use according to claim 1, wherein in the compound of formula (I) Z¹ is selected from the group consisting of 1-methylpyrazol-4-yl, 2,3,4-trifluorophenyl, 2,3-difluorophenyl, 3,4-difluorophenyl, 2,4,6-trifluorophenyl, 2,4-difluorophenyl, 2,5-difluorophenyl, 2-fluoro-4-methoxy-phenyl, 2-fluoro-4-methylsulfonyl-phenyl, 2-fluorophenyl, 3-fluorophenyl, 4-fluorophenyl, 2-furyl, 2-thienyl, 3-thienyl, 2-methylphenyl, 3-methylphenyl, 4-methylphenyl, 2-chlorophenyl, 3-chlorophenyl, 4-chlorophenyl, 3-methoxyphenyl, 4-ethynyl-2-fluoro-phenyl, 4-fluoro-2-methoxy-phenyl, cyclopropyl, 1-methylcyclopropyl, cyclobutyl, cyclohexyl, cyclopentyl, methyl, n-propyl, and phenyl.

13. A compound of formula (I): 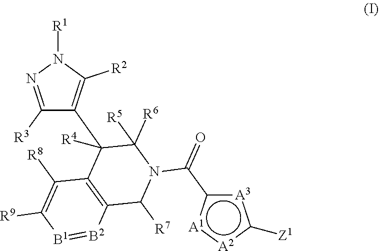
wherein
R¹ is selected from the group consisting of hydrogen, C₁-C₄ alkyl, C₂-C₄ alkenyl, C₂-C₄ alkynyl and C₃-C₆ cycloalkyl;
R² is selected from the group consisting of hydrogen, halogen, C₁-C₄ alkyl, C₂-C₄ alkenyl, C₂-C₄ alkynyl, C₁-C₄ haloalkyl, C₃-C₆ cycloalkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl and C₁-C₄ alkoxycarbonyl;
R³ and R⁴ are independently selected from the group consisting of hydrogen, halogen and C₁-C₄ alkyl;
R⁵ and R⁶ are independently selected from the group consisting of hydrogen and C₁-C₄ alkyl;
R⁷ is selected from the group consisting of hydrogen and C₁-C₄ alkyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, C₁-C₄ alkoxycarbonyl, N-methoxy-N-methyl-carbonyl, C₁-C₄ alkylaminocarbonyl, di(C₁-C₄ alkylamino)carbonyl, phenyl, 5- or 6-membered heteroaryl and C₃-C₆ cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl or C₁-C₄ alkoxy;
B¹ is CR¹⁰ or N;
B² is CR¹¹ or N;
R¹, R⁹, R¹⁰ and R¹¹ are independently selected from the group consisting of hydrogen, halogen, C₁-C₄ alkyl, C₁-C₄haloalkyl, C₁-C₄ alkoxy, C₁-C₄haloalkoxy, C₂-C₄ alkenyloxy, C₂-C₄ alkynyloxy, C₁-C₄ alkylsulfanyl, C₁-C₄ alkylsulfinyl, C₁-C₄ alkylsulfonyl, C₁-C₄ alkoxy-C₁-C₄ alkyl, C₁-C₄ alkoxycarbonyl, C₁-C₄ alkylcarbonyl, N—C₁-C₄ alkoxy-C—C₁-C₄ alkyl-carbonimidoyl, N-hydroxy-C—C₁-C₄ alkyl-carbonimidoyl, hydroxy,
trifluoromethylsulfonyloxy, cyano, carboxy, phenyl, 5- or 6-membered heteroaryl and C₃-C₆ cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl or C₁-C₄ alkoxy;
A¹, A² and A³ are independently selected from the group consisting of CR¹², N, NR¹³, O and S, with the proviso that at least one of A¹, A² and A³ is selected from N, O and S, and that no more than one of A¹, A² and A³ is O or S;
R¹² is selected from the group consisting of hydrogen, C₁-C₄ alkyl, C₂-C₄ alkenyl and C₂-C₄ alkynyl;
R¹³ is selected from the group consisting of hydrogen, C₁-C₄ alkyl, C₂-C₄ alkenyl and C₂-C₄ alkynyl; and
Z¹ is selected from the group consisting of C₃-C₄ alkyl, phenyl, 5- or 6-membered heteroaryl and C₃-C₆-cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆-cycloalkyl are optionally substituted by 1, 2 or 3 substituents independently selected from halogen, cyano, C₁-C₄ alkyl, C₁-C₄ haloalkyl, C₁-C₄ alkoxy, C₁-C₄ haloalkoxy, C₁-C₄ alkylsulfanyl, C₁-C₄ alkylsulfinyl, C₁-C₄ alkylsulfonyl or C₂-C₄ alkynyl;
or an agrochemically acceptable salt, stereoisomer, enantiomer, tautomer or N-oxide thereof, with the proviso that said compound of formula (I) is not

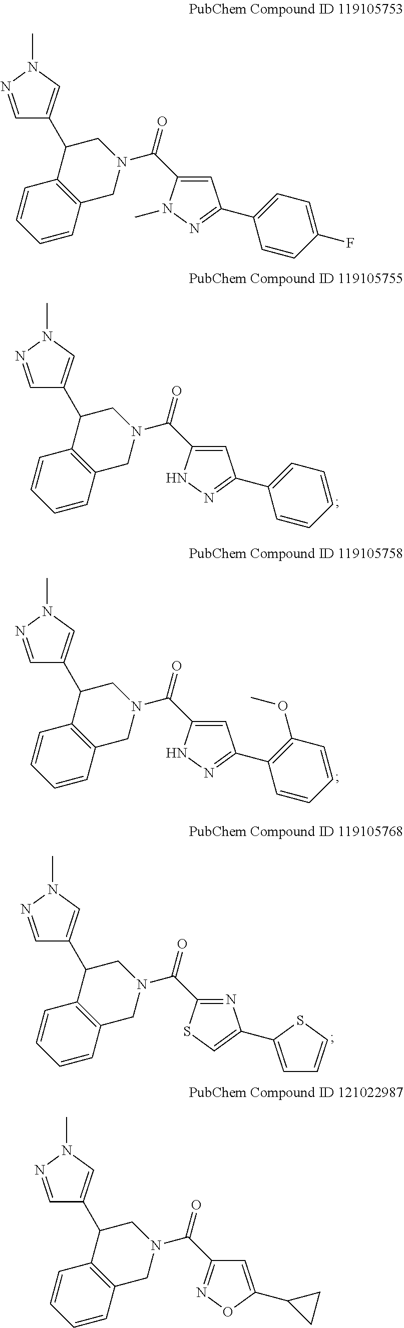 [SMILES: Cn1cc(C2CN(C(=O)c3cc(-c4ccc(F)cc4)nn3C)Cc3ccccc32)cn1 ; Cn1cc(C2CN(C(=O)c3cc(-c4ccccc4)n[nH]3)Cc3ccccc32)cn1 ; COc1ccccc1-c1cc(C(=O)N2Cc3ccccc3C(c3cnn(C)c3)C2)[nH]n1 ; Cn1cc(C2CN(C(=O)c3nc(-c4cccs4)cs3)Cc3ccccc32)cn1 ; Cn1cc(C2CN(C(=O)c3cc(C4CC4)on3)Cc3ccccc32)cn1]

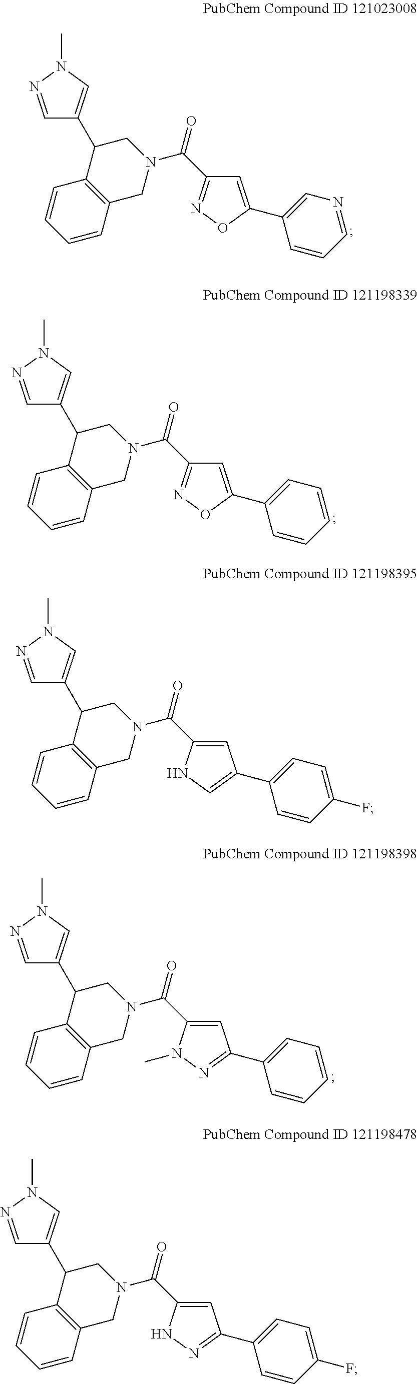 [SMILES: Cn1cc(C2CN(C(=O)c3cc(-c4cccnc4)on3)Cc3ccccc32)cn1 ; Cn1cc(C2CN(C(=O)c3cc(-c4ccccc4)on3)Cc3ccccc32)cn1 ; Cn1cc(C2CN(C(=O)c3cc(-c4ccc(F)cc4)c[nH]3)Cc3ccccc32)cn1 ; Cn1cc(C2CN(C(=O)c3cc(-c4ccccc4)nn3C)Cc3ccccc32)cn1 ; Cn1cc(C2CN(C(=O)c3cc(-c4ccc(F)cc4)n[nH]3)Cc3ccccc32)cn1]

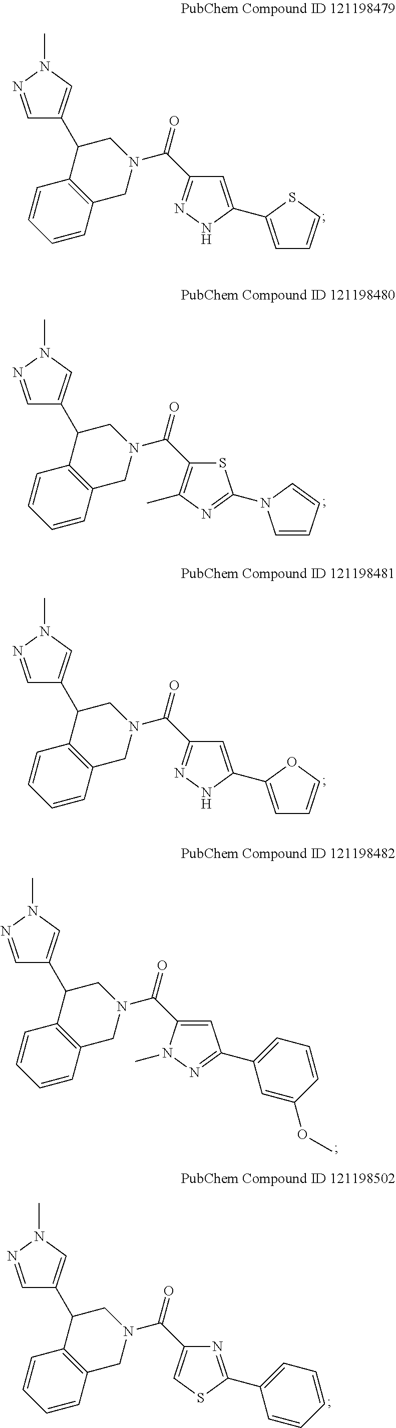 [SMILES: Cn1cc(C2CN(C(=O)c3cc(-c4cccs4)[nH]n3)Cc3ccccc32)cn1 ; Cc1nc(-n2cccc2)sc1C(=O)N1Cc2ccccc2C(c2cnn(C)c2)C1 ; Cn1cc(C2CN(C(=O)c3cc(-c4ccco4)[nH]n3)Cc3ccccc32)cn1 ; COc1cccc(-c2cc(C(=O)N3Cc4ccccc4C(c4cnn(C)c4)C3)n(C)n2)c1 ; Cn1cc(C2CN(C(=O)c3csc(-c4ccccc4)n3)Cc3ccccc32)cn1]

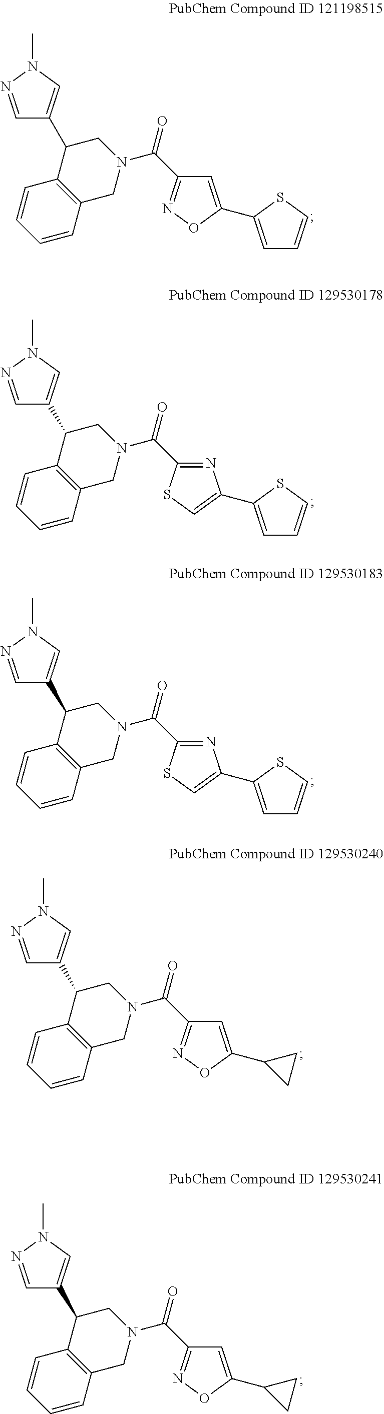 [SMILES: Cn1cc(C2CN(C(=O)c3cc(-c4cccs4)on3)Cc3ccccc32)cn1 ; Cn1cc([C@@H]2CN(C(=O)c3nc(-c4cccs4)cs3)Cc3ccccc32)cn1 ; Cn1cc([C@H]2CN(C(=O)c3nc(-c4cccs4)cs3)Cc3ccccc32)cn1 ; Cn1cc([C@@H]2CN(C(=O)c3cc(C4CC4)on3)Cc3ccccc32)cn1 ; Cn1cc([C@H]2CN(C(=O)c3cc(C4CC4)on3)Cc3ccccc32)cn1]

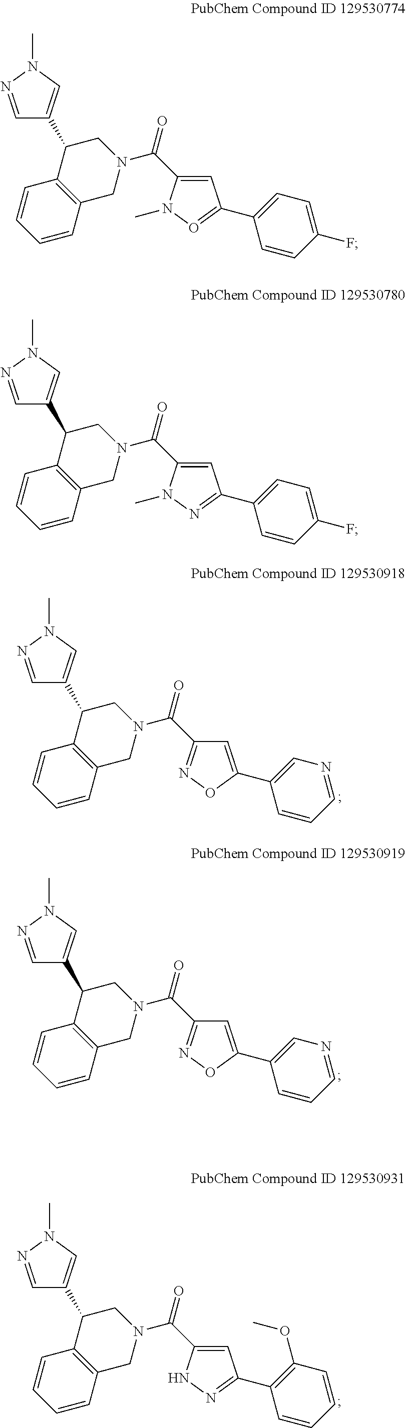

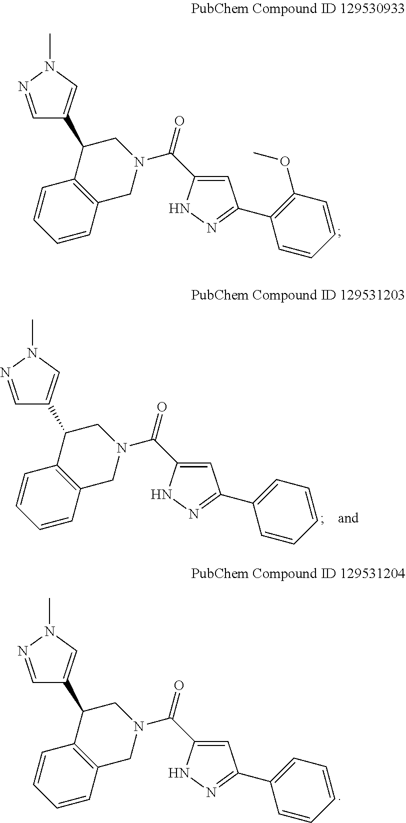 [SMILES: COc1ccccc1-c1cc(C(=O)N2Cc3ccccc3[C@@H](c3cnn(C)c3)C2)[nH]n1 ; Cn1cc([C@@H]2CN(C(=O)c3cc(-c4ccccc4)n[nH]3)Cc3ccccc32)cn1 ; Cn1cc([C@H]2CN(C(=O)c3cc(-c4ccccc4)n[nH]3)Cc3ccccc32)cn1]

14. An intermediate compound of formula (III) or a salt thereof 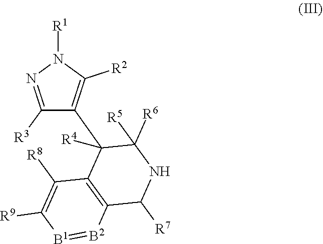
wherein R¹, R², R³, R⁴, R⁵, R⁶, R⁷, R¹, R⁹, B¹ and B² correspond to the same definitions as for the compounds of formula (I) as defined in claim 1.

15. An intermediate compound of formula (VXIII) 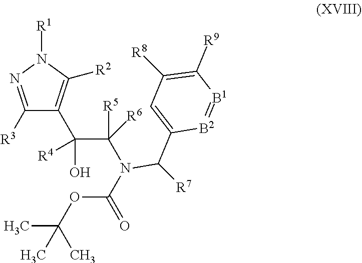
wherein R¹, R², R³, R⁴, R⁵, R⁶, R⁷, R¹, R⁹, B^{r} and B² correspond to the same definitions as for the compounds of formula (I) as defined in claim 1.

16. A method of controlling or preventing infestation of useful plants by phytopathogenic microorganisms, wherein a fungicidally effective amount of a compound of formula (I) as defined in claim 1, or a composition comprising the compound of formula (I), is applied to the plants, to parts thereof or the locus thereof.

17. An agrochemical composition comprising a fungicidally effective amount of a compound of formula (I) as defined in claim 1.
